(19) United States
(12) Patent Application Publication
(10) Pub. No.: US 2007/0112015 A1
(43) Pub. Date: May 17, 2007

(54) Substituted dihydropyridines and methods of use

(75) Inventors: Clarence Ray Hurt, San Ramon, CA (US); Andrew M.K. Pennell, San Francisco, CA (US); John Jessen Wright, Redwood City, CA (US); Manmohan Reddy Leleti, Sunnyvale, CA (US); Qiang Wang, Sunnyvale, CA (US); William D. Thomas, San Jose, CA (US); Yandong Li, San Jose, CA (US); Dean R. Dragoli, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, Inc., Mountain View, CA (US)

(21) Appl. No.: 11/590,658

(22) Filed: Oct. 30, 2006

Related U.S. Application Data
(60) Provisional application No. 60/731,298, filed on Oct. 28, 2005.

(51) Int. Cl. A61K 31/519; A61K 31/4745; A61K 31/4741; C07D 491/02; C07D 487/02; C07D 471/02

(52) U.S. Cl. 514/264.1; 514/300; 514/302; 544/279; 546/115; 546/122; 546/123

(57) ABSTRACT

Compounds are provided that are modulators of the C5a receptor. The compounds are substituted dihydropyridines and are useful in pharmaceutical compositions, methods for the treatment of diseases and disorders involving the pathologic activtation of C5a receptors.

### CROSS-REFERENCES TO RELATED APPLICATIONS

This application claims benefit of Provisional Patent Application Ser. No. 60/731,298, filed Oct. 28, 2005, the content of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The complement system plays a central role in the clearance of immune complexes and in immune responses to infectious agents, foreign antigens, virus infected cells and tumor cells. Inappropriate or excessive activation of the complement system can lead to harmful, and even potentially life-threatening consequences due to severe inflammation and resulting tissue destruction. These consequences are clinically manifested in various disorders including septic shock; myocardial, as well as, intestinal ischemia/reperfusion injury; graft rejection; organ failure; nephritis; pathological inflammation; and autoimmune diseases.

The complement system is composed of a group of proteins that are normally present in the serum in an inactive state. Activation of the complement system encompasses mainly three distinct pathways, i.e., the classical, the alternative, and the lectin pathway (V. M. Holers, In Clinical Immunology: Principles and Practice, ed. R. R. Rich, Mosby Press; 1996, 363-391): 1) The classical pathway is a calcium/magnesium-dependent cascade, which is normally activated by the formation of antigen-antibody complexes. It can also be activated in an antibody-independent manner by the binding of C-reactive protein, complexed with ligand, and by many pathogens including gram-negative bacteria. 2) The alternative pathway is a magnesium-dependent cascade which is activated by deposition and activation of C3 on certain susceptible surfaces (e.g. cell wall polysaccharides of yeast and bacteria, and certain biopolymer materials). 3) The lectin pathway involves the initial binding of mannose-binding lectin and the subsequent activation of C2 and C4, which are common to the classical pathway (Matsushita, M. et al., J. Exp. Med. 176: 1497-1502 (1992); Suankratay, C. et al., J. Immunol. 160: 3006-3013 (1998)).

The activation of the complement pathway generates biologically active fragments of complement proteins, e.g. C3a, C4a and C5a anaphylatoxins and C5b-9 membrane attack complexes (MAC), all which mediate inflammatory responses by affecting leukocyte chemotaxis; activating macrophages, neutrophils, platelets, mast cells and endothelial cells; and increasing vascular permeability, cytolysis and tissue injury.

Complement C5a is one of the most potent proinflammatory mediators of the complement system. (The anaphylactic C5a peptide is 100 times more potent, on a molar basis, in eliciting inflammatory responses than C3a.) C5a is the activated form of C5 (190 kD, molecular weight). C5a is present in human serum at approximately 80 μg/ml (Kohler, P. F. et al., J. Immunol. 99: 1211-1216 (1967)). It is composed of two polypeptide chains, α and β, with approximate molecular weights of 115 kD and 75 kD, respectively (Tack, B. F. et al., Biochemistry 18: 1490-1497 (1979)). Biosynthesized as a single-chain promolecule, C5 is enzymatically cleaved into a two-chain structure during processing and secretion. After cleavage, the two chains are held together by at least one disulphide bond as well as noncovalent interactions (Ooi, Y. M. et al., J. Immunol. 124: 2494-2498(1980)).

C5 is cleaved into the C5a and C5b fragments during activation of the complement pathways. The convertase enzymes responsible for C5 activation are multi-subunit complexes of C4b, C2a, and C3b for the classical pathway and of (C3b)₂, Bb, and P for the alternative pathway (Goldlust, M. B. et al., J. Immunol. 113: 998-1007 (1974); Schreiber, R. D. et al, Proc. Natl. Acad. Sci. 75: 3948-3952 (1978)). C5 is activated by cleavage at position 74-75 (Arg-Leu) in the (α-chain. After activation, the 11.2 kD, 74 amino acid peptide C5a from the amino-terminus portion of the α-chain is released. Both C5a and C3a are potent stimulators of neutrophils and monocytes (Schindler, R. et al., Blood 76: 1631-1638 (1990); Haeffner-Cavaillon, N. et al., J. Immunol. 138: 794-700 (1987); Cavaillon, J. M. et al., Eur. J. Immunol. 20: 253-257 (1990)).

In addition to its anaphylatoxic properties, C5a induces chemotactic migration of neutrophils (Ward, P. A. et al., J. Immunol. 102: 93-99 (1969)), eosinophils (Kay, A. B. et al., Immunol. 24: 969-976 (1973)), basophils (Lett-Brown, M. A. et al., J. Immunol. 117: 246-252 1976)), and monocytes (Snyderman, R. et al., Proc. Soc. Exp. Biol. Med. 138: 387-390 1971)). Both C5a and C5b-9 activate endothelial cells to express adhesion molecules essential for sequestration of activated leukocytes, which mediate tissue inflammation and injury (Foreman, K. E. et al., J. Clin. Invest. 94: 1147-1155 (1994); Foreman, K. E. et al., Inflammation 20: 1-9 (1996); Rollins, S. A. et al., Transplantation 69: 1959-1967 (2000)). C5a also mediates inflammatory reactions by causing smooth muscle contraction, increasing vascular permeability, inducing basophil and mast cell degranulation and inducing release of lysosomal proteases and oxidative free radicals (Gerard, C. et al., Ann. Rev. Immunol. 12: 775-808 (1994)). Furthermore, C5a modulates the hepatic acute-phase gene expression and augments the overall immune response by increasing the production of TNF-α, IL-1-β, IL-6, IL-8, prostaglandins and leukotrienes (Lambris, J. D. et al., In: The Human Complement System in Health and Disease, Volanakis, J. E. ed., Marcel Dekker, New York, pp. 83-118).

The anaphylactic and chemotactic effects of C5a are believed to be mediated through its interaction with the C5a receptor. The human C5a receptor (C5aR) is a 52 kD membrane bound G protein-coupled receptor, and is expressed on neutrophils, monocytes, basophils, eosinophils, hepatocytes, lung smooth muscle and endothelial cells, and renal glomerular tissues (Van-Epps, D. E. et al., J. Immunol. 132: 2862-2867 (1984); Haviland, D. L. et al., J. Immunol. 154:1861-1869 (1995); Wetsel, R. A., Immunol. Leff. 44: 183-187 (1995); Buchner, R. R. et al., J. Immunol. 155: 308-315 (1995); Chenoweth, D. E. et al., Proc. Natl. Acad. Sci. 75: 3943-3947 (1978); Zwirner, J. et al., Mol. Immunol. 36:877-884 (1999)). The ligand-binding site of C5aR is complex and consists of at least two physically separable binding domains. One binds the C5a amino terminus (amino acids 1-20) and disulfide-linked core (amino acids 21-61), while the second binds the C5a carboxy-terminal end (amino acids 62-74) (Wetsel, R. A., Curr. Opin. Immunol. 7: 48-53 (1995)).

C5a plays important roles in inflammation and tissue injury. In cardiopulmonary bypass and hemodialysis, C5a is formed as a result of activation of the alternative complement pathway when human blood makes contact with the artificial surface of the heart-lung machine or kidney dialysis machine (Howard, R. J. et al., Arch. Surg. 123: 1496-1501 (1988); Kirklin, J. K. et al., J. Cardiovasc. Surg. 86: 845-857 (1983); Craddock, P. R. et al., N. Engl. J. Med. 296: 769-774 (1977)). C5a causes increased capillary permeability and edema, bronchoconstriction, pulmonary vasoconstriction, leukocyte and platelet activation and infiltration to tissues, in particular the lung (Czermak, B. J. et al., J. Leukoc. Biol. 64: 40-48 (1998)). Administration of an anti-C5a monoclonal antibody was shown to reduce cardiopulmonary bypass and cardioplegia-induced coronary endothelial dysfunction (Tofukuji, M. et al., J. Thorac. Cardiovasc. Surg. 116: 1060-1068 (1998)).

C5a is also involved in acute respiratory distress syndrome (ARDS), Chronic Obstructive Pulmonary Disorder (COPD) and multiple organ failure (MOF) (Hack, C. E. et al., Am. J. Med. 1989: 86: 20-26; Hammerschmidt DE et al. Lancet 1980; 1: 947-949; Heideman M. et al. J. Trauma 1984; 4: 1038-1043; Marc, M M, et al., Am. J. Respir. Cell and Mol. Biol., 2004: 31: 216-219). C5a augments monocyte production of two important pro-inflammatory cytokines, TNF-α and IL-1. C5a has also been shown to play an important role in the development of tissue injury, and particularly pulmonary injury, in animal models of septic shock (Smedegard G et al. Am. J. Pathol. 1989; 135: 489-497; Markus, S., et al., FASEB Journal (2001), 15: 568-570). In sepsis models using rats, pigs and non-human primates, anti-C5a antibodies administered to the animals before treatment with endotoxin or E. coli resulted in decreased tissue injury, as well as decreased production of IL-6 (Smedegard, G. et al., Am. J. Pathol. 135: 489-497 (1989); Hopken, U. et al., Eur. J. Immunol. 26: 1103-1109 (1996); Stevens, J. H. et al., J. Clin. Invest. 77: 1812-1816 (1986)). More importantly, blockade or C5a with anti-C5a polyclonal antibodies has been shown to significantly imprive survival rates in a caecal ligation/puncture model of sepsis in rats (Czermak, B. J. et al., Nat. Med. 5: 788-792 (1999)). This model share many aspects of the clinical manifestation of sepsis in humans. (Parker, S. J. et al., Br. J. Surg. 88: 22-30 (2001)). In the same sepsis model, anti-C5a antibodies were shown to inhibit apoptosis of thymocytes (Guo, R. F. et al., J. Clin. Invest. 106: 1271-1280 (2000)) and prevent MOF (Huber-Lang, M. et al., J. Immunol. 166: 1193-1199 (2001)). Anti-C5a antibodies were also protective in a cobra venom factor model of lung injury in rats, and in immune complex-induced lung injury (Mulligan, M. S. et al. J. Clin. Invest. 98: 503-512 (1996)). The importance of C5a in immune complex-mediated lung injury was later confirmed in mice (Bozic, C. R. et al., Science 26: 1103-1109 (1996)).

C5a is found to be a major mediator in myocardial ischemia-reperfusion injury. Complement depletion reduced myocardial infarct size in mice (Weisman, H. F. et al., Science 249: 146-151 (1990)), and treatment with anti-C5a antibodies reduced injury in a rat model of hindlimb ischemia-reperfusion (Bless, N. M. et al., Am. J. Physiol. 276: L57-L63 (1999)). Reperfusion injury during myocardial infarction was also markedly reduced in pigs that were retreated with a monoclonal anti-C5a IgG (Amsterdam, E. A. et al., Am. J. Physiol. 268:H448-H457 (1995)). A recombinant human C5aR antagonist reduces infarct size in a porcine model of surgical revascularization (Riley, R. D. et al., J. Thorac. Cardiovasc. Surg. 120: 350-358 (2000)).

Complement levels are elevated in patients with rheumatoid arthritis (Jose, P. J. et al., Ann. Rheum. Dis. 49: 747-752 (1990); Grant, E. P., et al., J. of Exp. Med., 196(11): 1461-1471, (2002)), lupus nephritis (Bao, L., et al., Eur. J. of Immunol., 35(8), 2496-2506, (2005)) and systemic lupus erythematosus (SLE) (Porcel, J. M. et al., Clin. Immunol. Immunopathol. 74: 283-288 (1995)). C5a levels correlate with the severity of the disease state. Collagen-induced arthritis in mice and rats resembles the rheumatoid arthritic disease in human. Mice deficient in the C5a receptor demonstrated a complete protection from arthritis induced by injection of monoclonal anti-collagen Abs (Banda, N. K., et al., J. of Immunol., 2003, 171: 2109-2115). Therefore, inhibition of C5a and/or C5a receptor (C5aR) could be useful in treating these chronic diseases.

The complement system is believed to be activated in patients with inflammatory bowel disease (IBD) and is thought to play a role in the disease pathogenesis. Activated complement products were found at the luminal face of surface epithelial cells, as well as in the muscularis mucosa and submucosal blood vessels in IBD patients (Woodruff, T. M., et al., J of lmmunol., 2003, 171: 5514-5520).

C5aR expression is upregulated on reactive astrocytes, microglia, and endothelial cells in an inflamed human central nervous system (Gasque, P. et al., Am. J. Pathol. 150: 31-41 (1997)). C5a might be involved in neurodegenerative diseases, such as Alzheimer disease (Mukherjee, P. et al., J. Neuroimmunol. 105: 124-130 (2000); O'Barr, S. et al., J. Neuroimmunol. (2000) 105: 87-94; Farkas, I., et al. J. Immunol. (2003) 170:5764-5771). Activation of neuronal C5aR may induce apoptosis (Farkas I et al. J. Physiol. 1998; 507: 679-687). Therefore, inhibition of C5a and/or C5aR could also be useful in treating neurodegenerative diseases.

There is some evidence that C5a production worsens inflammation associated with atopic dermatitis (Neuber, K., et al., Immunology 73:83-87, (1991)), and chronic urticaria (Kaplan, A. P., J. Allergy Clin. Immunol. 114; 465-474, (2004).

Psoriasis is now known to be a T cell-mediated disease (Gottlieb, E. L. et al., Nat. Med. 1: 442-447 (1995)). However, neutrophils and mast cells may also be involved in the pathogenesis of the disease (Terui, T. et al., Exp. Dermatol. 9: 1-10; 2000); Werfel, T. et al., Arch. Dermatol. Res. 289: 83-86 (1997)). High levels of C5a des Arg are found in psoriatic cales, indicating that complement activation is involved. T cells and neutrophils are chemo-attracted by C5a (Nataf, S. et al., J. Immunol. 162: 4018-4023 (1999); Tsuji, R. F. et al., J. Immunol. 165: 1588-1598 (2000); Cavaillon, J. M. et al., Eur. J. Immunol. 20: 253-257 (1990)). Therefore C5a could be an important therapeutic target for treatment of psoriasis.

Immunoglobulin G-containing immune complexes (IC) contribute to the pathophysiology in a number of autoimmune diseases, such as systemic lupus erthyematosus, rheumatoid arthritis, Goodpasture's syndrome, and hypersensitivity pneumonitis (Madaio, M. P., Semin. Nephrol. 19: 48-56 (1999); Korganow, A. S. et al., Immunity 10: 451-459 (1999); Bolten, W. K., Kidney Int. 50: 1754-1760 (1996); Ando, M. et al., Curr. Opin. Pulm. Med. 3: 391-399 (1997)). The classical animal model for the inflammatory response in these IC diseases is the Arthus reaction, which features the infiltration of polymorphonuclear cells, hemorrhage, and plasma exudation (Arthus, M., C.R. Soc. Biol. 55: 817-824 (1903)). Recent studies show that C5aR deficient mice are protected from tissue injury induced by IC (Kohl, J. et al., Mol. Immunol. 36: 893-903 (1999); Baumann, U. et al., J. Immunol. 164: 1065-1070 (2000)). The results are consistent with the observation that a small peptidic anti-C5aR antagonist inhibits the inflammatory response caused by IC deposition (Strachan, A. J. et al., J. Immunol. 164: 6560-6565 (2000)). Together with its receptor, C5a plays an important role in the pathogenesis of IC diseases. Inhibitors of C5a and C5aR could be useful to treat these diseases.

Only recently have non-peptide based C5a receptor antagonists been described in the literature (e.g., Sumichika, H., et al., J. Biol. Chem. (2002), 277, 49403-49407). Non-peptide based C5a receptor antagonist have been reported as being effective for treating endotoxic shock in rats (Stracham, A. J., et al., J. of Immunol. (2000), 164(12): 6560-6565); and for treating IBD in a rat model (Woodruff, T. M., et al., J of Immunol., 2003, 171: 5514-5520). Non-peptide based C5a receptor modulators also have been described in the patent literature by Neurogen Corporation, (e.g., WO2004/043925, WO2004/018460, WO2005/007087, WO03/082826, WO03/08828, WO02/49993, WO03/084524); Dompe S. P. A. (WO02/029187); and The University of Queenland (WO2004/100975).

Age-related macular degeneration (AMD) is the leading cause of blindness in the industrialized world, affecting up to 50 million people worldwide. Drusen, extracellular deposits that accumulate beneath the retinal pigmented epithelium, are an early hallmark of the disease. Drusen usually precedes and increases the risk of choroidal neovascularization (CNV), the later stage of AMD. In both patients with AMD and the mouse animal model expressing the disease, the presence of drusen was found in early subretinal pigmented epithelium deposition of complement components C3 and C5. Evidence points to the bioactive complement components (C3a and C5a) present in drusen, as both C3a and C5a induce VEGF expression in vivo and in vitro. It has also been demonstrated that disruption of C3a and C5a interaction with their receptors (C3aR and C5aR) can reduce CNV and can alleviate the symptoms associated with AMD. (Ambati, J. et. al., PNAS, 2005, 103, 7, 2328-2333).

Targeting of C5a Receptor prior to initial allergen sensitization in murine models of inhalation tolerance or allergic asthma resulted in reduction or marked enhancement of Th2-polarized immune response, airway inhalation and AHR. The C5aR knock-out mice express a similar phenotype. C5aR targeting in sensitized mice led to suppressed airway inflammation and AHR but was still associated with enhanced production of Th2 effectors cytokines. It has been shown that C5a possesses a dual role in allergic asthma, i.e., protection from the development of maladaptive type2 immune response during allergen sensitization but also enhancement of airway inflammation and AHR in an established inflammatory environment. (Kohl, J. et. al., J. Clinical Investigation, 2006, 116, 3, 783-796).

There is considerable experimental evidence in the literature that implicates increased levels of C5a with a number of diseases and disorders, in particular in autoimmune and inflammatory diseases and disorders. Thus, there remains a need in the art for new small organic molecule modulators, e.g., agonists, preferably antagonists, partial agonists, of the C5a receptor (C5aR) that are useful for inhibiting pathogenic events, e.g., chemotaxis, associated with increased levels anaphylatoxin activity. The present invention fulfills this and other needs.

### BRIEF SUMMARY OF THE INVENTION

In one aspect, the present invention provides for compounds having formula: 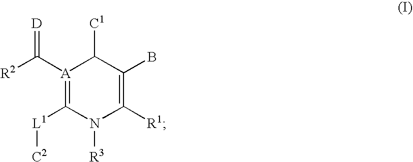
wherein A is nitrogen or carbon. In formula I, B is selected from the group consisting of halogen, —CN, —NO₂, —C(═NOR^{a})R^{c}, —C(═NR^{c})R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}—C(O)NR^{a}R^{b}, —NH—C(NH₂)═NH, —NR^{c}C(NH₂)═NH, —NH—C(NH₂)═NR^{c}, —NHC(NHR^{c})═NH, —NR^{a}C(O)NR^{a}R^{b}, —C(NR^{a}W)═NW, —N(W)C(R^{a})═NW, —X¹C(NR^{a}W)═NW, —X¹N(W)C(R^{a})═NW, —X¹—(NR^{a}R^{b}), —X¹—(OR^{a}), —X¹—(SR^{a}), —S(O)R^{c}, —S(O)₂R^{c}, —NR^{a}S(O)₂R^{c}, —S(O)₂NR^{a}R^{b}, —NR^{a}S(O)₂R^{c} and —NR^{a}S(O)₂NR^{a}R^{b}; wherein X¹ is a C₁₋₄ alkylene, C₁₋₄ heteroalkylene, C₂₋₄ alkenylene or C₂₋₄ alkynylene. Each R^{a} and R^{b} substituent is independently selected from hydrogen, C₁₋₈ alkyl, C₁₋₈ haloalkyl, C₂₋₈ heteroalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl, or when attached to the same nitrogen atom can be combined with the nitrogen atom to form a five or six-membered ring having from 0 to 2 additional heteroatoms as ring members selected from N, O or S. The R^{c} group is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl and aryloxy-C₁₋₄ alkyl. The substituent W is independently selected from the group consisting of —R^{c}, —CN, —CO₂R^{e} and —NO₂, and wherein the aliphatic portions of X¹, R^{a}, R^{b} and R^{c} are optionally further substituted with from one to three members selected from the group consisting of halogen, —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{o}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —CN, —NO₂, —NH₂, —NHR^{m}, —N(R^{m})₂, —NR^{m}S(O)NH₂ and —NR^{m}S(O)₂NHR^{m}, wherein R^{m} is unsubstituted C₁₋₆ alkyl. The substituent R¹ is selected from the group consisting of hydrogen, amino, C₁₋₈ alkylamino, C₁₋₈ dialkylamino, C₁₋₈ alkoxy, C₁₋₈ alkyl, C₁₋₈ haloalkyl, C₃₋₆ cycloalkyl, C₃₋₆ heterocycloalkyl, C₃₋₆ cycloalkyl-C₁₋₆ alkyl, C₃₋₆ heterocycloalkyl-C₁₋₆ alkyl. The aliphatic portions of each R¹ substituent is optionally substituted with from one to three members selected from the group consisting of —OH, —ORⁿ, —OC(O)NHRⁿ, —OC(O)N(Rⁿ)₂, —SH, —SRⁿ, —S(O)Rⁿ, —S(O)₂Rⁿ, —SO₂NH₂, —S(O)₂NHRⁿ, —S(O)₂N(Rⁿ)₂, —NHS(O)₂Rⁿ, —NRⁿS(O)₂Rⁿ, —C(O)NH₂, —C(O)NHRⁿ, —C(O)N(Rⁿ)₂, —C(O)Rⁿ, —NHC(O)Rⁿ, —NRⁿC(O)Rⁿ, —NHC(O)NH₂, —NRⁿC(O)NH₂, —NRⁿC(O)NHRⁿ, —NHC(O)NHRⁿ, —NRⁿC(O)N(Rⁿ)₂, —NHC(O)N(Rⁿ)₂, —CO₂H, —CO₂Rⁿ, —NHCO₂Rⁿ, —NRⁿCO₂Rⁿ, —Rⁿ, —CN, —NO₂, —NH₂, —NHRⁿ, —N(Rⁿ)₂, —NRⁿS(O)NH₂ and —NRⁿS(O)₂NHRⁿ, wherein each Rⁿ is independently an unsubstituted C₁₋₆ alkyl. Each of C¹ and C² are independently aryl, aryl-C₁₋₄ alkyl, heteroaryl, heteroaryl-C₁₋₄ alkyl, cycloalkyl, cycloalkyl-C₁₋₄ alkyl, heterocycloalkyl or heterocycloalkyl-C₁₋₄ alkyl, wherein the heterocycloalkyl group has from 1-3 heteroatoms selected from N, O and S. C¹ and C² has from 0 to 7 R⁴ substituents selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}dV)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{d}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; and optionally any two substituents located on adjacent atoms of C¹ or C² are combined to form a 5- to 7-membered ring. For C¹ and C², X² is C₁₋₄ alkylene, C₁₋₄ heteroalkylene, C₂₋₄ alkenylene or C₂₋₄ alkynylene and each R^{d} and R^{e} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl, and optionally, optionally R^{d} and R^{e} when attached to the same nitrogen atom are combined to form a five or six-membered ring having from 0 to 2 additional heteroatoms as ring members. R^{f} at each occurrence is independently selected from the group consisting of C₁₋₈ alkyl, C₁₋₈ haloalkyl, C₃₋₆ cycloalkyl, C₂₋₈ alkenyl, C₂₋₈ alkynyl, aryl and heteroaryl. Each of X², R^{d}, R^{e} and R^{f} at each occurrence is optionally further substituted with from one to three members selected from the group consisting of halogen, —OH, —OR^{o}, —OC(O)NHR^{o}, —OC(O)N(R^{o})₂, —SH, —SR^{o}, —S(O)R^{o}, —S(O)₂R^{o}, —SO₂NH₂, —S(O)₂NHR^{o}, —S(O)₂N(R^{o})₂, —NHS(O)₂R^{o}, —NR^{o}S(O)₂R^{o}, —C(O)NH₂, —C(O)NHR^{o}, —C(O)N(R^{o})₂, —C(O)R^{o}, —NHC(O)R^{o}, —NR^{o}C(O)R^{o}, —NHC(O)NH₂, —NR^{o}C(O)NH₂, —NR^{o}C(O)NHR^{o}, —NHC(O)NHR^{o}, —NR^{o}C(O)N(R^{o})₂, —NHC(O)N(R^{o})₂, —CO₂H, —CO₂R^{o}, —NHCO₂R^{o}, —NR^{o}CO₂R^{o}, —CN, —NO₂, —NH₂, —NHR^{o}, —N(R^{o})₂, —NR^{o}S(O)NH₂ and —NR^{o}S(O)₂NHR^{o}, wherein each R^{o} is independently an unsubstituted C₁₋₆ alkyl. The symbol V is independently selected from the group consisting of —R^{f}, —CN, —CO₂R^{e} and —NO₂. In formula I, the L¹ substituent is a direct bond, C₁₋₈ alkylene, C₂₋₈ alkenylene, C₂₋₈ alkynylene, C₁₋₈ heteroalkylene, —S—, —S(O)—, —S(O)₂—, —O—, —NH—, or —NR^{j}—; wherein R^{j} is C₁₋₆ alkyl, C₁₋₆ acyl or C₁₋₆ haloalkyl. The the aliphatic portions of L¹ is optionally further substituted with from one to three members selected from the group consisting of halogen —OH, —OR^{p}, —OC(O)NHR^{p}, —OC(O)N(R^{p})₂, —SH, —SR^{p}, —S(O)R^{p}, —S(O)₂R^{p}, —SO₂NH₂, —S(O)₂NHR^{p}, —S(O)₂N(R^{p})₂, —NHS(O)₂R^{p}, —NR^{t}S(O)₂R^{p}, —C(O)NH₂, —C(O)NHR^{p}, —C(O)N(R^{p})₂, —C(O)R^{p}, —NHC(O)R^{p}, —NR^{p}C(O)R^{p}, —NHC(O)NH₂, —NR^{p}C(O)NH₂, —NR^{p}C(O)NHR^{p}, —NHC(O)NHR^{p}, —NR^{p}C(O)N(R^{p})₂, —NHC(O)N(R^{p})₂, —CO₂H, —CO₂R^{p}, —NHCO₂R^{p}, —NR^{p}CO₂R^{p}, —CN, —NO₂, —NH₂, —NHR^{p}, —N(R^{p})₂, —NR^{p}S(O)NH₂ and —NR^{p}S(O)₂NHR^{p}, wherein each R^{p} is independently an unsubstituted C₁₋₆ galkyl or C₁₋₆ haloalkyl. The symbol R² is selected from the group consisting of —OR^{g}, —NR^{g}R^{h} and —Rⁱ, wherein each R^{g} and R^{h} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl and aryloxy-C₁₋₄ alkyl, or when attached to the same nitrogen atom can be combined with the nitrogen atom to form a five or six-membered ring having from 0 to 2 additional heteroatoms as ring members. The substituent Rⁱ is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl. The aliphatic portions of R^{g}, R^{h} and Rⁱ are optionally further substituted with from one to three members selected from the group consisting of halogen, —OH, —OR^{q}, —OC(O)NHR^{q}, —OC(O)N(R^{q})₂, —SH, —SR^{q}, —S(O)R^{q}, —S(O)₂R^{q}, —SO₂NH₂, —S(O)₂NHR^{q}, —S(O)₂N(R^{q})₂, —NHS(O)₂R^{q}, —NR^{q}(O)₂R^{q}, —C(O)NH₂, —C(O)NHR^{q}, —C(O)N(R^{q})₂, —C(O)R^{q}, —NHC(O)R^{q}, —NR^{q}C(O)R^{q}, —NHC(O)NH₂, —NR^{q}C(O)NH₂, —NR^{q}C(O)NHR^{q}, —NHC(O)NHR^{q}, —NR^{q}C(O)N(R^{q})₂, —NHC(O)N(R^{q})₂, —CO₂H, —CO₂R^{q}, —NHCO₂R^{q}, —NR^{q}CO₂R^{q}, —CN, —NO₂, —NH₂, —NHR^{q}, —N(R^{q})₂, —NR^{q}S(O)NH₂ and —NR^{q}S(O)₂NHR^{q}, wherein R^{q} is unsubstituted C₁₋₆ alkyl. Optionally, the substituents R² and L¹, together with the atoms to which they are attached, are combined to form a 5- to 7-membered carbocyclic or heterocyclic ring. The R³ substituent in formula I is hydrogen, C₁₋₆ alkyl, C₁₋₆ haloalkyl, C₁₋₆ acyl, C₃₋₈ cycloalkyl, aryl, heteroaryl, aryl-C₁₋₄ alkyl, heteroaryl-C₁₋₄ alkyl or C₂₋₆ alkenyl, wherein the aliphatic portions of R³ are optionally substituted with 1 to 3 substituents selected from the group consisting of —OH, —OR^{s}, —OC(O)NHR^{s}, —OC(O)N(R^{s})₂, —SH, —SR^{s}, —S(O)R^{s}, —S(O)₂R^{s}, —SO₂NH₂, —S(O)₂NHR^{s}, —S(O)₂N(R^{s})₂, —NHS(O)₂R^{s}, —NR^{s}S(O)₂R^{s}, —C(O)NH₂, —C(O)NHR^{s}, —C(O)N(R^{s})₂, —C(O)R^{s}, —NHC(O)R^{s}, —NR^{s}C(O)R^{s}, —NHC(O)NH₂, —NR^{s}C(O)NH₂, —NR^{s}C(O)NHR^{s}, —NHC(O)NHR^{s}, —NR^{s}C(O)N(R^{s})₂, —NHC(O)N(R^{s})₂, —CO₂H, —CO₂R^{s}, —NHCO₂R^{s}, —NR^{s}CO₂R^{s}, —CN, —NO₂, —NH₂, —NHR^{s}, —N(R^{s})₂, —NR^{s}S(O)NH₂ and —NR^{s}S(O)₂NHR^{s}, wherein each R^{s} is independently an unsubstituted C₁₋₆ alkyl. The symbol D is O, S or N—OR^{t}, wherein R^{t} is selected from the group consisting of hydrogen, C₁₋₈ alkyl, C₁₋₈ haloalkyl and C₁₋₈ heteroalkyl.

In addition to the compounds provided herein, the present invention further provides pharmaceutical compositions containing one or more of these compounds, as well as methods for the use of these compounds in therapeutic methods, primarily to treat diseases associated C5a signalling activity.

### BRIEF DESCRIPTION OF THE DRAWINGS

FIG. 1. C5aR antagonists with inhibition concentration less than 1 μM in either the binding or functional assays described below.

### DETAILED DESCRIPTION OF THE INVENTION

I. Abbreviation and Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The term “heterocycloalkyl” refers to a cycloalkyl group that contain from one to five heteroatoms selected from N, O, and S, wherein the nitrogen and sulfur atoms are optionally oxidized, and the nitrogen atom(s) are optionally quaternized, the remaining ring atoms being C, where one or two C atoms may optionally be replaced by a carbonyl. The heterocycloalkyl may be a monocyclic, a bicyclic or a polycylic ring system. Non limiting examples of heterocycloalkyl groups include pyrrolidine, piperidiny, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms. Similarly, “alkenylene” and “alkynylene” refer to the unsaturated forms of “alkylene” having double or triple bonds, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quatemized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the terms “heteroalkenyl” and “heteroalkynyl” by itself or in combination with another term, means, unless otherwise stated, an alkenyl group or alkynyl group, respectively, that contains the stated number of carbons and having from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group.

The term “heteroalkylene” by itself or as part of another substituent means a divalent radical, saturated or unsaturated or polyunsaturated, derived from heteroalkyl, as exemplified by —CH₂—CH₂—S—CH₂CH₂— and —CH₂—S—CH₂—CH₂—NH—CH₂—, —O—CH₂—CH═CH—, —CH₂—CH═C(H)CH₂—O—CH₂— and —S—CH₂—C≡C—. For heteroalkylene groups, heteroatoms can also occupy either or both of the chain termini (e.g., alkyleneoxy, alkylenedioxy, alkyleneamino, alkylenediamino, and the like).

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalaziniyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like).

The above terms (e.g., “alkyl,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below, while the term “alkyl” and related aliphatic radicals is meant to refer to unsubstituted version, unless indicated to be substituted.

Substituents for the alkyl radicals (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂ in a number ranging from zero to (2 m′+1), where m′ is the total number of carbon atoms in such radical. R′, R″ and R″′ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to an alkyl radical wherein two substitutents on the carbon that is closest to the point of attachment for the radical is replaced with the substitutent ═O (e.g., —C(O)CH₃, —C(O)CH₂CH₂OR′ and the like).

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ-B-, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The term “ionic liquid” refers to any liquid that contains mostly ions. Preferably, in the present invention, “ionic liquid” refers to the salts whose melting point is relatively low (e.g., below 250° C). Examples of ionic liquids include but are not limited to 1-butyl-3-methylimidazolium tetrafluoroborate, 1-hexyl-3-methylimidazolium tetrafluoroborate, 1-octyl-3-methylimidazolium tetrafluoroborate, 1-nonyl-3-methylimidazolium tetrafluoroborate, 1-decyl-3-methylimidazolium tetrafluoroborate, 1-hexyl-3-methylimidazolium hexafluorophosphate and 1-hexyl-3-methylimidazolium bromide, and the like.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

II. Compounds

In one aspect, the present invention provides compounds having the formula I: 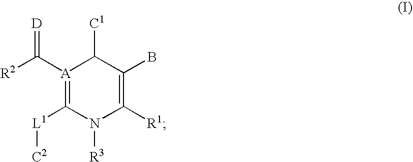
wherein A is nitrogen or carbon. In one embodiment, A is nitrogen. In another embodiment A is carbon. In yet another embodiment, R² and L¹ are combined to form a 6-membered carbocyclic or heterocyclic ring.

In formula I, B is selected from the group consisting of halogen, —CN, —NO₂, —C(═NOR^{a})R^{c}, —C(═NR^{c})R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}—C(O)NR^{a}R^{b}, —NH—C(NH₂)═NH, —NR^{c}C(NH₂)═NH, —NH—C(NH₂)═NR^{c}, —NHC(NHR^{c})═NH, —NR^{a}C(O)NR^{a}R^{b}, —C(NR^{a}W)═NW, —N(W)C(R^{a})═NW, —X¹C(NR^{a}W)═NW, —X¹N(W)C(R^{a})═NW, —X¹—(NR^{a}R^{b}), —X¹—(OR^{a}), —X¹—(SR^{a}), —S(O)R^{c}, —S(O)₂R^{c}, —NR^{a}S(O)₂R^{c}, —S(O)₂NR^{a}R^{b}, —NR^{a}S(O)₂R^{c} and —NR^{a}S(O)₂NR^{a}R^{b}; wherein X¹ is a C₁₋₄ alkylene, C₁₋₄ heteroalkylene, C₂₋₄ alkenylene or C₂₋₄ alkynylene. Each R^{a} and R^{b} substituent is independently selected from hydrogen, C₁₋₈ alkyl, C₁₋₈ haloalkyl, C₂₋₈ heteroalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl, or when attached to the same nitrogen atom can be combined with the nitrogen atom to form a five or six-membered ring having from 0 to 2 additional heteroatoms as ring members selected from N, O or S. The R^{c} group is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl and aryloxy-C₁₋₄ alkyl. The substituent W is independently selected from the group consisting of —R^{c}, —CN, —CO₂R^{e} and —NO₂, and wherein the aliphatic portions of X¹, R^{a}, R^{b} and R^{c} are optionally further substituted with from one to three members selected from the group consisting of halogen, —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{o}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —CN, —NO₂, —NH₂, —NHR^{m}, —N(R^{m})₂, —NR^{m}S(O)NH₂ and —NR^{m}S(O)₂NHR^{m}, wherein R^{m} is unsubstituted C₁₋₆ alkyl. In one embodiment of of formula I, B is selected from the group consisting of —CN, —CO₂R^{a}, —CONR^{a}R^{b} and —C(O)R^{a}. In another embodiment of formula I, B is —CO₂R^{a}, wherein R^{a} is an optionally substituted member selected from the group consisting of C₁₋₈ alkyl, C₃₋₈ cycloalkyl, C₂₋₈ heteroalkyl and aryloxy-C₁₋₄ alkyl. In yet another embodiment, R^{a} and R^{b} are each independently selected from the group consisting of H, Me, Et, i-Pr, t-Bu, CH₂CH₂OCH₃, CH₂CH₂NHCH₃, CH₂CH₂N(CH₃)₂, cycloalkyl, heterocycloalkyl, alkoxyalkoxy alkyl.

In formula I, R¹ is selected from the group consisting of hydrogen, amino, C₁₋₈ alkylamino, C₁₋₈ dialkylamino, C₁₋₈ alkoxy, C₁₋₈ alkyl, C₁₋₈ haloalkyl, C₃₋₆ cycloalkyl, C₃₋₆ heterocycloalkyl, C₃₋₆ cycloalkyl-C₁₋₆ alkyl, C₃₋₆ heterocycloalkyl-C₁₋₆ alkyl. The aliphatic portions of each R¹ substituent is optionally substituted with from one to three members selected from the group consisting of —OH, —ORⁿ, —OC(O)NHRⁿ, —OC(O)N(Rⁿ)₂, —SH, —SRⁿ, —S(O)Rⁿ, —S(O)₂Rⁿ, —SO₂NH₂, —S(O)₂NHRⁿ, —S(O)₂N(Rⁿ)₂, —NHS(O)₂Rⁿ, —NRⁿS(O)₂Rⁿ, —C(O)NH₂, —C(O)NHRⁿ, —C(O)N(Rⁿ)₂, —C(O)Rⁿ, —NHC(O)Rⁿ, —NRⁿC(O)Rⁿ, —NHC(O)NH₂, —NRⁿC(O)NH₂, —NRⁿC(O)NHRⁿ, —NHC(O)NHRⁿ, —NRⁿC(O)N(Rⁿ)₂, —NHC(O)N(Rⁿ)₂, —CO₂H, —CO₂Rⁿ, —NHCO₂Rⁿ, —NRⁿCO₂Rⁿ, —Rⁿ, —CN, —NO₂, —NH₂, —NHRⁿ, —N(Rⁿ)₂, —NRⁿS(O)NH₂ and —NRⁿS(O)₂NHRⁿ, wherein each Rⁿ is independently an unsubstituted C₁₋₆ alkyl. In one embodiment, R¹ is C₁₋₈ alkyl, C₁₋₈ haloalkyl, C₁₋₈ alkoxy, C₃₋₈ cycloalkyl-C₁₋₈ alkyl, C₃₋₆ heterocycloalkyl, C₃₋₆ heterocycloalkyl-C₁₋₆ alkyl or amino; each of which is optionally substituted with from 1-3 members selected from the group consisting of —ORⁿ, —CO₂Rⁿ, —N(Rⁿ)₂, —C(O)N(Rⁿ)₂, and —C(O)NHRR. In another embodiment, R¹ is —CH₃, —CF₃, cyclopentyl, cyclopentylethyl, or —NH₂.

Each of C¹ and C², in formula I, are independently aryl, aryl-C₁₋₄ alkyl, heteroaryl, heteroaryl-C₁₋₄ alkyl, cycloalkyl, cycloalkyl-C₁₋₄ alkyl, heterocycloalkyl or heterocycloalkyl-C₁₋₄ alkyl, wherein the heterocycloalkyl group has from 1-3 heteroatoms selected from N, O and S. C¹ and C² has from 0 to 7 R⁴ substituents selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{d}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; and optionally any two substituents located on adjacent atoms of C¹ or C² are combined to form a 5- to 7-membered ring. For C¹ and C², X² is C₁₋₄ alkylene, C₁₋₄ heteroalkylene, C₂₋₄ alkenylene or C₂₋₄ alkynylene and each R^{d} and R^{e} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl, and optionally, optionally R^{d} and R^{e} when attached to the same nitrogen atom are combined to form a five or six-membered ring having from 0 to 2 additional heteroatoms as ring members. R^{f} at each occurrence is independently selected from the group consisting of C₁₋₈ alkyl, C₁₋₈ haloalkyl, C₃₋₆ cycloalkyl, C₂₋₈ alkenyl, C₂₋₈ alkynyl, aryl and heteroaryl. Each of X², R^{d}, R^{e} and R^{f} at each occurrence is optionally further substituted with from one to three members selected from the group consisting of halogen, —OH, —OR^{o}, —OC(O)NHR^{o}, —OC(O)N(R^{o})₂, —SH, —SR^{o}, —S(O)R^{o}, —S(O)₂R^{o}, —SO₂NH₂, —S(O)₂NHR^{o}, —S(O)₂N(R^{o})₂, —NHS(O)₂R^{o}, —NR^{o}S(O)₂R^{o}, —C(O)NH₂, —C(O)NHR^{o}, —C(O)N(R^{o})₂, —C(O)R^{o}, —NHC(O)R^{o}, —NR^{o}C(O)R^{o}, —NHC(O)NH₂, —NR^{o}C(O)NH₂, —NR^{o}C(O)NHR^{o}, —NHC(O)NHR^{o}, —NR^{o}C(O)N(R^{o})₂, —NHC(O)N(R^{o})₂, —CO₂H, —CO₂R^{o}, —NHCO₂R^{o}, —NR^{o}CO₂R^{o}, —CN, —NO₂, —NH₂, —NHR^{o}, —N(R^{o})₂, —NR^{o}S(O)NH₂ and —NR^{o}S(O)₂NHR^{o}, wherein each R^{o} is independently an unsubstituted C₁₋₆ alkyl. The symbol V is independently selected from the group consisting of —R^{f}, —CN, —CO₂R^{e} and —NO₂.

In one group of embodiments of formula I, C¹ is a member selected from the group consisting of phenyl, 2-pyridyl, 3-pyridyl, 4-pyridyl, 2-thienyl, 3-thienyl, 2-furanyl, 3-furanyl, 3-indolyl, 4-indolyl, 5-indolyl, 6-indolyl, 7-indolyl, 3-dihydro-1H-indolyl, 3-indazolyl, 4-indazolyl, 5-indazolyl, 6-indazolyl, 7-indazolyl, 1H-benzotriazolyl, 2-oxo-2,3-dihydro-benzooxazolyl, 2-oxo-2,3-dihydro-benzothiazolyl, 2-oxo-1H-benzoimidazoly, 3-oxo-3,4-dihydro-2H-benzo[1,4]oxazinyl, 5-azabenzpyrazolyl, benzopyrrolidinyl, 2-pyrrolyl and 3-pyrrolyl; each of which is optionally substituted with from 1-3 R⁴ substituents. In certain instances, C¹ is a phenyl group optionally substituted with 1 to 2 substituents selected from the group consisting of —OH, —NO₂, —NR^{d}R^{e}, halogen, C₁₋₈ alkyl, C₁₋₈ haloalkyl, C₁₋₈ alkoxy, C₂₋₈ alkenyl, X²OR^{d}, —NR^{d}—C(O)NR^{d}R^{e}, —NR^{d}—S(O)₂NR^{d}R^{e}, —NR^{d}S(O)₂R^{f}, X²CO₂R^{d}, X²C(O)NR^{d}R^{e}, —C(O)NR^{d}R^{e}, —NR^{e}C(O)R^{d} and heterocycloalkyl; and optionally any two substituents located on adjacent atoms of C¹ are combined to form a 5- to 6-membered ring. In certain other instances, C¹ is a monocyclic ring selected from the group consisting of phenyl, pyridyl, furanyl, thienyl and pyrrolyl; each of which is optionally substituted with from 1-2 R⁴ substituents. In yet certain other instances, C¹ is a bicyclic ring selected from the group consisting of indolyl, indazolyl, 1H-benzotriazolyl, 2-oxo-2,3-dihydro-benzooxazolyl, 2-oxo-2,3-dihydro-benzothiazolyl, 2-oxo-2,3-dihydro-1H-benzoimidazolyl, 3-oxo-3,4-dihydro-2H-benzo[1,4]oxazinyl and 5-azabenzpyrazolyl, benzopyrrolidinyl; each of which is optionally substituted with from 1-3 R⁴ substituents. In still other instances, C¹ is selected from the group consisting of:

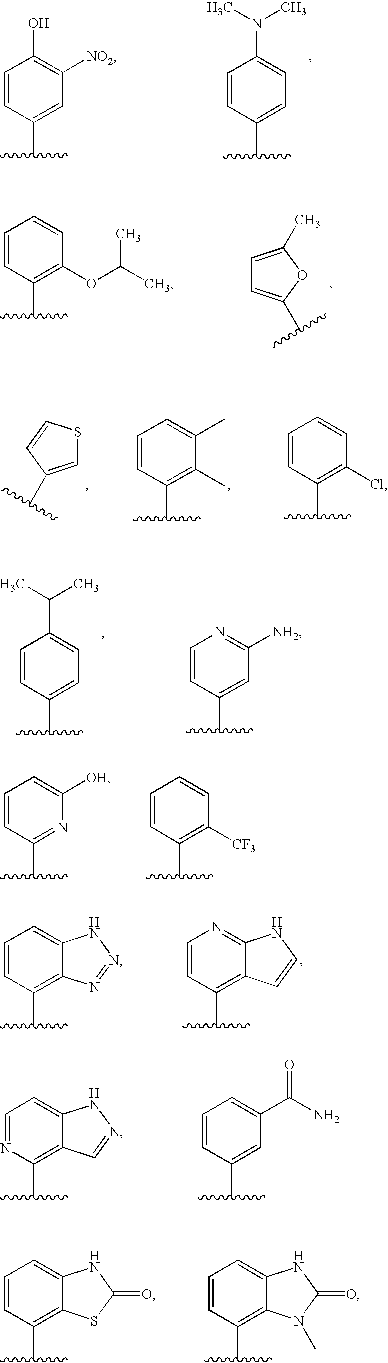

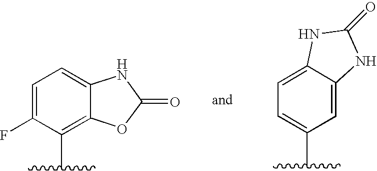

In certain preferred embodiments, C¹ is a member selected from the group consisting of:

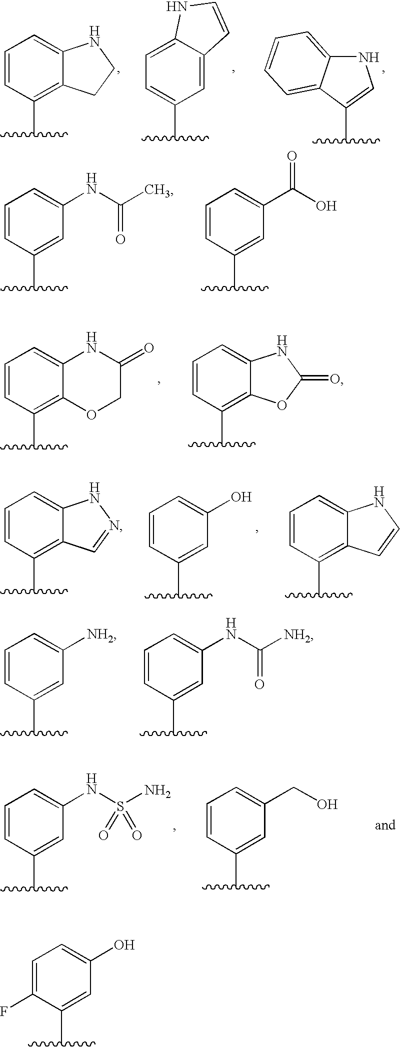

In a second group of embodiments of formula I, C² is a substituted member selected from the group consisting of phenyl, 2-pyridyl, 3-pyridyl, 4-pyridyl, 2-thienyl, 3-thienyl, 2-furanyl, 3-furanyl, 2-pyrrolyl, 3-pyrrolyl, 3-indolyl, 4-indolyl, 5-indolyl, 6-indolyl and 7-indolyl; each of which is optionally substituted with from 1-3 R⁴ substituents. In certain instances, C² is a phenyl group optionally having 1 to 5 substituents selected from the group consisting of halogen, cyano, —NR^{d}R^{e}, C₁₋₈ alkyl, C₁₋₈ alkoxy, C₁₋₈ haloalkyl, C₃₋₆ cycloalkyl, C₂₋₈ alkenyl, C₂₋₈ alkynyl, aryl-C₁₋₄ alkyl, and optionally any two substituents located on adjacent atoms of C² are combined to form a 5- to 6-membered ring. In certain other instances, C² is a compound selected from the group consisting of:

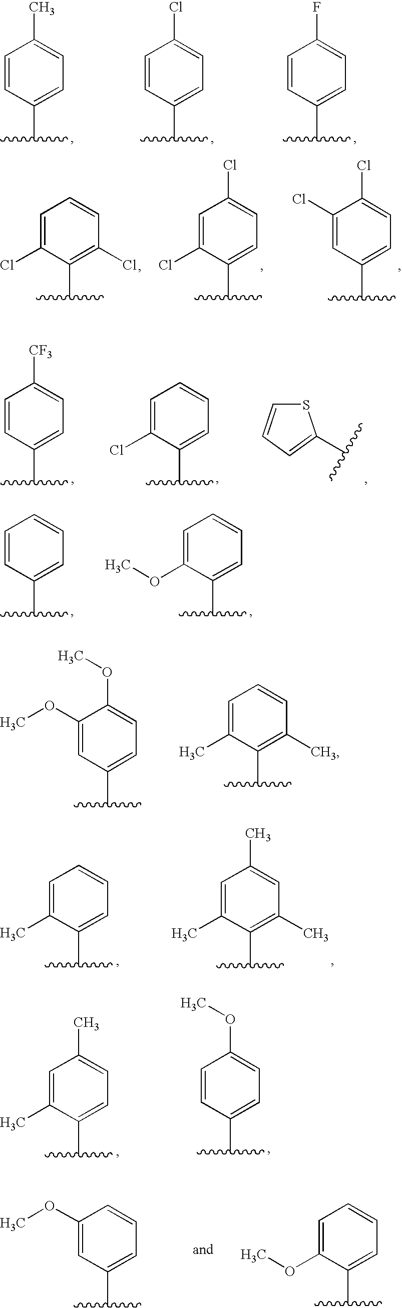

In formula I, the L¹ substituent is a direct bond, C₁₋₈ alkylene, C₂₋₈ alkenylene, C₂₋₈ alkynylene, C₁₋₈ heteroalkylene, —S—, —S(O)—, —S(O)₂—, —O—, —NH—, or —NR^{j}—; wherein R^{j} is C₁₋₆ alkyl, C₁₋₆ acyl or C₁₋₆ haloalkyl. The the aliphatic portions of L¹ is optionally further substituted with from one to three members selected from the group consisting of halogen —OH, —OR^{p}, —OC(O)NHR^{p}, —OC(O)N(R^{p})₂, —SH, —SR^{p}, —S(O)R^{p}, —S(O)₂R^{p}, —SO₂NH₂, —S(O)₂NHR^{p}, —S(O)₂N(R^{p})₂, —NHS(O)₂R^{p}, —NR^{t}S(O)₂R^{p}, —C(O)NH₂, —C(O)NHR^{p}, —C(O)N(R^{p})₂, —C(O)R^{p}, —NHC(O)R^{p}, —NR^{p}C(O)R^{p}, —NHC(O)NH₂, —NR^{p}C(O)NH₂, —NR^{p}C(O)NHR^{p}, —NHC(O)NHR^{p}, —NR^{p}C(O)N(R^{p})₂, —NHC(O)N(R^{p})₂, —CO₂H, —CO₂R^{p}, —NHCO₂R^{p}, —NR^{p}CO₂R^{p}, —CN, —NO₂, —NH₂, —NHR^{p}, —N(R^{p})₂, —NR^{p}S(O)NH₂ and —NR^{p}S(O)₂NHR^{p}, wherein each R^{p} is independently an unsubstituted C₁₋₆ alkyl or C₁₋₆ haloalkyl.

In formula I, the symbol R² is selected from the group consisting of —OR^{g}, —NR^{g}R^{h} and —Rⁱ, wherein each R^{g} and R^{h} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl and aryloxy-C₁₋₄ alkyl, or when attached to the same nitrogen atom can be combined with the nitrogen atom to form a five or six-membered ring having from 0 to 2 additional heteroatoms as ring members. The substituent Rⁱ is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl. The aliphatic portions of R^{g}, R^{h} and Rⁱ are optionally further substituted with from one to three members selected from the group consisting of halogen, —OH, —OR^{q}, —OC(O)NHR^{q}, —OC(O)N(R^{q})₂, —SH, —SR^{q}, —S(O)R^{q}, —S(O)₂R^{q}, —SO₂NH₂, —S(O)₂NHR^{q}, —S(O)₂N(R^{q})₂, —NHS(O)₂R^{q}, —NR^{q}S(O)₂R^{q}, —C(O)NH₂, —C(O)NHR^{q}, —C(O)N(R^{q})₂, —C(O)R^{q}, —NHC(O)R^{q}, —NR^{q}C(O)R^{q}, —NHC(O)NH₂, —NR^{q}C(O)NH₂, —NR^{q}C(O)NHR^{q}, —NHC(O)NHR^{q}, —NR^{q}C(O)N(R^{q})₂, —NHC(O)N(R^{q})₂, —CO₂H, —CO₂R^{q}, —NHC₂R^{q}, —NR^{q}CO₂R^{q},—CN, —NO₂, —NH₂, —NHR^{q}, —N(R^{q})₂, —NR^{q}S(O)NH₂ and —NR^{q}S(O)₂NHR^{q}, wherein R^{q} is unsubstituted C₁₋₆ alkyl. Optionally, the substituents R² and L¹, together with the atoms to which they are attached, are combined to form a 5- to 7-membered carbocyclic or heterocyclic ring.

In one embodiment of formula I, R² is Rⁱ selected from C₁₋₈ alkyl and C₁₋₈ haloalkyl; and R² and L¹ together with the atoms to which they are attached are combined to form a 6-membered carbocyclic ring.

The R³ substituent in formula I is hydrogen, C₁₋₆ alkyl, C₁₋₆ haloalkyl, C₁₋₆ acyl, C₃₋₈ cycloalkyl, aryl, heteroaryl, aryl-C₁₋₄ alkyl, heteroaryl-C₁₋₄ alkyl or C₂₋₆ alkenyl, wherein the aliphatic portions of R³ are optionally substituted with 1 to 3 substituents selected from the group consisting of —OH, —OR^{s}, —OC(O)NHR^{s}, —OC(O)N(R^{s})₂, —SH, —SR^{s}, —S(O)R^{s}, —S(O)₂R^{s}, —SO₂NH₂, —S(O)₂NHR^{s}, —S(O)₂N(R^{s})₂, —NHS(O)₂R^{s}, —NR^{s}S(O)₂R^{s}, —C(O)NH₂, —C(O)NHR^{s}, —C(O)N(R^{s})₂, —C(O)R^{s}, —NHC(O)R^{s}, —NR^{s}C(O)R^{s}, —NHC(O)NH₂, —NR^{s}C(O)NH₂, —NR^{s}C(O)NHR^{s}, —NHC(O)NHR^{s}, —NR^{s}C(O)N(R^{s})₂, —NHC(O)N(R^{s})₂, —CO₂H, —CO₂R^{s}, —NHCO₂R^{s}, —NR^{s}CO₂R^{s}, —CN, —NO₂, —NH₂—, —NHR^{s}, —N(R^{s})₂, —NR^{s}S(O)NH₂ and —NR^{s}S(O)₂NHR^{s}, wherein each R^{s} is independently an unsubstituted C₁₋₆ alkyl. In one embodiment of formula I, R³ is selected from the group consisting of hydrogen, optionally substituted C₃₋₈ cycloalkyl and optionally substituted C₁₋₆ alkyl.

The substituent D, in formula I, is O, S or N—OR^{t}, wherein R^{t} is selected from the group consisting of hydrogen, C₁₋₈ alkyl, C₁₋₈ haloalkyl and C₁₋₈ heteroalkyl. In one embodiment, the substituent D is O.

Subformulae of Formula I.

In one embodiment of the present invention, compounds of formula I have subformula Ia: 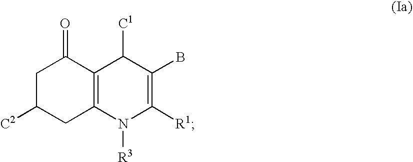
wherein the substituents R¹, R³, C¹, C² and B are as defined above for formula I. In one embodiment, R³ in subformula Ia is preferably hydrogen. In another embodiment of subformula Ia, R³ is hydrogen, C¹ and C² are each optionally substituted aryl or heteroaryl, B is —CO₂R^{a}, —CONR^{a}R^{b}, —C(O)R^{a}, and R¹ is an optionally substituted member selected from the group consisting of C₁₋₈ alkyl. C₃₋₈ cycloalkyl, C₃₋₈ cycloalkyl-C₁₋₈ alkyl and C₁₋₈ haloalkyl. In yet another embodiment, compounds having subformula Ia, B is selected from the group consisting of halogen, —CN, —CO₂R^{a}, —CONR^{a}R^{b}, and —C(O)R^{a}; R¹ is C₁₋₈ alkyl or C₁₋₈ haloalkyl; C¹ and C² are each independently an optionally substituted phenyl; and R³ is hydrogen or C₁₋₆ alkyl.

In a second embodiment of the invention, compounds of formula I have subformula Ib 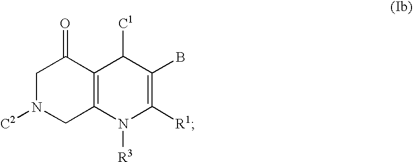
wherein the substituents R¹, R³, C¹, C² and B are as defined above for formula I.

In a third embodiment of the invention, compounds of formula I have subformula Ic; 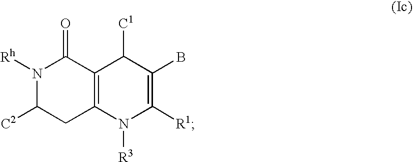
wherein the substituents R¹, R³, C¹, C², R^{h} and B are as defined above for formula I.

In a third embodiment of the invention, compounds of formula I have subformula Id 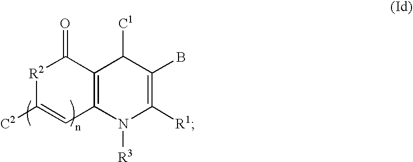
wherein the substituents R¹, R², R³, C¹, C² and B are as defined above for formula I, and wherein the subscript n is an integer from 1-3.

In a fourth embodiment, compounds of formula I have subformula Ie: 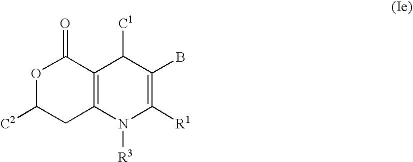
wherein the substituents R¹, R³, C¹, C² and B are as defined above for formula I.

In a fifth embodiment, compounds of formula I have subformula If: 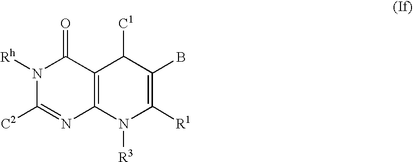
wherein the substituents R¹, R³, C¹, C², R^{h} and B are as defined above for formula I.

Compounds of the invention having formula I can exist in different diastereomeric forms, e.g., the substituents C¹ and C² in subformulae Ia and Ic can be cis to each other or trans to each other. As used herein, the terms cis or trans are used in their conventional sense in the chemical arts, i.e., referring to the position of the substituents to one another relative to a reference plane, e.g., a double bond, or a ring system, such as a decalin-type ring system or a hydroquinolone ring system: in the cis isomer, the substituents are on the same side of the reference plane, in the trans isomer the substituents are on opposite sides. In one embodiment, the substituents C¹ and C² in formulae Ia, and Ic are cis to each other. In another embodiment, the subsituents C¹ and C² in formulae Ia and Ic are trans to each other.

The C¹ Substituent:

In one embodiment of formula I, C¹ is preferably a 5- to 10-membered aryl or heteroaryl group. In one embodiment, C¹ is an optionally substituted member selected from the group consisting phenyl, 2-pyridyl, 3-pyridyl, 4-pyridyl, 2-thienyl, 3-thienyl, 2-furanyl, 3-furanyl, 3-indolyl, 4-indolyl, 5-indolyl, 6-indolyl, 7-indolyl, 2-pyrrolyl and 3-pyrrolyl.

In another embodiment of formula I, C¹ is a phenyl group further optionally substituted with 1 to 3 substituents selected from the group consisting of —OH, —NO₂, —NR^{d}R^{e}, halogen, C₁₋₈ alkyl, C₁₋₈ haloalkyl, C₁₋₈ alkoxy, and C₂₋₈ alkenyl. In another embodiment, C¹ phenyl ring having formula 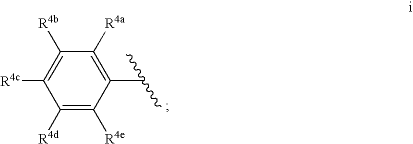
wherein R^{4a}, R^{4b}, R^{4c} and R^{4e} are each hydrogen and R^{4d} is selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{c}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}. Preferably, R^{4d} is selected from the group consisting of —OH, —OR^{f}, —NO₂, —NR^{d}R^{e}, halogen and C₁₋₈ alkyl.

In yet another embodiment of formula I, C¹ is selected from the group consisting of: 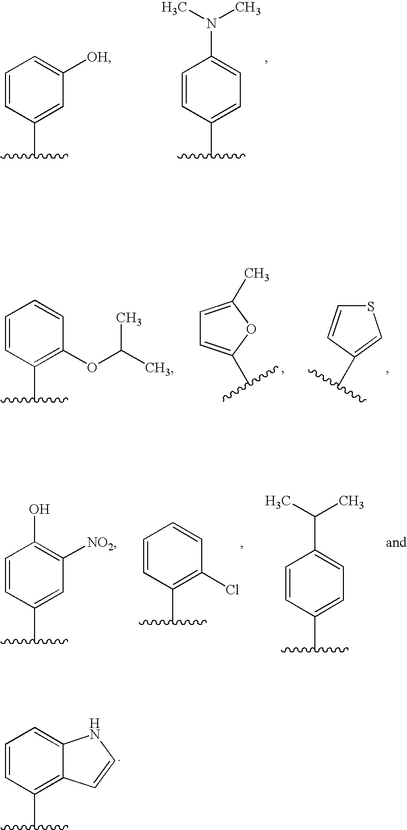
The C² Substituent:

In a certain embodiment of compounds having formula I, the C² substituent is preferably a 5- to 10-membered aryl or heteroaryl group. In one embodiment, C² is an optionally substituted member selected from the group consisting of phenyl, 2-pyridyl, 3-pyridyl, 4-pyridyl, 2-thienyl, 3-thienyl, 2-furanyl, 3-furanyl, 2-pyrrolyl and 3-pyrrolyl.

In another embodiment of formula I, C² is a phenyl group optionally having 1 to 5 substituents selected from the group consisting of halogen, cyano, —NR^{d}R^{e} and —R^{f}; wherein R^{f} is independently selected from the group consisting of C₁₋₈ alkyl, C₁₋₈ haloalkyl, C₃₋₆ cycloalkyl, C₂₋₈ alkenyl and C₂₋₈ alkynyl.

In another embodiment, C² is a phenyl group having the formula 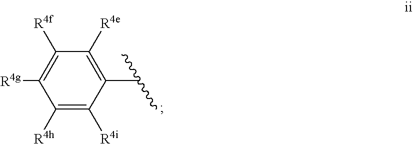
wherein R^{4f} and R^{4h} each hydrogen and R^{4e}, R^{4g} and R⁴ⁱ are each independently selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{c}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; optionally any two substituents located on adjacent atoms of C¹ or C² are combined to form a 5- to 7-membered ring.

In another embodiment R^{4e}, R^{4f}, R^{4h} and R⁴ⁱ are each hydrogen and R^{4g} is selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —XC(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{c}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; optionally any two substituents located on adjacent atoms of C¹ or C² are combined to form a 5- to 7-membered ring.

In another embodiment R^{4f}, R^{4g} and R^{4h} are each hydrogen and R^{4e} and R⁴ⁱ are each independently selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{c}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}X²CO₂R^{d}; optionally any two substituents located on adjacent atoms of C¹ or C² are combined to form a 5- to 7-membered ring.

In another embodiment R^{4e}, R^{4h} and R⁴ⁱ are each hydrogen and R^{4f} and R^{4g} are each independently selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{c}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; optionally any two substituents located on adjacent atoms of C¹ or C² are combined to form a 5- to 7-membered ring.

In yet another embodiment, C² is selected from the group consisting of: 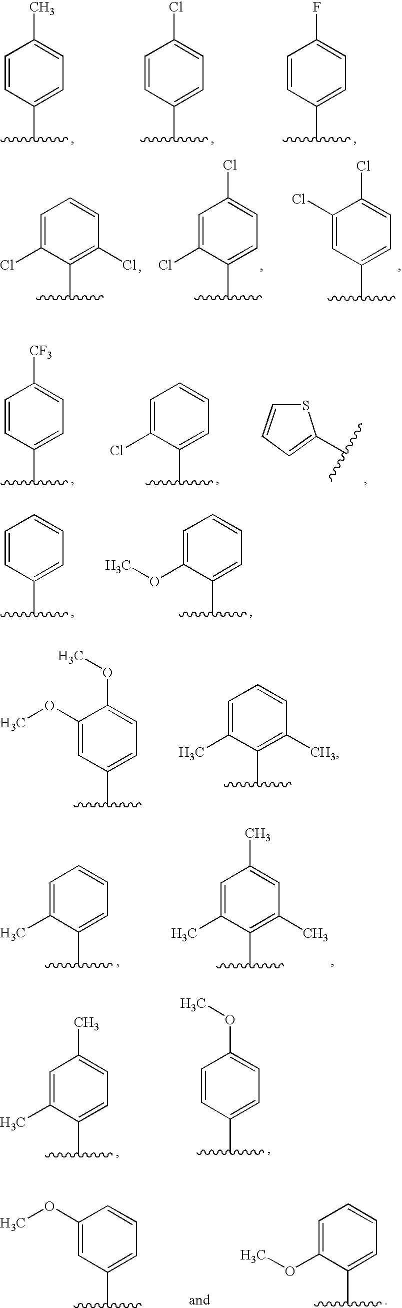
Preparation of Compounds

The compounds of the invention can be prepared, for example, by the synthetic route shown in Scheme 1. In Scheme 1, TMS represents a trimethylsilyl group, BuLi is butyl lithium, LDA is lithium diisopropylamide, M is a metal ion, such as a sodium or potassium ion, and the like; and non-interferring substituents are provided as —R, —R′, —R″, R″′ and —R″″.

As shown in Scheme 1, dihydropyridine compounds of the invention can be prepared by a three step synthetic route. In the first step, aldehyde 1 and ketone 2 are condensed under base or acid catalyzed conditions to provide the dehydrated enone product 3. A Michael-type reaction can be performed using enone 3 and a malonate species, such as diethyl malonate, and the like, under basic conditions followed by decarboxylation of a cyclized intermediate 3.5 can produce diketone 4. The final dihydropyridine product 7 can be prepared by using a modified Hantzsch reaction between dione 4, aldehyde 5, β-ketoester 6 and a nitrogen source, such as, ammonium acetate. Other routes or modification of the route presented below would be readily apparent to a skilled artisan and within the scope of the present invention. For example, although Scheme 1 shows the Hantzsch-type reaction to make the polyhydroquinolone ring system using dione 4, a skilled artisan would recognized that dione 4 can be replaced with an β-amino-enone species, such as 3-aminocyclohexen-2-one that could also participate in the Hantzsch-type reaction.

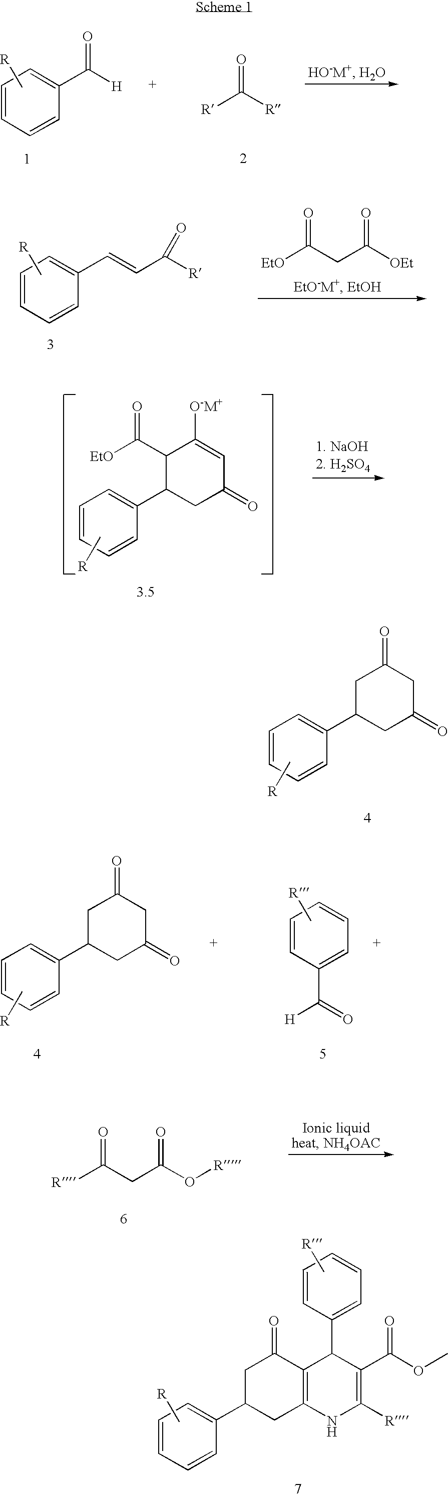

In addition, lactone compounds of the present invention can be prepared by the synthetic route shown in Scheme 2. As shown in Scheme 2, a Lewis acid catalyzed aldol reaction between aldehyde 1 and diketene 16 will produce the aldol product 13-rac as a mixture of enantiomers. Subsequent cyclization of 13-rac under basic conditions will produce diketone 14-rac. Diketone 14-rac can be used in the Hanztch type cyclization reaction (described in Scheme 1) to form a lactone compound 17 of the present invention.

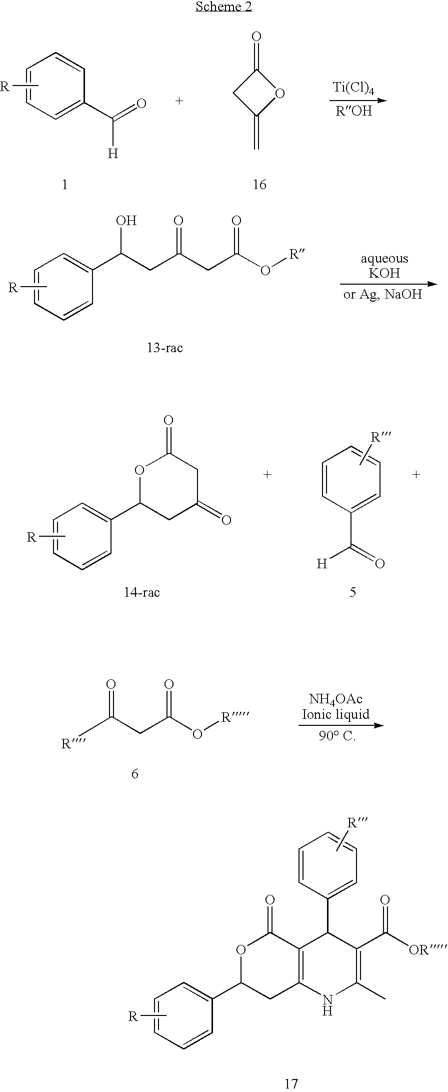

Following the procedure described by Soriente, A., et al.(Tetrahedron: Asymmetry (2001), 12(6), 959-963) and as shown in Scheme 3, enantiopure aldol intermediate 13 (the symbol * denotes the chiral center) can be prepared by the asymmetric aldol reaction between trimethylsilyl enol ether 12 and aldehyde 1 catalyzed by a Lewis acid in the presence of a chiral ligand, such as, R-Binol (R-(+)-1,1′-Bi-2-naphthol), and the like. Cyclization of aldol product 13 under basic conditions can yield lactone 14 as primarily a single enantiomer. Enantiopure lactone 14 can be used in the Hantzch-type cyclization reaction as describe in Scheme 2 to provide compounds of the invention.

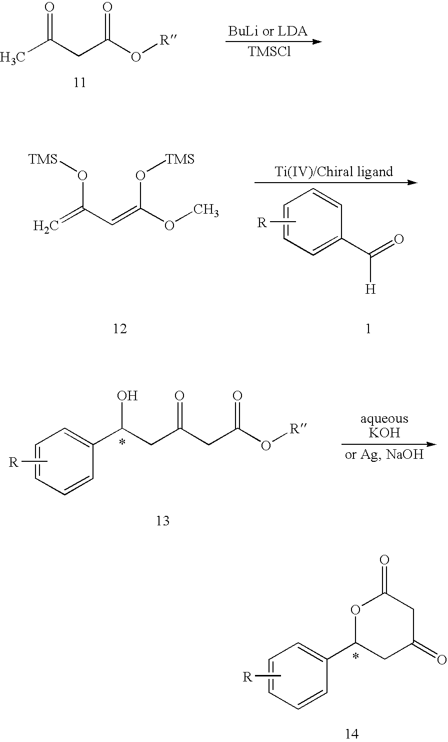

Lactam compounds of the present invention can be prepared by the synthetic routes shown in Scheme 3.

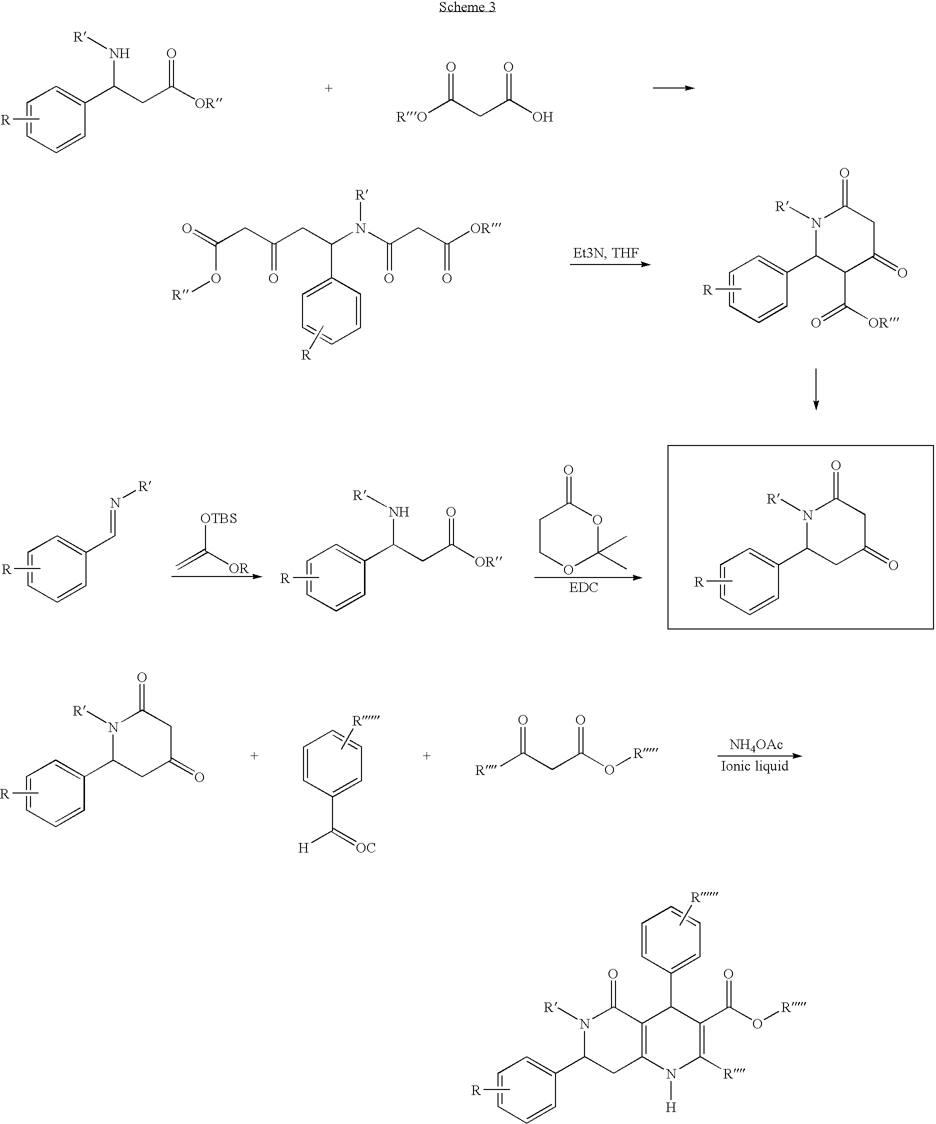

The 3-oxo-piperidine compounds (Ib) of the present invention can be prepared according to the synthetic routes shown in Scheme 4.

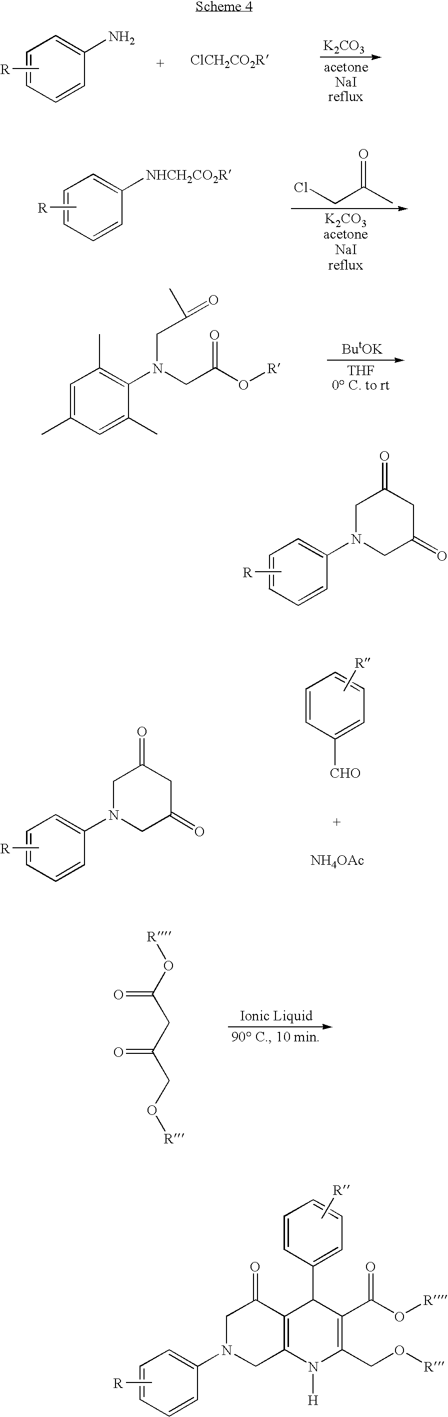

The oxo-pyriridine compounds (If) of the present invention can be prepared according to the synthetic routes shown in Scheme 5.

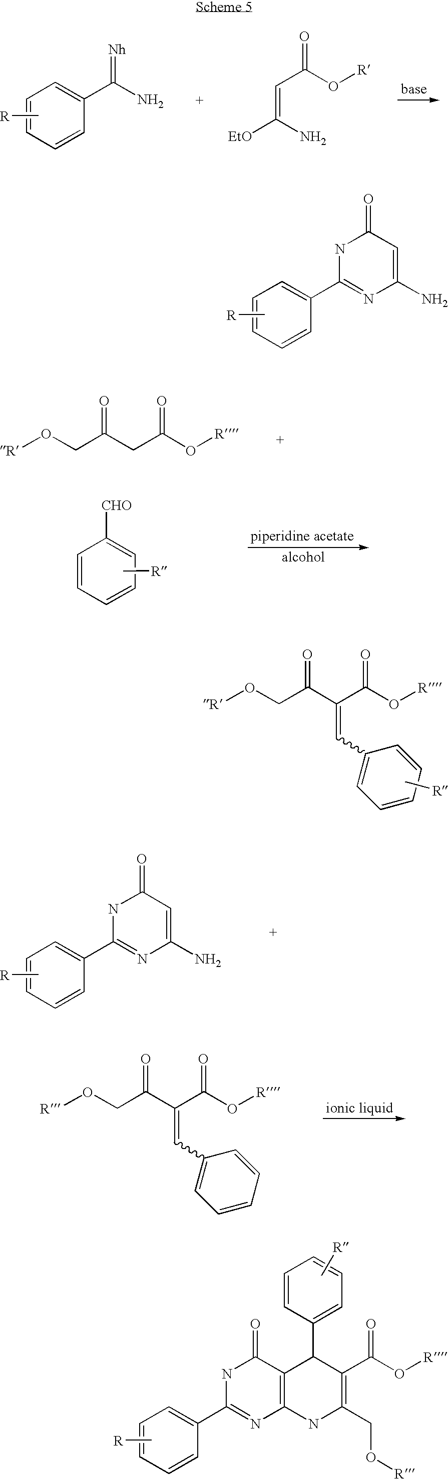

A family of specific compound of particular interest having formula I consists of compounds, pharmaceutically acceptable salts, hydrates thereof, as set forth in Table 1.

TABLE 1

| | |
|---|---|
| 1. | 2-Cyclopentyl-4-(3-hydroxy-phenyl)-5-oxo-7-(2,4,6-trimethyl-phenyl)-1,4,5,6,7,8- |
| | hexahydro-quinoline-3-carboxylic acid cyclopentyl ester |
| 2. | 2-Cyclopentylmethyl-4-(3-hydroxy-phenyl)-5-oxo-7-(2,4,6-trimethyl-phenyl)- |
| | 1,4,5,6,7,8-hexahydro-quinoline-3-carboxylic acid cyclopentyl ester |
| 3. | 7-Benzo[1,3]dioxol-5-yl-2-cyclopentylmethyl-4-(3-hydroxy-phenyl)-5-oxo- |
| | 1,4,5,6,7,8-hexahydro-quinoline-3-carboxylic acid cyclopentyl ester |
| 4. | 2-Ethyl-4-(3-hydroxy-phenyl)-5-oxo-7-(2,4,6-trimethyl-phenyl)-1,4,5,6,7,8- |
| | hexahydro-quinoline-3-carboxylic acid cyclopentyl ester |
| 5. | 4-(3-Hydroxy-phenyl)-2-isobutyl-5-oxo-7-(2,4,6-trimethyl-phenyl)-1,4,5,6,7,8- |
| | hexahydro-quinoline-3-carboxylic acid cyclopentyl ester |
| 6. | 7-Benzo[1,3]dioxol-5-yl-4-(3-hydroxy-phenyl)-2-isobutyl-5-oxo-1,4,5,6,7,8- |
| | hexahydro-quinoline-3-carboxylic acid cyclopentyl ester |
| 7. | 7-(2,6-Dimethyl-phenyl)-4-(3-hydroxy-phenyl)-2-isobutyl-5-oxo-1,4,5,6,7,8- |
| | hexahydro-quinoline-3-carboxylic acid cyclopentyl ester |
| 8. | 7-Benzo[1,3]dioxol-5-yl-4-(3-hydroxy-phenyl)-2-isopropyl-5-oxo-1,4,5,6,7,8- |
| | hexahydro-quinoline-3-carboxylic acid cyclopentyl ester |
| 9. | 7-(2,6-Dichloro-phenyl)-4-(3-hydroxy-phenyl)-2-isopropyl-5-oxo-1,4,5,6,7,8- |
| | hexahydro-quinoline-3-carboxylic acid cyclopentyl ester |
| 10. | 7-(2,6-Dimethyl-phenyl)-2-ethyl-4-(3-hydroxy-phenyl)-5-oxo-1,4,5,6,7,8-hexahydro- |
| | quinoline-3-carboxylic acid cyclopentyl ester |
| 11. | 7-(2,2-Difluoro-benzo[1,3]dioxol-5-yl)-2-ethyl-4-(3-hydroxy-phenyl)-5-oxo- |
| | 1,4,5,6,7,8-hexahydro-quinoline-3-carboxylic acid cyclopentyl ester |
| 12. | 2-Cyclopropyl-4-(3-hydroxy-phenyl)-5-oxo-7-(2,4,6-trimethyl-phenyl)-1,4,5,6,7,8- |
| | hexahydro-quinoline-3-carboxylic acid cyclopentyl ester |
| 13. | 7-Benzo[1,3]dioxol-5-yl-2-ethyl-4-(3-hydroxy-phenyl)-5-oxo-1,4,5,6,7,8-hexahydro- |
| | quinoline-3-carboxylic acid cyclopentyl ester |
| 14. | 2-Ethyl-7-(3-fluoro-4-methyl-phenyl)-4-(3-hydroxy-phenyl)-5-oxo-1,4,5,6,7,8- |
| | hexahydro-quinoline-3-carboxylic acid cyclopentyl ester |
| 15. | 7-(2,3-Dihydro-benzo[1,4]dioxin-6-yl)-2-ethyl-4-(3-hydroxy-phenyl)-5-oxo- |
| | 1,4,5,6,7,8-hexahydro-quinoline-3-carboxylic acid cyclopentyl ester |
| 16. | 7-(2,3-Dihydro-benzofuran-6-yl)-2-ethyl-4-(3-hydroxy-phenyl)-5-oxo-1,4,5,6,7,8- |
| | hexahydro-quinoline-3-carboxylic acid cyclopentyl ester |
| 17. | 2-Ethyl-4-(3-hydroxy-phenyl)-5-oxo-7-p-tolyl-1,4,5,6,7,8-hexahydro-quinoline-3- |
| | carboxylic acid cyclopentyl ester |
| 18. | 7-(2,6-Dichloro-phenyl)-2-ethyl-4-(3-hydroxy-phenyl)-5-oxo-1,4,5,6,7,8-hexahydro- |
| | quinoline-3-carboxylic acid cyclopentyl ester |
| 19. | 2-Ethyl-4-(3-hydroxy-phenyl)-5-oxo-7-p-tolyl-1,4,5,6,7,8-hexahydro-quinoline-3- |
| | carboxylic acid cyclopentyl ester |
| 20. | 2-Cyclopropyl-7-(2,6-dimethyl-phenyl)-4-(3-hydroxy-phenyl)-5-oxo-1,4,5,6,7,8- |
| | hexahydro-quinoline-3-carboxylic acid cyclopentyl ester |
| 21. | 4-(3-Hydroxy-phenyl)-2-isopropyl-5-oxo-7-p-tolyl-1,4,5,6,7,8-hexahydro-quinoline- |
| | 3-carboxylic acid cyclopentyl ester |
| 22. | 7-(4-Ethyl-phenyl)-4-(3-hydroxy-phenyl)-2-methyl-5-oxo-1,4,5,6,7,8-hexahydro- |
| | quinoline-3-carboxylic acid cyclopentyl ester |
| 23. | 4-(3-Hydroxy-phenyl)-7-(4-isopropyl-phenyl)-2-methyl-5-oxo-1,4,5,6,7,8-hexahydro- |
| | quinoline-3-carboxylic acid cyclopentyl ester |
| 24. | 7-(3-Fluoro-4-methyl-phenyl)-4-(3-hydroxy-phenyl)-2-methyl-5-oxo-1,4,5,6,7,8- |
| | hexahydro-quinoline-3-carboxylic acid cyclopentyl ester |
| 25. | 7-(2,6-Dichloro-phenyl)-4-(3-hydroxy-phenyl)-2-methyl-5-oxo-1,4,5,6,7,8- |
| | hexahydro-quinoline-3-carboxylic acid cyclopentyl ester |
| 26. | 2-Ethyl-7-(3-fluoro-4-methyl-phenyl)-4-(3-hydroxy-phenyl)-5-oxo-1,4,5,6,7,8- |
| | hexahydro-quinoline-3-carboxylic acid cyclopentyl ester |
| 27. | 4-(3-Hydroxy-phenyl)-2-methyl-5-oxo-7-p-tolyl-1,4,5,6,7,8-hexahydro-quinoline-3- |
| | carboxylic acid cyclopentyl ester |
| 28. | 7-Benzo[1,3]dioxol-4-yl-2-ethyl-4-(3-hydroxy-phenyl)-5-oxo-1,4,5,6,7,8-hexahydro- |
| | quinoline-3-carboxylic acid cyclopentyl ester |
| 29. | 7-(2,4-Dichloro-phenyl)-4-(3-hydroxy-phenyl)-2-methyl-5-oxo-1,4,5,6,7,8- |
| | hexahydro-quinoline-3-carboxylic acid cyclopentyl ester |
| 30. | 7-(3,5-Dichloro-pyridin-4-yl)-4-(3-hydroxy-phenyl)-2-isopropyl-5-oxo-1,4,5,6,7,8- |
| | hexahydro-quinoline-3-carboxylic acid cyclopentyl ester |
| 31. | 2-Ethyl-4-(3-hydroxy-phenyl)-5-oxo-7-p-tolyl-1,4,5,6,7,8-hexahydro-quinoline-3- |
| | carboxylic acid cyclopentyl ester |
| 32. | 7-(4-Fluoro-phenyl)-4-(3-hydroxy-phenyl)-2-methyl-5-oxo-1,4,5,6,7,8-hexahydro- |
| | quinoline-3-carboxylic acid cyclopentyl ester |
| 33. | 7-(2,2-Difluoro-benzo[1,3]dioxol-5-yl)-2-ethyl-4-(3-hydroxy-phenyl)-5-oxo- |
| | 1,4,5,6,7,8-hexahydro-quinoline-3-carboxylic acid cyclopentyl ester |
| 34. | 7-(4-Chloro-3-fluoro-phenyl)-4-(3-hydroxy-phenyl)-2-methyl-5-oxo-1,4,5,6,7,8- |
| | hexahydro-quinoline-3-carboxylic acid cyclopentyl ester |
| 35. | 7-(4-Chloro-3-fluoro-phenyl)-4-(3-hydroxy-phenyl)-2-methyl-5-oxo-1,4,5,6,7,8- |
| | hexahydro-quinoline-3-carboxylic acid cyclopentyl ester |
| 36. | 7-(3,5-Dichloro-pyridin-4-yl)-2-ethyl-4-(3-hydroxy-phenyl)-5-oxo-1,4,5,6,7,8- |
| | hexahydro-quinoline-3-carboxylic acid cyclopentyl ester |
| 37. | 4-(3-Hydroxy-phenyl)-2-methyl-5-oxo-7-p-tolyl-1,4,5,6,7,8-hexahydro-quinoline-3- |
| | carboxylic acid ethyl ester |
| 38. | 7-(3,4-Dichloro-phenyl)-4-(3-hydroxy-phenyl)-2-methyl-5-oxo-1,4,5,6,7,8- |
| | hexahydro-quinoline-3-carboxylic acid cyclopentyl ester |
| 39. | 7-Cyclohexyl-4-(3-hydroxy-phenyl)-2-methyl-5-oxo-1,4,5,6,7,8-hexahydro- |
| | quinoline-3-carboxylic acid cyclopentyl ester |
| 40. | 4-(3-Hydroxy-phenyl)-2-methyl-5-oxo-7-phenyl-1,4,5,6,7,8-hexahydro-quinoline-3- |
| | carboxylic acid isobutyl ester |
| 41. | 7-(4-Chloro-3-fluoro-phenyl)-4-(3-hydroxy-phenyl)-2-methyl-5-oxo-1,4,5,6,7,8- |
| | hexahydro-quinoline-3-carboxylic acid cyclopentyl ester |
| 42. | 7-(3,4-Dichloro-phenyl)-4-(3-hydroxy-phenyl)-2-methyl-5-oxo-1,4,5,6,7,8- |
| | hexahydro-quinoline-3-carboxylic acid cyclopentyl ester |
| 43. | 4-(3-Hydroxy-phenyl)-2-methyl-7-naphthalen-2-yl-5-oxo-1,4,5,6,7,8-hexahydro- |
| | quinoline-3-carboxylic acid cyclopentyl ester |
| 44. | 7-(4-Fluoro-phenyl)-4-(3-hydroxy-phenyl)-2-methyl-5-oxo-1,4,5,6,7,8-hexahydro- |
| | quinoline-3-carboxylic acid cyclopentyl ester |
| 45. | 7-(4-Dimethylamino-phenyl)-4-(3-hydroxy-phenyl)-2-methyl-5-oxo-1,4,5,6,7,8- |
| | hexahydro-quinoline-3-carboxylic acid cyclopentyl ester |
| 46. | 7-(4-tert-Butyl-phenyl)-4-(3-hydroxy-phenyl)-2-methyl-5-oxo-1,4,5,6,7,8-hexahydro- |
| | quinoline-3-carboxylic acid cyclopentyl ester |
| 47. | 4-(3-Hydroxy-phenyl)-2-methyl-5-oxo-7-(4-trifluoromethyl-phenyl)-1,4,5,6,7,8- |
| | hexahydro-quinoline-3-carboxylic acid cyclopentyl ester |
| 48. | 4-(3-Hydroxy-phenyl)-2-methyl-7-naphthalen-1-yl-5-oxo-1,4,5,6,7,8-hexahydro- |
| | quinoline-3-carboxylic acid cyclopentyl ester |
| 49. | 7-Cyclopentyl-4-(3-hydroxy-phenyl)-2-methyl-5-oxo-1,4,5,6,7,8-hexahydro- |
| | quinoline-3-carboxylic acid cyclopentyl ester |
| 50. | 7-Biphenyl-4-yl-4-(3-hydroxy-phenyl)-2-methyl-5-oxo-1,4,5,6,7,8-hexahydro- |
| | quinoline-3-carboxylic acid cyclopentyl ester |
| 51. | 4-(3-Hydroxy-phenyl)-2-methyl-5-oxo-7-(4-trifluoromethyl-phenyl)-1,4,5,6,7,8- |
| | hexahydro-quinoline-3-carboxylic acid cyclopentyl ester |
| 52. | 4-(3-Hydroxy-phenyl)-2-methyl-5-oxo-7-phenyl-1,4,5,6,7,8-hexahydro- |
| | [1,7]naphthyridine-3-carboxylic acid cyclopentyl ester |
| 53. | 4-(4-Fluoro-phenyl)-2-methyl-5-oxo-7-p-tolyl-1,4,5,6,7,8-hexahydro-quinoline-3- |
| | carboxylic acid cyclopentyl ester |
| 54. | 4-(4-Fluoro-phenyl)-2-methyl-5-oxo-7-p-tolyl-1,4,5,6,7,8-hexahydro-quinoline-3- |
| | carboxylic acid cyclopentyl ester |
| 55. | 4-(3-Hydroxy-phenyl)-2-methyl-5-oxo-7-pyridin-2-yl-1,4,5,6,7,8-hexahydro- |
| | quinoline-3-carboxylic acid cyclopentyl ester |
| 56. | 4-(3-Hydroxy-phenyl)-2-methyl-5-oxo-7-pyridin-3-yl-1,4,5,6,7,8-hexahydro- |
| | quinoline-3-carboxylic acid cyclopentyl ester |
| 57. | 4-(3-Hydroxy-phenyl)-2-methyl-5-oxo-7-pyridin-4-yl-1,4,5,6,7,8-hexahydro- |
| | quinoline-3-carboxylic acid cyclopentyl ester |
| 58. | 2-(2-Cyclopentyl-ethyl)-4-(1H-indol-4-yl)-5-oxo-7-(2,4,6-trimethyl-phenyl)- |
| | 1,4,5,6,7,8-hexahydro-quinoline-3-carboxylic acid ethyl ester |
| 59. | 7-(2,6-Dimethyl-phenyl)-2-ethyl-5-hydroxyimino-4-(3-hydroxy-phenyl)-1,4,5,6,7,8- |
| | hexahydro-quinoline-3-carboxylic acid cyclopentyl ester |
| 60. | 7-(2,6-Dimethyl-phenyl)-2-ethyl-4-(3-hydroxy-phenyl)-5-methoxyimino-1,4,5,6,7,8- |
| | hexahydro-quinoline-3-carboxylic acid cyclopentyl ester |
| 61. | 4-(3-Hydroxy-phenyl)-2-methyl-5-oxo-7-phenyl-1,4,5,6,7,8-hexahydro- |
| | [1,7]naphthyridine-3-carboxylic acid cyclopentyl ester |
| 62. | 7-Cyclohexyl-4-(3-hydroxy-phenyl)-2-methyl-5-oxo-1,4,5,6,7,8-hexahydro- |
| | quinoline-3-carboxylic acid cyclopentyl ester |
| 63. | 7-(2,6-Dichloro-phenyl)-4-(2,4-dioxo-1,2,3,4-tetrahydro-pyrimidin-5-yl)-2-methyl-5- |
| | oxo-1,4,5,6,7,8-hexahydro-quinoline-3-carboxylic acid cyclopentyl ester |
| 64. | 2-(2-Cyclopentyl-ethyl)-4-(3-hydroxy-phenyl)-5-oxo-7-(2,4,6-trimethyl-phenyl)- |
| | 1,4,5,6,7,8-hexahydro-quinoline-3-carboxylic acid cyclopentyl ester |
| 65. | 2-(2-Cyclopentyl-ethyl)-4-(3-hydroxy-phenyl)-5-oxo-7-(2,4,6-trimethyl-phenyl)- |
| | 1,4,5,6,7,8-hexahydro-quinoline-3-carboxylic acid ethyl ester |
| 66. | 2-Methyl-4-(3-nitro-phenyl)-5-oxo-7-(2,4,6-trimethyl-phenyl)-1,4,5,6,7,8-hexahydro- |
| | quinoline-3-carboxylic acid cyclopentyl ester |
| 67. | 4-(3-Amino-phenyl)-2-methyl-5-oxo-7-(2,4,6-trimethyl-phenyl)-1,4,5,6,7,8- |
| | hexahydro-quinoline-3-carboxylic acid cyclopentyl ester |
| 68. | 4-(3-Acetylamino-phenyl)-2-methyl-5-oxo-7-(2,4,6-trimethyl-phenyl)-1,4,5,6,7,8- |
| | hexahydro-quinoline-3-carboxylic acid cyclopentyl ester |
| 69. | 4-(3-Methanesulfonylamino-phenyl)-2-methyl-5-oxo-7-(2,4,6-trimethyl-phenyl)- |
| | 1,4,5,6,7,8-hexahydro-quinoline-3-carboxylic acid cyclopentyl ester |
| 70. | 7-Benzo[1,3]dioxol-5-yl-2-(2-cyclopentyl-ethyl)-4-(3-hydroxy-phenyl)-5-oxo- |
| | 1,4,5,6,7,8-hexahydro-quinoline-3-carboxylic acid ethyl ester |
| 71. | 7-Benzo[1,3]dioxol-5-yl-2-methyl-4-(3-nitro-phenyl)-5-oxo-1,4,5,6,7,8-hexahydro- |
| | quinoline-3-carboxylic acid cyclopentyl ester |
| 72. | 4-(3-Amino-phenyl)-7-benzo[1,3]dioxol-5-yl-2-methyl-5-oxo-1,4,5,6,7,8-hexahydro- |
| | quinoline-3-carboxylic acid cyclopentyl ester |
| 73. | 7-Benzo[1,3]dioxol-5-yl-4-(3-carbamoyl-phenyl)-2-methyl-5-oxo-1,4,5,6,7,8- |
| | hexahydro-quinoline-3-carboxylic acid cyclopentyl ester |
| 74. | 7-Benzo[1,3]dioxol-5-yl-4-(3-carboxy-phenyl)-2-methyl-5-oxo-1,4,5,6,7,8- |
| | hexahydro-quinoline-3-carboxylic acid cyclopentyl ester |
| 75. | 7-Benzo[1,3]dioxol-5-yl-4-(1H-indol-6-yl)-2-methyl-5-oxo-1,4,5,6,7,8-hexahydro- |
| | quinoline-3-carboxylic acid cyclopentyl ester |
| 76. | 7-Benzo[1,3]dioxol-5-yl-4-(1H-indol-3-yl)-2-methyl-5-oxo-1,4,5,6,7,8-hexahydro- |
| | quinoline-3-carboxylic acid cyclopentyl ester |
| 77. | 7-(4-Ethyl-phenyl)-4-(3-hydroxy-phenyl)-2-methyl-5-oxo-1,4,5,6,7,8-hexahydro- |
| | quinoline-3-carboxylic acid cyclopentylamide |
| 78. | 4-(3-Hydroxy-phenyl)-2-methyl-5-oxo-7-phenyl-5,6,7,8-tetrahydro-quinoline-3- |
| | carboxylic acid cyclopentyl ester |
| 79. | 4-(1H-Indol-6-yl)-2-methyl-5-oxo-7-phenyl-1,4,5,6,7,8-hexahydro-quinoline-3- |
| | carboxylic acid cyclopentyl ester |
| 80. | 4-(1H-Indol-3-yl)-2-methyl-5-oxo-7-phenyl-1,4,5,6,7,8-hexahydro-quinoline-3- |
| | carboxylic acid cyclopentyl ester |
| 81. | 4-(3-Carboxy-phenyl)-2-methyl-5-oxo-7-phenyl-1,4,5,6,7,8-hexahydro-quinoline-3- |
| | carboxylic acid cyclopentyl ester |
| 82. | 4-(3-Carbamoyl-phenyl)-2-methyl-5-oxo-7-phenyl-1,4,5,6,7,8-hexahydro-quinoline- |
| | 3-carboxylic acid cyclopentyl ester |
| 83. | 4-(6-Hydroxy-pyridin-2-yl)-2-methyl-5-oxo-7-phenyl-1,4,5,6,7,8-hexahydro- |
| | quinoline-3-carboxylic acid cyclopentyl ester |
| 84. | 4-(3-Methanesulfonylamino-phenyl)-2-methyl-5-oxo-7-phenyl-1,4,5,6,7,8- |
| | hexahydro-quinoline-3-carboxylic acid cyclopentyl ester |

Excluded from the above generic formula, as well as each of the formulae below, are those compounds that are either commercially available or known in the literature, including: 4-(3-Hydroxy-phenyl)-2-methyl-5-oxo-7-thiophen-2-yl-1,4,5,6,7,8-hexahydro-quinoline-3-carboxylic acid cyclopentyl ester; 7-(4-Chloro-phenyl)-4-(3-hydroxy-phenyl)-2-methyl-5-oxo-1,4,5,6,7,8-hexahydro-quinoline-3-carboxylic acid isobutyl ester; 4-(3-Hydroxy-phenyl)-7-(4-methoxy-phenyl)-2-methyl-5-oxo-1,4,5,6,7,8-hexahydro-quinoline-3-carboxylic acid cyclopentyl ester; 4-(3-Hydroxy-phenyl)-2-methyl-5-oxo-7-phenyl-1,4,5,6,7,8-hexahydro-quinoline-3-carboxylic acid cyclopentyl ester; 4-(3-Hydroxy-phenyl)-2-methyl-5-oxo-7-phenyl-1,4,5,6,7,8-hexahydro-quinoline-3-carboxylic acid propyl ester; 4-(3-Hydroxy-phenyl)-2-methyl-5-oxo-7-phenyl-1,4,5,6,7,8-hexahydro-quinoline-3-carboxylic acid 2-phenoxy-ethyl ester; 2-Methyl-4-(5-methyl-furan-2-yl)-5-oxo-7-phenyl-1,4,5,6,7,8-hexahydro-quinoline-3-carboxylic acid cyclohexyl ester; 4-(3-Hydroxy-phenyl)-2-methyl-5-oxo-7-phenyl-1,4,5,6,7,8-hexahydro-quinoline-3-carboxylic acid ethyl ester; 4-(3-Hydroxy-phenyl)-2-methyl-5-oxo-7-thiophen-2-yl-1,4,5,6,7,8-hexahydro-quinoline-3-carboxylic acid isobutyl ester; 4-(3-Hydroxy-phenyl)-2-methyl-5-oxo-7-thiophen-2-yl-1,4,5,6,7,8-hexahydro-quinoline-3-carboxylic acid cyclopentyl ester; 7-(2-Methoxy-phenyl)-2-methyl-5-oxo-4-thiophen-3-yl-1,4,5,6,7,8-hexahydro-quinoline-3-carboxylic acid cyclopentyl ester; 7-(4-Chloro-phenyl)-4-(3-hydroxy-phenyl)-2-methyl-5-oxo-1,4,5,6,7,8-hexahydro-quinoline-3-carboxylic acid methyl ester; 7-(4-Chloro-phenyl)-4-(3-hydroxy-phenyl)-2-methyl-5-oxo-1,4,5,6,7,8-hexahydro-quinoline-3-carboxylic acid 2-phenoxy-ethyl ester; 7-(3,4-Dimethoxy-phenyl)-2-methyl-5-oxo-4-phenyl-1,4,5,6,7,8-hexahydro-quinoline-3-carboxylic acid cyclopentyl ester; 4-(3-Hydroxy-phenyl)-2-methyl-5-oxo-7-phenyl-1,4,5,6,7,8-hexahydro-quinoline-3-carboxylic acid benzyl ester; 4-(3-Hydroxy-phenyl)-7-(4-methoxy-phenyl)-2-methyl-5-oxo-1,4,5,6,7,8-hexahydro-quinoline-3-carboxylic acid 2-ethylsulfanyl-ethyl ester; 4-(3-Hydroxy-phenyl)-7-(4-methoxy-phenyl)-2-methyl-5-oxo-1,4,5,6,7,8-hexahydro-quinoline-3-carboxylic acid 2-phenoxy-ethyl ester; 7-(3,4-Dimethoxy-phenyl)-4-(3-hydroxy-phenyl)-2-methyl-5-oxo-1,4,5,6,7,8-hexahydro-quinoline-3-carboxylic acid sec-butyl ester; 7-(3,4-Dimethoxy-phenyl)-4-(3-hydroxy-phenyl)-2-methyl-5-oxo-1,4,5,6,7,8-hexahydro-quinoline-3-carboxylic acid 2-ethylsulfanyl-ethyl ester; 7-(3,4-Dimethoxy-phenyl)-4-(3-hydroxy-phenyl)-2-methyl-5-oxo-1,4,5,6,7,8-hexahydro-quinoline-3-carboxylic acid isopropyl ester; 7-(2-Methoxy-phenyl)-2-methyl-5-oxo-4-thiophen-3-yl-1,4,5,6,7,8-hexahydro-quinoline-3-carboxylic acid 2-ethylsulfanyl-ethyl ester; 7-(3,4-Dimethoxy-phenyl)-4-(3-hydroxy-phenyl)-2-methyl-5-oxo-1,4,5,6,7,8-hexahydro-quinoline-3-carboxylic acid propyl ester; 7-(3,4-Dimethoxy-phenyl)-4-(3-hydroxy-phenyl)-2-methyl-5-oxo-1,4,5,6,7,8-hexahydro-quinoline-3-carboxylic acid isobutyl ester; 4-Furan-2-yl-2-methyl-5-oxo-7-phenyl-1,4,5,6,7,8-hexahydro-quinoline-3-carboxylic acid butyl ester; 4-(3-Hydroxy-phenyl)-2-methyl-5-oxo-7-phenyl-1,4,5,6,7,8-hexahydro-quinoline-3-carboxylic acid methyl ester; 4-(3-Hydroxy-phenyl)-7-(4-methoxy-phenyl)-2-methyl-5-oxo-1,4,5,6,7,8-hexahydro-quinoline-3-carboxylic acid methyl ester; 7-(3,4-Dimethoxy-phenyl)-4-(3-hydroxy-phenyl)-2-methyl-5-oxo-1,4,5,6,7,8-hexahydro-quinoline-3-carboxylic acid butyl ester; 2-Methyl-5-oxo-7-phenyl-4-thiophen-2-yl-1,4,5,6,7,8-hexahydro-quinoline-3-carboxylic acid isopropyl ester; 4-(3-Hydroxy-phenyl)-2,7,7-trimethyl-5-oxo-1,4,5,6,7,8-hexahydro-quinoline-3-carboxylic acid cyclopentyl ester; 7-(3,4-Dimethoxy-phenyl)-4-(3-hydroxy-phenyl)-2-methyl-5-oxo-1,4,5,6,7,8-hexahydro-quinoline-3-carboxylic acid ethyl ester; 2-Methyl-4-(5-methyl-furan-2-yl)-5-oxo-7-phenyl-1,4,5,6,7,8-hexahydro-quinoline-3-carboxylic acid ethyl ester; 7-(3,4-Dimethoxy-phenyl)-4-(3-hydroxy-phenyl)-2-methyl-5-oxo-1,4,5,6,7,8-hexahydro-quinoline-3-carboxylic acid 2-phenoxy-ethyl ester; 4-(3-Hydroxy-phenyl)-2,7-dimethyl-5-oxo-1,4,5,6,7,8-hexahydro-quinoline-3,6-dicarboxylic acid 3-cyclopentyl ester 6-methyl ester; 7-(3,4-Dimethoxy-phenyl)-4-(3-hydroxy-phenyl)-2-methyl-5-oxo-1,4,5,6,7,8-hexahydro-quinoline-3-carboxylic acid 2-ethoxy-ethyl ester; 7-(2-Methoxy-phenyl)-2-methyl-5-oxo-4-thiophen-3-yl-1,4,5,6,7,8-hexahydro-quinoline-3-carboxylic acid 2-isopropoxy-ethyl ester; 7-(3-Methoxy-phenyl)-2-methyl-5-oxo-4-thiophen-2-yl-1,4,5,6,7,8-hexahydro-quinoline-3-carboxylic acid methyl ester; 7-(3,4-Dimethoxy-phenyl)-4-(3-hydroxy-phenyl)-2-methyl-5-oxo-1,4,5,6,7,8-hexahydro-quinoline-3-carboxylic acid phenethyl ester; 7-(3-Methoxy-phenyl)-2-methyl-4-(5-methyl-furan-2-yl)-5-oxo-1,4,5,6,7,8-hexahydro-quinoline-3-carboxylic acid methyl ester; 4-(2-Isopropoxy-phenyl)-1-(tetrahydro-furan-2-ylmethyl)-1,4-dihydro-pyridine-3,5-dicarboxylic acid dimethyl ester; 7-(4-Methoxy-phenyl)-2-methyl-5-oxo-4-pyridin-3-yl-1,4,5,6,7,8-hexahydro-quinoline-3-carboxylic acid cyclopentyl ester; 7-(2-Methoxy-phenyl)-2-methyl-5-oxo-4-thiophen-3-yl-1,4,5,6,7,8-hexahydro-quinoline-3-carboxylic acid 2-methoxy-ethyl ester; 4-(2-Isopropoxy-phenyl)-2-methyl-5-oxo-7-phenyl-1,4,5,6,7,8-hexahydro-quinoline-3-carboxylic acid methyl ester; 4-(2-Isopropoxy-phenyl)-7-(4-methoxy-phenyl)-2-methyl-5-oxo-1,4,5,6,7,8-hexahydro-quinoline-3-carboxylic acid isopropyl ester; 4-(2-Isopropoxy-phenyl)-2-methyl-5-oxo-7-phenyl-1,4,5,6,7,8-hexahydro-quinoline-3-carboxylic acid benzyl ester; 4-(2-Isopropoxy-phenyl)-2-methyl-5-oxo-7-phenyl-1,4,5,6,7,8-hexahydro-quinoline-3-carboxylic acid cyclohexyl ester; 4-(2-Isopropoxy-phenyl)-2-methyl-5-oxo-7-phenyl-1,4,5,6,7,8-hexahydro-quinoline-3-carboxylic acid 2-phenoxy-ethyl ester; 7-(4-Chloro-phenyl)-4-(2-isopropoxy-phenyl)-2-methyl-5-oxo-1,4,5,6,7,8-hexahydro-quinoline-3-carboxylic acid methyl ester; 4-(2-Isopropoxy-phenyl)-1,4-dihydro-pyridine-3,5-dicarboxylic acid dimethyl ester; 4-(2-Isopropoxy-phenyl)-1-methyl-1,4-dihydro-pyridine-3,5-dicarboxylic acid dimethyl ester; 4-(2-Isopropoxy-phenyl)-7-(4-methoxy-phenyl)-2-methyl-5-oxo-1,4,5,6,7,8-hexahydro-quinoline-3-carboxylic acid ethyl ester; 2-Methyl-5-oxo-7-phenyl-4-pyridin-3-yl-1,4,5,6,7,8-hexahydro-quinoline-3-carboxylic acid sec-butyl ester; 2-Methyl-4-(5-methyl-furan-2-yl)-5-oxo-7-phenyl-1,4,5,6,7,8-hexahydro-quinoline-3-carboxylic acid methyl ester; 2-Methyl-5-oxo-7-phenyl-4-pyridin-3-yl-1,4,5,6,7,8-hexahydro-quinoline-3-carboxylic acid propyl ester; 7-(4-Chloro-phenyl)-2-methyl-5-oxo-4-pyridin-3-yl-1,4,5,6,7,8-hexahydro-quinoline-3-carboxylic acid ethyl ester; 4-(3-Hydroxy-phenyl)-2,7-dimethyl-5-oxo-1,4,5,6,7,8-hexahydro-quinoline-3,6-dicarboxylic acid 3-ethyl ester 6-methyl ester; 7-(3,4-Dimethoxy-phenyl)-4-(3-hydroxy-phenyl)-2-methyl-5-oxo-1,4,5,6,7,8-hexahydro-quinoline-3-carboxylic acid 2-isopropoxy-ethyl ester; 2-Methyl-5-oxo-7-phenyl-4-pyridin-3-yl-1,4,5,6,7,8-hexahydro-quinoline-3-carboxylic acid phenethyl ester; 2-Methyl-5-oxo-7-phenyl-4-pyridin-3-yl-1,4,5,6,7,8-hexahydro-quinoline-3-carboxylic acid methyl ester; 7-(4-Methoxy-phenyl)-2-methyl-5-oxo-4-pyridin-3-yl-1,4,5,6,7,8-hexahydro-quinoline-3-carboxylic acid isopropyl ester; 2-Methyl-5-oxo-7-phenyl-4-pyridin-3-yl-1,4,5,6,7,8-hexahydro-quinoline-3-carboxylic acid cyclopentyl ester; 2-Methyl-5-oxo-7-phenyl-4-pyridin-3-yl-1,4,5,6,7,8-hexahydro-quinoline-3-carboxylic acid ethyl ester; 7-(4-Chloro-phenyl)-2-methyl-5-oxo-4-pyridin-3-yl-1,4,5,6,7,8-hexahydro-quinoline-3-carboxylic acid isopropyl ester; 2-Methyl-5-oxo-7-phenyl-4-pyridin-3-yl-1,4,5,6,7,8-hexahydro-quinoline-3-carboxylic acid 2-methoxy-ethyl ester; 2-Methyl-5-oxo-7-phenyl-4-pyridin-3-yl-1,4,5,6,7,8-hexahydro-quinoline-3-carboxylic acid isopropyl ester; 2-Methyl-5-oxo-7-phenyl-4-pyridin-3-yl-1,4,5,6,7,8-hexahydro-quinoline-3-carboxylic acid benzyl ester; 4-(2,3-Dimethoxy-phenyl)-2,7-dimethyl-5-oxo-1,4,5,6,7,8-hexahydro-quinoline-3,6-dicarboxylic acid 3-ethyl ester 6-methyl ester; 4-(2-Ethoxy-phenyl)-2,7-dimethyl-5-oxo-1,4,5,6,7,8-hexahydro-quinoline-3,6-dicarboxylic acid 3-ethyl ester 6-methyl ester; 4-(2-Chloro-phenyl)-2,7-dimethyl-5-oxo-1,4,5,6,7,8-hexahydro-quinoline-3,6-dicarboxylic acid 3-ethyl ester 6-methyl ester; 4-(3-Ethoxy-phenyl)-2,7-dimethyl-5-oxo-1,4,5,6,7,8-hexahydro-quinoline-3,6-dicarboxylic acid 3-ethyl ester 6-methyl ester; 4-(3-Methoxy-phenyl)-2,7-dimethyl-5-oxo-1,4,5,6,7,8-hexahydro-quinoline-3,6-dicarboxylic acid 3-ethyl ester 6-methyl ester; 2,7-Dimethyl-4-(5-methyl-furan-2-yl)-5-oxo-1,4,5,6,7,8-hexahydro-quinoline-3,6-dicarboxylic acid 3-(2-ethylsulfanyl-ethyl)ester 6-methyl ester; 4-(4-Hydroxy-phenyl)-2,7-dimethyl-5-oxo-1,4,5,6,7,8-hexahydro-quinoline-3,6-dicarboxylic acid 3-ethyl ester 6-methyl ester; 4-(3-Fluoro-phenyl)-2,7-dimethyl-5-oxo-1,4,5,6,7,8-hexahydro-quinoline-3,6-dicarboxylic acid 3-ethyl ester 6-methyl ester; 2,7-Dimethyl-4-(5-methyl-furan-2-yl)-5-oxo-1,4,5,6,7,8-hexahydro-quinoline-3,6-dicarboxylic acid 3-ethyl ester 6-methyl ester; 2,7-Dimethyl-5-oxo-4-thiophen-2-yl-1,4,5,6,7,8-hexahydro-quinoline-3,6-dicarboxylic acid 3-ethyl ester 6-methyl ester; 4-(4-Fluoro-phenyl)-2,7-dimethyl-5-oxo-1,4,5,6,7,8-hexahydro-quinoline-3,6-dicarboxylic acid 3-ethyl ester 6-methyl ester; 4-(4-Dimethylamino-phenyl)-2,7-dimethyl-5-oxo-1,4,5,6,7,8-hexahydro-quinoline-3,6-dicarboxylic acid 3-ethyl ester 6-methyl ester; 4-(2-Chloro-6-fluoro-phenyl)-2,7-dimethyl-5-oxo-1,4,5,6,7,8-hexahydro-quinoline-3,6-dicarboxylic acid 3-ethyl ester 6-methyl ester; 4-(4-Hydroxy-3-methoxy-phenyl)-2,7-dimethyl-5-oxo-1,4,5,6,7,8-hexahydro-quinoline-3,6-dicarboxylic acid 3-ethyl ester 6-methyl ester; 4-(4-Methoxycarbonyl-phenyl)-2,7-dimethyl-5-oxo-1,4,5,6,7,8-hexahydro-quinoline-3,6-dicarboxylic acid 3-ethyl ester 6-methyl ester; 2,7-Dimethyl-5-oxo-4-phenyl-1,4,5,6,7,8-hexahydro-quinoline-3,6-dicarboxylic acid 3-ethyl ester 6-methyl ester; 4-(4-Methoxy-phenyl)-2,7-dimethyl-5-oxo-1,4,5,6,7,8-hexahydro-quinoline-3,6-dicarboxylic acid 3-ethyl ester 6-methyl ester; 4-(2-Methoxy-phenyl)-2,7-dimethyl-5-oxo-1,4,5,6,7,8-hexahydro-quinoline-3,6-dicarboxylic acid 3-ethyl ester 6-methyl ester; 4-(4-Chloro-phenyl)-2,7-dimethyl-5-oxo-1,4,5,6,7,8-hexahydro-quinoline-3,6-dicarboxylic acid 3-ethyl ester 6-methyl ester; 4-(3,4-Dimethoxy-phenyl)-2,7-dimethyl-5-oxo-1,4,5,6,7,8-hexahydro-quinoline-3,6-dicarboxylic acid 3-ethyl ester 6-methyl ester; 4-(3-Ethoxy-4-hydroxy-phenyl)-2,7-dimethyl-5-oxo-1,4,5,6,7,8-hexahydro-quinoline-3,6-dicarboxylic acid 3-ethyl ester 6-methyl ester; 2,7-Dimethyl-5-oxo-4-phenyl-1,4,5,6,7,8-hexahydro-quinoline-3,6-dicarboxylic acid 3-(2-ethylsulfanyl-ethyl)ester 6-methyl ester; 2,7-Dimethyl-5-oxo-4-o-tolyl-1,4,5,6,7,8-hexahydro-quinoline-3,6-dicarboxylic acid 3-ethyl ester 6-methyl ester; 4-Benzo[1,3]dioxol-5-yl-2,7-dimethyl-5-oxo-1,4,5,6,7,8-hexahydro-quinoline-3,6-dicarboxylic acid 3-ethyl ester 6-methyl ester; 4-(2-Isopropoxy-phenyl)-2,7-dimethyl-5-oxo-1,4,5,6,7,8-hexahydro-quinoline-3,6-dicarboxylic acid 3-ethyl ester 6-methyl ester; 4-(2-Fluoro-phenyl)-2,7-dimethyl-5-oxo-1,4,5,6,7,8-hexahydro-quinoline-3,6-dicarboxylic acid 3-ethyl ester 6-methyl ester; 2,7-Dimethyl-5-oxo-4-(4-propoxy-phenyl)-1,4,5,6,7,8-hexahydro-quinoline-3,6-dicarboxylic acid 3-ethyl ester 6-methyl ester; 4-(3-Hydroxy-4-methoxy-phenyl)-2,7-dimethyl-5-oxo-1,4,5,6,7,8-hexahydro-quinoline-3,6-dicarboxylic acid 3-ethyl ester 6-methyl ester; 2,7-Dimethyl-4-(4-methylsulfanyl-phenyl)-5-oxo-1,4,5,6,7,8-hexahydro-quinoline-3,6-dicarboxylic acid 3-ethyl ester 6-methyl ester; 4-(3-Chloro-phenyl)-2,7-dimethyl-5-oxo-1,4,5,6,7,8-hexahydro-quinoline-3,6-dicarboxylic acid 3-ethyl ester 6-methyl ester; 4-(4-Acetoxy-phenyl)-2,7-dimethyl-5-oxo-1,4,5,6,7,8-hexahydro-quinoline-3,6-dicarboxylic acid 3-ethyl ester 6-methyl ester; 2,7-Dimethyl-4-naphthalen-1-yl-5-oxo-1,4,5,6,7,8-hexahydro-quinoline-3,6-dicarboxylic acid 3-ethyl ester 6-methyl ester; 2,7-Dimethyl-5-oxo-4-p-tolyl-1,4,5,6,7,8-hexahydro-quinoline-3,6-dicarboxylic acid 3-ethyl ester 6-methyl ester; 4-(2,4-Dimethoxy-phenyl)-2,7-dimethyl-5-oxo-1,4,5,6,7,8-hexahydro-quinoline-3,6-dicarboxylic acid 3-ethyl ester 6-methyl ester; 2,7-Dimethyl-4-(5-methyl-thiophen-2-yl)-5-oxo-1,4,5,6,7,8-hexahydro-quinoline-3,6-dicarboxylic acid 3-ethyl ester 6-methyl ester; 2,7-Dimethyl-5-oxo-4-thiophen-2-yl-1,4,5,6,7,8-hexahydro-quinoline-3,6-dicarboxylic acid 3-(2-ethylsulfanyl-ethyl)ester 6-methyl ester; 2,7-Dimethyl-4-(5-methyl-furan-2-yl)-5-oxo-1,4,5,6,7,8-hexahydro-quinoline-3,6-dicarboxylic acid 3-(2-isopropoxy-ethyl) ester 6-methyl ester; 4-(3-Chloro-phenyl)-2,7-dimethyl-5-oxo-1,4,5,6,7,8-hexahydro-quinoline-3,6-dicarboxylic acid 3-isopropyl ester 6-methyl ester; 2,7-Dimethyl-4-(5-methyl-furan-2-yl)-5-oxo-1,4,5,6,7,8-hexahydro-quinoline-3,6-dicarboxylic acid 3-(2-methoxy-ethyl) ester 6-methyl ester; 4-(3-Fluoro-phenyl)-2,7-dimethyl-5-oxo-1,4,5,6,7,8-hexahydro-quinoline-3,6-dicarboxylic acid 6-methyl ester 3-propyl ester; 4-(3-Fluoro-phenyl)-2,7-dimethyl-5-oxo-1,4,5,6,7,8-hexahydro-quinoline-3,6-dicarboxylic acid 3-(2-methoxy-ethyl) ester 6-methyl ester; 2,7-Dimethyl-5-oxo-4-p-tolyl-1,4,5,6,7,8-hexahydro-quinoline-3,6-dicarboxylic acid 3-cyclopentyl ester 6-methyl ester; 2,7-Dimethyl-4-(5-methyl-thiophen-2-yl)-5-oxo-1,4,5,6,7,8-hexahydro-quinoline-3,6-dicarboxylic acid 3-cyclopentyl ester 6-methyl ester; 2,7-Dimethyl-4-(5-methyl-furan-2-yl)-5-oxo-1,4,5,6,7,8-hexahydro-quinoline-3,6-dicarboxylic acid 3-cyclohexyl ester 6-methyl ester; 4-Benzo[1,3]dioxol-5-yl-2,7-dimethyl-5-oxo-1,4,5,6,7,8-hexahydro-quinoline-3,6-dicarboxylic acid 6-methyl ester 3-propyl ester; 4-(4-Dimethylarnino-phenyl)-2,7-dimethyl-5-oxo-1,4,5,6,7,8-hexahydro-quinoline-3,6-dicarboxylic acid 3-isopropyl ester 6-methyl ester; 4-(3-Ethoxy-phenyl)-2,7-dimethyl-5-oxo-1,4,5,6,7,8-hexahydro-quinoline-3,6-dicarboxylic acid 6-methyl ester 3-propyl ester; 4-(4-Dimethylamino-phenyl)-2,7-dimethyl-5-oxo-1,4,5,6,7,8-hexahydro-quinoline-3,6-dicarboxylic acid 6-methyl ester 3-propyl ester; 4-(2-Fluoro-phenyl)-2,7-dimethyl-5-oxo-1,4,5,6,7,8-hexahydro-quinoline-3,6-dicarboxylic acid 3-(2-methoxy-ethyl)ester 6-methyl ester; 2,7-Dimethyl-5-oxo-4-thiophen-2-yl-1,4,5,6,7,8-hexahydro-quinoline-3,6-dicarboxylic acid 3-cyclopentyl ester 6-methyl ester; 4-(4-Chloro-phenyl)-2,7-dimethyl-5-oxo-1,4,5,6,7,8-hexahydro-quinoline-3,6-dicarboxylic acid 6-methyl ester 3-propyl ester; 4-Benzo[1,3]dioxol-5-yl-2,7-dimethyl-5-oxo-1,4,5,6,7,8-hexahydro-quinoline-3,6-dicarboxylic acid 3-isopropyl ester 6-methyl ester; 2,7-Dimethyl-4-naphthalen-2-yl-5-oxo-1,4,5,6,7,8-hexahydro-quinoline-3,6-dicarboxylic acid 3-ethyl ester 6-methyl ester; 4-(4-Chloro-3-fluoro-phenyl)-2,7-dimethyl-5-oxo-1,4,5,6,7,8-hexahydro-quinoline-3,6-dicarboxylic acid 3-ethyl ester 6-methyl ester; 4-(3-Hydroxy-4-methoxy-phenyl)-2,7-dimethyl-5-oxo-1,4,5,6,7,8-hexahydro-quinoline-3,6-dicarboxylic acid 6-methyl ester 3-propyl ester; 4-(3-Fluoro-phenyl)-2,7-dimethyl-5-oxo-1,4,5,6,7,8-hexahydro-quinoline-3,6-dicarboxylic acid 3-isopropyl ester 6-methyl ester; 2,7-Dimethyl-4-(5-methyl-furan-2-yl)-5-oxo-1,4,5,6,7,8-hexahydro-quinoline-3,6-dicarboxylic acid 3-(2-ethoxy-ethyl)ester 6-methyl ester; 2,7-Dimethyl-4-(5-methyl-furan-2-yl)-5-oxo-1,4,5,6,7,8-hexahydro-quinoline-3,6-dicarboxylic acid 3-benzyl ester 6-methyl ester; 4-(4-Fluoro-phenyl)-2,7-dimethyl-5-oxo-1,4,5,6,7,8-hexahydro-quinoline-3,6-dicarboxylic acid 6-methyl ester 3-propyl ester; 2,7-Dimethyl-5-oxo-4-o-tolyl-1,4,5,6,7,8-hexahydro-quinoline-3,6-dicarboxylic acid 6-methyl ester 3-propyl ester; 4-(2-Chloro-6-fluoro-phenyl)-2,7-dimethyl-5-oxo-1,4,5,6,7,8-hexahydro-quinoline-3,6-dicarboxylic acid 6-methyl ester 3-propyl ester; 4-(3-Hydroxy-4-methoxy-phenyl)-2,7-dimethyl-5-oxo-1,4,5,6,7,8-hexahydro-quinoline-3,6-dicarboxylic acid 3-isopropyl ester 6-methyl ester; 4-(4-Chloro-phenyl)-2,7-dimethyl-5-oxo-1,4,5,6,7,8-hexahydro-quinoline-3,6-dicarboxylic acid 3-isopropyl ester 6-methyl ester; 4-(2-Fluoro-phenyl)-2,7-dimethyl-5-oxo-1,4,5,6,7,8-hexahydro-quinoline-3,6-dicarboxylic acid 6-methyl ester 3-propyl ester; 2,7-Dimethyl-5-oxo-4-o-tolyl-1,4,5,6,7,8-hexahydro-quinoline-3,6-dicarboxylic acid 3-(2-methoxy-ethyl)ester 6-methyl ester; 4-(3-Fluoro-phenyl)-2,7-dimethyl-5-oxo-1,4,5,6,7,8-hexahydro-quinoline-3,6-dicarboxylic acid 3-(2-ethoxy-ethyl)ester 6-methyl ester; 2,7-Dimethyl-5-oxo-4-p-tolyl-1,4,5,6,7,8-hexahydro-quinoline-3,6-dicarboxylic acid 3-(2-methoxy-ethyl)ester 6-methyl ester; 4-(3-Chloro-phenyl)-2,7-dimethyl-5-oxo-1,4,5,6,7,8-hexahydro-quinoline-3,6-dicarboxylic acid 3-(2-methoxy-ethyl)ester 6-methyl ester; 4-(4-Fluoro-phenyl)-2,7-dimethyl-5-oxo-1,4,5,6,7,8-hexahydro-quinoline-3,6-dicarboxylic acid 3-(2-methoxy-ethyl)ester 6-methyl ester; 4-(3-Methoxy-phenyl)-2,7-dimethyl-5-oxo-1,4,5,6,7,8-hexahydro-quinoline-3,6-dicarboxylic acid 6-methyl ester 3-propyl ester; 4-(2-Fluoro-phenyl)-2,7-dimethyl-5-oxo-1,4,5,6,7,8-hexahydro-quinoline-3,6-dicarboxylic acid 3-(2-ethoxy-ethyl)ester 6-methyl ester; 4-(2-Methoxy-phenyl)-2,7-dimethyl-5-oxo-1,4,5,6,7,8-hexahydro-quinoline-3,6-dicarboxylic acid 3-isopropyl ester 6-methyl ester; 2,7-Dimethyl-5-oxo-4-thiophen-2-yl-1,4,5,6,7,8-hexahydro-quinoline-3,6-dicarboxylic acid 3-(2-isopropoxy-ethyl)ester 6-methyl ester; 4-(4-Fluoro-phenyl)-2,7-dimethyl-5-oxo-1,4,5,6,7,8-hexahydro-quinoline-3,6-dicarboxylic acid 3-cyclopentyl ester 6-methyl ester; 2,7-Dimethyl-5-oxo-4-thiophen-2-yl-1,4,5,6,7,8-hexahydro-quinoline-3,6-dicarboxylic acid 6-methyl ester 3-propyl ester; 4-(4-Methoxy-phenyl)-2,7-dimethyl-5-oxo-1,4,5,6,7,8-hexahydro-quinoline-3,6-dicarboxylic acid 6-methyl ester 3-propyl ester; 2,7-Dimethyl-5-oxo-4-o-tolyl-1,4,5,6,7,8-hexahydro-quinoline-3,6-dicarboxylic acid 3-cyclopentyl ester 6-methyl ester; 4-(3-Hydroxy-phenyl)-2,7-dimethyl-5-oxo-1,4,5,6,7,8-hexahydro-quinoline-3,6-dicarboxylic acid 3-(2-methoxy-ethyl)ester 6-methyl ester; 4-(2-Methoxy-phenyl)-2,7-dimethyl-5-oxo-1,4,5,6,7,8-hexahydro-quinoline-3,6-dicarboxylic acid 6-methyl ester 3-propyl ester; 4-(2-Fluoro-phenyl)-2,7-dimethyl-5-oxo-1,4,5,6,7,8-hexahydro-quinoline-3,6-dicarboxylic acid 3-isopropyl ester 6-methyl ester; 4-Benzo[1,3]dioxol-5-yl-2,7-dimethyl-5-oxo-1,4,5,6,7,8-hexahydro-quinoline-3,6-dicarboxylic acid dimethyl ester; 4-(2-Fluoro-phenyl)-2,7-dimethyl-5-oxo-1,4,5,6,7,8-hexahydro-quinoline-3,6-dicarboxylic acid 3-cyclopentyl ester 6-methyl ester; 7-(3,4-Dimethoxy-phenyl)-2-methyl-5-oxo-4-thiophen-2-yl-1,4,5,6,7,8-hexahydro-quinoline-3-carboxylic acid cyclohexyl ester; 7-(3,4-Dimethoxy-phenyl)-2-methyl-5-oxo-4-thiophen-2-yl-1,4,5,6,7,8-hexahydro-quinoline-3-carboxylic acid 2-ethylsulfanyl-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid methyl ester; 7-(3,4-Dimethoxy-phenyl)-4-(3-hydroxy-phenyl)-2-methyl-5-oxo-1,4,5,6,7,8-hexahydro-quinoline-3-carboxylic acid methyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid ethyl ester; 7-(3,4-Dimethoxy-phenyl)-2-methyl-5-oxo-4-thiophen-2-yl-1,4,5,6,7,8-hexahydro-quinoline-3-carboxylic acid phenethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid 2-isopropoxy-ethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid 2-phenoxy-ethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid cyclohexyl ester; 7-(3,4-Dimethoxy-phenyl)-2-methyl-4-(5-methyl-furan-2-yl)-5-oxo-1,4,5,6,7,8-hexahydro-quinoline-3-carboxylic acid sec-butyl ester; 7-(3,4-Dimethoxy-phenyl)-2-methyl-4-(5-methyl-furan-2-yl)-5-oxo-1,4,5,6,7,8-hexahydro-quinoline-3-carboxylic acid propyl ester; 7-(3,4-Dimethoxy-phenyl)-2-methyl-4-(5-methyl-furan-2-yl)-5-oxo-1,4,5,6,7,8-hexahydro-quinoline-3-carboxylic acid phenethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid benzyl ester; 7-(3,4-Dimethoxy-phenyl)-2-methyl-4-(5-methyl-furan-2-yl)-5-oxo-1,4,5,6,7,8-hexahydro-quinoline-3-carboxylic acid cyclopentyl ester; 7-(3,4-Dimethoxy-phenyl)-2-methyl-4-(5-methyl-furan-2-yl)-5-oxo-1,4,5,6,7,8-hexahydro-quinoline-3-carboxylic acid ethyl ester; 7-(3,4-Dimethoxy-phenyl)-2-methyl-5-oxo-4-thiophen-2-yl-1,4,5,6,7,8-hexahydro-quinoline-3-carboxylic acid propyl ester; 7-(4-Chloro-phenyl)-2-methyl-5-oxo-4-pyridin-3-yl-1,4,5,6,7,8-hexahydro-quinoline-3-carboxylic acid 2-phenoxy-ethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid isopropyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid isopropyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid cyclohexyl ester; 7-(3,4-Dimethoxy-phenyl)-2-methyl-4-(5-methyl-furan-2-yl)-5-oxo-1,4,5,6,7,8-hexahydro-quinoline-3-carboxylic acid butyl ester; 7-(3,4-Dimethoxy-phenyl)-2-methyl-5-oxo-4-thiophen-2-yl-1,4,5,6,7,8-hexahydro-quinoline-3-carboxylic acid ethyl ester; 7-(3,4-Dimethoxy-phenyl)-2-methyl-5-oxo-4-thiophen-2-yl-1,4,5,6,7,8-hexahydro-quinoline-3-carboxylic acid cyclopentyl ester; 7-(3,4-Dimethoxy-phenyl)-2-methyl-4-(5-methyl-furan-2-yl)-5-oxo-1,4,5,6,7,8-hexahydro-quinoline-3-carboxylic acid 2-methoxy-ethyl ester; 7-(3,4-Dimethoxy-phenyl)-2-methyl-5-oxo-4-thiophen-2-yl-1,4,5,6,7,8-hexahydro-quinoline-3-carboxylic acid sec-butyl ester; 7-(3,4-Dimethoxy-phenyl)-2-methyl-4-(5-methyl-furan-2-yl)-5-oxo-1,4,5,6,7,8-hexahydro-quinoline-3-carboxylic acid 2-ethoxy-ethyl ester; 7-(3,4-Dimethoxy-phenyl)-2-methyl-5-oxo-4-thiophen-2-yl-1,4,5,6,7,8-hexahydro-quinoline-3-carboxylic acid 2-ethoxy-ethyl ester; 7-(3,4-Dimethoxy-phenyl)-2-methyl-5-oxo-4-thiophen-2-yl-1,4,5,6,7,8-hexahydro-quinoline-3-carboxylic acid methyl ester; 7-(3,4-Dimethoxy-phenyl)-2-methyl-4-(5-methyl-furan-2-yl)-5-oxo-1,4,5,6,7,8-hexahydro-quinoline-3-carboxylic acid 2-ethylsulfanyl-ethyl ester; 7-(3,4-Dimethoxy-phenyl)-2-methyl-4-(5-methyl-furan-2-yl)-5-oxo-1,4,5,6,7,8-hexahydro-quinoline-3-carboxylic acid isobutyl ester; 7-(3,4-Dimethoxy-phenyl)-2-methyl-5-oxo-4-thiophen-2-yl-1,4,5,6,7,8-hexahydro-quinoline-3-carboxylic acid benzyl ester; 7-(3,4-Dimethoxy-phenyl)-2-methyl-5-oxo-4-thiophen-2-yl-1,4,5,6,7,8-hexahydro-quinoline-3-carboxylic acid isobutyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid isobutyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid sec-butyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid 2-ethylsulfanyl-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid 2-phenoxy-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid 2-ethoxy-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid butyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid propyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid 2-methoxy-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid cyclopentyl ester; 7-(3,4-Dimethoxy-phenyl)-2-methyl-5-oxo-4-thiophen-2-yl-1,4,5,6,7,8-hexahydro-quinoline-3-carboxylic acid 2-methoxy-ethyl ester; 7-(3,4-Dimethoxy-phenyl)-4-(3-hydroxy-phenyl)-2-methyl-5-oxo-1,4,5,6,7,8-hexahydro-quinoline-3-carboxylic acid 2-methoxy-ethyl ester; 7-(3,4-Dimethoxy-phenyl)-2-methyl-5-oxo-4-thiophen-2-yl-1,4,5,6,7,8-hexahydro-quinoline-3-carboxylic acid butyl ester; 7-(3,4-Dimethoxy-phenyl)-2-methyl-5-oxo-4-thiophen-2-yl-1,4,5,6,7,8-hexahydro-quinoline-3-carboxylic acid isopropyl ester; 7-(2-Methoxy-phenyl)-2-methyl-5-oxo-4-thiophen-2-yl-1,4,5,6,7,8-hexahydro-quinoline-3-carboxylic acid 2-ethylsulfanyl-ethyl ester; 7-(3,4-Dimethoxy-phenyl)-2-methyl-5-oxo-4-thiophen-2-yl-1,4,5,6,7,8-hexahydro-quinoline-3-carboxylic acid 2-phenoxy-ethyl ester; 7-(3,4-Dimethoxy-phenyl)-2-methyl-5-oxo-4-thiophen-2-yl-1,4,5,6,7,8-hexahydro-quinoline-3-carboxylic acid 2-isopropoxy-ethyl ester; 7-(2-Methoxy-phenyl)-2-methyl-5-oxo-4-thiophen-2-yl-1,4,5,6,7,8-hexahydro-quinoline-3-carboxylic acid isopropyl ester; 7-(2-Methoxy-phenyl)-2-methyl-5-oxo-4-thiophen-2-yl-1,4,5,6,7,8-hexahydro-quinoline-3-carboxylic acid methyl ester; 7-(2-Methoxy-phenyl)-2-methyl-5-oxo-4-thiophen-2-yl-1,4,5,6,7,8-hexahydro-quinoline-3-carboxylic acid propyl ester; 7-(2-Methoxy-phenyl)-2-methyl-5-oxo-4-thiophen-2-yl-1,4,5,6,7,8-hexahydro-quinoline-3-carboxylic acid 2-methoxy-ethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid benzyl ester; 7-(2-Methoxy-phenyl)-2-methyl-5-oxo-4-thiophen-2-yl-1,4,5,6,7,8-hexahydro-quinoline-3-carboxylic acid tetrahydro-furan-2-ylmethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid phenethyl ester; 7-(3,4-Dimethoxy-phenyl)-4-(3-hydroxy-4-methoxy-phenyl)-2-methyl-5-oxo-1,4,5,6,7,8-hexahydro-quinoline-3-carboxylic acid 2-isopropoxy-ethyl ester; 7-(2-Chloro-phenyl)-2-methyl-4-(5-methyl-thiophen-2-yl)-5-oxo-1,4,5,6,7,8-hexahydro-quinoline-3-carboxylic acid methyl ester; 7-(3-Methoxy-phenyl)-2-methyl-4-(5-methyl-thiophen-2-yl)-5-oxo-1,4,5,6,7,8-hexahydro-quinoline-3-carboxylic acid methyl ester; 7-(4-Chloro-phenyl)-4-(2-isopropoxy-phenyl)-2-methyl-5-oxo-1,4,5,6,7,8-hexahydro-quinoline-3-carboxylic acid 2-ethylsulfanyl-ethyl ester; 7-(4-Chloro-phenyl)-4-(2-isopropoxy-phenyl)-2-methyl-5-oxo-1,4,5,6,7,8-hexahydro-quinoline-3-carboxylic acid isobutyl ester; 4-(2-Isopropoxy-phenyl)-7-(4-methoxy-phenyl)-2-methyl-5-oxo-1,4,5,6,7,8-hexahydro-quinoline-3-carboxylic acid phenethyl ester; 4-(2-Isopropoxy-phenyl)-7-(4-methoxy-phenyl)-2-methyl-5-oxo-1,4,5,6,7,8-hexahydro-quinoline-3-carboxylic acid propyl ester

III. Pharmaceutical Compositions

In addition to the compounds provided above, compositions for modulating C5a activity in humans and animals will typically contain a pharmaceutical carrier or diluent.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present invention are employed. As used herein, topical application is also meant to include the use of mouth washes and gargles.

The compounds of this invention may also be coupled a carrier that is a suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the invention may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the invention, the compound of the invention is coupled to a polymer or semipermeable polymer matrix that is formed as a stent or stent-graft device.

IV. Methods of Treating Diseases and Disorders Modulated by C5a

The compounds of the invention may be used as agonists, (preferably) antagonists, partial agonists, inverse agonists, of C5a receptors in a variety of contexts, both in vitro and in vivo. In one embodiment, the compounds of the invention are C5aR antagonist that can be used to inhibit the binding of C5a receptor ligand (e.g., C5a) to C5a receptor in vitro or in vivo. In general, such methods comprise the step of contacting a C5a receptor with a sufficient amount of one or more C5a receptor modulators as provided herein, in the presence of C5a receptor ligand in aqueous solution and under conditions otherwise suitable for binding of the ligand to C5a receptor. The C5a receptor may be present in suspension (e.g., in an isolated membrane or cell preparation), or in a cultured or isolated cell.

Preferably, the amount of C5a receptor modulator contacted with the receptor should be sufficient to inhibit C5a binding to C5a receptor in vitro as measured, for example, using a radioligand binding assay, calcium mobilization assay, or chemotaxis assay as described herein.

In one embodiment of the invention, the C5a modulators of the invention are used to modulate, preferably inhibit, the signal-transducing activity of a C5a receptor, for example, by contacting one or more compound(s) of the invention with a C5a receptor (either in vitro or in vivo) under conditions suitable for binding of the modulator(s) to the receptor. The receptor may be present in solution or suspension, in a cultured or isolated cell preparation or within a patient. Any modulation of the signal transducing activity may be assessed by detecting an effect on calcium ion calcium mobilization or by detecting an effect on C5a receptor-mediated cellular chemotaxis. In general, an effective amount of C5a modulator(s) is an amount sufficient to modulate C5a receptor signal transducing activity in vitro within a calcium mobilization assay or C5a receptor-mediated cellular chemotaxis within a migration assay.

When compounds of the invention are used to inhibit C5a receptor-mediated cellular chemotaxis, preferably leukocyte (e.g., neutrophil) chemotaxis, in an in vitro chemotaxis assay, such methods comprise contacting white blood cells (particularly primate white blood cells, especially human white blood cells) with one or more compounds of the invention. Preferably the concentration is sufficient to inhibit chemotaxis of white blood cells in an in vitro chemotaxis assay, so that the levels of chemotaxis observed in a control assay are significantly higher, as described above, than the levels observed in an assay to which a compound of the invention has been added.

In another embodiment, the compounds of the present invention further can be used for treating patients suffering from conditions that are responsive to C5a receptor modulation. As used herein, the term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms). As used herein, a condition is considered “responsive to C5a receptor modulation” if modulation of C5a receptor activity results in the reduction of inappropriate activity of a C5a receptor. As used herein, the term “patients” include primates (especially humans), domesticated companion animals (such as dogs, cats, horses, and the like) and livestock (such as cattle, pigs, sheep, and the like), with dosages as described herein.

Conditions that can be Treated by C5a Modulation:

Autoimmune disorders—e.g., Rheumatoid arthritis, systemic lupus erythematosus, Guillain-Barre syndrome, pancreatitis, lupus nephritis, lupus glomerulonephritis, psoriasis, Crohn's disease, vasculitis, irritable bowel syndrome, dermatomyositis, multiple sclerosis, bronchial asthma, pemphigus, pemphigoid, scleroderma, myasthenia gravis, autoimmune hemolytic and thrombocytopenic states, Goodpasture's syndrome (and associated glomerulonephritis and pulmonary hemorrhage), immunovasculitis, tissue graft rejection, hyperacute rejection of transplanted organs; and the like.

Inflammatory disorders and related conditions—e.g., Neutropenia, sepsis, septic shock, Alzheimer's disease, multiple sclerosis, stroke, inflammatory bowel disease (IBD), inflammation associated with severe burns, lung injury, and ischemia-reperfusion injury, osteoarthritis, as well as acute (adult) respiratory distress syndrome (ARDS), chronic pulmonary obstructive disorder (COPD), systemic inflammatory response syndrome (SIRS), atopic dermatitis, chronic urticaria and multiple organ dysfunction syndrome (MODS). Also included are pathologic sequellae associated with insulin-dependent diabetes mellitus (including diabetic retinopathy), lupus nephropathy, Heyman nephritis, membranous nephritis and other forms of glomerulonephritis, contact sensitivity responses, and inflammation resulting from contact of blood with artificial surfaces that can cause complement activation, as occurs, for example, during extracorporeal circulation of blood (e.g., during hemodialysis or via a heart-lung machine, for example, in association with vascular surgery such as coronary artery bypass grafting or heart valve replacement), or in association with contact with other artificial vessel or container surfaces (e.g., ventricular assist devices, artificial heart machines, transfusion tubing, blood storage bags, plasmapheresis, plateletpheresis, and the like).

Cardiovascular and Cerebrovascular Disorders—e.g., myocardial infarction, coronary thrombosis, vascular occlusion, post-surgical vascular reocclusion, atherosclerosis, traumatic central nervous system injury, and ischemic heart disease. In one embodiment, an effective amount of a compound of the invention may be administered to a patient at risk for myocardial infarction or thrombosis (i.e., a patient who has one or more recognized risk factor for myocardial infarction or thrombosis, such as, but not limited to, obesity, smoking, high blood pressure, hypercholesterolemia, previous or genetic history of myocardial infarction or thrombosis) in order reduce the risk of myocardial infarction or thrombosis.

HIV infection and AIDS—C5a receptor modulators provided herein may be used to inhibit HIV infection, delay AIDS progression or decrease the severity of symptoms or HIV infection and AIDS.

Neurodegenerative disorders and related diseases—Within further aspects, C5a antagonists provided herein may be used to treat Alzheimer's disease, multiple sclerosis, and cognitive function decline associated with cardiopulmonary bypass surgery and related procedures.

In one embodiment of the invention, the compounds of the invention can be used for the treatment of diseases selected from the group consisting of sepsis (and associated disorders), COPD, rheumatoid arthritis, lupus nephritis and multiple sclerosis.

Treatment methods provided herein include, in general, administration to a patient an effective amount of one or more compounds provided herein. Suitable patients include those patients suffering from or susceptible to (i.e., prophylactic treatment) a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

In general, treatment methods provided herein comprise administering to a patient an effective amount of a compound one or more compounds provided herein. In a preferred embodiment, the compound(s) of the invention are preferably administered to a patient (e.g., a human) orally or topically. The effective amount may be an amount sufficient to modulate C5a receptor activity and/or an amount sufficient to reduce or alleviate the symptoms presented by the patient. Preferably, the amount administered is sufficient to yield a plasma concentration of the compound (or its active metabolite, if the compound is a pro-drug) high enough to detectably inhibit white blood cell (e.g., neutrophil) chemotaxis in vitro. Treatment regimens may vary depending on the compound used and the particular condition to be treated; for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, rate of excretion, drug combination (i.e., other drugs being administered to the patient) and the severity of the particular disease undergoing therapy, as well as the judgment of the prescribing medical practitioner. In general, the use of the minimum dose sufficient to provide effective therapy is preferred. Patients may generally be monitored for therapeutic effectiveness using medical or veterinary criteria suitable for the condition being treated or prevented.

Dosage levels of the order of from about 0.1 mg to about 140 mg per kilogram of body weight per day are useful in the treatment or preventions of conditions involving pathogenic C5a activity (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. For compounds administered orally, transdermally, intravaneously, or subcutaneously, it is preferred that sufficient amount of the compound be administered to achieve a serum concentration of 5 ng (nanograms)/mL-10 μg (micrograms)/mL serum, more preferably sufficient compound to achieve a serum concentration of 20 ng-1 μg/ml serum should be administered, most preferably sufficient compound to achieve a serum concentration of 50 ng/ml-200 ng/ml serum should be administered. For direct injection into the synovium (for the treatment of arthritis) sufficient compounds should be administered to achieve a local concentration of approximately 1 micromolar.

Frequency of dosage may also vary depending on the compound used and the particular disease treated. However, for treatment of most disorders, a dosage regimen of 4 times daily, three times daily, or less is preferred, with a dosage regimen of once daily or 2 times daily being particularly preferred. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

In another aspect of the invention, the compounds of the invention can be used in a variety of non-pharmaceutical in vitro and in vivo application. For example, the compounds of the invention may be labeled and used as probes for the detection and localization of C5a receptor (cell preparations or tissue sections samples). The compounds of the invention may also be used as positive controls in assays for C5a receptor activity, i.e., as standards for determining the ability of a candidate agent to bind to C5a receptor, or as radiotracers for positron emission tomography (PET) imaging or for single photon emission computerized tomography (SPECT). Such methods can be used to characterize C5a receptors in living subjects. For example, a C5a receptor modulator may be labeled using any of a variety of well known techniques (e.g., radiolabeled with a radionuclide such as tritium), and incubated with a sample for a suitable incubation time (e.g., determined by first assaying a time course of binding). Following incubation, unbound compound is removed (e.g., by washing), and bound compound detected using any method suitable for the label employed (e.g., autoradiography or scintillation counting for radiolabeled compounds; spectroscopic methods may be used to detect luminescent groups and fluorescent groups). As a control, a matched sample containing labeled compound and a greater (e.g., 10-fold greater) amount of unlabeled compound may be processed in the same manner. A greater amount of detectable label remaining in the test sample than in the control indicates the presence of C5a receptor in the sample. Detection assays, including receptor autoradiography (receptor mapping) of C5a receptor in cultured cells or tissue samples may be performed as described by Kuhar in sections 8.1.1 to 8.1.9 of Current Protocols in Pharmacology (1998) John Wiley & Sons, New York.

The compounds provided herein may also be used within a variety of well known cell separation methods. For example, modulators may be linked to the interior surface of a tissue culture plate or other support, for use as affinity ligands for immobilizing and thereby isolating, C5a receptors (e.g., isolating receptor-expressing cells) in vitro. In one preferred application, a modulator linked to a fluorescent marker, such as fluorescein, is contacted with the cells, which are then analyzed (or isolated) by fluorescence activated cell sorting (FACS).

In addition to the compounds set forth in Table 1, a second set of specific compounds having formula I that are of particular interest for use in the methods of the invention consists of compositions comprising the compounds set forth in Table 2.

TABLE 2

| | |
|---|---|
| 1. | 4-(3-Hydroxy-phenyl)-2-methyl-5-oxo-7-thiophen-2-yl-1,4,5,6,7,8-hexahydro- |
| | quinoline-3-carboxylic acid cyclopentyl ester |
| 2. | 7-(4-Chloro-phenyl)-4-(3-hydroxy-phenyl)-2-methyl-5-oxo-1,4,5,6,7,8-hexahydro- |
| | quinoline-3-carboxylic acid isobutyl ester |
| 3. | 4-(3-Hydroxy-phenyl)-7-(4-methoxy-phenyl)-2-methyl-5-oxo-1,4,5,6,7,8-hexahydro- |
| | quinoline-3-carboxylic acid cyclopentyl ester |
| 4. | 4-(3-Hydroxy-phenyl)-2-methyl-5-oxo-7-phenyl-1,4,5,6,7,8-hexahydro-quinoline-3- |
| | carboxylic acid cyclopentyl ester |
| 5. | 4-(3-Hydroxy-phenyl)-2-methyl-5-oxo-7-phenyl-1,4,5,6,7,8-hexahydro-quinoline-3- |
| | carboxylic acid propyl ester |
| 6. | 4-(3-Hydroxy-phenyl)-2-methyl-5-oxo-7-phenyl-1,4,5,6,7,8-hexahydro-quinoline-3- |
| | carboxylic acid 2-phenoxy-ethyl ester |
| 7. | 2-Methyl-4-(5-methyl-furan-2-yl)-5-oxo-7-phenyl-1,4,5,6,7,8-hexahydro-quinoline-3- |
| | carboxylic acid cyclohexyl ester |
| 8. | 4-(3-Hydroxy-phenyl)-2-methyl-5-oxo-7-phenyl-1,4,5,6,7,8-hexahydro-quinoline-3- |
| | carboxylic acid ethyl ester |
| 9. | 4-(3-Hydroxy-phenyl)-2-methyl-5-oxo-7-thiophen-2-yl-1,4,5,6,7,8-hexahydro- |
| | quinoline-3-carboxylic acid isobutyl ester |
| 10. | 4-(3-Hydroxy-phenyl)-2-methyl-5-oxo-7-thiophen-2-yl-1,4,5,6,7,8-hexahydro- |
| | quinoline-3-carboxylic acid cyclopentyl ester |
| 11. | 7-(2-Methoxy-phenyl)-2-methyl-5-oxo-4-thiophen-3-yl-1,4,5,6,7,8-hexahydro- |
| | quinoline-3-carboxylic acid cyclopentyl ester |
| 12. | 7-(4-Chloro-phenyl)-4-(3-hydroxy-phenyl)-2-methyl-5-oxo-1,4,5,6,7,8-hexahydro- |
| | quinoline-3-carboxylic acid methyl ester |
| 13. | 7-(4-Chloro-phenyl)-4-(3-hydroxy-phenyl)-2-methyl-5-oxo-1,4,5,6,7,8-hexahydro- |
| | quinoline-3-carboxylic acid 2-phenoxy-ethyl ester |
| 14. | 7-(3,4-Dimethoxy-phenyl)-2-methyl-5-oxo-4-phenyl-1,4,5,6,7,8-hexahydro- |
| | quinoline-3-carboxylic acid cyclopentyl ester |
| 15. | 4-(3-Hydroxy-phenyl)-2-methyl-5-oxo-7-phenyl-1,4,5,6,7,8-hexahydro-quinoline-3- |
| | carboxylic acid benzyl ester |
| 16. | 4-(3-Hydroxy-phenyl)-7-(4-methoxy-phenyl)-2-methyl-5-oxo-1,4,5,6,7,8-hexahydro- |
| | quinoline-3-carboxylic acid 2-ethylsulfanyl-ethyl ester |
| 17. | 4-(3-Hydroxy-phenyl)-7-(4-methoxy-phenyl)-2-methyl-5-oxo-1,4,5,6,7,8-hexahydro- |
| | quinoline-3-carboxylic acid 2-phenoxy-ethyl ester |
| 18. | 7-(3,4-Dimethoxy-phenyl)-4-(3-hydroxy-phenyl)-2-methyl-5-oxo-1,4,5,6,7,8- |
| | hexahydro-quinoline-3-carboxylic acid sec-butyl ester |
| 19. | 7-(3,4-Dimethoxy-phenyl)-4-(3-hydroxy-phenyl)-2-methyl-5-oxo-1,4,5,6,7,8- |
| | hexahydro-quinoline-3-carboxylic acid 2-ethylsulfanyl-ethyl ester |
| 20. | 7-(3,4-Dimethoxy-phenyl)-4-(3-hydroxy-phenyl)-2-methyl-5-oxo-1,4,5,6,7,8- |
| | hexahydro-quinoline-3-carboxylic acid isopropyl ester |
| 21. | 7-(2-Methoxy-phenyl)-2-methyl-5-oxo-4-thiophen-3-yl-1,4,5,6,7,8-hexahydro- |
| | quinoline-3-carboxylic acid 2-ethylsulfanyl-ethyl ester |
| 22. | 7-(3,4-Dimethoxy-phenyl)-4-(3-hydroxy-phenyl)-2-methyl-5-oxo-1,4,5,6,7,8- |
| | hexahydro-quinoline-3-carboxylic acid propyl ester |
| 23. | 7-(3,4-Dimethoxy-phenyl)-4-(3-hydroxy-phenyl)-2-methyl-5-oxo-1,4,5,6,7,8- |
| | hexahydro-quinoline-3-carboxylic acid isobutyl ester |
| 24. | 4-Furan-2-yl-2-methyl-5-oxo-7-phenyl-1,4,5,6,7,8-hexahydro-quinoline-3-carboxylic |
| | acid butyl ester |
| 25. | 4-(3-Hydroxy-phenyl)-2-methyl-5-oxo-7-phenyl-1,4,5,6,7,8-hexahydro-quinoline-3- |
| | carboxylic acid methyl ester |
| 26. | 4-(3-Hydroxy-phenyl)-7-(4-methoxy-phenyl)-2-methyl-5-oxo-1,4,5,6,7,8-hexahydro- |
| | quinoline-3-carboxylic acid methyl ester |
| 27. | 7-(3,4-Dimethoxy-phenyl)-4-(3-hydroxy-phenyl)-2-methyl-5-oxo-1,4,5,6,7,8- |
| | hexahydro-quinoline-3-carboxylic acid butyl ester |
| 28. | 2-Methyl-5-oxo-7-phenyl-4-thiophen-2-yl-1,4,5,6,7,8-hexahydro-quinoline-3- |
| | carboxylic acid isopropyl ester |
| 29. | 4-(3-Hydroxy-phenyl)-2,7,7-trimethyl-5-oxo-1,4,5,6,7,8-hexahydro-quinoline-3- |
| | carboxylic acid cyclopentyl ester |
| 30. | 7-(3,4-Dimethoxy-phenyl)-4-(3-hydroxy-phenyl)-2-methyl-5-oxo-1,4,5,6,7,8- |
| | hexahydro-quinoline-3-carboxylic acid ethyl ester |
| 31. | 2-Methyl-4-(5-methyl-furan-2-yl)-5-oxo-7-phenyl-1,4,5,6,7,8-hexahydro-quinoline-3- |
| | carboxylic acid ethyl ester |
| 32. | 7-(3,4-Dimethoxy-phenyl)-4-(3-hydroxy-phenyl)-2-methyl-5-oxo-1,4,5,6,7,8- |
| | hexahydro-quinoline-3-carboxylic acid 2-phenoxy-ethyl ester |
| 33. | 4-(3-Hydroxy-phenyl)-2,7-dimethyl-5-oxo-1,4,5,6,7,8-hexahydro-quinoline-3,6- |
| | dicarboxylic acid 3-cyclopentyl ester 6-methyl ester |
| 34. | 7-(3,4-Dimethoxy-phenyl)-4-(3-hydroxy-phenyl)-2-methyl-5-oxo-1,4,5,6,7,8- |
| | hexahydro-quinoline-3-carboxylic acid 2-ethoxy-ethyl ester |

In the table below, structures and activity are provided for representative compounds described herein. Activity is provided as follows for the binding assay as described herein: +++, 1000 nM<IC₅₀<10000 nM; and ++++, IC₅₀<1000 nM.

Structure

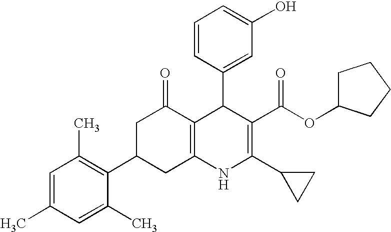

1.001/++++

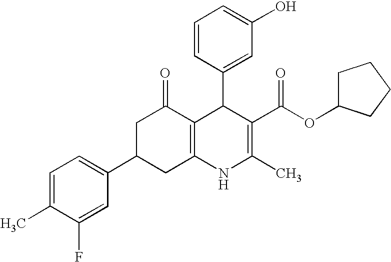

1.002/++++

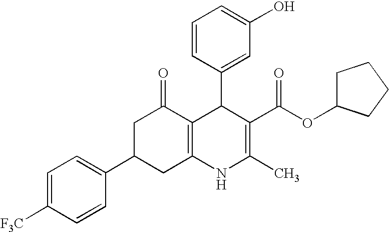

1.003/++++

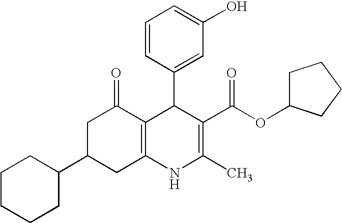

1.004/++++

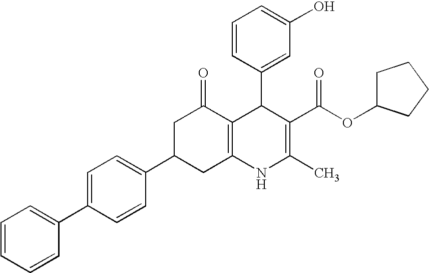

1.005/+++

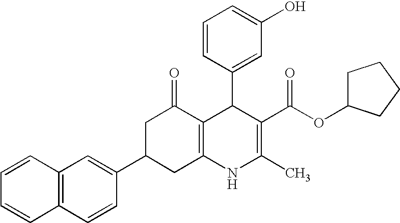

1.006/++++

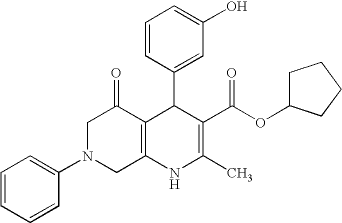

1.007/+++

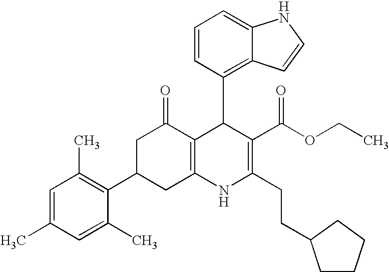

1.008/++++

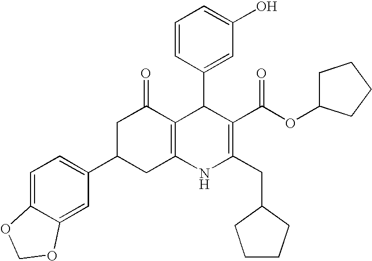

1.009/++++

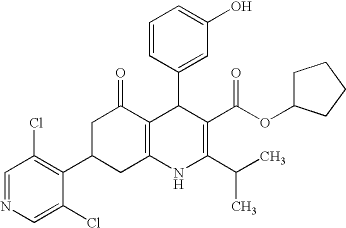

1.010/+++

### V. EXAMPLES

The following examples are offered to illustrate, but not to limit the claimed invention.

Reagents and solvents used below can be obtained from commercial sources such as Aldrich Chemical Co. (Milwaukee, Wis., USA). ¹H-NMR spectra were recorded on a Varian Mercury 400 MHz NMR spectrometer. Significant peaks are provided relative to TMS and are tabulated in the order: multiplicity (s, singlet; d, doublet; t, triplet; q, quartet; m, multiplet) and number of protons. Mass spectrometry results are reported as the ratio of mass over charge, followed by the relative abundance of each ion (in parenthesis). In the examples, a single m/e value is reported for the M+H (or, as noted, M-H) ion containing the most common atomic isotopes. Isotope patterns correspond to the expected formula in all cases. Electrospray ionization (ESI) mass spectrometry analysis was conducted on a Hewlett-Packard MSD electrospray mass spectrometer using the HP1100 HPLC for sample delivery. Normally the analyte was dissolved in methanol at 0.1 mg/mL and 1 microlitre was infused with the delivery solvent into the mass spectrometer, which scanned from 100 to 1500 daltons. All compounds could be analyzed in the positive ESI mode, using acetonitrile/water with 1% formic acid as the delivery solvent. The compounds provided below could also be analyzed in the negative ESI mode, using 2 mM NH₄OAc in acetonitrile/water as delivery system.

The following abbreviations are used in the Examples and throughout the description of the invention:

| | |
|---|---|
| EtOH: | Ethanol |
| EtONa: | Sodium ethoxide |
| THF: | Tetrahydrofuran |
| TLC: | Thin layer chromatography |
| MeOH: | Methanol |

Compounds within the scope of this invention can be synthesized as described below, using a variety of reactions known to the skilled artisan. One skilled in the art will also recognize that alternative methods may be employed to synthesize the target compounds of this invention, and that the approaches described within the body of this document are not exhaustive, but do provide broadly applicable and practical routes to compounds of interest.

Certain molecules claimed in this patent can exist in different enantiomeric and diastereomeric forms and all such variants of these compounds are claimed.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

### Example 1

### Preparation of 4-(3-Hydroxy-phenyl)-2-methyl-5-oxo-7-p-tolyl-1,4,5,6,7,8-hexahydro-quinoline-3-carboxylic acid cyclopentyl ester (7a)

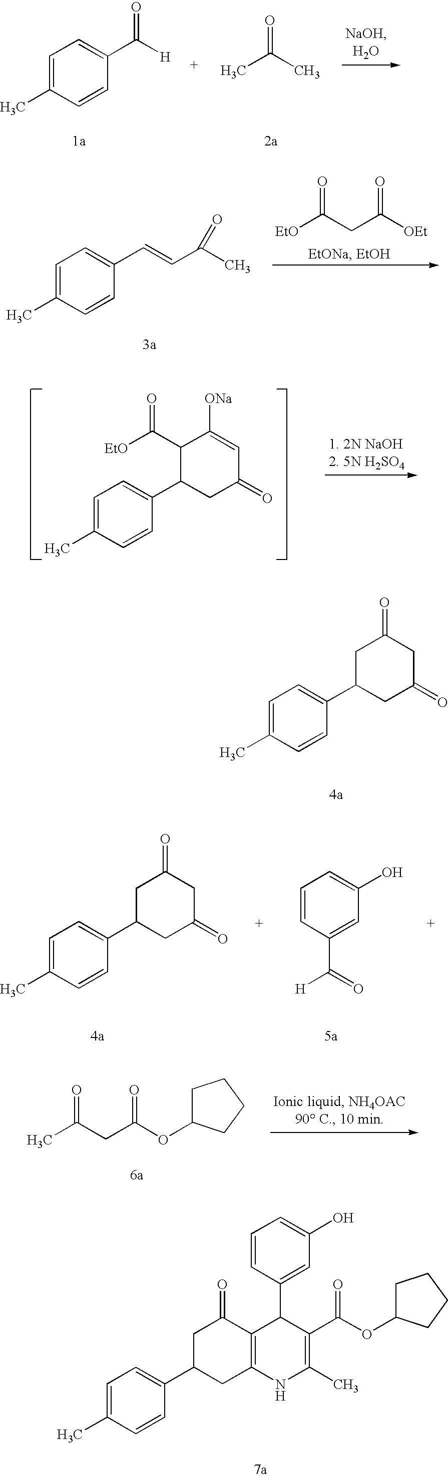

Synthesis of (E)-4-p-Tolyl-but-3-en-2-one (3a): To a solution of 4-methyl-benzaldehyde 1a (2.5 g, 20.8 mmol) in acetone (12 mL) and water (67 mL) was added 1.9 g (23.7 mmol) of 50% aqueous NaOH. The reaction mixture was stirred for three days at room temperature, and then extracted with chloroform (2×15 mL). The combined organic layers were dried over MgSO₄, filtered, and concentrated in vacuo. The product 3a (3.0 g, 91%) was used directly for the next step without further purification: MS (ES) M+H expected=161.2, found=161.2.

Synthesis of 5-p-Tolyl-cyclohexane-1,3-dione (4a): To a solution of sodium ethoxide (0.5 g, 7.5 mmol) in 5 mL of anhydrous ethanol was added diethyl malonate (1.1 mL, 7.5 mmol) followed by 4-p-tolyl-but-3-en-2-one 3a (1.2 g, 7.5 mmol). The reaction mixture was heated under reflux for 6 hours. After cooling down to room temperature, the reaction mixture was poured into a separatory funnel containing chloroform and water. The water layer was collected and condensed under reduced pressure. The residue was dissolved in 5 mL of 2 N NaOH and heated under reflux for 4 hours. After cooling down to room temperature, sulfuric acid (5 N, 5 mL) was added and the reaction mixture was heated under reflux for 2 hours. After cooling down to room temperature, the mixture was filtered and washed with water and the product 4a was collected as a pale yellow solid (0.75 g, 50%): MS (ES) M+H expected=203.3, found=203.4.

Synthesis of 4-(3-Hydroxy-phenyl)-2-methyl-5-oxo-7-p-tolyl-1,4,5,6,7,8-hexahydro-quinoline-3-carboxylic acid cyclopentyl ester (7a): To a small vial was added 3-hydroxybenzaldehyde 5a (48 mg, 0.40 mmol) followed by dione 4a (80 mg, 0.40 mmol), ammonium acetate (46 mg, 0.60 mmol), cyclopentyl acetoacetate 6a (68 mg, 0.40 mmol) and 1-butyl-3-methylimidazolium tetrafluoroborate (11 mg, 0.048 mmol). The reaction mixture was then heated at 90° C. for 10 minutes, then allowed to cool to room temperature. The residue was purified flash silica gel chromatography (50% ethyl acetate/hexanes) to obtain the desired product 7a (110 mg, 60%) as a yellow solid: MS (ES) M+H expected=458.6, found=458.6.

### Example 2

### Preparation of 4-(3-Hydroxy-phenyl)-2-methyl-5-oxo-7-p-tolyl-1,4,5,6,7,8-hexahydro-quinoline-3-carboxylic acid ethyl ester (7b)

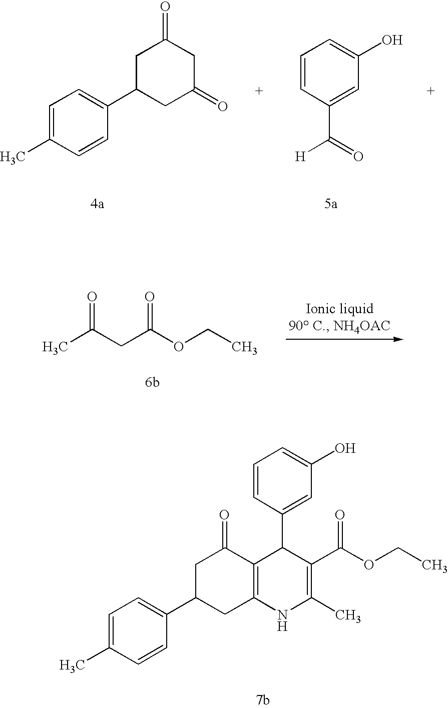

Synthesis of 4-(3-Hydroxy-phenyl)-2-methyl-5-oxo-7-p-tolyl-1,4,5,6,7,8-hexahydro-quinoline-3-carboxylic acid ethyl ester (7b): Compound 7b was prepared according to the same procedure as described for Example 1 using ethyl acetoacetate 6b as the ketoester in the condensation reaction: MS (ES) M+H expected=418.5, found=418.6.

### Example 3

### Preparation of 7-(4-Fluoro-phenyl)-4-(3-hydroxy-phenyl)-2-methyl-5-oxo-1,4,5,6,7,8-hexahydro-quinoline-3-carboxylic acid cyclopentyl ester (7c)

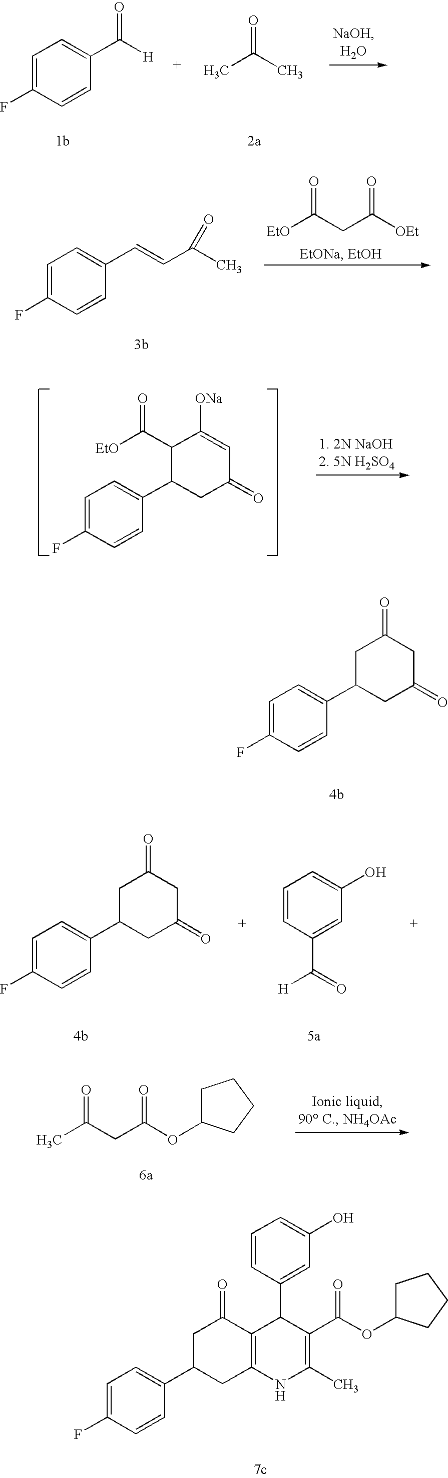

Synthesis of (E)-4-(4-Fluoro-phenyl)-but-3-en-2-one (3b): Compound 3b was prepared following the procedure described in Example 1 for the synthesis of 3a.

Synthesis of 5-(4-Fluoro-phenyl)-cyclohexane-1,3-dione (4b): Compound 4b was prepared following the procedure described in Example 1 for the synthesis of 4a: MS (ES) M+H expected=207.2, found=207.4.

Synthesis of 7-(4-Fluoro-phenyl)-4-(3-hydroxy-phenyl)-2-methyl-5-oxo-1,4,5,6,7,8-hexahydro-quinoline-3-carboxylic acid cyclopentyl ester (7c): Compound 7c was prepared following the procedure described in Example 1 for the synthesis of 7a: diastereomer A (higher Rf), Diastereomer A (higher Rf): ¹HNMR ((CD₃)₂SO) δ 9.12 (s, 1H), 9.07 (t, J=1.4 Hz, 1H), 7.39 (t, J=5.8 Hz, 2H), 7.13 (t, J=7.6 Hz, 2H), 6.96 (td, J=8.0, 2.8 Hz, 1H), 6.61 (m, 2H), 6.46 (d, J=8.0 Hz, 1H), 5.01 (m, 1H), 4.82 (s, 1H), 3.18 (t, J=12.0 Hz, 1H), 2.74 (dd, J=17.2, 11.6 Hz, 1H); MS (ES) M+H expected=462.5, found=462.6. diastereomer B (lower Rf), MS (ES) M+H expected=462.5, found=462.6.

### Example 4

### Preparation of 4-(3-Hydroxy-phenyl)-2-methyl-5-oxo-7-(4-trifluoromethyl-phenyl)-1,4,5,6,7,8-hexahydro-quinoline-3-carboxylic acid cyclopentyl ester (7d)

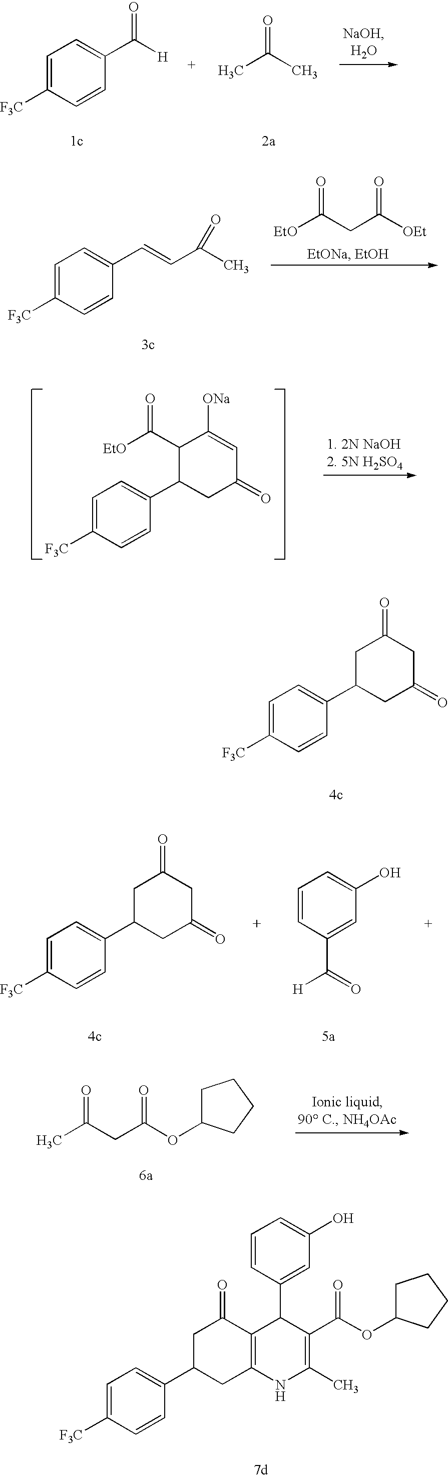

Synthesis of (E)-4-(4-Trifluoromethyl-phenyl)-but-3-en-2-one (3c): Compound 3c was prepared following the procedure described in Example 1 for the synthesis of 3a.

Synthesis of 5-(4-Trifluoromethyl-phenyl)-cyclohexane-1,3-dione (4c): Compound 4c was prepared following the procedure described in Example 1 for the synthesis of 4a.

Synthesis of 4-(3-Hydroxy-phenyl)-2-methyl-5-oxo-7-(4-trifluoromethyl-phenyl)-1,4,5,6,7,8-hexahydro-quinoline-3-carboxylic acid cyclopentyl ester (7d): Compound 7d was prepared following the procedure described in Example 1 for the synthesis of 7a: Diastereomer A (higher Rf), MS (ES) M+H expected=512.5, found=512.6. Diastereomer B (lower Rf): MS (ES) M+H expected=512.5, found=512.6.

### Example 5

### Preparation of 7-Cyclohexyl-4-(3-hydroxy-phenyl)-2-methyl-5-oxo-1,4,5,6,7,8-hexahydro-quinoline-3-carboxylic acid cyclopentyl ester (7e)

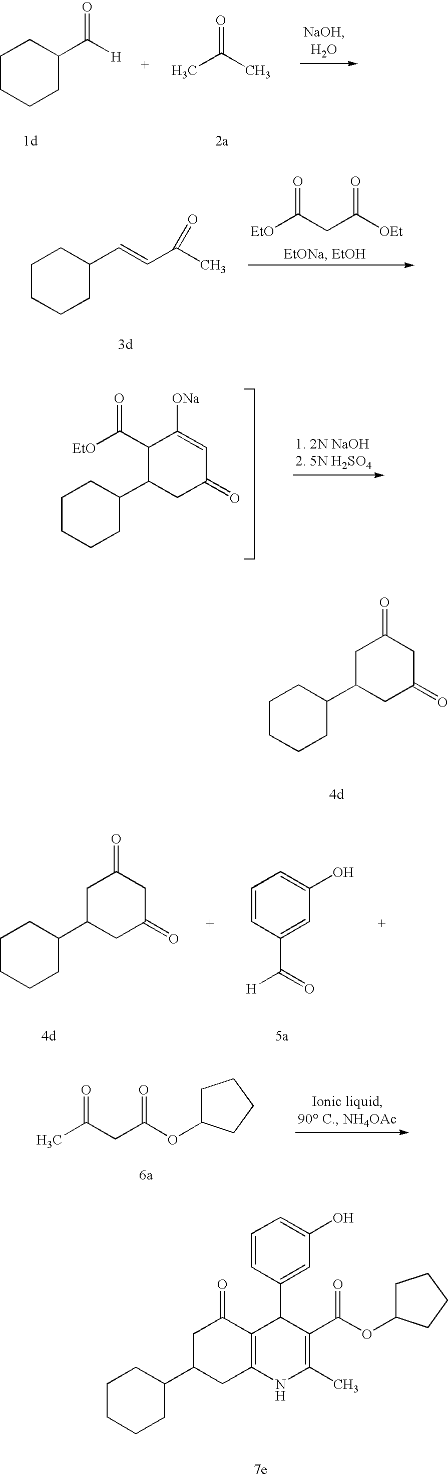

Synthesis of (E)-4-Cyclohexyl-but-3-en-2-one (1d): Compound 1d was prepared following the procedure described in Example 1 for the synthesis of 1a.

Synthesis of Bicyclohexyl-3,5-dione (3d): Compound 3d was prepared following the procedure described in Example 1 for the synthesis of 3a

Synthesis of 7-Cyclohexyl-4-(3-hydroxy-phenyl)-2-methyl-5-oxo-1,4,5,6,7,8-hexahydro-quinoline-3-carboxylic acid cyclopentyl ester (7e): Compound 7e was prepared following the procedure described in Example 1 for the synthesis of 7a: MS (ES) M+H, expected=450.6, found=450.6.

### Example 6

### Preparation of 2-Ethyl-4-(3-hydroxy-phenyl)-5-oxo-7-p-tolyl-1,4,5,6,7,8-hexahydro-quinoline-3-carboxylic acid cyclopentyl ester (7f)

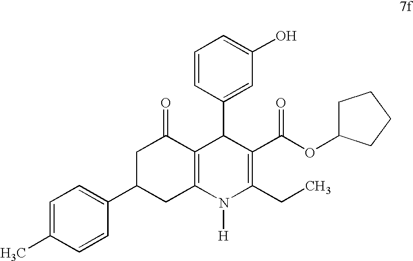

Synthesis 2-Ethyl-4-(3-hydroxy-phenyl)-5-oxo-7-p-tolyl-1,4,5,6,7,8-hexahydro-quinoline-3-carboxylic acid cyclopentyl ester (7f): Compound 7f was prepared following the procedure described in Example 1 for the synthesis of 7a: Diastereomer A (higher Rf), MS (ES) M+H, expected=472.6, found=472.6. Diastereomer B (lower Rf), MS (ES) M+H expected=472.6, found=472.6.

### Example 7

### Preparation of 4-(3-Hydroxy-phenyl)-2-isopropyl-5-oxo-7-p-tolyl-1,4,5,6,7,8-hexahydro-quinoline-3-carboxylic acid cyclopentyl ester (7g)

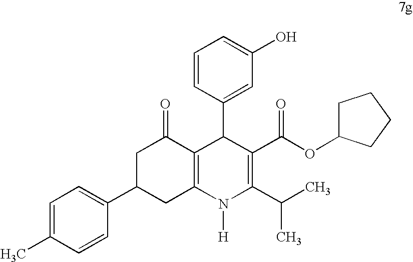

Synthesis 4-(3-Hydroxy-phenyl)-2-isopropyl-5-oxo-7-p-tolyl-1,4,5,6,7,8-hexahydro-quinoline-3-carboxylic acid cyclopentyl ester (7g): Compound 7g was prepared following the procedure described in Example 1 for the synthesis of 7a: Diastereomer A (higher Rf), MS (ES) M+H expected=486.6, found=486.7. Diastereomer B (lower Rf), MS (ES) M+H expected=486.6, found=486.8.

### Example 8

### Preparation of 4-(4-Fluoro-phenyl)-2-methyl-5-oxo-7-p-tolyl-1,4,5,6,7,8-hexahydro-quinoline-3-carboxylic acid cyclopentyl ester (7h)

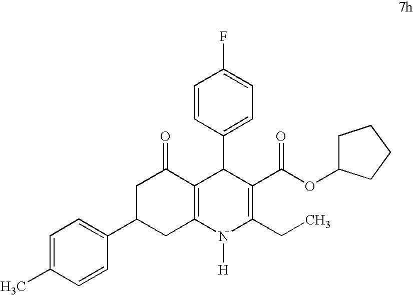

Synthesis 4-(4-Fluoro-phenyl)-2-methyl-5-oxo-7-p-tolyl-1,4,5,6,7,8-hexahydro-quinoline-3-carboxylic acid cyclopentyl ester (7h): Compound 7h was prepared following the procedure described in Example 1 for the synthesis of 7a: Diastereomer A (higher Rf), MS (ES) M+H expected=460.5, found=460.6. Diastereomer B (lower Rf): MS (ES) M+H expected=460.5, found=460.6.

### Example 9

### Preparation of 7-(3-Fluoro-4-methyl-phenyl)-4-(3-hydroxy-phenyl)-2-methyl-5-oxo-1,4,5,6,7,8-hexahydro-quinoline-3-carboxylic acid cyclopentyl ester (7i)

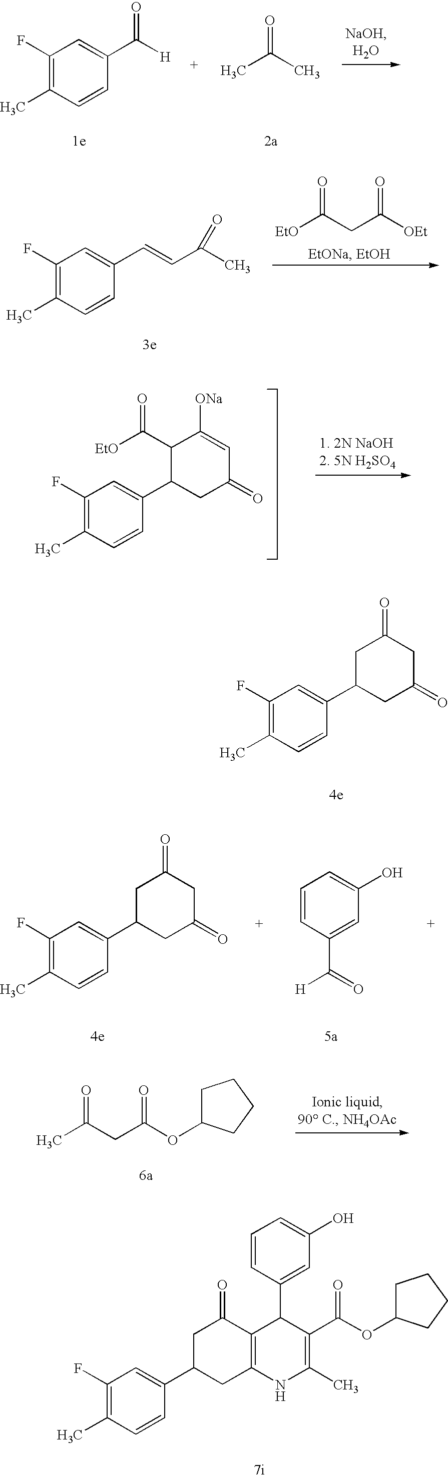

Synthesis of (E)-4-(3-Fluoro-4-methyl-phenyl)-but-3-en-2-one (3e): Compound 3e was prepared following the procedure described in Example 1 for the synthesis of 3a.

Synthesis of 5-(3-Fluoro-4-methyl-phenyl)-cyclohexane-1,3-dione (4e): Compound 4e was prepared following the procedure described in Example 1 for the synthesis of 4a.

Synthesis of 7-(3-Fluoro-4-methyl-phenyl)-4-(3-hydroxy-phenyl)-2-methyl-5-oxo-1,4,5,6,7,8-hexahydro-quinoline-3-carboxylic acid cyclopentyl ester (7i): Compound 7i was prepared following the procedure described in Example 1 for the synthesis of 7a: Diastereomer A (higher Rf), MS (ES) M+H expected=476.5, found=476.6. Diastereomer B (lower Rt): MS (ES) M+H expected=476.5, found=476.6.

### Example 10

### Preparation of 2-Ethyl-7-(3-fluoro-4-methyl-phenyl)-4-(3-hydroxy-phenyl)-5-oxo-1,4,5,6,7,8-hexahydro-quinoline-3-carboxylic acid cyclopentyl ester (7j)

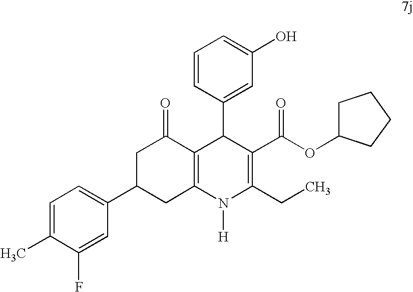

Synthesis 2-Ethyl-7-(3-fluoro-4-methyl-phenyl)-4-(3-hydroxy-phenyl)-5-oxo-1,4,5,6,7,8-hexahydro-quinoline-3-carboxylic acid cyclopentyl ester (7j): Compound 7j was prepared following the procedure described in Example 1 for the synthesis of 7a: Diastereomer A (higher Rf), MS (ES) M+H expected=490.6, found=490.6. Diastereomer B (lower Rf), MS (ES) M+H expected=490.6, found=490.6.

### Example 11

### Preparation of 7-(2,6-Dichloro-phenyl)-4-(3-hydroxy-phenyl)-2-methyl-5-oxo-1,4,5,6,7,8-hexahydro-quinoline-3-carboxylic acid cyclopentyl ester (7k)

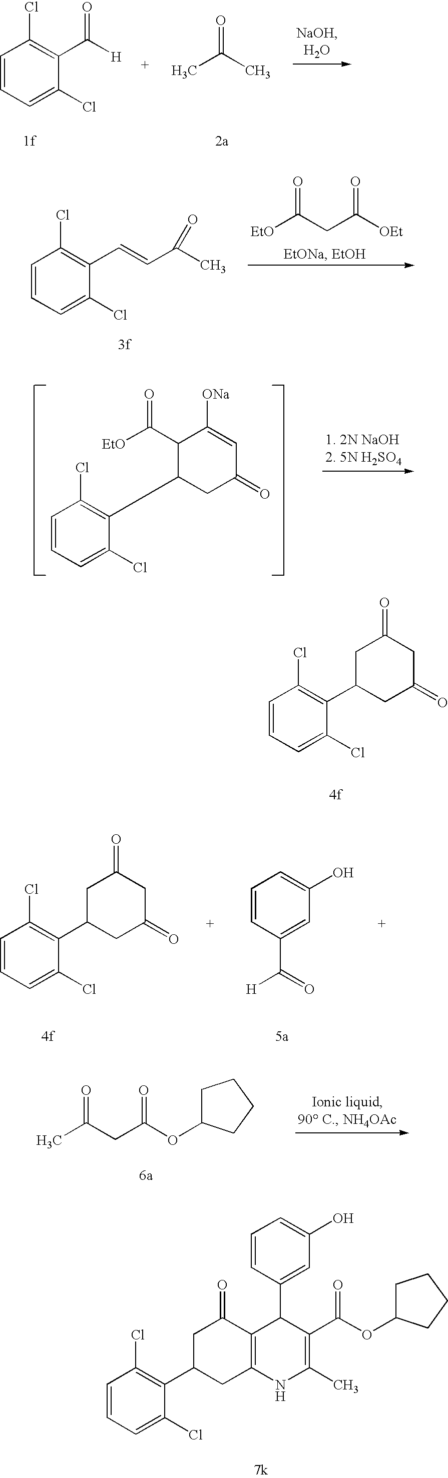

Synthesis of (E)-4-(2,6-Dichloro-phenyl)-but-3-en-2-one (3f): Compound 3f was prepared following the procedure described in Example 1 for the synthesis of 3a.

Synthesis of 5-(2,6-Dichloro-phenyl)-cyclohexane -1,3-dione (4f): Compound 4f was prepared following the procedure described in Example 1 for the synthesis of 4a.

Synthesis of 7-(2,6-Dichloro-phenyl)-4-(3-hydroxy-phenyl)-2-methyl-5-oxo-1,4,5,6,7,8-exahydro-quinoline-3-carboxylic acid cyclopentyl ester (7k): Compound 7k was prepared following the procedure described in Example 1 for the synthesis of 7a: MS (ES) M+H expected=513.4, found=513.5.

### Example 12

### Preparation of 7-(2,6-Dichloro-phenyl)-2-ethyl-4-(3-hydroxy-phenyl)-5-oxo-1,4,5,6,7,8-hexahydro-quinoline-3-carboxylic acid cyclopentyl ester (7l)

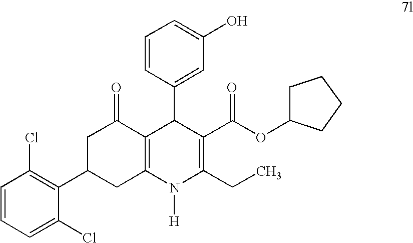

Synthesis of 7-(2,6-Dichloro-phenyl)-2-ethyl-4-(3-hydroxy-phenyl)-5-oxo-1,4,5,6,7,8-hexahydro-quinoline-3-carboxylic acid cyclopentyl ester (7l): Compound 7l was prepared following the procedure described in Example 1 for the synthesis of 7a: MS (ES) M+H expected=527.4, found=527.5.

### Example 13

### Preparation of 7-(2,6-Dichloro-phenyl)-4-(3-hydroxy-phenyl)-2-isopropyl-5-oxo-1,4,5,6,7,8-hexahydro-quinoline-3-carboxylic acid cyclopentyl ester (7m)

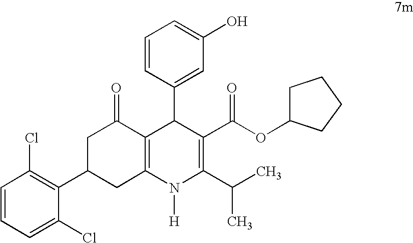

Synthesis of 7-(2,6-Dichloro-phenyl)-4-(3-hydroxy-phenyl)-2-isopropyl-5-oxo-1,4,5,6,7,8-hexahydro-quinoline-3-carboxylic acid cyclopentyl ester (7m): Compound 7m was prepared following the procedure described in Example 1 for the synthesis of 7a: MS (ES) M+H expected=540.5, found=540.6.

### Example 14

### Preparation of 7-(2,4-Dichloro-phenyl)-4-(3-hydroxy-phenyl)-2-methyl-5-oxo-1,4,5,6,7,8-hexahydro-quinoline-3-carboxylic acid cyclopentyl ester (7n)

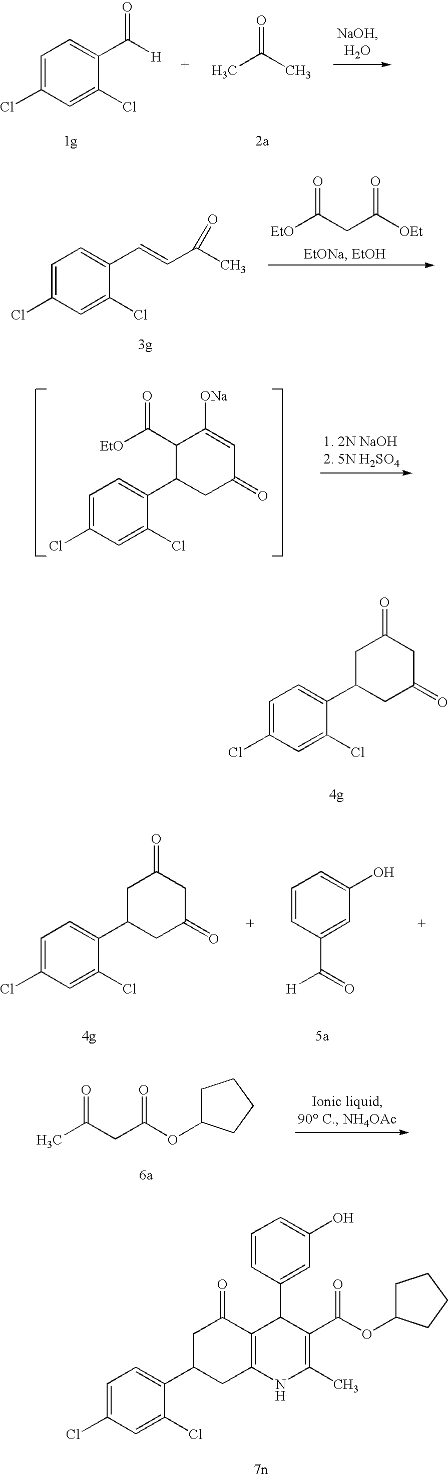

Synthesis of (E)-4-(2,4-Dichloro-phenyl)-but-3-en-2-one (3g): Compound 3g was prepared following the procedure described in Example 1 for the synthesis of 3a.

Synthesis of 5-(2,4-Dichloro-phenyl)-cyclohexane-1,3-dione (4g): Compound 4g was prepared following the procedure described in Example 1 for the synthesis of 4a.

Synthesis of 7-(2,4-Dichloro-phenyl)-4-(3-hydroxy-phenyl)-2-methyl-5-oxo-1,4,5,6,7,8-hexahydro-quinoline-3-carboxylic acid cyclopentyl ester (7n): Compound 7n was prepared following the procedure described in Example 1 for the synthesis of 7a: MS (ES) M+H expected=513.4, found=513.5.

### Example 15

### Preparation of 7-(3,4-Dichloro-phenyl)-4-(3-hydroxy-phenyl)-2-methyl-5-oxo-1,4,5,6,7,8-hexahydro-quinoline-3-carboxylic-acid cyclopentyl ester (7o)

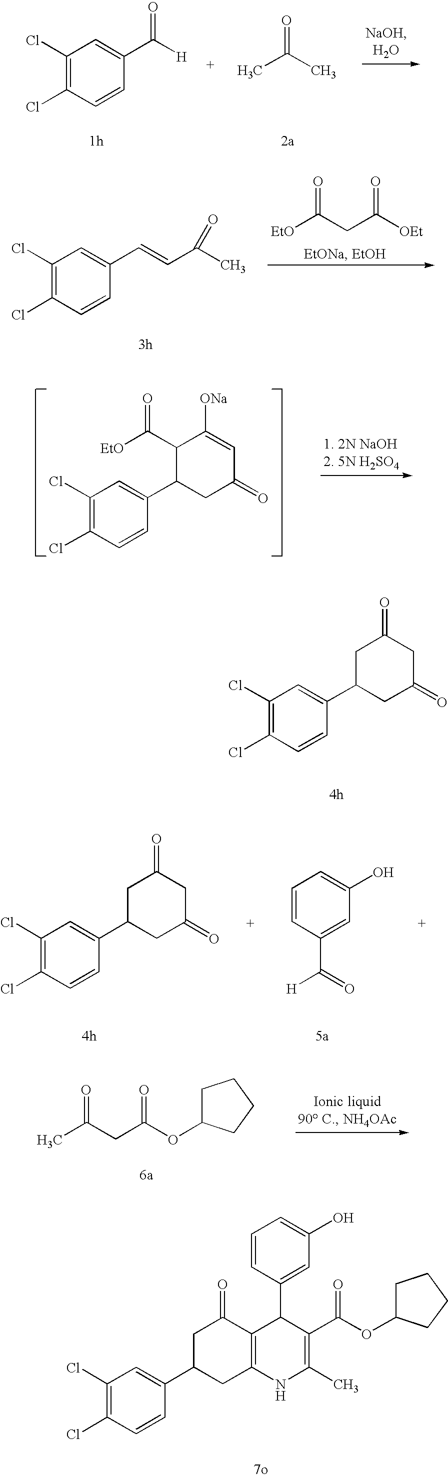

Synthesis of (E)-4-(3,4-Dichloro-phenyl)-but-3-en-2-one (3h): Compound 3h was prepared following the procedure described in Example 1 for the synthesis of 3a.

Synthesis of 5-(3,4-Dichloro-phenyl)-cyclohexane-1,3-dione (4h): Compound 4h was prepared following the procedure described in Example 1 for the synthesis of 4a.

Synthesis of 7-(3,4-Dichloro-phenyl)-4-(3-hydroxy-phenyl)-2-methyl-5-oxo-1,4,5,6,7,8-hexahydro-quinoline-3-carboxylic acid cyclopentyl ester (7o): Compound 7o was prepared following the procedure described in Example 1 for the synthesis of 7a: Diastereomer A (higher Rf), MS (ES) M+H expected=513.4, found=513.5. Diastereomer B (lower Rf): MS (ES) M+H expected=513.4, found=513.6.

### Example 16

### Preparation of 7-(2,6-Dichloro-phenyl)-4-(2,4-dioxo-1,2,3,4-tetrahydro-pyrimidin-5-yl)-2-methyl-5-oxo-1,4,5,6,7,8-hexahydro-quinoline-3-carboxylic acid cyclopentyl ester (7p)

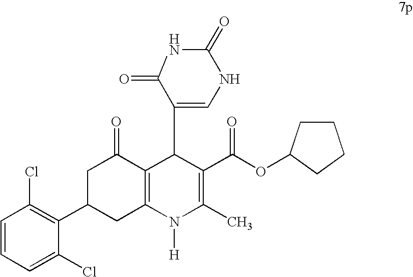

Synthesis of 7-(2,6-Dichloro-phenyl)-4-(2,4-dioxo-1,2,3,4-tetrahydro-pyrimidin-5-yl)-2-methyl-5-oxo-1,4,5,6,7,8-hexahydro-quinoline-3-carboxylic acid cyclopentyl ester (7p): Compound 7p was prepared following the procedure described in Example 1 for the synthesis of 7a: MS (ES) M+H expected=531.4, found=531.5.

### Example 17

### Preparation of 7-(4-Chloro-3-fluoro-phenyl)-4-(3-hydroxy-phenyl)-2-methyl-5-oxo-1,4,5,6,7,8-hexahydro-quinoline-3-carboxylic acid cyclopentyl ester (7q)

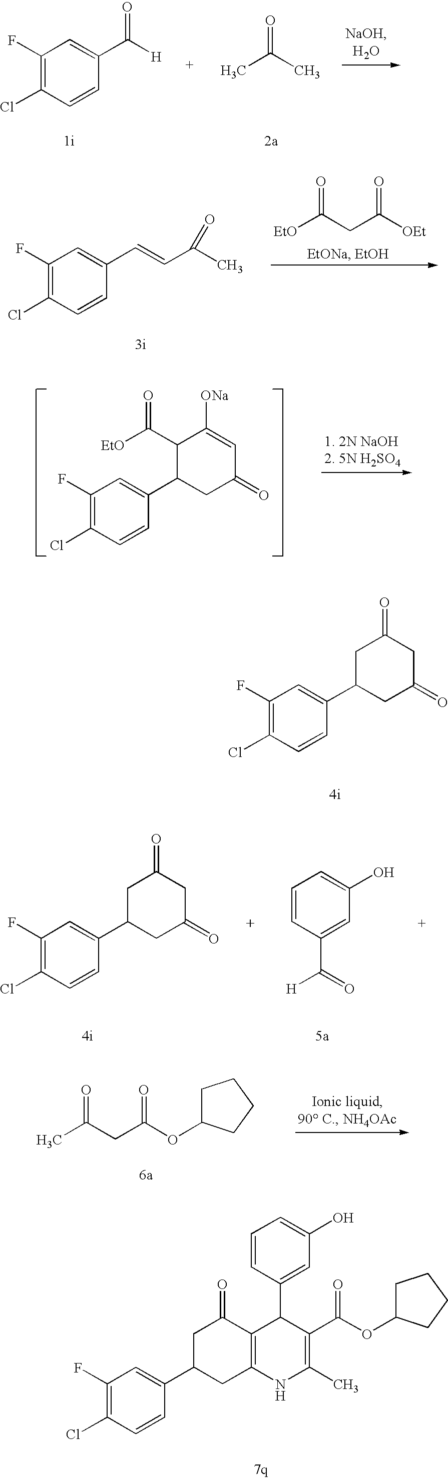

Synthesis of (E)-4-(4-Chloro-3-fluoro-phenyl)-but-3-en-2-one (3i): Compound 3i was prepared following the procedure described in Example 1 for the synthesis of 3a.

Synthesis of 5-(4-Chloro-3-fluoro-phenyl)-cyclohexane-1,3-dione (4i): Compound 4i was prepared following the procedure described in Example 1 for the synthesis of 4a.

Synthesis of 7-(4-Chloro-3-fluoro-phenyl)-4-(3-hydroxy-phenyl)-2-methyl-5-oxo-1,4,5,6,7,8-hexahydro-quinoline-3-carboxylic acid cyclopentyl ester (7q): Compound 7p was prepared following the procedure described in Example 1 for the synthesis of 7a: Diastereomer A (higher Rf), ¹HNMR ((CD₃)₂SO) δ 9.15 (s, 1H), 9.07 (s, 1H), 7.51 (t, J=8.0 Hz, 1H), 7.47 (dd, J=8.4, 1.2 Hz, 1H), 7.23 (dd, J=8.4, 1.2 Hz, 1H), 6.95 (t, J=8.4 Hz, 1H), 6.61 (m, 2H), 6.46 (m, 1H), 5.01 (m, 1H), 4.82 (s, 1H), 3.22 (m, 1H), 2.76 (dd, J=16.8, 12.0 Hz, 1H), 2.30 (s, 3H); MS (ES) M+H expected=496.9, found=496.5. Diastereomer B (lower Rf): MS (ES) M+H expected=496.9, found=496.5.

### Example 18

### Preparation of 7-(2,6-Dimethyl-phenyl)-2-ethyl-4-(3-hydroxy-phenyl)-5-oxo-1,4,5,6,7,8-hexahydro-quinoline-3-carboxylic acid cyclopentyl ester (7r)

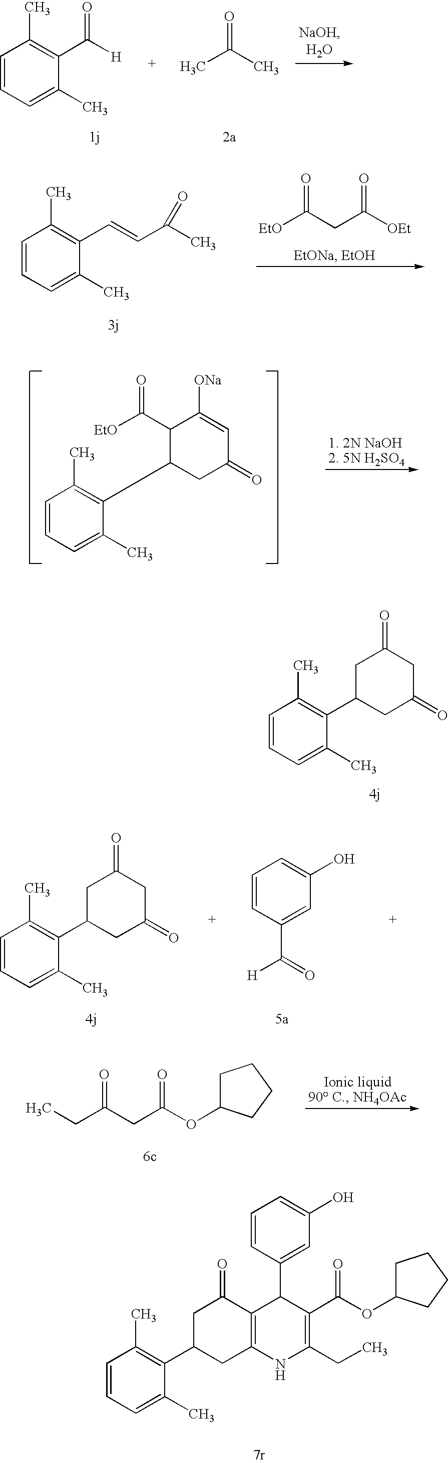

Synthesis of (E)-4-(2,6-Dimethyl-phenyl)-but-3-en-2-one (3j): Compound 3j was prepared following the procedure described in Example 1 for the synthesis of 3a.

Synthesis of 5-(2,6-Dimethyl-phenyl)-cyclohexane-1,3-dione (4j): Compound 4j was prepared following the procedure described in Example 1 for the synthesis of 4a.

Synthesis of 7-(2,6-Dimethyl-phenyl)-2-ethyl-4-(3-hydroxy-phenyl)-5-oxo-1,4,5,6,7,8-hexahydro-quinoline-3-carboxylic acid cyclopentyl ester (7r): Compound 7r was prepared following the procedure described in Example 1 for the synthesis of 7a: MS (ES) M+H expected=486.6, found=486.7.

### Example 19

### Preparation of 2-Cyclopropyl-7-(2,6-dimethyl-phenyl)-4-(3-hydroxy-phenyl)-5-oxo-1,4,5,6,7,8-hexahydro-quinoline-3-carboxylic acid cyclopentyl ester (7s)

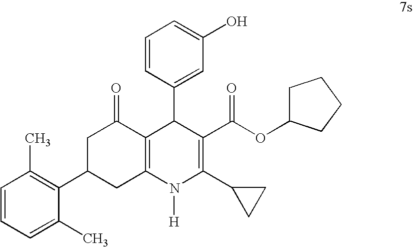

Synthesis of 2-Cyclopropyl-7-(2,6-dimethyl-phenyl)-4-(3-hydroxy-phenyl)-5-oxo-1,4,5,6,7,8-hexahydro-quinoline-3-carboxylic acid cyclopentyl ester (7s): Compound 7s was prepared following the procedure described in Example 1 for the synthesis of 7a: MS (ES) M+H expected=498.6, found=498.6.

### Example 20

### Preparation of 7-(2,6-Dimethyl-phenyl)-4-(3-hydroxy-phenyl)-2-isobutyl-5-oxo-1,4,5,6,7,8-hexahydro-quinoline-3-carboxylic acid cyclopentyl ester (7t)

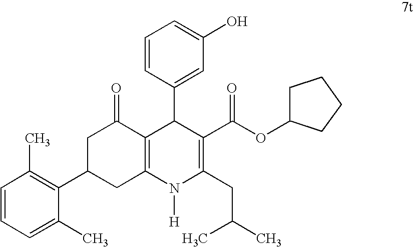

Synthesis of 7-(2,6-Dimethyl-phenyl)-4-(3-hydroxy-phenyl)-2-isobutyl-5-oxo-1,4,5,6,7,8-hexahydro-quinoline-3-carboxylic acid cyclopentyl ester (7t): Compound 7t was prepared following the procedure described in Example 1 for the synthesis of 7a: MS (ES) M+H expected=514.7, found=514.7.

### Example 21

### Preparation of 7-(2,6-Dimethyl-phenyl)-2-ethyl-5-hydroxyimino-4-(3-hydroxy-phenyl)-1,4,5,6,7,8-hexahydro-quinoline-3-carboxylic acid cyclopentyl ester (7u)

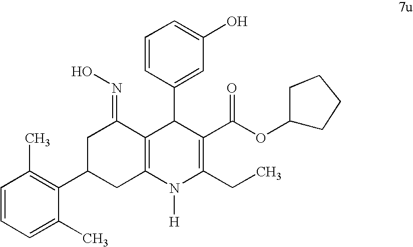

Synthesis of 7-(2,6-Dimethyl-phenyl)-2-ethyl-5-hydroxyimino-4-(3-hydroxy-phenyl)-1,4,5,6,7,8-hexahydro-quinoline-3-carboxylic acid cyclopentyl ester (7u): Compound 7u was prepared following the procedure described in Example 1 for the synthesis of 7a: MS (ES) M+H expected=501.6, found=501.3.

### Example 22

### Preparation of 7-(2,6-Dimethyl-phenyl)-2-ethyl-4-(3-hydroxy-phenyl)-5-methoxyimino-1,4,5,6,7,8-hexahydro-quinoline-3-carboxylic acid cyclopentyl ester (7v)

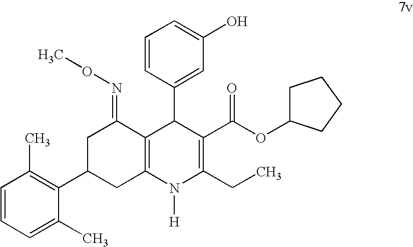

Synthesis of 7-(2,6-Dimethyl-phenyl)-2-ethyl-4-(3-hydroxy-phenyl)-5-methoxyimino-1,4,5,6,7,8-hexahydro-quinoline-3-carboxylic acid cyclopentyl ester (7v): Compound 7v was prepared following the procedure described in Example 1 for the synthesis of 7a: MS (ES) M+H expected=515.6, found=515.7.

### Example 23

### Preparation of 2-Ethyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7w)

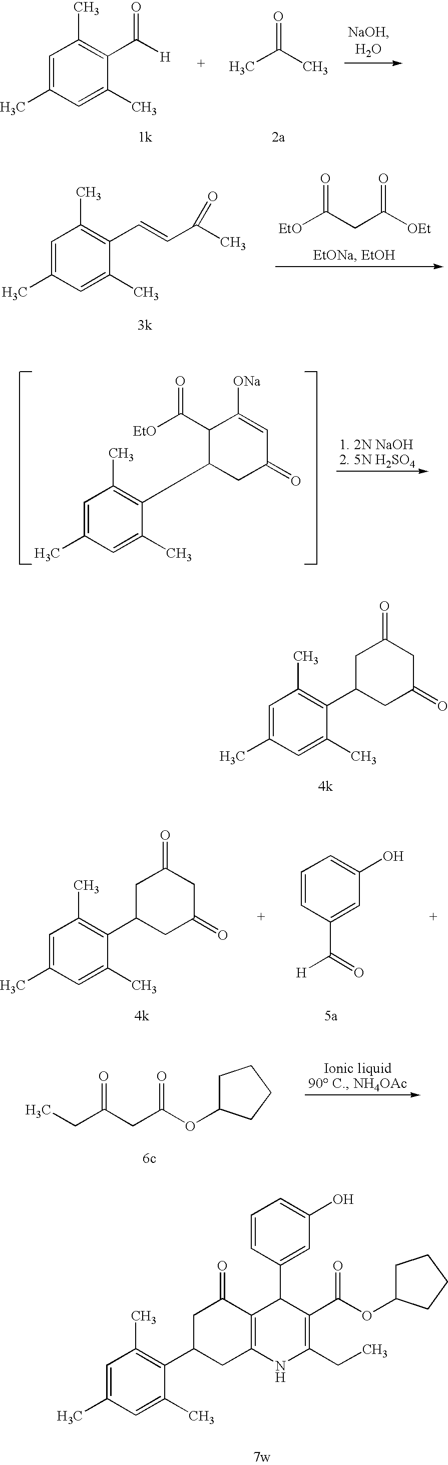

Synthesis of (E)-4-(2,4,6-Trimethyl-phenyl)-but-3-en-2-one (3k): Compound 3k was prepared following the procedure described in Example 1 for the synthesis of 3a.

Synthesis of 5-(2,4,6-Trimethyl-phenyl)-cyclohexane-1,3-dione (4k): Compound 4v was prepared following the procedure described in Example 1 for the synthesis of 4a: MS (ES) M+H expected=231.3, found=231.4.

ynthesis of 2-Ethyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7w): Compound 7w was prepared following the procedure described in Example 1 for the synthesis of 7a: MS (ES) M+H expected=500.6, found=500.6.

### Example 24

### Preparation of 2-Cyclopropyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7x)

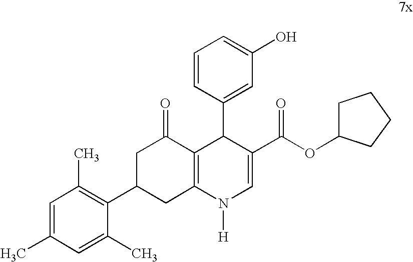

Synthesis of 2-Cyclopropyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7x): Compound 7x was prepared following the procedure described in Example 1 for the synthesis of 7a: MS (ES) M+H expected=512.6, found=512.7.

### Example 25

### Preparation of 4-(3-Hydroxy-phenyl)-2-isobutyl-5-oxo-7-(2,4,6-trimethyl-phenyl)-1,4,5,6,7,8-hexahydro-quinoline-3-carboxylic acid cyclopentyl ester (7y)

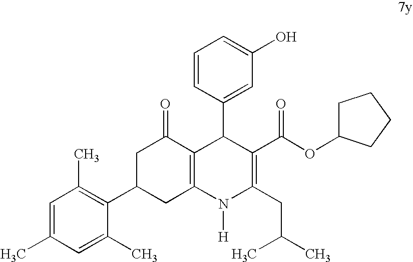

Synthesis of 4-(3-Hydroxy-phenyl)-2-isobutyl-5-oxo-7-(2,4,6-trimethyl-phenyl)-1,4,5,6,7,8-hexahydro-quinoline-3-carboxylic acid cyclopentyl ester (7y): Compound 7y was prepared following the procedure described in Example 1 for the synthesis of 7a: MS (ES) M+H expected=528.7, found=528.7.

### Example 26

### Preparation of 2-Cyclopentyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7z)

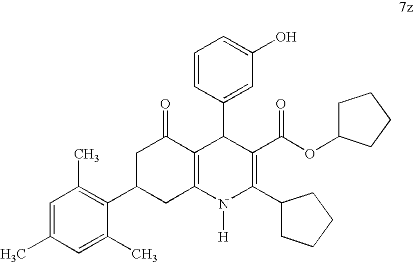

Synthesis of 2-Cyclopentyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7z): Compound 7z was prepared following the procedure described in Example 1 for the synthesis of 7a: MS (ES) M+H expected=540.7, found=540.7.

### Example 27

### Preparation of 2-Cyclopentylmethyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7aa)

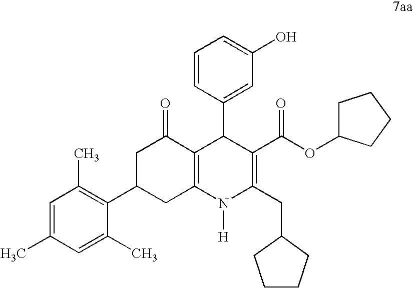

Synthesis of 2-Cyclopentylmethyl-4-(3-hydroxy-phenyl)-5-oxo-7-(2,4,6-trimethyl-phenyl)-1,4,5,6,7,8-hexahydro-quinoline-3-carboxylic acid cyclopentyl ester (7aa): Compound 7aa was prepared following the procedure described in Example 1 for the synthesis of 7a: MS (ES) M+H expected=554.7, found=554.7.

### Example 28

### Preparation of 2-(2-Cyclopentyl-ethyl)-4-(3-hydroxy-phenyl)-5-oxo-7-(2,4,6-trimethyl-phenyl)-1,4,5,6,7,8-hexahydro-quinoline-3-carboxylic acid cyclopentyl ester (7bb)

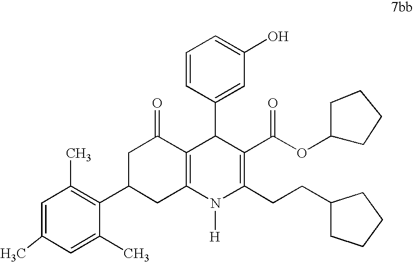

Synthesis of 2-(2-Cyclopentyl-ethyl)-4-(3-hydroxy-phenyl)-5-oxo-7-(2,4,6-trimethyl-phenyl)-1,4,5,6,7,8-hexahydro-quinoline-3-carboxylic acid cyclopentyl ester (7bb): Compound 7bb was prepared following the procedure described in Example 1 for the synthesis of 7a: MS (ES) M+H expected=568.7, found=568 .7.

### Example 29

### Preparation of 2-(2-Cyclopentyl-ethyl)-4-(3-hydroxy-phenyl)-5-oxo-7-(2,4,6-trimethyl-phenyl)-1,4,5,6,7,8-hexahydro-quinoline-3-carboxylic acid ethyl ester (7cc)

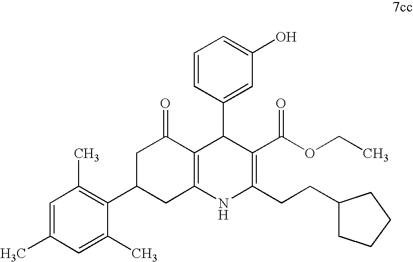

Synthesis of 2-(2-Cyclopentyl-ethyl)-4-(3-hydroxy-phenyl)-5-oxo-7-(2,4,6-trimethyl-phenyl)-1,4,5,6,7,8-hexahydro-quinoline-3-carboxylic acid ethyl ester (7cc): Compound 7cc was prepared following the procedure described in Example 1 for the synthesis of 7a: MS (ES) M+H expected=528.7, found=528.5.

### Example 30

### Preparation of 2-Methyl-4-(3-nitro-phenyl)-5-oxo-7-(2,4,6-trimethyl-phenyl)-1,4,5,6,7,8-hexahydro-quinoline-3-carboxylic acid cyclopentyl ester (7dd)

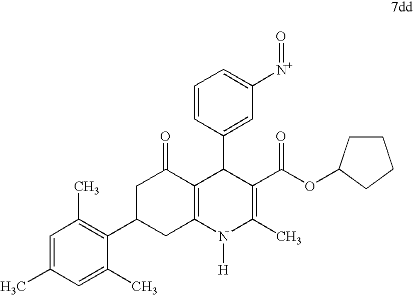

Synthesis of 2-Methyl-4-(3-nitro-phenyl)-5-oxo-7-(2,4,6-trimethyl-phenyl)-1,4,5,6,7,8-hexahydro-quinoline-3-carboxylic acid cyclopentyl ester (7dd): Compound 7dd was prepared following the procedure described in Example 1 for the synthesis of 7a: MS (ES) M+H expected=515.6, found=515.5.

### Example 31

### Preparation of 4-(3-Amino-phenyl)-2-methyl-5-oxo-7-(2,4,6-trimethyl-phenyl)-1,4,5,6,7,8-hexahydro-quinoline-3-carboxylic acid cyclopentyl ester (7ee)

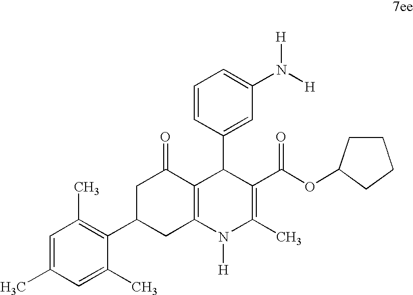

Synthesis of 4-(3-Amino-phenyl)-2-methyl-5-oxo-7-(2,4,6-trimethyl-phenyl)-1,4,5,6,7,8-hexahydro-quinoline-3-carboxylic acid cyclopentyl ester (7ee). Compound 7ee was prepared following the procedure described in Example 1 for the synthesis of 7a: MS (ES) M+H expected=485.6, found=485.5.

### Example 31

### Preparation of 4-(3-Acetylamino-phenyl)-2-methyl-5-oxo-7-(2,4,6-trimethyl-phenyl)-1,4,5,6,7,8-hexahydro-quinoline-3-carboxylic acid cyclopentyl ester (7ff)

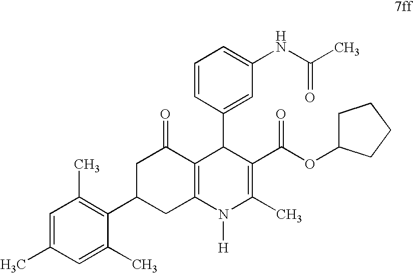

Synthesis of 4-(3-Acetylamino-phenyl)-2-methyl-5-oxo-7-(2,4,6-trimethyl-phenyl)-1,4,5,6,7,8-hexahydro-quinoline-3-carboxylic acid cyclopentyl ester (7ff): Compound 7ff was prepared following the procedure described in Example 1 for the synthesis of 7a: MS (ES) M+H expected=527.7, found=527.6.

### Example 32

### Preparation of 4-(3-Methanesuffonylamino-phenyl)-2-methyl-5-oxo-7-(2,4,6-trimethyl-phenyl)-1,4,5,6,7,8-hexahydro-quinoline-3-carboxylic acid cyclopentyl ester (7gg)

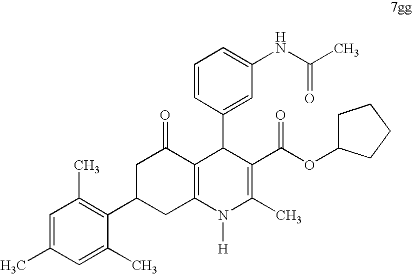

Synthesis of 4-(3-Methanesulfonylamino-phenyl)-2-methyl-5-oxo-7-(2,4,6-trimethyl-phenyl)-1,4,5,6,7,8-hexahydro-quinoline-3-carboxylic acid cyclopentyl ester (7gg): Compound 7gg was prepared following the procedure described in Example 1 for the synthesis of 7a: MS (ES) M+H expected=563.7, found=563.5.

### Example 33

### Preparation of 7-Benzo[1,3]dioxol-5-yl-2-ethyl-4-(3-hydroxy-phenyl)-5-oxo-1,4,5,6,7,8-hexahydro-quinoline-3-carboxylic acid cyclopentyl ester (7hh)

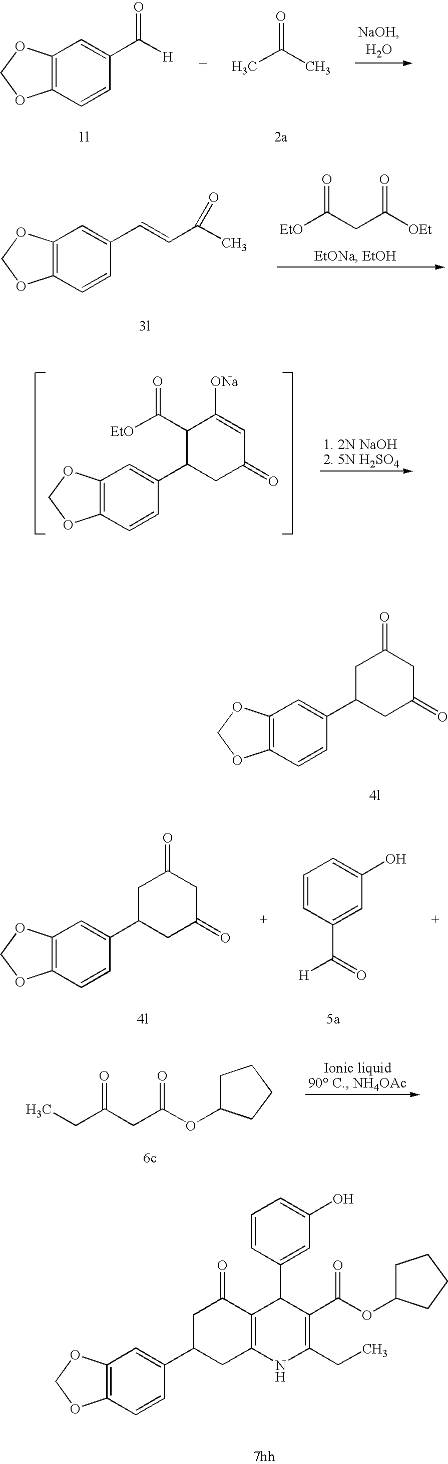

Synthesis of (E)-4-Benzo[1,3]dioxol-5-yl-but-3-en-2-one (3l): Compound 3l was prepared following the procedure described in Example 1 for the synthesis of 3a.

Synthesis of 5-Benzo[1,3]dioxol-5-yl-cyclohexane-1,3-dione (4l): Compound 4l was prepared following the procedure described in Example 1 for the synthesis of 4a.

Synthesis of 7-Benzo[1,3]dioxol-5-yl-2-ethyl-4-(3-hydroxy-phenyl)-5-oxo-1,4,5,6,7,8-hexahydro-quinoline-3-carboxylic acid cyclopentyl ester (7hh): Compound 7hh was prepared following the procedure described in Example 1 for the synthesis of 7a: MS (ES) M+H expected=502.6, found=502.6.

### Example 34

### Preparation of 7-Benzo[1,3]dioxol-4-yl-2-ethyl-4-(3-hydroxy-phenyl)-5-oxo-1,4,5,6,7,8-hexahydro-quinoline-3-carboxylic acid cyclopentyl ester (7ii)

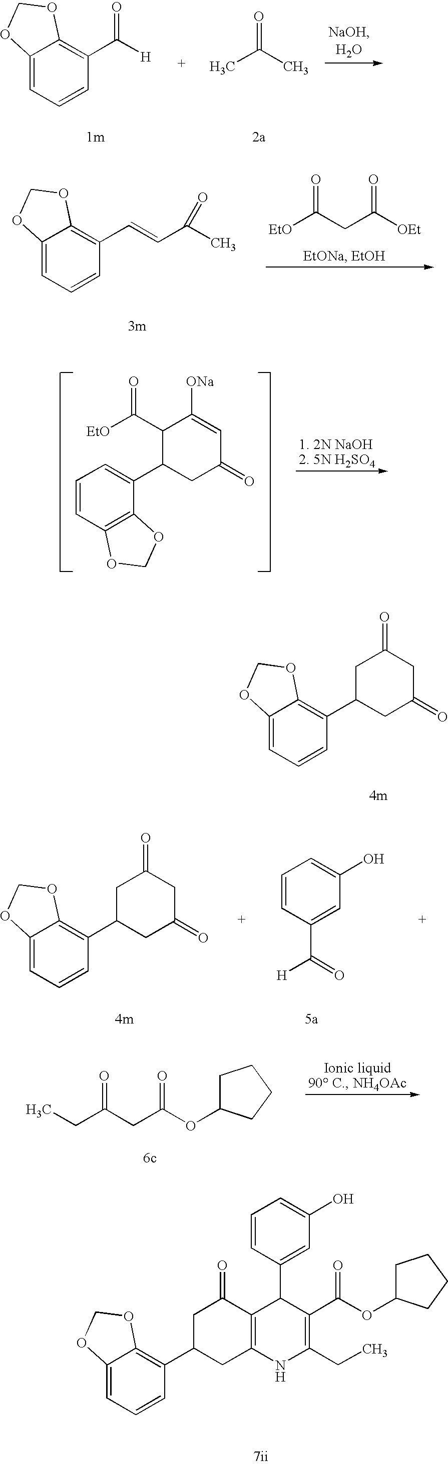

Synthesis of (E)-4-Benzo[1,3]dioxol-4-yl-but-3-en-2-one (3m): Compound 3m was prepared following the procedure described in Example 1 for the synthesis of 3a.

Synthesis of 5-Benzo[1,3]dioxol-4-yl-cyclohexane-1,3-dione (4m): Compound 4m was prepared following the procedure described in Example 1 for the synthesis of 4a.

Synthesis of 7-Benzo[1,3]dioxol-4-yl-2-ethyl-4-(3-hydroxy-phenyl)-5-oxo-1,4,5,6,7,8-hexahydro-quinoline-3-carboxylic acid cyclopentyl ester (7ii): Compound 7ii is prepared following the procedure described in Example 1 for the synthesis of 7a: MS (ES) M+H expected=502.6, found=502.6.

### Example 35

### Preparation of 7-Benzo[1,3]dioxol-5-yl-4-(3-hydroxy-phenyl)-2-isopropyl-5-oxo-1,4,5,6,7,8-hexahydro-quinoline-3-carboxylic acid cyclopentyl ester (7jj)

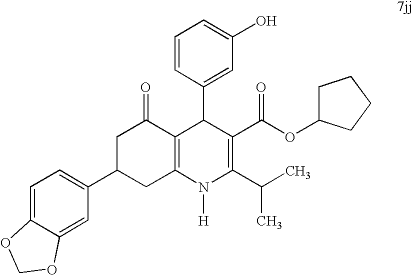

Synthesis of 7-Benzo[1,3]dioxol-5-yl-4-(3-hydroxy-phenyl)-2-isopropyl-5-oxo-1,4,5,6,7,8-hexahydro-quinoline-3-carboxylic acid cyclopentyl ester (7jj): Compound 7jj was prepared following the procedure described in Example 1 for the synthesis of 7a: MS (ES) M+H expected=516.6, found=516.6.

### Example 36

### Preparation of 7-Benzo[1,3]dioxol-5-yl-4-(3-hydroxy-phenyl)-2-isobutyl-5-oxo-1,4,5,6,7,8-hexahydro-quinoline-3-carboxylic acid cyclopentyl ester (7kk)

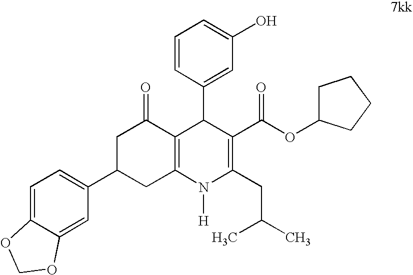

Synthesis of 7-Benzo[1,3]dioxol-5-yl-4-(3-hydroxy-phenyl)-2-isobutyl-5-oxo-1,4,5,6,7,8-hexahydro-quinoline-3-carboxylic acid cyclopentyl ester (7kk): Compound 7kk was prepared following the procedure described in Example 1 for the synthesis of 7a: MS (ES) M+H expected=530.6, found=530.7.

### Example 37

### Preparation of 7-Benzo[1,3]dioxol-5-yl-2-cyclopentylmethyl-4-(3-hydroxy-phenyl)-5-oxo-1,4,5,6,7,8-hexahydro-quinoline-3-carboxylic acid cyclopentyl ester (7ll)

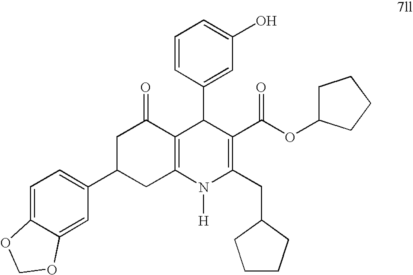

Synthesis of 7-Benzo[1,3]dioxol-5-yl-2-cyclopentylmethyl-4-(3-hydroxy-phenyl)-5-oxo-1,4,5,6,7,8-hexahydro-quinoline-3-carboxylic acid cyclopentyl ester (7ll): Compound 7ll was prepared following the procedure described in Example 1 for the synthesis of 7a: MS (ES) M+H expected=556.7, found=556.7.

### Example 38

### Preparation of 7-Benzo[1,3]dioxol-5-yl-2-(2-cyclopentyl-ethyl)-4-(3-hydroxy-phenyl)-5-oxo-1,4,5,6,7,8-hexahydro-quinoline-3-carboxylic acid cyclopentyl ester (7mm)

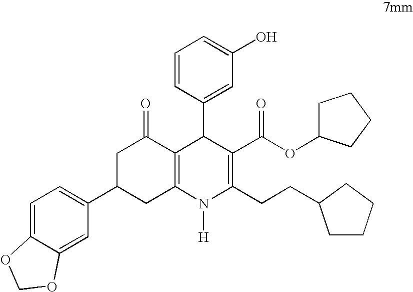

Synthesis of 7-Benzo[1,3]dioxol-5-yl-2-(2-cyclopentyl-ethyl)-4-(3-hydroxy-phenyl)-5-oxo-1,4,5,6,7,8-hexahydro-quinoline-3-carboxylic acid cyclopentyl ester (7mm): Compound 7mm was prepared following the procedure described in Example 1 for the synthesis of 7a: MS (ES) M+H expected=570.7, found=570.6.

### Example 39

### Preparation of 7-Benzo[1,3]dioxol-5-yl-2-(2-cyclopentyl-ethyl)-4-(3-hydroxy-phenyl)-5-oxo-1,4,5,6,7,8-hexahydro-quinoline-3-carboxylic acid ethyl ester (7nn)

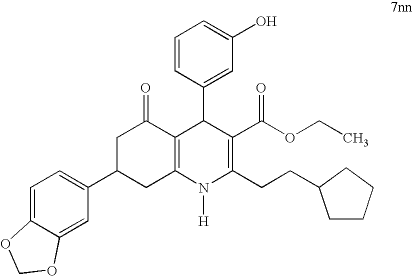

Synthesis of 7-Benzo[1,3]dioxol-5-yl-2-(2-cyclopentyl-ethyl)-4-(3-hydroxy-phenyl)-5-oxo-1,4 5,6,7,8-hexahydro-quinoline-3-carboxylic acid ethyl ester (7nn): Compound 7nn was prepared following the procedure described in Example 1 for the synthesis of 7a MS (ES) M+H expected=530.6, found=530.5.

### Example 40

### Preparation of 7-(2,3-Dihydro-benzofuran-6-yl)-2-ethyl-4-(3-hydroxy-phenyl)-5-oxo-1,4,5,6,7,8-hexahydro-quinoline-3-carboxylic acid cyclopentyl ester (7oo)

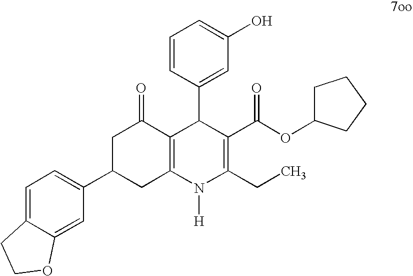

Synthesis of 7-(2,3-Dihydro-benzofuran-6-yl)-2-ethyl-4-(3-hydroxy-phenyl)-5-oxo-1,4,5,6,7,8-hexahydro-quinoline-3-carboxylic acid cyclopentyl ester (7oo): Compound 7oo was prepared following the procedure described in Example 1 for the synthesis of 7a: MS (ES) M+H expected=500.6, found=500.6.

### Example 41

### Preparation of 7-(2,3-Dihydro-benzo[1,4]dioxin-6-yl)-2-ethyl-4-(3-hydroxy-phenyl)-5-oxo-1,4,5,6,7,8-hexahydro-quinoline-3-carboxylic acid cyclopentyl ester (7pp)

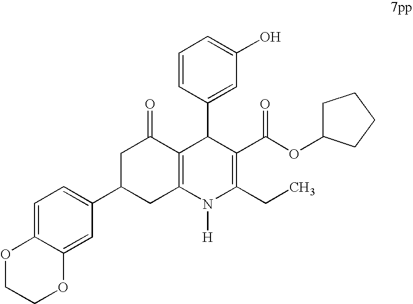

Synthesis of 7-(2,3-Dihydro-benzo[1,4]dioxin-6-yl)-2-ethyl-4-(3-hydroxy-phenyl)-5-oxo-1,4,5,6,7,8-hexahydro-quinoline-3-carboxylic acid cyclopentyl ester (7pp): Compound 7pp was prepared following the procedure described in Example 1 for the synthesis of 7a: MS (ES) M+H expected=516.6, found=516.6.

### Example 42

### Preparation of 7-(2,2-Difluoro-benzo[1,3]dioxol-5-yl)-2-ethyl-4-(3-hydroxy-phenyl)-5-oxo-1,4,5,6,7,8-hexahydro-quinoline-3-carboxylic acid cyclopentyl ester (7qq)

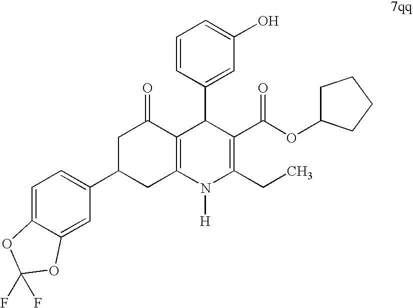

Synthesis of 7-(2,2-Difluoro-benzo[1,3]dioxol-5-yl)-2-ethyl-4-(3-hydroxy-phenyl)-5-oxo-1,4,5,6,7,8-hexahydro-quinoline-3-carboxylic acid cyclopentyl ester (7qq): Compound 7qq was prepared following the procedure described in Example 1 for the synthesis of 7a. Diastereomer A (higher Rf), MS (ES) M+H expected=538.5, found=538.6;. Diastereomer B (lower Rf), MS (ES) M+H expected=538.5, found=538.6.

### Example 43

### Preparation of 7-(2,2-Difluoro-benzo[1,3]dioxol-5-yl)-4-(3-hydroxy-phenyl)-2-isobutyl-5-oxo-1,4,5,6,7,8-hexahydro-quinoline-3-carboxylic acid cyclopentyl ester (7rr)

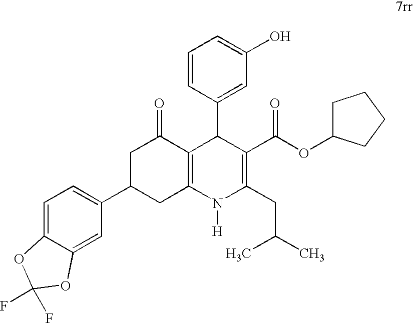

Synthesis of 7-(2,2-Difluoro-benzo[1,3]dioxol-5-yl)-4-(3-hydroxy-phenyl)-2-isobutyl-5-oxo-1,4,5,6,7,8-hexahydro-quinoline-3-carboxylic acid cyclopentyl ester (7rr): Compound 7rr was prepared following the procedure described in Example 1 for the synthesis of 7a: Diastereomer A (higher Rf), MS (ES) M+H expected=566.6, found=566.5. Diastereomer B (lower Rf): MS (ES) M+H expected=566.6, found=566.5.

### Example 44

### reparation of 7-Benzo[1,3]dioxol-5-yl-2-methyl-4-(3-nitro-phenyl)-5-oxo-1,4,5,6,7,8-hexahydro-quinoline-3-carboxylic acid cyclopentyl ester (7ss)

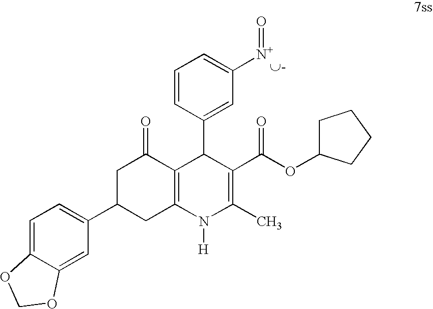

Synthesis of 7-Benzo[1,3]dioxol-5-yl-2-methyl-4-(3-nitro-phenyl)-5-oxo-1,4,5,6,7,8-hexahydro-quinoline-3-carboxylic acid cyclopentyl ester (7ss): Compound 7ss was prepared following the procedure described in Example 1 for the synthesis of 7a: Diastereomer A (higher Rf), ¹HNMR ((CD₃)₂SO) δ 9.15 (s, 1H), 9.08 (s, 1H), 9.00 (s, 1H), 7.49 (s, 1H), 7.32 (d, J=8.4 Hz, 1H), 7.20 (dd, J=8.4, 1.2 Hz, 1H), 6.95 (t, J=7.6 Hz, 1H), 6.60 (m, 2H), 6.46 (m, 1H), 5.01 (m, 1H), 4.85 (s, 1H), 3.23 (m, 1H), 0.91 (d, J=6.4 Hz, 3H); MS (ES) M+H expected=517.6, found=517.5. Diastereomer B (lower Rf), MS (ES) M+H expected=517.6, found=517.5.

### Example 45

### Preparation of 4-(3-Amino-phenyl)-7-benzo[1,3]dioxol-5-yl-2-methyl-5-oxo-1,4,5,6,7,8-hexahydro-quinoline-3-carboxylic acid cyclopentyl ester (7tt)

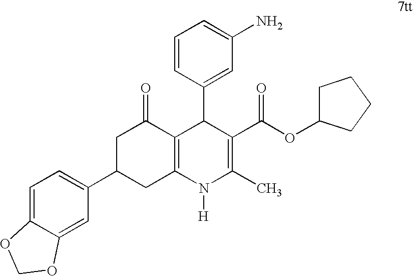

Synthesis of 4-(3-Amino-phenyl)-7-benzo[1,3]dioxol-5-yl-2-methyl-5-oxo-1,4,5,6,7,8-hexahydro-quinoline-3-carboxylic acid cyclopentyl ester (7tt): Compound 7tt was prepared following the procedure described in Example 1 for the synthesis of 7a: Diastereomer A (higher Rf), MS (ES) M+H expected=487.6, found=487.4. Diastereomer B (lower Rf): MS (ES) M+H expected=487.6, found=487.5.

### Example 46

### Preparation of 7-Benzo[1,3]dioxol-5-yl-4-(3-carbamoyl-phenyl)-2-methyl-5-oxo-1,4,5,6,7,8-hexahydro-quinoline-3-carboxylic acid cyclopentyl ester (7uu)

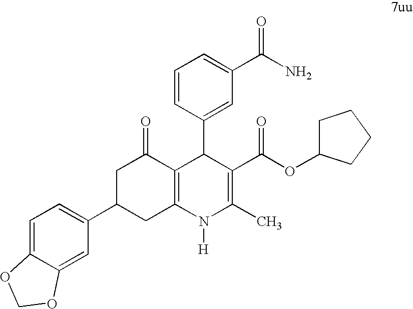

Synthesis of 7-Benzo[1,3]dioxol-5-yl-4-(3-carbamoyl-phenyl)-2-methyl-5-oxo-1,4,5,6,7,8-hexahydro-quinoline-3-carboxylic acid cyclopentyl ester (7uu): Compound 7uu was prepared following the procedure described in Example 1 for the synthesis of 7a: MS (ES) M+H expected=515.6, found=515.5.

### Example 47

### Preparation of 7-Benzo[1,3]dioxol-5-yl-4-(3-carboxy-phenyl)-2-methyl-5-oxo-1,4,5,6,7,8-hexahydro-quinoline-3-carboxylic acid cyclopentyl ester (7vv)

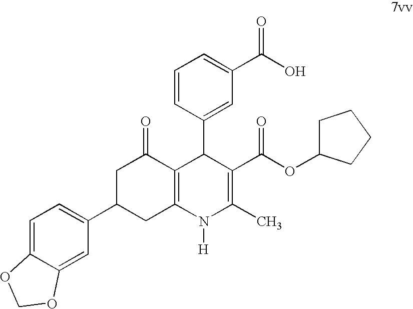

Synthesis of 7-Benzo[1,3]dioxol-5-yl-4-(3-carboxy-phenyl)-2-methyl-5-oxo-1,4,5,6,7,8-hexahydro-quinoline-3-carboxylic acid cyclopentyl ester (7vv): Compound 7vv was prepared following the procedure described in Example 1 for the synthesis of 7a: MS (ES) M+H expected=516.6, found=516.5.

### Example 48

### Preparation of 7-Benzo[1,3]dioxol-5-yl-4-(1H-indol-6-yl)-2-methyl-5-oxo-1,4,5,6,7,8-hexahydro-quinoline-3-carboxylic acid cyclopentyl ester (7ww)

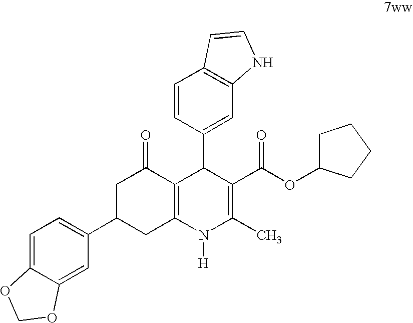

Synthesis of 7-Benzo[1,3]dioxol-5-yl-4-(1H-indol-6-yl)-2-methyl-5-oxo-1,4,5,6,7,8-hexahydro-quinoline-3-carboxylic acid cyclopentyl ester (7ww): Compound 7ww was prepared following the procedure described in Example 1 for the synthesis of 7a: MS (ES) M+H expected=511.6, found=511.4.

### Example 49

### Preparation of 7-Benzo[1,3]dioxol-5-yl-4-(1H-indol-3-yl)-2-methyl-5-oxo-1,4,5,6,7,8-hexahydro-quinoline-3-carboxylic acid cyclopentyl ester (7xx)

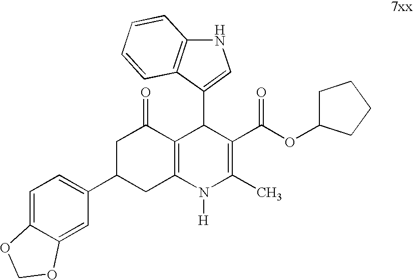

Synthesis of 7-Benzo[1,3]dioxol-5-yl-4-(1H-indol-3-yl)-2-methyl-5-oxo-1,4,5,6,7,8-hexahydro-quinoline-3-carboxylic acid cyclopentyl ester (7xx): Compound 7xx was prepared following the procedure described in Example 1 for the synthesis of 7a: MS (ES) M+H expected=511.6, found=511.4.

### Example 50

### Preparation of 7-(4-Ethyl-phenyl)-4-(3-hydroxy-phenyl)-2-methyl-5-oxo-1,4,5,6,7,8-hexahydro-quinoline-3-carboxylic acid cyclopentyl ester (7yy)

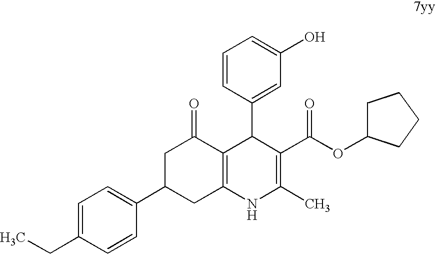

Synthesis of 7-(4-Ethyl-phenyl)-4-(3-hydroxy-phenyl)-2-methyl-5-oxo-1,4,5,6,7,8-hexahydro-quinoline-3-carboxylic acid cyclopentyl ester (7yy): Compound 7yy was prepared following the procedure described in Example 1 for the synthesis of 7a: MS (ES) M+H expected=471.6, found=471.2.

### Example 51

### Preparation of 4-(3-Hydroxy-phenyl)-7-(4-isopropyl-phenyl)-2-methyl-5-oxo-1,4,5,6,7,8-hexahydro-quinoline-3-carboxylic acid cyclopentyl ester (7zz)

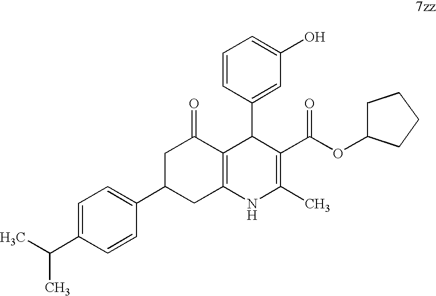

Synthesis of 4-(3-Hydroxy-phenyl)-7-(4-isopropyl-phenyl)-2-methyl-5-oxo-1,4,5,6,7,8-hexahydro-quinoline-3-carboxylic acid cyclopentyl ester (7zz): Compound 7zz was prepared following the procedure described in Example 1 for the synthesis of 7a: MS (ES) M+H expected=486.6, found=486.2.

### Example 52

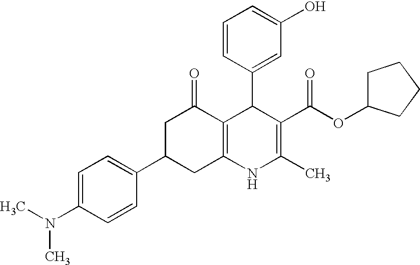

Synthesis of 7-(4-Dimethylamino-phenyl)-4-(3-hydroxy-phenyl)-2-methyl-5-oxo-1,4,5,6,7,8-hexahydro-quinoline-3-carboxylic acid cyclopentyl ester (7aaa): Compound 7aaa was prepared following the procedure described in Example 1 for the synthesis of 7a: MS (ES) M+H expected=487.6, found=486.2.

### Example 53

### Preparation of 7-(4-Ethyl-phenyl)-4-(3-hydroxy-phenyl)-2-methyl-5-oxo-1,4,5,6,7,8-hexahydro-quinoline-3-carboxylic acid cyclopentylamide (7bbb)

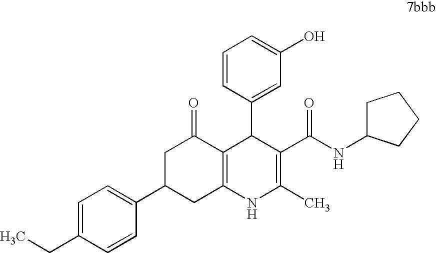

Synthesis of 7-(4-Ethyl-phenyl)-4-(3-hydroxy-phenyl)-2-methyl-5-oxo-1,4,5,6,7,8-hexahydro-quinoline-3-carboxylic acid cyclopentylamide (7bbb): Compound 7bbb was prepared following the procedure described in Example 1 for the synthesis of 7a: MS (ES) M+H expected=471.6, found=471.2.

### Example 54

### Preparation of 7-Biphenyl-4-yl-4-(3-hydroxy-phenyl)-2-methyl-5-oxo-1,4,5,6,7,8-hexahydro-quinoline-3-carboxylic acid cyclopentyl ester (7ccc)

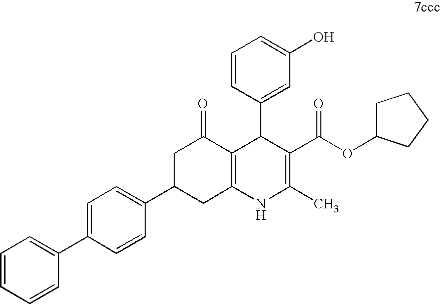

Synthesis of 7-Biphenyl-4-yl-4-(3-hydroxy-phenyl)-2-methyl-5-oxo-1,4,5,6,7,8-hexahydro-quinoline-3-carboxylic acid cyclopentyl ester (7ccc): Compound 7ccc was prepared following the procedure described in Example 1 for the synthesis of 7a: MS (ES) M+H expected=519.62, found=519.6.

### Example 55

### Preparation of 7-(4-tert-Butyl-phenyl)-4-(3-hydroxy-phenyl)-2-methyl-5-oxo-1,4,5,6,7,8-hexahydro-quinoline-3-carboxylic acid cyclopentyl ester (7ddd)

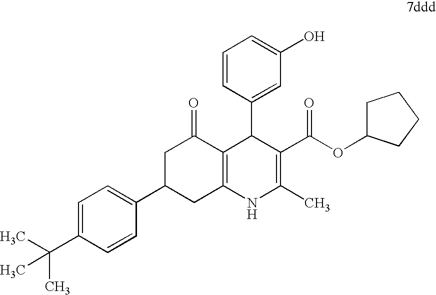

Synthesis of 7-(4-tert-Butyl-phenyl)-4-(3-hydroxy-phenyl)-2-methyl-5-oxo-1,4,5,6,7,8-hexahydro-quinoline-3-carboxylic acid cyclopentyl ester (7ddd): Compound 7ccc was prepared following the procedure described in Example 1 for the synthesis of 7a: MS (ES) M+H expected=499.27, found=499.6.

### Example 56

### Preparation of 7-Cyclopentyl-4-(3-hydroxy-phenyl)-2-methyl-5-oxo-1,4,5,6,7,8-hexahydro-quinoline-3-carboxylic acid cyclopentyl ester (7eee)

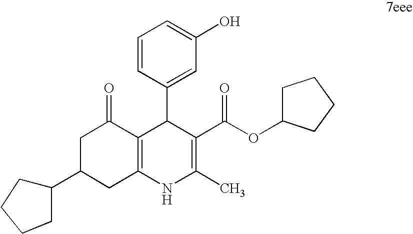

Synthesis of 7-Cyclopentyl-4-(3-hydroxy-phenyl)-2-methyl-5-oxo-1,4,5,6,7,8-hexahydro-quinoline-3-carboxylic acid cyclopentyl ester (7eee): Compound 7eee was prepared following the procedure described in Example 1 for the synthesis of 7a: MS (ES) M+H expected=435.8, found=436.7.

### Example 57

### Preparation of 4-(3-Hydroxy-phenyl)-2-methyl-7-naphthalen-1-yl-5-oxo-1,4,5,6,7,8-hexahydro-quinoline-3-carboxylic acid cyclopentyl ester (7fff)

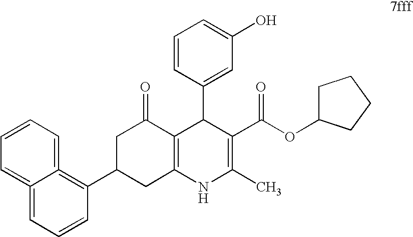

Synthesis of 4-(3-Hydroxy-phenyl)-2-methyl-7-naphthalen-1-yl-5-oxo-1,4,5,6,7,8-hexahydro-quinoline-3-carboxylic acid cyclopentyl ester: Compound 7fff was prepared following the procedure described in Example 1 for the synthesis of 7a: MS (ES) M+H expected=493.6, found=494.6.

### Example 58

### Preparation of 4-(3-Hydroxy-phenyl)-2-methyl-7-naphthalen-2-yl-5-oxo-1,4,5,6,7,8-hexahydro-quinoline-3-carboxylic acid cyclopentyl ester (7ggg)

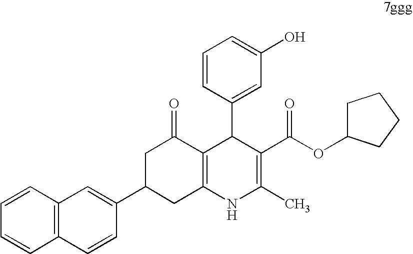

Synthesis of 4-(3-Hydroxy-phenyl)-2-methyl-7-naphthalen-2-yl-5-oxo-1,4,5,6,7,8-hexahydro-quinoline-3-carboxylic acid cyclopentyl ester (7ggg): Compound 7ggg was prepared following the procedure described in Example 1 for the synthesis of 7a: MS (ES) M+H expected=493.3, found=494.2.

### Example 59

### Preparation of 4-(3-Hydroxy-phenyl)-2-methyl-5-oxo-7-pyridin-2-yl-1,4,5,6,7,8-hexahydro-quinoline-3-carboxylic acid cyclopentyl ester (7hhh)

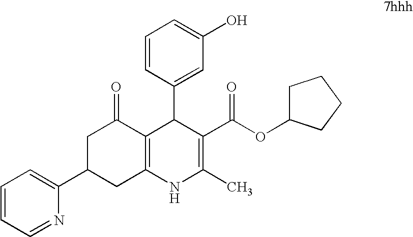

Synthesis of 4-(3-Hydroxy-phenyl)-2-methyl-5-oxo-7-pyridin-2-yl-1,4,5,6,7,8-hexahydro-quinoline-3-carboxylic acid cyclopentyl ester (7hhh): Compound 7hhh was prepared following the procedure described in Example 1 for the synthesis of 7a: MS (ES) M+H expected=445.5, found=445.2.

### Example 60

### Preparation of 4-(3-Hydroxy-phenyl)-2-methyl-5-oxo-7-pyridin-3-yl-1,4,5,6,7,8-hexahydro-quinoline-3-carboxylic acid cyclopentyl ester (7iii)

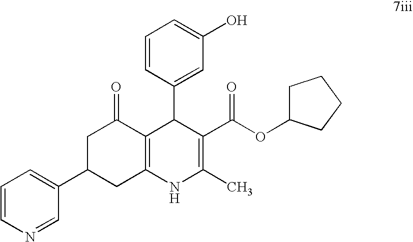

Synthesis of 4-(3-Hydroxy-phenyl)-2-methyl-5-oxo-7-pyridin-3-yl-1,4,5,6,7,8-hexahydro-quinoline-3-carboxylic acid cyclopentyl ester (7iii): Compound 7iii was prepared following the procedure described in Example 1 for the synthesis of 7a: MS (ES) M+H expected=445.5, found=445.6.

### Example 61

### Preparation of 4-(3-Hydroxy-phenyl)-2-methyl-5-oxo-7-pyridin-4-yl-1,4,5,6,7,8-hexahydro-quinoline-3-carboxylic acid cyclopentyl ester (7jjj)

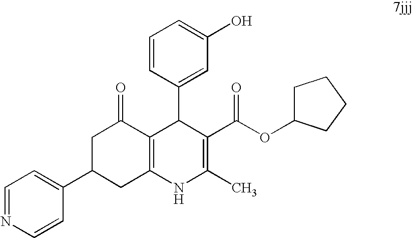

Synthesis of 4-(3-Hydroxy-phenyl)-2-methyl-5-oxo-7-pyridin-4-yl-1,4,5,6,7,8-hexahydro-quinoline-3-carboxylic acid cyclopentyl ester (7jjj): Compound 7jjj was prepared following the procedure described in Example 1 for the synthesis of 7a: MS (ES) M+H expected=445.5, found=445.2.

### Example 62

### Preparation of 4-(3-Hydroxy-phenyl)-2-methyl-5-oxo-7-phenyl-5,6,7,8-tetrahydro-quinoline-3-carboxylic acid cyclopentyl ester (7kkk)

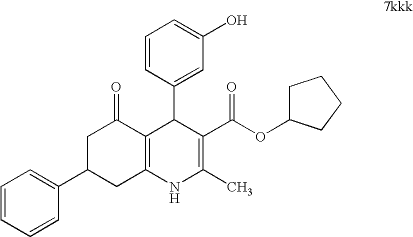

Synthesis of 4-(3-Hydroxy-phenyl)-2-methyl-5-oxo-7-phenyl-5,6,7,8-tetrahydro-quinoline-3-carboxylic acid cyclopentyl ester (7kkk): Compound 7kkk was prepared following the procedure described in Example 1 for the synthesis of 7a: MS (ES) M+H expected=441.5, found=442.2.

### Example 63

### Preparation of 4-(1H-Indol-6-yl)-2-methyl-5-oxo-7-phenyl-1,4,5,6,7,8-hexahydro-quinoline-3-carboxylic acid cyclopentyl ester (7lll)

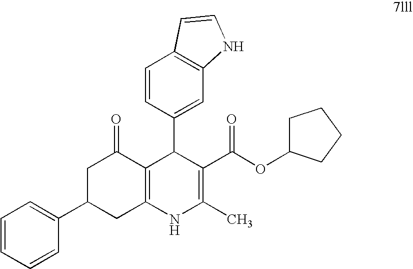

Synthesis of 4-(1H-Indol-6-yl)-2-methyl-5-oxo-7-phenyl-1,4,5,6,7,8-hexahydro-quinoline-3-carboxylic acid cyclopentyl ester (7lll): Compound 7lll was prepared following the procedure described in Example 1 for the synthesis of 7a: MS (ES) M+H expected=467.2, found=467.5.

### Example 64

### Preparation of 4-(1H-Indol-3-yl)-2-methyl-5-oxo-7-phenyl-1,4,5,6,7,8-hexahydro-quinoline-3-carboxylic acid cyclopentyl ester (7mmm)

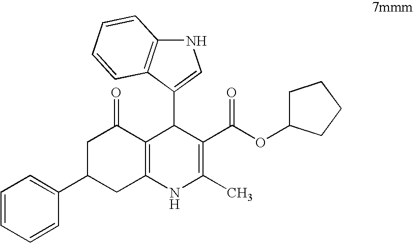

Synthesis of 4-(1H-Indol-3-yl)-2-methyl-5-oxo-7-phenyl-1,4,5,6,7,8-hexahydro-quinoline-3-carboxylic acid cyclopentyl ester (7mmm): Compound 7mmm was prepared following the procedure described in Example 1 for the synthesis of 7a: MS (ES) M+H expected=467.2, found=467.7.

### Example 65

### Preparation of 4-(3-Carboxy-phenyl)-2-methyl-5-oxo-7-phenyl-1,4,5,6,7,8-hexahydro-quinoline-3-carboxylic acid cyclopentyl ester (7nnn)

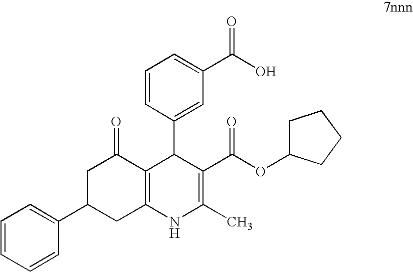

Synthesis of 4-(3-Carboxy-phenyl)-2-methyl-5-oxo-7-phenyl-1,4,5,6,7,8-hexahydro-quinoline-3-carboxylic acid cyclopentyl ester (7nnn): Compound 7nnn was prepared following the procedure described in Example 1 for the synthesis of 7a: MS (ES) M+H expected=471.2, found=472.2.

### Example 66

### Preparation of 4-(3-Carbamoyl-phenyl)-2-methyl-5-oxo-7-phenyl-1,4,5,6,7,8-hexahydro-quinoline-3-carboxylic acid cyclopentyl ester (7ooo)

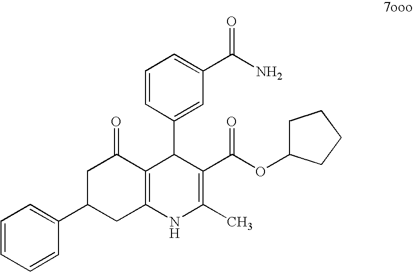

Synthesis of 4-(3-Carbamoyl-phenyl)-2-methyl-5-oxo-7-phenyl-1,4,5,6,7,8-hexahydro-quinoline-3-carboxylic acid cyclopentyl ester (7ooo): Compound 7ooo was prepared following the procedure described in Example 1 for the synthesis of 7a: MS (ES) M+H expected=471.2, found=471.2.

### Example 67

### Preparation of 4-(6-Hydroxy-pyridin-2-yl)-2-methyl-5-oxo-7-phenyl-1,4,5,6,7,8-hexahydro-quinoline-3-carboxylic acid cyclopentyl ester (7ppp)

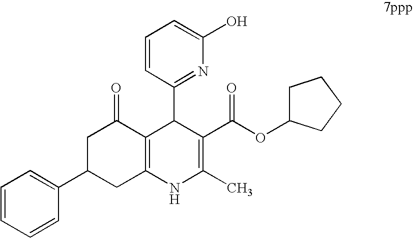

Synthesis of 4-(6-Hydroxy-pyridin-2-yl)-2-methyl-5-oxo-7-phenyl-1,4,5,6,7,8-hexahydro-quinoline-3-carboxylic acid cyclopentyl ester (7ppp): Compound 7ppp was prepared following the procedure described in Example 1 for the synthesis of 7a: MS (ES) M+H expected=445.2, found=445.2.

### Example 68

### Preparation of 4-(3-Methanesulfonylamino-phenyl)-2-methyl-5-oxo-7-phenyl-1,4,5,6,7,8-hexahydro-quinoline-3-carboxylic acid cyclopentyl ester (7qqq)

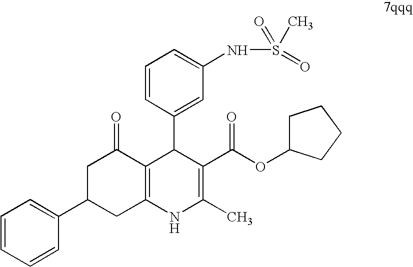

Synthesis of 4-(3-Methanesulfonylamino-phenyl)-2-methyl-5-oxo-7-phenyl-1,4,5,6,7,8-hexahydro-quinoline-3-carboxylic acid cyclopentyl ester (7qqq): Compound 7qqq was prepared following the procedure described in Example 1 for the synthesis of 7a: MS (ES) M+H expected=521.6, found=521.2.

### Example 69

### Preparation of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester (7rrr)

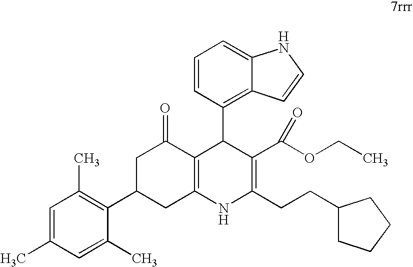

Synthesis of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester (7rrr): Compound 7rrr was prepared following the procedure described in Example 1 for the synthesis of 7a: MS (ES) M+H expected=550.3, found=550.7.

### Example 70

### Preparation of 4-(3-Hydroxy-phenyl)-2-methyl-5-oxo-7-phenyl-1,4,5,6,7,8-hexahydro-[1,7]naphthyridine-3-carboxylic acid cyclopentyl ester (7sss)

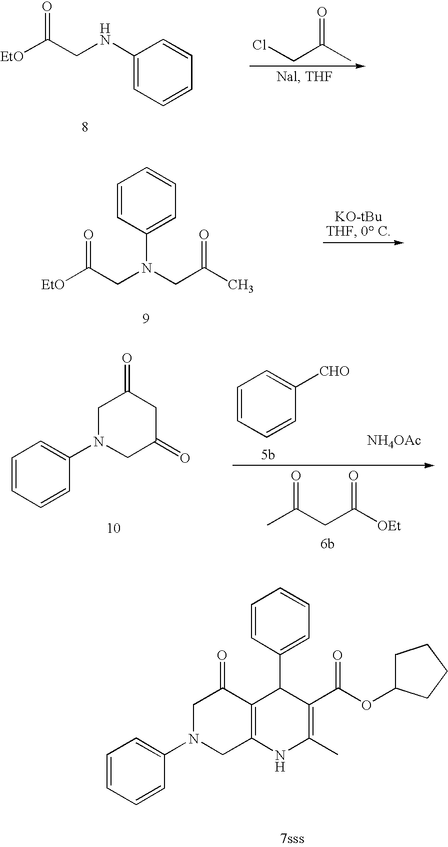

Synthesis of [(2-Oxo-propyl)-phenyl-amino]-acetic acid ethyl ester (9): N-Phenyl ethyl glycine 8 was dissolved in THF(80 mL) and K₂CO₃ (9.25 g, 67.2 mmol) was added, followed by chloroacetone (1.9 mL, 23.44 mmol) and NaI (3.75 g, 25.0 mmol). The mixture was heated to 60° C. with stirring for 48 hours. The reaction did not proceed past 33% by TLC and LC-MS. The reaction was allowed to cool to rt and was filtered. The filtrate was evaporated and the residue was purified by flash silica gel chromatography to give 1.7 g of an oil 9 for a 32% yield: MS (ES) M+H expected=235.3, found=236.4.

Synthesis of 1-Phenyl-piperidine-3,5-dione (10): The ketoester 9 was dissolved in THF (10 mL) and cooled to 0° C. To this cooled solution was added potassium tert-butoxide(4.5 mL, 4.5 mmol, 1.0 M in THF) dropwise. The reaction mixture was allowed to warm to room temperature overnight. The reaction was quenched with 1.0 M NaH₂PO₄ and extracted with a mixture of 3:1 methylene chloride: MeOH. The organic layer was dried over MgSO4, filtered and evaporated. The product 10 was pure enough to use in the next step: MS (ES) M+H expected=190.2, found=190.1.

Synthesis of 4-(3-Hydroxy-phenyl)-2-methyl-5-oxo-7-phenyl-1,4,5,6,7,8-hexahydro-[1,7]naphthyridine-3-carboxylic acid cyclopentyl ester (7sss): Compound 7sss was prepared following the procedure as described for compound 7a using compound 10 as the dione in the condensation reaction: MS (ES) M+H expected=445.5, found=445.2.

### Example 71

### Preparation of 2-Ethyl-4-(3-hydroxy-phenyl)-7-(1H-indol-3-yl)-5-oxo-1,4,5,6,7,8-hexahydro-quinoline-3-carboxylic acid cyclopentyl ester

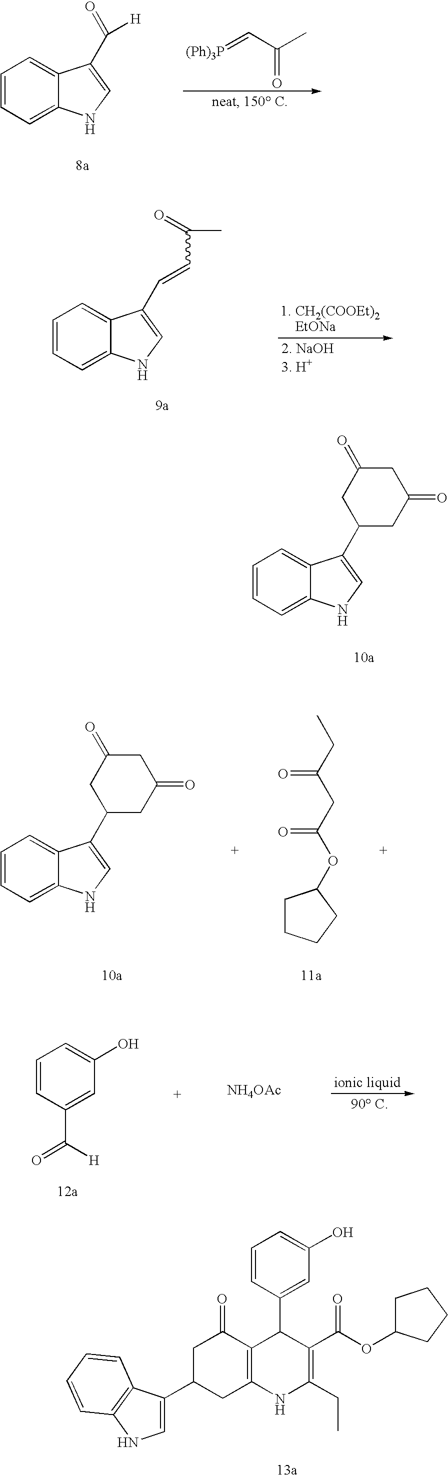

Synthesis of 4-(1H-indol-3-yl)-but-3-en-2-one (9a): A mixture of indole-3-carboxaldehyde (2.01 g, 13.8 mmol) and 1-triphenylphosphoranylidene-2-propanone (4.41 g, 13.8 mmol) was heated at 150° C. for one hour. After the mixture was cooled down to room temperature, 10 mL of ether was added in, the resulted rnixture was stirred vigorous for two hour at room temperature. Then, the solid was filtered away, and the filtrate was concentrated, purified by flash column chromatography on silica gel to provide 1.21 g of 4-(1H-indol-3-yl)-but-3-en-2-one as final product. ESMS m/z: 186.2 (M+H).

Synthesis of 5-(1H-indol-3-yl)-cyclohexane-1,3-dione (10a): To a suspension of sodium ethoxide (912.3 mg, 13.0 mmol) in 5 mL of ethanol was added diethyl malonate (1 mL, 6.5 mmol), followed by 4-(1H-Indol-3-yl)-but-3-en-2-one (1.21 g, 6.5 mmol). The resulted mixture was refluxed for overnight under nitrogen. The mixture was cooled down to room temperature, before the solid was filtered, dried, and dissolved in 2 N NaOH aqueous solution (3 mL). The solution was then refluxed for two hour before it was cooled down to room temperature. 5 N H₂SO₄ (3 mL) aqueous solution was added in the above basic solution, and the resulted mixture was refluxed for four hour. The mixture was cooled down again to room temperature, and the solid was filtered. The solid was then washed with water (5 m×3), dried over vacuum for overnight, and used as it was for next step reaction. ESMS m/z: 228.6 (M+H).

Synthesis of 2-Ethyl-4-(3-hydroxy-phenyl)-7-(1H-indol-3-yl)-5-oxo-1,4,5,6,7,8-hexahydro-quinoline-3-carboxylic acid cyclopentyl ester (13a): The titled compound was synthesized according to example 1. ESMS m/z: 497.3 (M+H).

### Example 72

### Preparation of 2-Ethyl-4-(3-hydroxy-phenyl)-7-(1H-indol-6-yl)-5-oxo-1,4,5,6,7,8-hexahydro-quinoline-3-carboxylic acid cyclopentyl ester

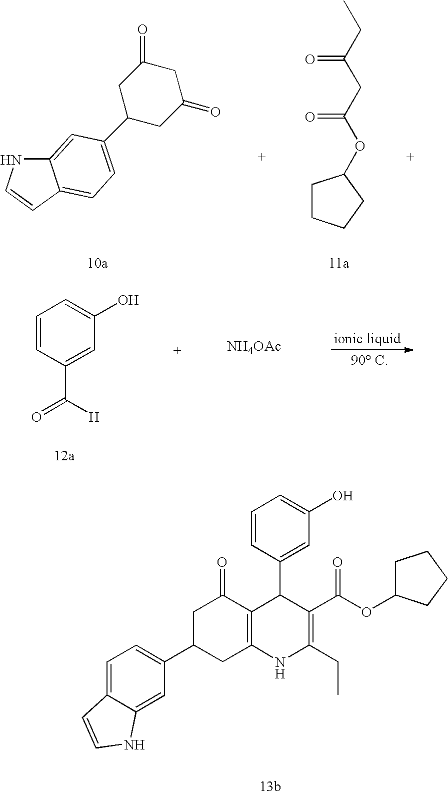

Synthesis of 2-Ethyl-4-(3-hydroxy-phenyl)-7-(1H-indol-6-yl)-5-oxo-1,4,5,6,7,8-hexahydro-quinoline-3-carboxylic acid cyclopentyl ester (13b): The synthesis of 2-Ethyl-4-(3-hydroxy-phenyl)-7-(1H-indol-6-yl)-5-oxo-1,4,5,6,7,8-hexahydro-quinoline-3-carboxylic acid cyclopentyl ester refers to synthesis of 2-Ethyl-4-(3-hydroxy-phenyl)-7-(1H-indol-3-yl)-5-oxo-1,4,5,6,7,8-hexahydro-quinoline-3-carboxylic acid cyclopentyl ester. ESMS m/z 497.5.

### Example 73

### Preparation of 2-Ethyl-4-(3-hydroxy-phenyl)-6-phenethyl-1,4-dihydro-pyridine-3,5-dicarboxylic acid 3-cyclopentyl ester 5-ethyl ester

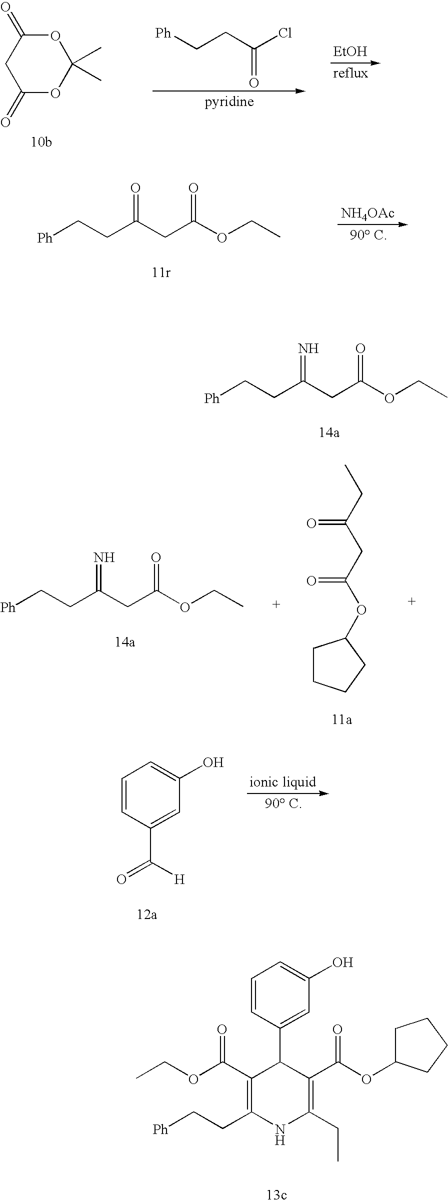

Synthesis of 3-Oxo-5-phenyl-pentanoic acid ethyl ester (11r):

To a mixture of meldrum's acid (6.05 g, 40.8 mmol) and pyridine (6.8 mL, 81.6 mmol) was added 3-Phenyl-propionyl chloride (6.86 g, 40.8 mmol) dropwise at 0° C., then continue to stir at 0° C. for another hour. The resulted mixture was warmed to room temperature, and the reaction was continued to be carried out for another hour. 30 mL brine was added in to above mixture, and the diluted mixture was extracted with 100 mL×3 dichloromethane. The combined dichloromethane solution was concentrated under reduced pressure, and the residue was redissolved into 30 mL of ethanol, and refluxed for two hour. The reaction was then cooled down to room temperature, concentrated, and purified by flash column chromatography to provide 5.24 g of 3-Oxo-5-phenyl-pentanoic acid ethyl ester as final product. ESMS m/z: 221.3 (M+H).

Synthesis of 2-Ethyl-4-(3-hydroxy-phenyl)-6-phenethyl-1,4-dihydro-pyridine-3,5-dicarboxylic acid 3-cyclopentyl ester 5-ethyl ester (13c): A mixture of 3-oxo-5-phenyl-pentanoic acid ethyl ester (80.3 mg, 0.35 mmol) and ammonium acetate (42.3 mg, 0.50 mmol) was heated at 95° C. for 15 min before it was cooled down to room temperature. To the above mixture was added 3-hydroxy benzoaldehyde (41.7 mg, 0.35 mmol), 3-oxo-pentanoic acid cyclopentyl ester (64 μL, 0.35 mmol), and ionic liquid (7.7 μL, 0.034 mmol). The resulted mixture was heated for 10 min at 90° C., before it was cooled down to room temperature. The resulted mixture was then directly purified by flash column chromatography to provide 15.1 mg of 2-ethyl-4-(3-hydroxy-phenyl)-6-phenethyl-1,4-dihydro-pyridine-3,5-dicarboxylic acid 3-cyclopentyl ester 5-ethyl ester as final product. ESMS m/z: 490.1 (M+H).

### Example 74

### Preparation of 2-tert-butoxymethyl-4-(3-hydroxy-phenyl)-5-oxo-7-(2,4,6-trimethyl-phenyl)-1,4,5,6,7,8-hexahydro-quinoline-3-carboxylic acid ethyl ester

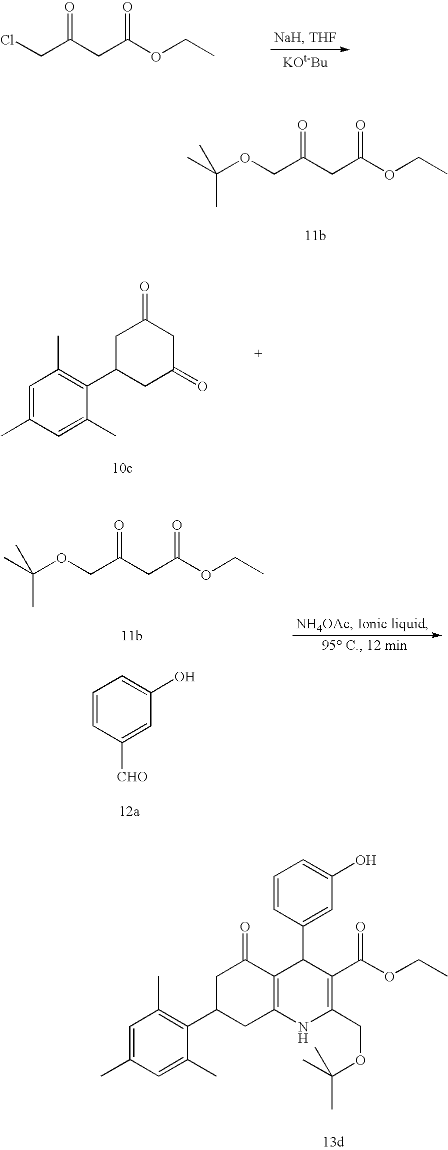

Synthesis of 4-tert-butoxy-3-oxo-butyric acid ethyl ester (11b): The β-ketoesters were prepared according to the published procedure (Seebach, Dieter; Eberle, Martin; Synthesis. 37, 1986). Sodium hydride (60%, 272 mg, 6.8 mmol) was added to a −78° C. stirred solution of 4-chloro-3-oxo-butyric acid ethyl ester (1 g, 6.07 mmol) in dry THF (10 mL) and the reaction mixture was stirred for 20 min. 1M solution of KO^{t-}Bu in THF (6.07 mL, 6.07 mmol) was added slowly over a period of 10 min. and the reaction mixture was allowed to attain room temperature over night. Then it was neutralized with 1M aqueous hydrochloric acid and extracted with ethyl acetate. The combined organic layers were washed with brine, dried (Na₂SO₄) and concentrated to get the crude product which was used as such in the following step. LC-MS (method: 20-100-5 min) retention time (Rt)=2.62 min. MS calc'd for C₁₀H₁₈O₄Na (MNa⁺): 225.1. Found 225.1

Synthesis of 2-tert-butoxymethyl-4-(3-hydroxy-phenyl)-5-oxo-7-(2,4,6-trimethyl-phenyl)-1,4,5,6,7,8-hexahydro-quinoline-3-carboxylic acid ethyl ester (13d): This compound was synthesized by using general Hantschz procedure. LC-MS (method: 20-100-5 min) retention time (Rt)=2.97 min. MS calc'd for C₃₂H₃₉NO₅ (MH⁺): 518.2. Found 518.2.

### Example 75

### Preparation of 4-(1H-indol-4-yl)-2-methoxymethyl-5-oxo-7-(2,4,6-trimethylphenyl)-1,4,5,6,7,8-hexahydro-quinoline-3-carboxylic acid methyl ester

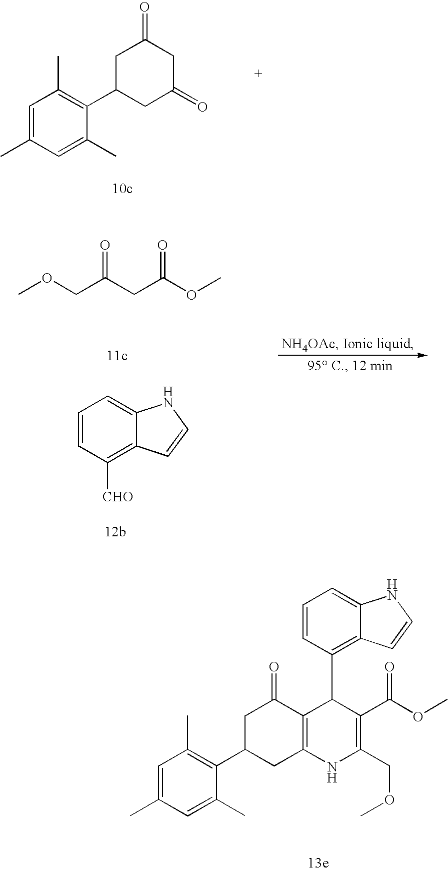

Synthesis of 4-(1H-indol-4-yl)-2-methoxymethyl-5-oxo-7-(2,4,6-trimethylphenyl)-1,4,5,6,7,8-hexahydro-quinoline-3-carboxylic acid methyl ester (13e): This compound was synthesized by using general Hantschz procedure. LC-MS (method: 20-100-5 min) retention time (Rt)=2.65 min. MS calc'd for C₃₀H₃₂N₂O₄ (MH⁺): 485.3. Found 485.3.

### Example 76

### Preparation of 2-tert-butoxymethyl-4-(3-hydroxymethylphenyl)-5-oxo-7-(2,4,6-trimethylphenyl)-1,4,5,6,7,8-hexahydro-quinoline-3-carboxylic acid ethyl ester

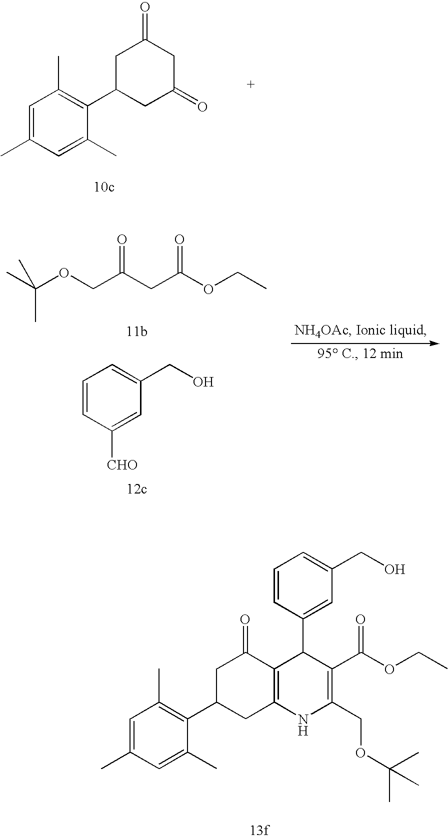

Synthesis of 2-tert-butoxymethyl-4-(3-hydroxymethylphenyl)-5-oxo-7-(2,4,6-trimethylphenyl)-1,4,5,6,7,8-hexahydro-quinoline-3-carboxylic acid ethyl ester (13f): This compound was synthesized by using general Hantschz procedure from 5-(2,4,6-Trimethyl-phenyl)-cyclohexane-1,3-dione, 3-hydroxymethylbenzaldehyde (Kobayashi, Yusuke; Fukuda, Akihiro; Kimachi, Tetsutaro; Ju-Ichi, Motoharu; Takemoto, Yoshiji; Tetrahedron 2005, 61, 2607), 4-tert-butoxy-3-oxo-butyric acid ethyl ester, ammonium acetate and in the presence of catalytic amount of ionic liquid. LC-MS (method: 20-100-5 min) retention time (Rt)=3.01 min. MS calc'd for C₃₃H₄₁NO₅ (MH⁺): 532.3. Found 532.3.

### Example 77

### Preparation of 2-Dimethoxymethyl-4-(3-hydroxy-phenyl)-5-oxo-7-(2,4,6-trimethyl-phenyl)-1,4,5,6,7,8-hexahydro-quinoline-3-carboxylic acid ethyl ester

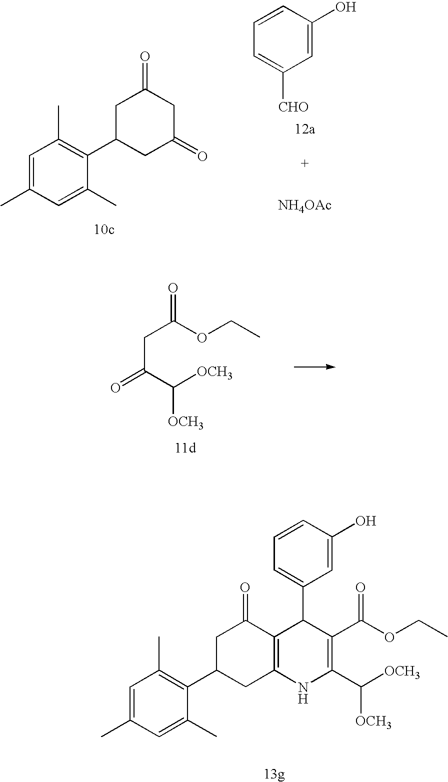

Synthesis of 2-Dimethoxymethyl-4-(3-hydroxy-phenyl)-5-oxo-7-(2,4,6-trimethyl-phenyl)-1,4,5,6,7,8-hexahydro-quinoline-3-carboxylic acid ethyl ester (13g): 5-(2,4,6-Trimethyl-phenyl)-cyclohexane-1,3-dione (1.00 g, 4.35 mmol) was combined with 3-hydroxybenzaldehyde (531 mg, 4.35 mmol), 4,4-dimethoxy-3-oxo-butyric acid ethyl ester (827 mg, 4.35 mmol) ammonium acetate (369 mmol, 4.78 mmol) and ionic liquid at 100° C. for 15 minutes. The product was used in the next reaction without purification. MS (ES) M+H expect 505.6, found 506.2.

### Example 78

### Preparation of 2-ethoxycarbonylmethyl-4-(3-hydroxyphenyl)-5-oxo-7-(2,4,6-trimethylphenyl)-1,4,5,6,7,8-hexahydroquinoline-3-carboxylic acid ethyl ester

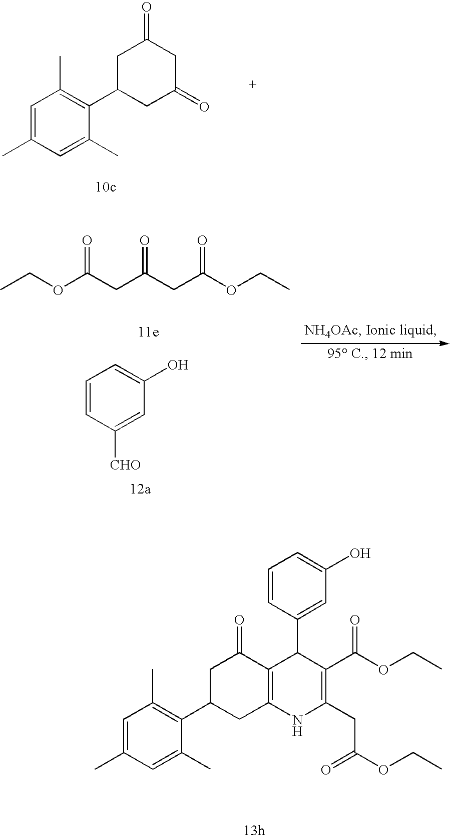

Synthesis of 2-ethoxycarbonylmethyl-4-(3-hydroxyphenyl)-5-oxo-7-(2,4,6-trimethylphenyl)-1,4,5,6,7,8-hexahydroquinoline-3-carboxylic acid ethyl ester (13h): This compound was synthesized by using general Hantschz procedure. LC-MS (method: 20-100-5 min) retention time (Rt)=2.57 min. MS calc'd for C₃₁H₃₆NO₆ (MH⁺): 518.3. Found 518.3.

### Example 79

### Preparation of 2-dimethylcarbamoylmethyl-4-(3-hydroxy-phenyl)-5-oxo-7-(2,4,6-trimethyl-phenyl)-1,4,5,6,7,8-hexahydro-quinoline-3-carboxylic acid ethyl ester

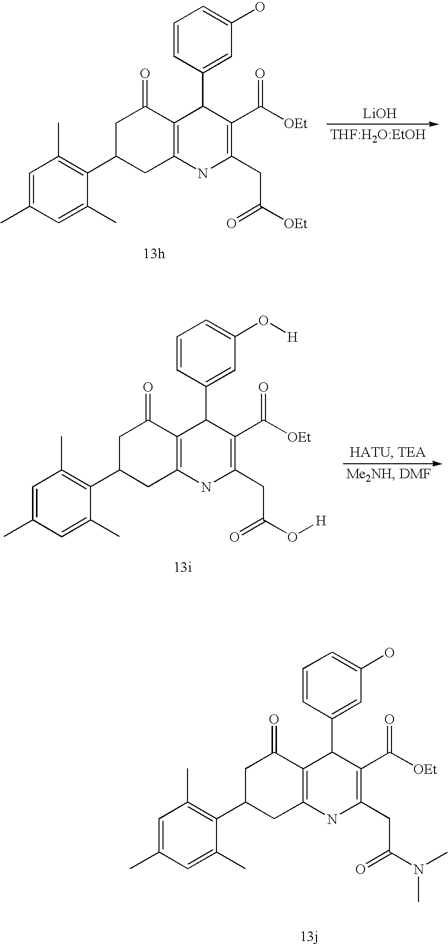

Synthesis of 2-Carboxymethyl-4-(3-hydroxy-phenyl)-5-oxo-7-(2,4,6-trimethyl-phenyl)-1,4,5,6,7,8-hexahydro-quinoline-3-carboxylic acid ethyl ester (13i): To a rt stirred solution of 2-ethoxycarbonylmethyl-4-(3-hydroxyphenyl)-5-oxo-7-(2,4,6-trimethylphenyl)-1,4,5,6,7,8-hexahydroquinoline-3-carboxylic acid ethyl ester (106 mg, 0.2 mmol) in a mixture of ethanol, tetrahydrofuran and water (1:2:1, 3 mL) was added 1M aqueous lithium hydroxide solution (0.4 mmol, 0.4 mL). Stirring continued for over night, the reaction mixture was neutralized with 1M aqueous hydrochloric acid and extracted with ethyl acetate (2×4\tilde{0} mL). The combined organic layers were dried (Na₂SO₄) and concentrated to get the crude product which was used as such in the following step. LC-MS (method: 20-100-5 min) retention time (Rt)=2.23 min. MS calc'd for C₂₉H₃₁NO₆ (MH⁺): 490.3. Found 490.3

Synthesis of 2-Dimethylcarbamoylmethyl-4-(3-hydroxy-phenyl)-5-oxo-7-(2,4,6-trimethyl-phenyl)-1,4,5,6,7,8-hexahydro-quinoline-3-carboxylic acid ethyl ester (13j): The above crude acid (42 mg, 0.086 mmol) was dissolved in DMF (1 mL). HATU (40 mg, 0.103 mmol), TEA (24 uL, 0.172 mmol) and dimethylamine solution in THF (43 uL, 0.086 mmol) were successively added. The reaction mixture was stirred for 1.5 h, excess solvent was removed in vacuum and residue was purified by HPLC. LC-MS (method: 20-100-5 min) retention time (Rt)=2.30 min. MS calc'd for C₃₁H₃₆N₂O₅ (MH⁺): 517.3. Found 517.3

### Example 80

### Preparation of 4-(2,3-Dimethyl-phenyl)-2-methyl-5-oxo-7-(2,4,6-trimethyl-phenyl)-1,4,5,6,7,8-hexahydro-quinoline-3-carbonitrile

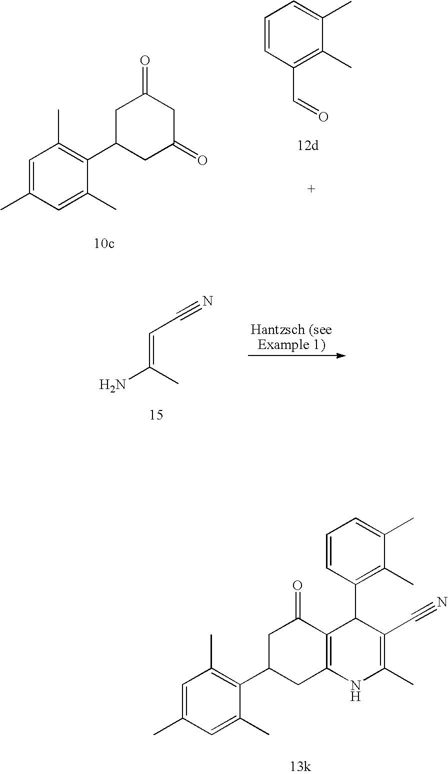

Synthesis of 4-(2,3-Dimethyl-phenyl)-2-methyl-5-oxo-7-(2,4,6-trimethyl-phenyl)-1,4,5,6,7,8-hexahydro-quinoline-3-carbonitrile (13k): The Hantzsch reaction was performed as described in Example 1 and purified by prep TLC to give product. (MS calc'd for C₂₈H₃₁N₂O (MH⁺): 411.2. Found 411.3.

### Example 81

### Preparation of 2-tert-Butoxymethyl-4-(3-carbamoyl-phenyl)-5-oxo-7-(2,4,6-trimethyl-phenyl)-1,4,5,6,7,8-hexahydro-quinoline-3-carboxylic acid ethyl ester

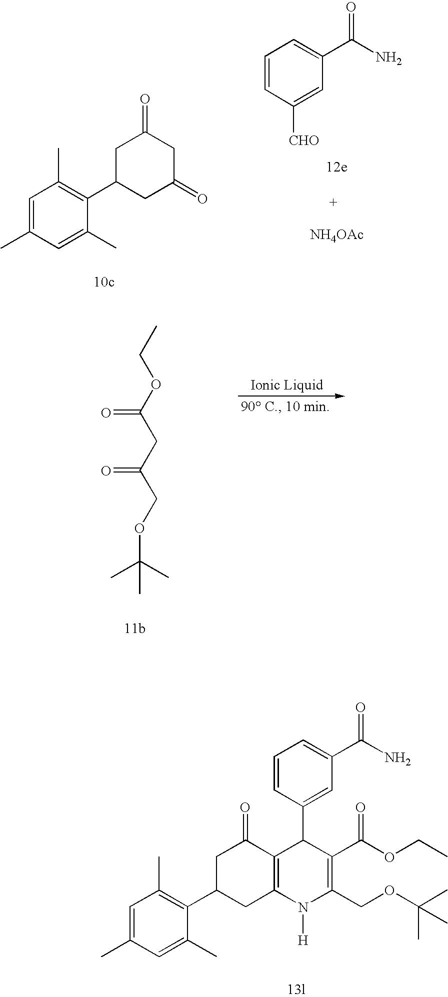

Synthesis of 2-tert-Butoxymethyl-4-(3-carbamoyl-phenyl)-5-oxo-7-(2,4,6-trimethyl-phenyl)-1,4,5,6,7,8-hexahydro-quinoline-3-carboxylic acid ethyl ester (13l): To a small vial was added 3-formyl-benzamide (26 mg, 0.17 mmol) followed by 5-(2,4,6-trimethyl-phenyl)-cyclohexane-1,3-dione (40 mg, 0.17 mmol), ammonium acetate (20 mg, 0.26 mmol), 4-tert-butoxy-3-oxo-butyric acid ethyl ester (35 mg, 0.17 mmol) and 1-butyl-3-methylimidazolium tetrafluoroborate (3.9 □L, 0.021 mmol). The reaction mixture was then heated at 90° C. for 10 minutes, cooled down to room temperature, and then loaded on column (50% ethyl acetate/hexanes) to get the desired product (57 mg, 60%) as a yellow solid. MS (ES) M+H expected=545.3, found=545.3.

### Example 82

### Preparation of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester

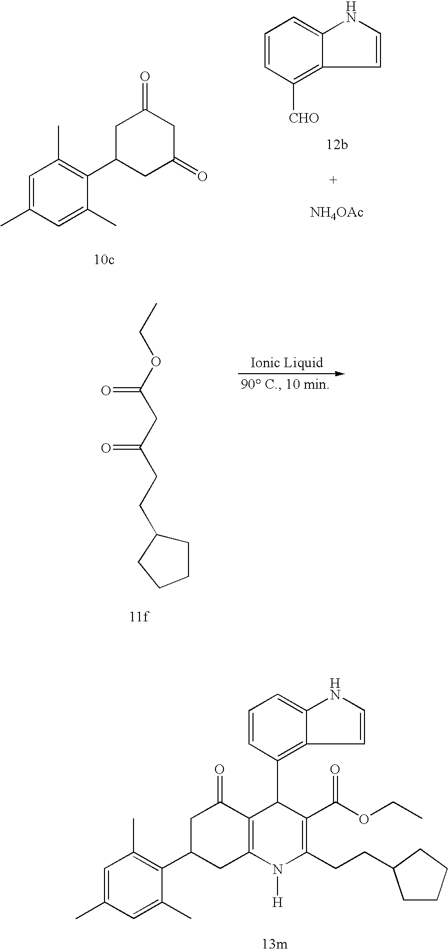

Synthesis of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester (13m): To a small vial was added 1H-indole-4-carbaldehyde (38 mg, 0.26 mmol) followed by 5-(2,4,6-trimethyl-phenyl)-cyclohexane-1,3-dione (60 mg, 0.26 mmol), ammonium acetate (30 mg, 0.39 mmol), 5-cyclopentyl-3-oxo-pentanoic acid ethyl ester (55 mg, 0.26 mmol) and 1-butyl-3-methylimidazolium tetrafluoroborate (5.8 μL, 0.031 mmol). The reaction mixture was then heated at 90° C. for 10 minutes, cooled down to room temperature, and then loaded on column (50% ethyl acetate/hexanes) to get the desired product (72 mg, 50%) as a yellow solid. MS (ES) M+H expected=551.3, found=551.2.

### Example 83

### Preparation of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester

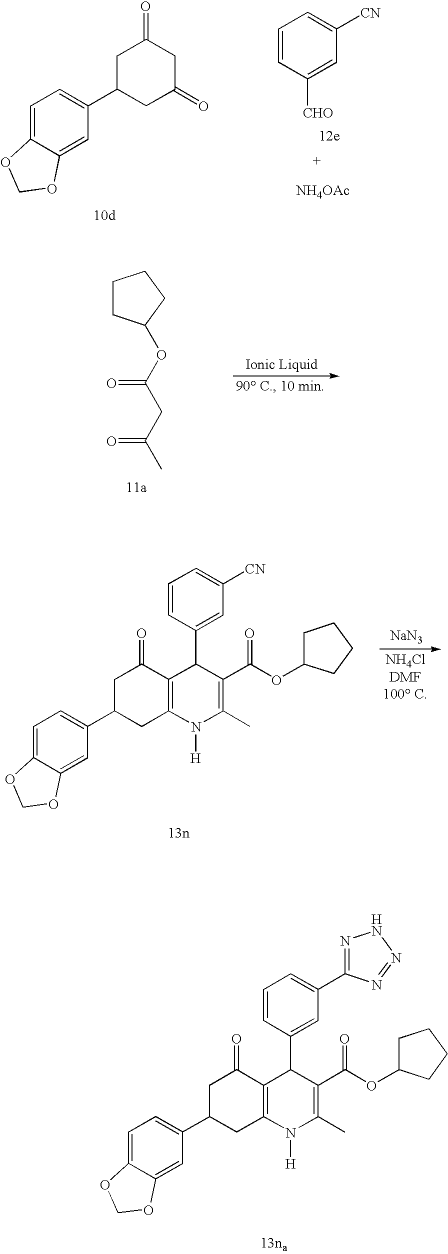

Synthesis of 7-Benzo[1,3]dioxol-5-yl-4-(3-cyano-phenyl)-2-methyl-5-oxo-1,4,5,6,7,8-hexahydro-quinoline-3-carboxylic acid cyclopentyl ester (13n): To a small vial was added 3-formyl-benzonitrile (85 mg, 0.65 mmol) followed by 5-(2,4,6-trimethyl-phenyl)-cyclohexane-1,3-dione (150 mg, 0.65 mmol), ammonium acetate (75 mg, 0.97 mmol), 3-oxo-butyric acid cyclopentyl ester (110 mg, 0.65 mmol) and 1-butyl-3-methylimidazolium tetrafluoroborate (14.5 μL, 0.077 mmol). The reaction mixture was then heated at 90° C. for 10 minutes, cooled down to room temperature, and then loaded on column (50% ethyl acetate/hexanes) to get two diastereomers. Diastereomer A (higher Rf): MS (ES) M+H expected=497.2, found=497.2. Diastereomer B (lower Rf): MS (ES) M+H expected=497.2, found=497.3.

Synthesis of 2-(2-Cyclopentyl-ethyl)-4-(1H-indol-4-yl)-5-oxo-7-(2,4,6-trimethyl-phenyl)-1,4,5,6,7,8-hexahydro-quinoline-3-carboxylic acid ethyl ester (13nₐ): Diastereomer A (66 mg, 0.13 mmol) was dissolved in 3.0 mL of DMF. Sodium azide (34 mg, 0.53 mmol) was added, followed by ammonium chloride (28 mg, 0.53 mmol). The reaction mixture was heated at 100° C. overnight. DMF was removed under high vacuum, and the residue was purified by column (50% ethyl acetate/hexanes) to get the desired product (14 mg, 20%) as a yellow solid. MS (ES) M+H expected=540.2, found=540.4.

### Example 84

### Preparation of 7-Benzo[1,3]dioxol-5-yl-4-(3-methanesulfonylamino-phenyl)-2-methyl-5-oxo-1,4,5,6,7,8-hexahydro-quinoline-3-carboxylic acid cyclopentyl ester

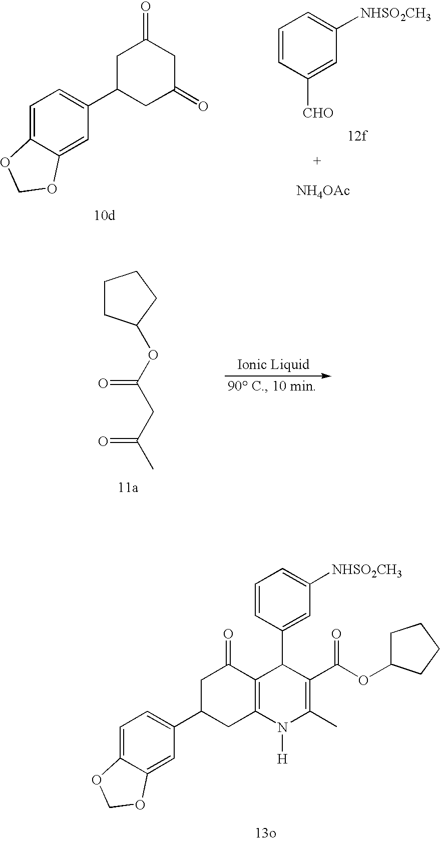

Synthesis of 7-Benzo[1,3]dioxol-5-yl-4-(3-methanesulfonylamino-phenyl)-2-methyl-5-oxo-1,4,5,6,7,8-hexahydro-quinoline-3-carboxylic acid cyclopentyl ester (13o): To a small vial was added N-(3-formyl-phenyl)-methanesulfonamide (86 mg, 0.43 mmol) followed by 5-benzo[1,3]dioxol-5-yl-cyclohexane-1,3-dione (100 mg, 0.43 mmol), ammonium acetate (50 mg, 0.65 mmol), 3-oxo-butyric acid cyclopentyl ester (73 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (50% ethyl acetate/hexanes) to get the desired product (121 mg, 50%) as a yellow solid. MS (ES) M+H expected=565.2, found=565.3.

### Example 85

### Preparation of 4-(3-Amino-phenyl)-2-methyl-5-oxo-7-(2,4,6-trimethyl-phenyl)-1,4,5,6,7,8-hexahydro-quinoline-3-carboxylic acid ethyl ester

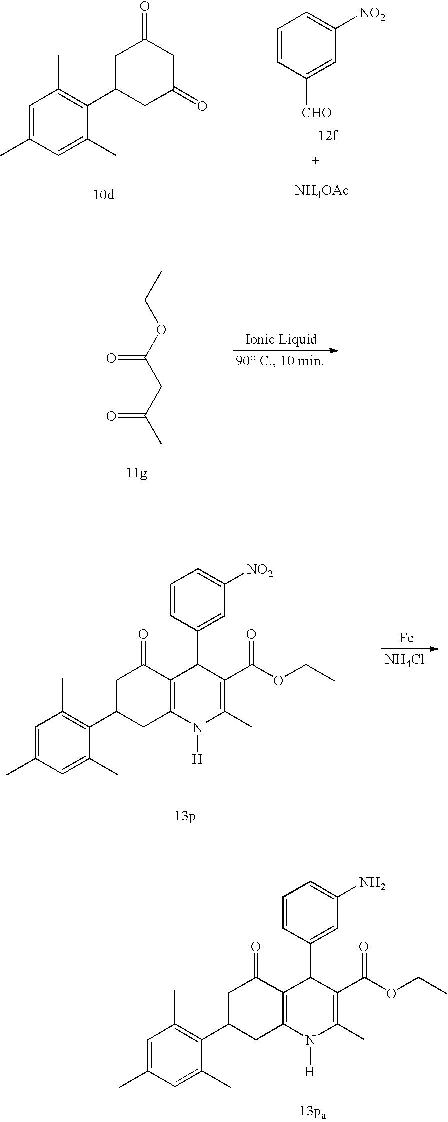

Synthesis of 2-Methyl-4-(3-nitro-phenyl)-5-oxo-7-(2,4,6-trimethyl-phenyl)-1,4,5,6,7,8-hexahydro-quinoline-3-carboxylicacid ethyl ester (13p): To a small vial was added 3-nitro-benzaldehyde (98 mg, 0.65 mmol) followed by 5-(2,4,6-trimethyl-phenyl)-cyclohexane-1,3-dione (150 mg, 0.65 mmol), ammonium acetate (75 mg, 0.98 mmol), 3-oxo-butyric acid ethyl ester (85 mg, 0.65 mmol) and 1-butyl-3-methylimidazolium tetrafluoroborate (14.6 μL, 0.078 mmol). The reaction mixture was then heated at 90° C. for 10 minutes, cooled down to room temperature, and then loaded on column (50% ethyl acetate/hexanes) to get the desired product (185 mg, 60%) as a yellow solid. MS (ES) M+H expected=475.2, found=475.3.

Synthesis of 4-(3-Amino-phenyl)-2-methyl-5-oxo-7-(2,4,6-trimethyl-phenyl)-1,4,5,6,7,8-hexahydro-quinoline-3-carboxylic acid ethyl ester: The nitro compound obtained above (150 mg, 0.32 mmol) was dissolved in 10 mL of EtOH-water (2:1) solution. Then ammonium chloride (169 mg, 3.2 mmol) was added, followed by iron powder (53 mg, 0.96 mmol). The reaction mixture was refluxed for one hour, cooled to room temperature, and then filtered. The filtrate was put on rotovap to remove most of the solvent. The residue was then dissolved in dichloromethane and washed with brine. The organic layers were combined and dried over MgSO₄. Solvent was then removed and the residue was purified by column chromatography (80% ethyl acetate/hexanes). The product (77 mg, 55%) was obtained as a yellow solid. MS (ES) M+H expected=445.2, found=445.1.

### Example 86

### Preparation of 4-(3-Hydroxy-phenyl)-3-methanesulfonyl-2-methyl-7-(2,4,6-trimethyl-phenyl)-4,6,7,8-tetrahydro-1H-quinolin-5-one

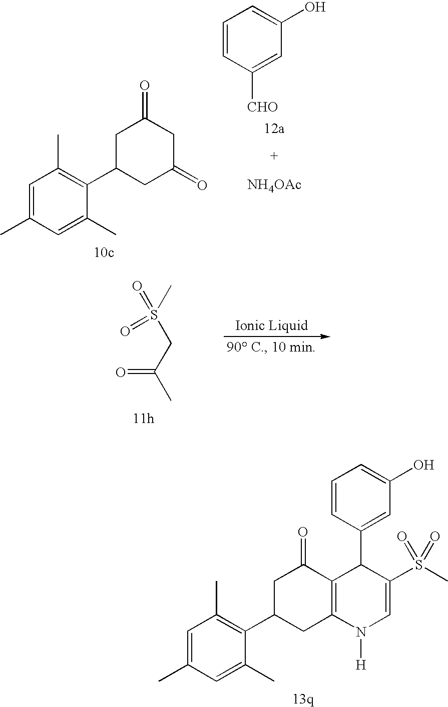

Synthesis of 4-(3-Hydroxy-phenyl)-3-methanesulfonyl-2-methyl-7-(2,4,6-trimethyl-phenyl)-4,6,7,8-tetrahydro-1H-quinolin-5-one (13q): To a small vial was added 3-hydroxy-benzaldehyde (42 mg, 0.35 mmol) followed by 5-(2,4,6-trimethyl-phenyl)-cyclohexane-1,3-dione (80 mg, 0.35 mmol), ammonium acetate (40 mg, 0.52 mmol), 1-methanesulfonyl-propan-2-one (47 mg, 0.35 mmol) and 1-butyl-3-methylimidazolium tetrafluoroborate (7.8 μL, 0.042 mmol). The reaction mixture was then heated at 90° C. for 10 minutes, cooled down to room temperature, and then loaded on column (70% ethyl acetate/hexanes) to get the desired product (63 mg, 40%) as a solid. MS (ES) M+H expected=452.2, found=452.3.

### Example 87

### Preparation of 3-Benzenesulfonyl-4-(3-hydroxy-phenyl)-2-methoxymethyl-7-(2,4,6-trimethyl-phenyl)-1,4,7,8-tetrahydro-pyrano[4,3-b]pyridin-5-one

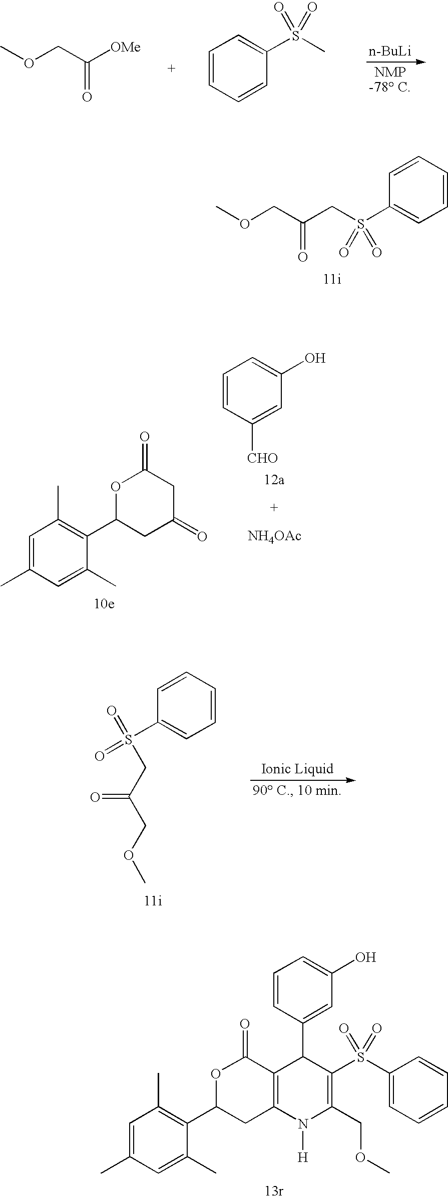

Synthesis of 1-Benzenesulfonyl-3-methoxy-propan-2-one (11i): Methanesulfonyl-benzene (180 mg, 1.15 mmol) was dissolved in 4 mL of THF and cooled to −78° C. To this mixture was added butyllithium (2.5 M solution in hexanes, 0.46 mL, 1.15 mmol). After stirring for 10 minutes at −78° C., 1-methyl-2-pyrrolidinone (NMP, 0.5 mL) was added. After another 10 minutes, a solution of methoxy-acetic acid methyl ester (120 mg, 1.15 mmol) in 1 mL of THF was added, and the reaction mixture was warmed up to room temperature. The reaction was quenched with sat. NH₄Cl solution and extracted with ethyl acetate. The organic layers were combined and washed with brine and dried over MgSO₄. Solvent was then removed and the residue was purified by column chromatography (80% ethyl acetate/hexanes). The product (77 mg, 55%) was obtained as a solid. MS (ES) M+H expected=229.0, found=229.1.

Synthesis of 3-Benzenesulfonyl-4-(3-hydroxy-phenyl)-2-methoxymethyl-7-(2,4,6-trimethyl-phenyl)-1,4,7,8-tetrahydro-pyrano[4,3-b]pyridin-5-one (13r): To a small vial was added 3-hydroxy-benzaldehyde (53 mg, 0.43 mmol) followed by 6-(2,4,6-trimethyl-phenyl)-dihydropyran-2,4-dione (100 mg, 0.43 mmol), ammonium acetate (50 mg, 0.65 mmol), 1-benzenesulfonyl-3-methoxy-propan-2-one (98 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (70% ethyl acetate/hexanes) to get the desired product (117 mg, 50%) as a yellow solid. MS (ES) M+H expected=546.2, found=546.2.

### Example 88

### Preparation of 2-tert-Butoxymethyl-5-oxo-7-(2,4,6-trimethyl-phenyl)-4-(3-ureido-phenyl)-1,4,5,6,7,8-hexahydro-quinoline-3-carboxylic acid ethyl ester

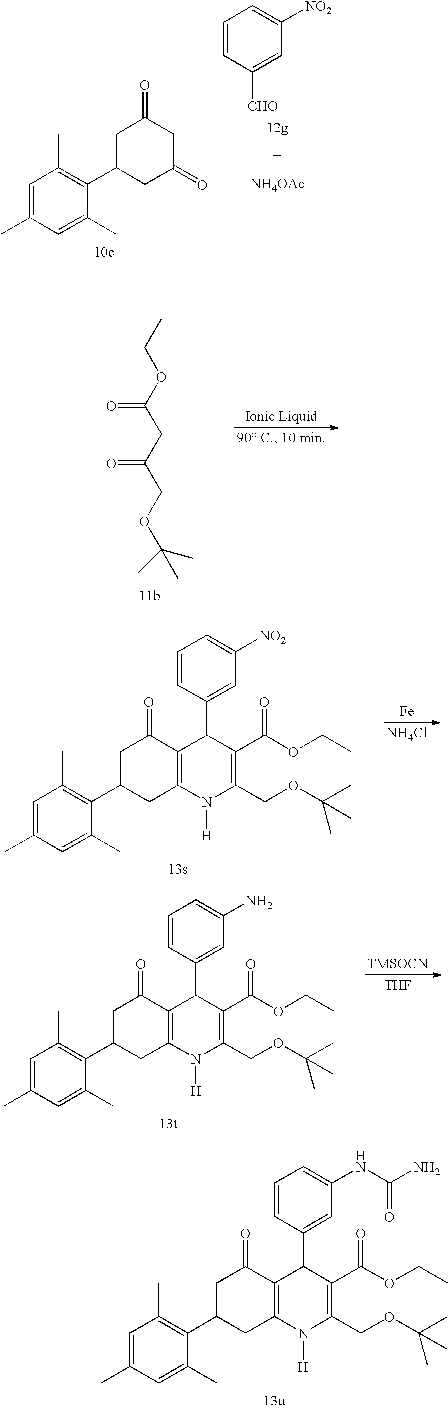

Synthesis of 2-tert-Butoxymethyl-4-(3-nitro-phenyl)-5-oxo-7-(2,4,6-trimethyl-phenyl)-1,4,5,6,7,8-hexahydro-quinoline-3-carboxylic acid ethyl ester (13s): To a small vial was added 3-nitro-benzaldehyde (66 mg, 0.43 mmol) followed by 5-(2,4,6-trimethyl-phenyl)-cyclohexane-1,3-dione (100 mg, 0.43 mmol), ammonium acetate (50 mg, 0.65 mmol), 4-tert-butoxy-3-oxo-butyric acid ethyl ester (88 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (50% ethyl acetate/hexanes) to get the desired product (119 mg, 60%) as a yellow solid. MS (ES) M+H expected=547.3, found=547.4.

Synthesis of 4-(3-Amino-phenyl)-2-tert-butoxymethyl-5-oxo-7-(2,4,6-trimethyl-phenyl)-1,4,5,6,7,8-hexahydro-quinoline-3-carboxylic acid ethyl ester (13t): The nitro compound obtained above (63 mg, 0.12 mmol) was dissolved in 6 mL of EtOH-water (2:1) solution. Then ammonium chloride (62 mg, 1.2 mmol) was added, followed by iron powder (19 mg, 0.36 mmol). The reaction mixture was refluxed for one hour, cooled to room temperature, and then filtered. The filtrate was put on rotovap to remove most of the solvent. The residue was then dissolved in dichloromethane and washed with brine. The organic layers were combined and dried over MgSO₄. Solvent was then removed and the residue was purified by column chromatography (80% ethyl acetate/hexanes). The product (36 mg, 60%) was obtained as a yellow solid. MS (ES) M+H expected=517.3, found=517.2.

Synthesis of 2-tert-Butoxymethyl-5-oxo-7-(2,4,6-trimethyl-phenyl)-4-(3-ureido-phenyl)-1,4,5,6,7,8-hexahydro-quinoline-3-carboxylic acid ethyl ester (13u): The aniline compound obtained above (20 mg, 0.039 mmol) was dissolved in 1 mL of THF. TMSOCN (0.5 mL) was then added and the reaction mixture was stirred overnight at room temperature. The reaction mixture was then loaded on column (70% ethyl acetate/hexanes) to get the desired product (17 mg, 80%) as a solid. MS (ES) M+H expected=560.3, found=560.3.

### Example 89

### Preparation of 2-tert-Butoxymethyl-5-oxo-4-(3-sulfamide-phenyl)-7-(2,4,6-trimethyl-phenyl)-1,4,5,6,7,8-hexahydro-quinoline-3-carboxylic acid ethyl ester

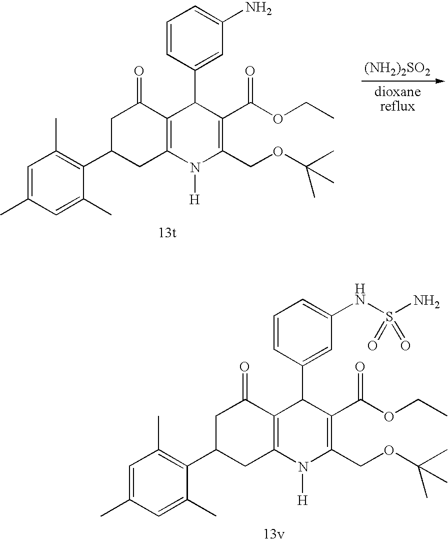

Synthesis of 2-tert-Butoxymethyl-5-oxo-4-(3-sulfamide-phenyl)-7-(2,4,6-trimethyl-phenyl)-1,4,5,6,7,8-hexahydro-quinoline-3-carboxylic acid ethyl ester (13v): 4-(3-Amino-phenyl)-2-tert-butoxymethyl-5-oxo-7-(2,4,6-trimethyl-phenyl)-1,4,5,6,7,8-hexahydro-quinoline-3-carboxylic acid ethyl ester (25 mg, 0.048 mmol) was dissolved in 1 mL of 1,4-dioxane. Sulfamide (5.1 mg, 0.053 mmol) was then added and the reaction mixture was heated to reflux overnight. The reaction mixture was then loaded on column (70% ethyl acetate/hexanes) to get the desired product (11 mg, 40%) as a solid. MS (ES) M+H expected=596.3, found=596.3.

### Example 90

### Preparation of 2-tert-Butoxymethyl-4-(2-fluoro-5-hydroxy-phenyl)-5-oxo-7-(2,4,6-trimethyl-phenyl)-1,4,5,6,7,8-hexahydro-quinoline-3-carboxylic acid ethyl ester

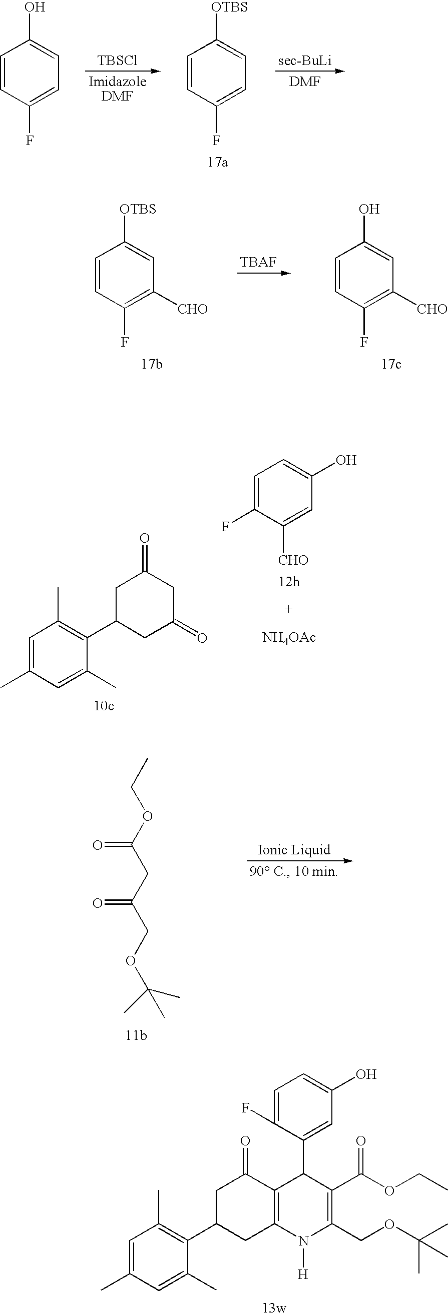

Synthesis of tert-Butyl-(4-fluoro-phenoxy)-dimethyl-silane (17a): 4-Fluoro-phenol (1.0 g, 8.93 mmol) was dissolved in 10 mL of DMF and cooled to 0° C. Immidazole (1.2 g, 17.86 mmol) was added followed by TBSCl (1.6 g, 10.72 g). The reaction mixture was warmed up to room temperature and stirred overnight. The reaction mixture was quenched with NH₄Cl and extracted with ethyl acetate. The organic layers were combined and dried over MgSO₄. Solvent was then removed and the residue was purified by column chromatography (10% ethyl acetate/hexanes). The product (1.82 g, 90%) was obtained as a colorless liquid. MS (ES) M+H expected=227.1, found=227.2.

Synthesis of 5-(tert-Butyl-dimethyl-silanyloxy)-2-fluoro-benzaldehyde (17b): tert-Butyl-(4-fluoro-phenoxy)-dimethyl-silane (500 mg, 2.21 mmol) was dissolved in 6 mL of THF and cooled to −78° C. A solution of sec-butyllithium in cyclohexanes (1.4 M, 1.7 mL, 2.4 mL) was added dropwise. After stirring for half an hour at −78° C., DMF (1 mL) was added and the reaction mixture was warmed up to room temperature and quenched with HCl (2.0 M). The reaction mixture was extracted with ethyl acetate. The organic layers were combined and dried over MgSO₄. Solvent was then removed and the residue was purified by column chromatography (15% ethyl acetate/hexanes). The product (337 mg, 60%) was obtained as a colorless liquid. MS (ES) M+H expected=255.1, found=255.1.

Synthesis of 2-Fluoro-5-hydroxy-benzaldehyde (17c): The above TBS-protected aldehyde (490 mg, 1.93 mmol) was dissolved in 5 mL of THF. Then a solution of TBAF in THF (1.0 M, 3.8 mL, 3.80 mmol) was added and the reaction mixture was stirred for two hours at room temperature. The reaction mixture was quenched with NH₄Cl and extracted with ethyl acetate. The organic layers were combined and dried over MgSO₄. Solvent was then removed and the residue was purified by column chromatography (50% ethyl acetate/hexanes). The product (216 mg, 80%) was obtained as a colorless liquid. MS (ES) M+H expected=141.0, found=141.1.

Synthesis of 2-tert-Butoxymethyl-4-(2-fluoro-5-hydroxy-phenyl)-5-oxo-7-(2,4,6-trimethyl-phenyl)-1,4,5,6,7,8-hexahydro-quinoline-3-carboxylic acid ethyl ester (13w): To a small vial was added 2-fluoro-5-hydroxy-benzaldehyde (49 mg, 0.35 mmol) followed by 5-(2,4,6-trimethyl-phenyl)-cyclohexane-1,3-dione (80 mg, 0.35 mmol), ammonium acetate (40 mg, 0.52 mmol), 4-tert-butoxy-3-oxo-butyric acid ethyl ester (51 mg, 0.35 mmol) and 1-butyl-3-methylimidazolium tetrafluoroborate (7.8 μL, 0.042 mmol). The reaction mixture was then heated at 90° C. for 10 minutes, cooled down to room temperature, and then loaded on column (50% ethyl acetate/hexanes) to get the desired product (75 mg, 45%) as a yellow solid. MS (ES) M+H expected=536.3, found=536.2.

### Example 91

### Preparation of 2-tert-Butoxymethyl-4-(1H-indazol-4-yl)-5-oxo-7-(2,4,6-trimethyl-phenyl)-1,4,5,6,7,8-hexahydro-quinoline-3-carboxylic acid ethyl ester

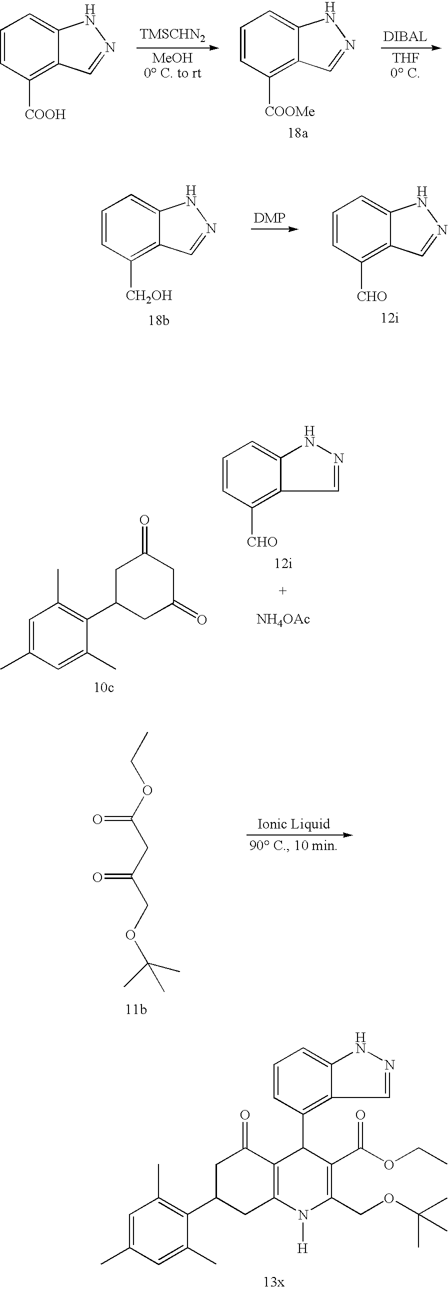

Synthesis of 1H-Indazole-4-carboxylic acid methyl ester (18a): To a solution of 1H-Indazole-4-carboxylic acid (100 mg, 0.62 mmol) in 6 mL of methanol-dichloromethane (1:1) was added trimethylsilyl diazomethane (2.0 M in ethyl ether) dropwise at room temperature. More trimethylsilyl diazomethane was added until the starting material disappeared. Solvent was removed carefully and the residue was purified by column chromatography (50% ethyl acetate/hexanes). The product (54 mg, 50%) was obtained as a colorless solid. MS (ES) M+H expected=177.1, found=177.2.

Synthesis of (1H-Indazol-4-yl)-methanol (18b): The ester obtained above (40 mg, 0.23 mmol) was dissolved in 1 mL of THF and cooled to 0° C. A solution of DIBAL in THF (1.0 M, 2.3 mL, 2.3 mmol) was added dropwise. More DIBAL was added until the starting material disappeared. The reaction was then quenched with saturated potassium sodium tartrate solution and warmed up to room temperature. The reaction mixture was extracted with ethyl acetate. The organic layers were combined and dried over MgSO₄. Solvent was then removed and the residue was purified by column chromatography (75% ethyl acetate/hexanes). The product (20 mg, 60%) was obtained as a white solid. MS (ES) M+H expected=149.1, found=149.1.

Synthesis of 1H-Indazole-4-carbaldehyde (12i): The alcohol obtained above (54 mg, 0.36 mmol) was dissolved in 3 mL of THF, and then Dess-Martin periodinine (247 mg, 0.58 mmol) was added. The reaction mixture was stirred at room temperature overnight and quenched with Na₂S₂O₃ (2.0 M) solution. The mixture was extracted with ethyl acetate, and the combined organic layers were combined and washed with saturated NaHCO₃ solution, brine, and dried over MgSO₄. Solvent was then removed and the residue was purified by column chromatography (75% ethyl acetate/hexanes). The product (37 mg, 70%) was obtained as a white solid. MS (ES) M+H expected=147.1, found=147.2.

Synthesis of 2-tert-Butoxymethyl-4-(1H-indazol-4-yl)-5-oxo-7-(2,4,6-trimethyl-phenyl)-1,4,5,6,7,8-hexahydro-quinoline-3-carboxylic acid ethyl ester(13x): To a small vial was added 1H-Indazole-4-carbaldehyde (30 mg, 0.21 mmol) followed by 5-(2,4,6-trimethyl-phenyl)-cyclohexane-1,3-dione (47 mg, 0.21 mmol), ammonium acetate (24 mg, 0.31 mmol), 4-tert-Butoxy-3-oxo-butyric acid ethyl ester (41 mg, 0.21 mmol) and 1-butyl-3-methylimidazolium tetrafluoroborate (4.6 μL, 0.025 mmol). The reaction mixture was then heated at 90° C. for 10 minutes, cooled down to room temperature, and then loaded on column (50% ethyl acetate/hexanes) to get the desired product (33 mg, 30%) as a solid. MS (ES) M+H expected=542.3, found=542.3.

### Example 92

### Preparation of 2-tert-Butoxymethyl-5-oxo-4-(2-oxo-2,3-dihydro-benzooxazol-7-yl)-7-(2,4,6-trimethyl-phenyl)-1,4,5,6,7,8-hexahydro-quinoline-3-carboxylic acid ethyl ester

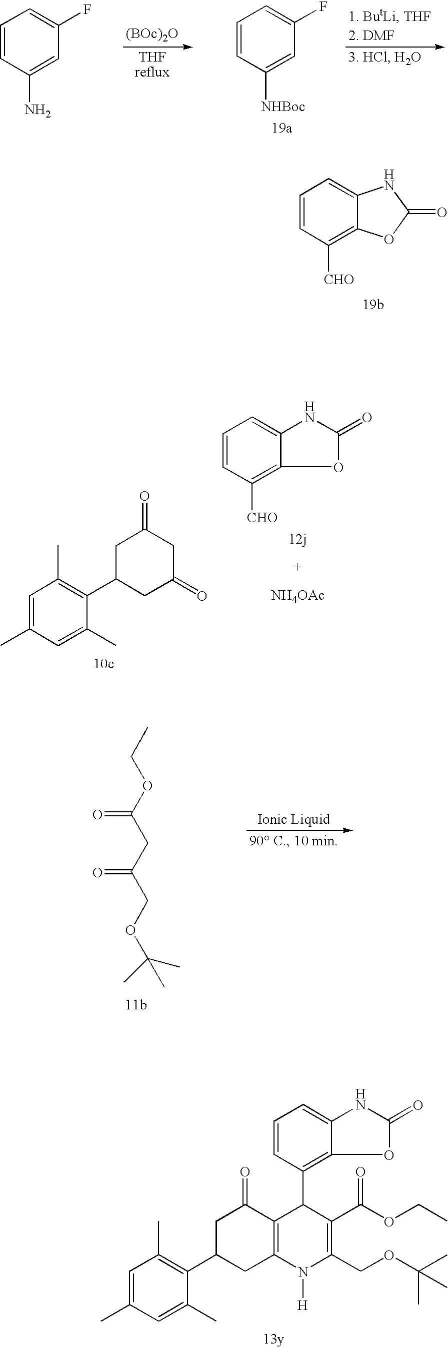

Synthesis of (3-Fluoro-phenyl)-carbamic acid tert-butyl ester (19a): 3-Fluoro-phenylamine (0.6 g, 5.4 mmol) and (Boc)₂O (3.1 mL, 13.5 mmol) were mixed and heated to reflux overnight. The reaction was then quenched with saturated ammonium chloride solution and extracted with ethyl acetate. The organic layers were combined, washed with brine and dried over MgSO₄. Solvent was then removed and the residue was purified by column chromatography (40% ethyl acetate/hexanes). The product (0.68 g, 60%) was obtained as a white solid. MS (ES) M+H expected=212.1, found=212.1.

Synthesis of 2-Oxo-2,3-dihydro-benzooxazole-7-carbaldehyde (19b): To a solution of (3-fluoro-phenyl)-carbamic acid tert-butyl ester (100 mg, 0.47 mmol) in THF (1 mL) was added tert-butyllithium (1.7 M in pentane, 0.84 mL, 1.42 mmol) at −40° C. After stirring for one hour, DMF (0.2 mL) was added and the reaction mixture was stirred for another hour at −40° C. before warming up to room temperature. The reaction was quenched with HCl solution (2.0 M) and extracted with ethyl acetate. The organic layers were combined, washed with brine and dried over MgSO₄. Solvent was then removed and the residue was purified by column chromatography (50% ethyl acetate/hexanes). The product (46 mg, 60%) was obtained as a white solid. MS (ES) M+H expected=164.0, found=164.1.

Synthesis of 2-tert-Butoxymethyl-5-oxo-4-(2-oxo-2,3-dihydro-benzooxazol-7-yl)-7-(2,4,6-trimethyl-phenyl)-1,4,5,6,7,8-hexahydro-quinoline-3-carboxylic acid ethyl ester (13y): To a small vial was added 2-oxo-2,3-dihydro-benzooxazole-7-carbaldehyde (22 mg, 0.14 mmol) followed by 5-(2,4,6-trimethyl-phenyl)-cyclohexane-1,3-dione (31 mg, 0.14 mmol), ammonium acetate (16 mg, 0.20 mmol), 4-tert-butoxy-3-oxo-butyric acid ethyl ester (27 mg, 0.14 mmol) and 1-butyl-3-methylimidazolium tetrafluoroborate (3.0 μL, 0.016 mmol). The reaction mixture was then heated at 90° C. for 10 minutes, cooled down to room temperature, and then loaded on column (50% ethyl acetate/hexanes) to get the desired product (23 mg, 30%) as a solid. MS (ES) M+H expected=559.3, found=559.3.

### Example 93

### Preparation of 2-tert-Butoxymethyl-5-oxo-4-(2-oxo-2,3-dihydro-benzothiazol-7-yl)-7-(2,4,6-trimethyl-phenyl)-1,4,5,6,7,8-hexahydro-quinoline-3-carboxylic acid ethyl ester

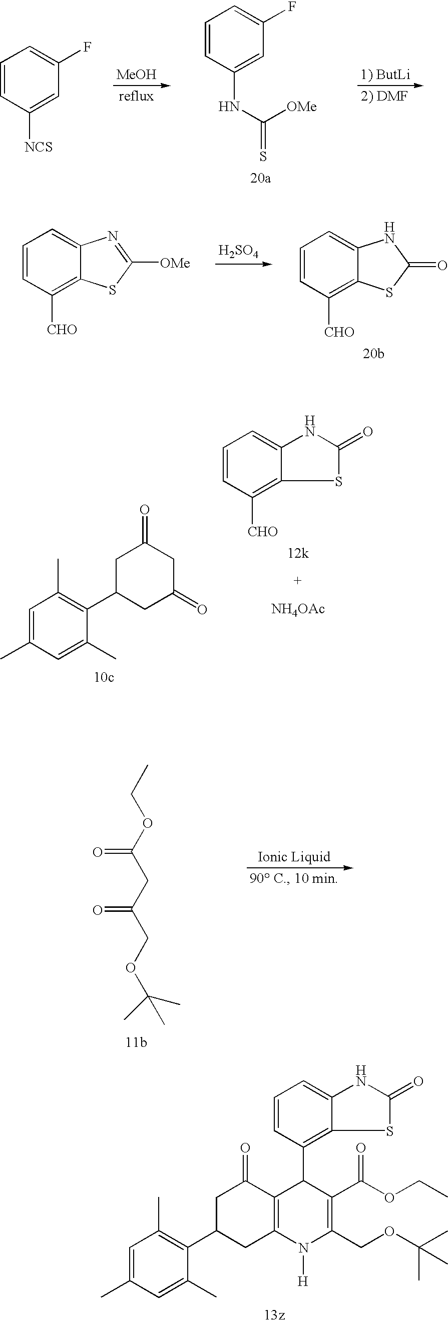

Synthesis of (3-Fluoro-phenyl)-thiocarbamic acid O-methyl ester (20a): 1-Fluoro-3-isothiocyanato-benzene (1 mL, 8.3 mmol) was dissolved in 5 mL of methanol and the reaction mixture was heated to reflux overnight. Solvent was removed and the product (1.5 g, 95%) was obtained as a white solid.

Synthesis of 2-Oxo-2,3-dihydro-benzothiazole-7-carbaldehyde (20b): To a solution of (3-fluoro-phenyl)-thiocarbamic acid O-methyl ester (300 mg, 1.62 mmol) in THF (2 mL) was added tert-butyllithium (1.7 M in pentane, 2.9 mL, 4.86 mmol) at ″40° C. After stirring for one hour, DMF (0.2 mL) was added and the reaction mixture was stirred for another hour at −40° C. before warming up to room temperature. To the reaction mixture was added a solution of H₂SO₄ (2.0 M) and the reaction mixture was heated to reflux overnight. The reaction was quenched with saturated NaHCO₃ solution and extracted with ethyl acetate. The organic layers were combined, washed with brine and dried over MgSO₄. Solvent was then removed and the residue was purified by column chromatography (50% ethyl acetate/hexanes). The product (116 mg, 40%) was obtained as a white solid. MS (ES) M+H expected=180.0, found=180.1.

Synthesis of 2-tert-Butoxymethyl-5-oxo-4-(2-oxo-2,3-dihydro-benzothiazol-7-yl)-7-(2,4,6-trimethyl-phenyl)-1,4,5,6,7,8-hexahydro-quinoline-3-carboxylic acid ethyl ester (13z): To a small vial was added 2-oxo-2,3-dihydro-benzothiazole-7-carbaldehyde (31 mg, 0.17 mmol) followed by 5-(2,4,6-trimethyl-phenyl)-cyclohexane-1,3-dione (40 mg, 0.17 mmol), ammonium acetate (20 mg, 0.26 mmol), 4-tert-butoxy-3-oxo-butyric acid ethyl ester (35 mg, 0.17 mmol) and 1-butyl-3-methylimidazolium tetrafluoroborate (3.9 μL, 0.021 mmol). The reaction mixture was then heated at 90° C. for 10 minutes, cooled down to room temperature, and then loaded on column (50% ethyl acetate/hexanes) to get the desired product (40 mg, 40%) as a solid. MS (ES) M+H expected=575.2, found=575.3.

### Example 94

### Preparation of 4-(1H-Benzotriazol-4-yl)-2-tert-butoxymethyl-5-oxo-7-(2,4,6-trimethyl-phenyl)-1,4,5,6,7,8-hexahydro-quinoline-3-carboxylic acid ethyl ester

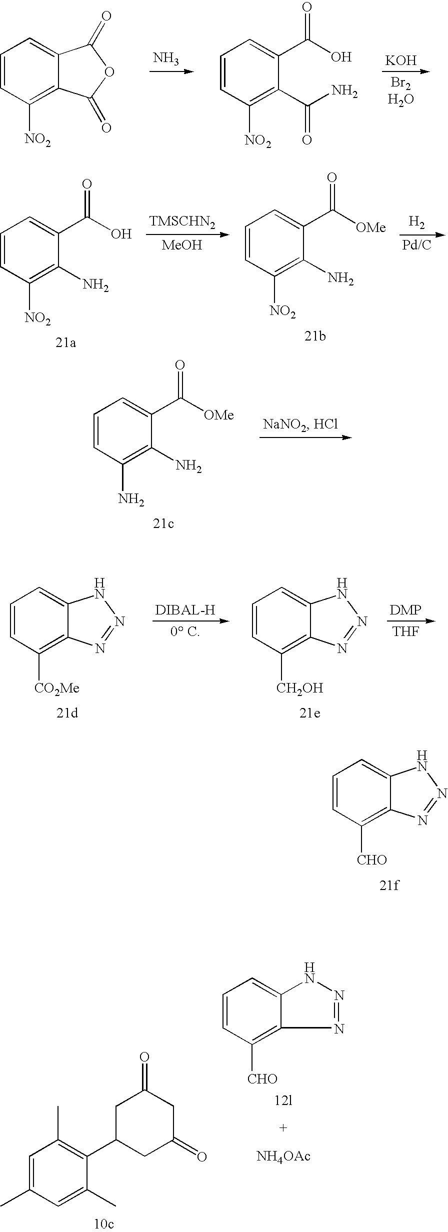

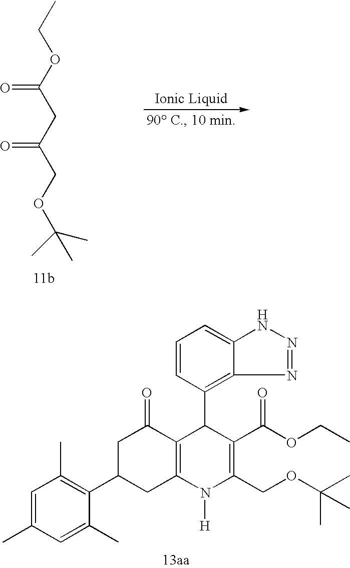

Synthesis of 2-Amino-3-nitro-benzoic acid (21a): 4-Nitro-isobenzofuran-1,3-dione (5 g, 25.91 mmol) was dissolved in an aqueous ammonia solution (10 mL) and the reaction mixture was stirred overnight at room temperature. Solvent was removed and the solid obtained was used directly for the next step. To a solution of 3-nitro-phthalamic acid (2.1 g, 0.01 mmol) in aqueous KOH (5.6 g, 27 mL) was added bromine (0.56 mL, 0.011 mmol) at 0° C. The reaction mixture was warmed at 60° C. for 3 hours, cooled to room temperature, and stirred overnight. The orange precipitate was filtered off and taken up in the minimum quantity of water to give a red solution, which was adjusted to pH4 with concentrated HCl, precipitating a yellow solid. Filtration yielded a yellow solid (0.73 g, 40%).

Synthesis of 2-Amino-3-nitro-benzoic acid methyl ester (21b): To a solution of 2-amino-3-nitro-benzoic acid (174 mg, 0.96 mmol) in 3 mL of methanol was added trimethylsilyl diazomethane (2.0 M in ethyl ether) dropwise at room temperature. More trimethylsilyl diazomethane was added until the starting material disappeared. Solvent was removed carefully and the residue was used directly for the next step.

Synthesis of 2,3-Diamino-benzoic acid methyl ester (21c): 2-Amino-3-nitro-benzoic acid methyl ester (100 mg, 0.51 mmol) was dissolved in 10 mL of methanol. Then pallidium on carbon (10 mg) was added. A balloon filled with hydrogen was put on top of the reaction flask and the reaction mixture was stirred overnight at room temperature. The mixture was filtered through a celite pad and washed with methanol. The filtrate was collected and condensed. The residue was used directly for the next step.

Synthesis of 1H-Benzotriazole-4-carboxylic acid methyl ester (21d): 2,3-Diamino-benzoic acid methyl ester (300 mg, 1.67 mmol) was dissolved in 6 mL of HCl solution and cooled to 0° C. A solution of sodium nitrite (138 mg, 2.00 mmol) in 3 mL of water was added slowly. The reaction mixture was then warmed up to room temperature and stirred for another hour. The reaction was then quenched with saturated NaHCO₃ solution and the reaction mixture was extracted with ethyl acetate. The organic layers were combined and dried over MgSO₄. Solvent was then removed and the residue was purified by column chromatography (75% ethyl acetate/hexanes). The product (191 mg, 60%) was obtained as a white solid. MS (ES) M+H expected=178.0, found=178.1.

Synthesis of (1H-Benzotriazol-4-yl)-methanol (21e): The ester obtained above (120 mg, 0.63 mmol) was dissolved in 1 mL of THF and cooled to 0° C. A solution of DIBAL in THF (1.0 M, 2.5 mL, 2.51 mmol) was added dropwise. More DIBAL was added until the starting material disappeared. The reaction was then quenched with saturated potassium sodium tartrate solution and warmed up to room temperature. The reaction mixture was extracted with ethyl acetate. The organic layers were combined and dried over MgSO₄. Solvent was then removed and the residue was purified by column chromatography (80% ethyl acetate/hexanes). The product (47 mg, 50%) was obtained as a white solid. MS (ES) M+H expected=150.1, found=150.2.

Synthesis of 1H-Benzotriazole-4-carbaldehyde (21f): The alcohol obtained above (68 mg, 0.46 mmol) was dissolved in 2 mL of THF, and then Dess-Martin periodinine (310 mg, 0.73 mmol) was added. The reaction mixture was stirred at room temperature overnight and quenched with Na₂S₂O₃ (2.0 M) solution. The mixture was extracted with ethyl acetate, and the combined organic layers were combined and washed with saturated NaHCO₃ solution, brine, and dried over MgSO₄. Solvent was then removed and the residue was purified by column chromatography (75% ethyl acetate/hexanes). The product (47 mg, 70%) was obtained as a white solid. MS (ES) M+H expected=148.0, found=148.1.

Synthesis of 4-(1H-Benzotriazol-4-yl)-2-tert-butoxymethyl-5-oxo-7-(2,4,6-trimethyl-phenyl)-1,4,5,6,7,8-hexahydro-quinoline-3-carboxylic acid ethyl ester (13aa): To a small vial was added 1H-benzotriazole-4-carbaldehyde (17 mg, 0.12 mmol) followed by 5-(2,4,6-trimethyl-phenyl)-cyclohexane-1,3-dione (26 mg, 0.12 mmol), ammonium acetate (13 mg, 0.17 mmol), 4-tert-butoxy-3-oxo-butyric acid ethyl ester (23 mg, 0.12 mmol) and 1-butyl-3-methylimidazolium tetrafluoroborate (2.6 μL, 0.014 mmol). The reaction mixture was then heated at 90° C. for 10 minutes, cooled down to room temperature, and then loaded on column (50% ethyl acetate/hexanes) to get the desired product (25 mg, 40%) as a yellow solid. MS (ES) M+H expected=543.3, found=543.4.

### Example 95

### Preparation of 2-(2-Cyclopentyl-ethyl)-5-oxo-4-(2-oxo-2,3-dihydro-1H-benzoimidazol-5-yl)-7-(2,4,6-trimethyl-phenyl)-1,4,5,6,7,8-hexahydro-quinoline-3-carboxylic acid ethyl ester

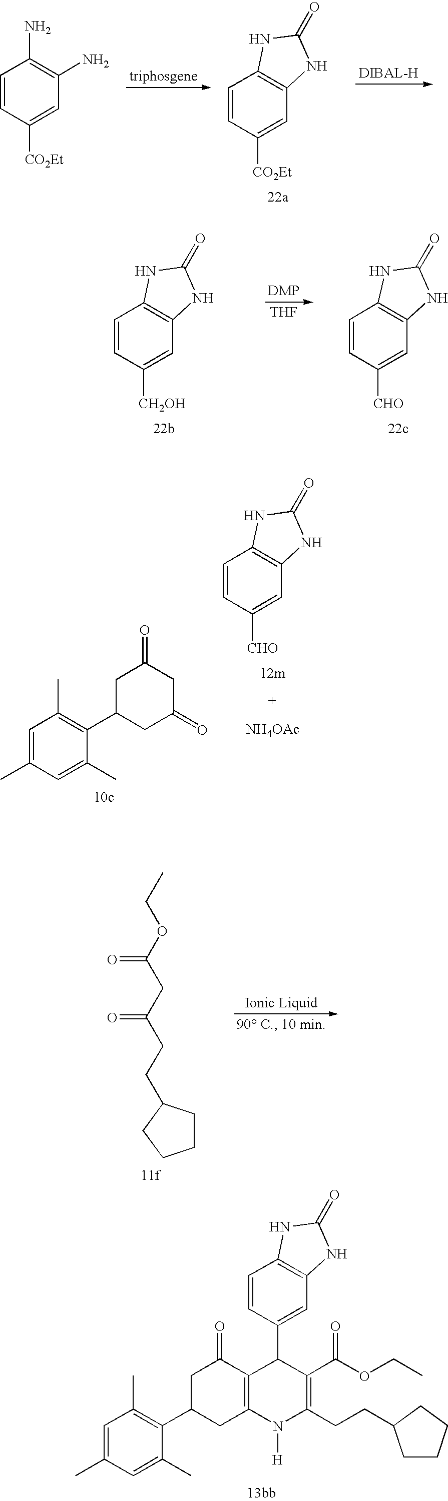

Synthesis of 2-Oxo-2,3-dihydro-1H-benzoimidazole-5-carboxylic acid ethyl ester (22a): 3,4-Diamino-benzoic acid ethyl ester (100 mg, 0.56 mmol) was dissolved in 5 mL of THF and cooled to 0° C. Triethyamine (77 μL, 0.56 mmol) was added followed by potassium carbonate (230 mg, 1.68 mmol). Triphosgene (82 mg, 0.28 mmol) was added in one portion, and the reaction mixture was then warmed up to room temperature and quenched with saturated ammonium chloride solution. The mixture was extracted with ethyl acetate. The organic layers were combined, washed with brine and dried over MgSO₄. Solvent was then removed and the residue was purified by column chromatography (60% ethyl acetate/hexanes). The product (92 mg, 80%) was obtained as a white solid. MS (ES) M+H expected=207.1, found=207.1.

Synthesis of 5-Hydroxymethyl-1,3-dihydro-benzoirnidazol-2-one (22b): The ester obtained above (100 mg, 0.48 mmol) was dissolved in 1 mL of THF and cooled to 0° C. A solution of DIBAL in toluene (1.5 M, 1.3 mL, 1.94 mmol) was added dropwise. More DEBAL was added until the starting material disappeared. The reaction was then quenched with saturated potassium sodium tartrate solution and warmed up to room temperature. The reaction mixture was extracted with ethyl acetate. The organic layers were combined and dried over MgSO₄. Solvent was then removed and the residue was purified by column chromatography (75% ethyl acetate/hexanes). The product (48 mg, 60%) was obtained as a white solid. MS (ES) M+H expected=165.1, found=165.2.

Synthesis of 2-Oxo-2,3-dihydro-1H-benzoimidazole-5-carbaldehyde (22c): The alcohol obtained above (60 mg, 0.37 mmol) was dissolved in 3 mL of THF, and then Dess-Martin periodinine (310 mg, 0.74 mmol) was added. The reaction mixture was stirred at room temperature overnight and quenched with Na₂S₂O₃ (2.0 M) solution. The mixture was extracted with ethyl acetate, and the combined organic layers were combined and washed with saturated NaHCO₃ solution, brine, and dried over MgSO₄. Solvent was then removed and the residue was purified by column chromatography (75% ethyl acetate/hexanes). The product (41 mg, 70%) was obtained as a white solid. MS (ES) M+H expected=163.0, found=163.1.

Synthesis of 2-(2-Cyclopentyl-ethyl)-5-oxo-4-(2-oxo-2,3-dihydro-1H-benzoimidazol-5-yl)-7-(2,4,6-trimethyl-phenyl)-1,4,5,6,7,8-hexahydro-quinoline-3-carboxylic acid ethyl ester (13bb): To a small vial was added 2-oxo-2,3-dihydro-1H-benzoimidazole-5-carbaldehyde (16 mg, 0.099 mmol) followed by 5-(2,4,6-trimethyl-phenyl)-cyclohexane-1,3-dione (23 mg, 0.099 mmol), ammonium acetate (11 mg, 0.15 mmol), 5-cyclopentyl-3-oxo-pentanoic acid ethyl ester (21 mg, 0.099 mmol) and 1-butyl-3-methylimidazolium tetrafluoroborate (2.2 μL, 0.012 mmol). The reaction mixture was then heated at 90° C. for 10 minutes, cooled down to room temperature, and then loaded on column (50% ethyl acetate/hexanes) to get the desired product (22 mg, 40%) as a solid. MS (ES) M+H expected=568.3, found=568.3.

### Example 96

### Preparation of 4-(2-Chloro-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester

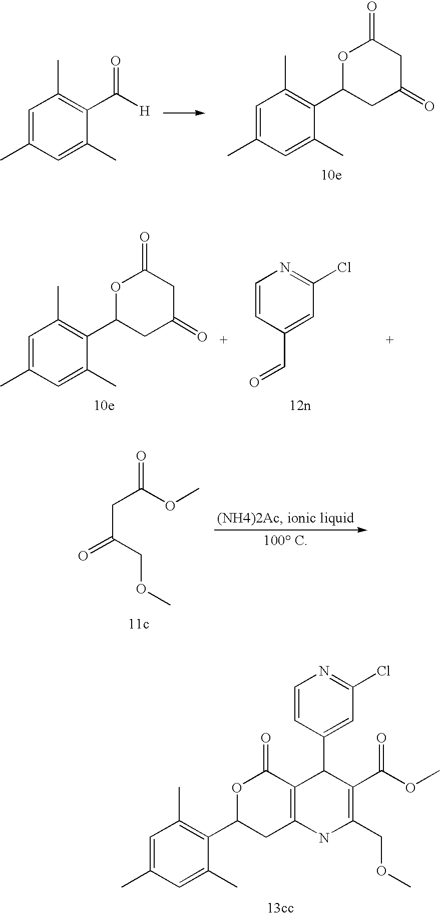

Synthesis of 6-(2,4,6-Trimethyl-phenyl)-dihydro-pyran-2,4-dione (10e): Sodium hydride (7.2 g, 182.5 mmol, 60%) was suspended in THF and cooled to −10° C. with stirring under nitrogen. To this mixture was added methyl acetoacetate (18.6 mL, 172.4 mmol) dropwise. The mixture was allowed to stir an additional 30 minutes to 1 hour after the addition. The mixture was maintained at −10° C. as LDA (from n-BuLi (68.8 mL, 172 mmol, 2.5 M in hexanes) and diisopropylamine (24.1 mL, 172 mmol)) was added dropwise. The mixture was stirred for 1 hour at −10° C. and cooled to −78° C. To this mixture was added 2,4,6-mesitylaldehyde (25.6 mL, 172 mmol). The mixture was allowed to warm to rt overnight. The mixture was cooled to −10° C. and 4M NaOH (250 mL) in water was added. The mixture was allowed to warm to rt and stir overnight. The organic layer was removed by evaporation and the resulting aqueous layer was cooled to −10° C. as it was acidified to pH 2-3 with concentrated HCl. The resulting mixture was extracted with CH₂Cl₂ and the organic layer washed with brine. The organic layer was dried over magnesium sulfate, filtered and evaporated to dryness. The residue was dried overnight under vacuum. Ether was added to precipitate the product as a light yellow solid. MS (ES) M+H expect 233.3, found 233.7.

Synthesis of 4-(2-Chloro-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester (13cc): The compound was synthesized analogous to example 1 with 141 mg (1.0 mmol) of 2-chloro-4-formyl pyridine, 232 mg (1.0 mmol) of 6-(2,4,6-Trimethyl-phenyl)-dihydro-pyran-2,4-dione, 146 mg (1.0 mmol) of 4-Methoxy-3-oxo-butyric acid methyl ester, 85 mg (1.1 mmol) of ammonium acetate and 20 mg of ionic liquid. The crude product was purified by flash chromatograph to yield 111 mg of colorless solid. LC-MSD, m/z for C₂₆H₂₇N₂O₅Cl [M+H]+: 482.5, [M+2H]+: 483.5. Reverse phase HPLC gradient acetonitrile 0.1% TFA 20-95% in 4 min: 3.308 min.

### Example 97

### Preparation of 4-(2-Hydroxy-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester

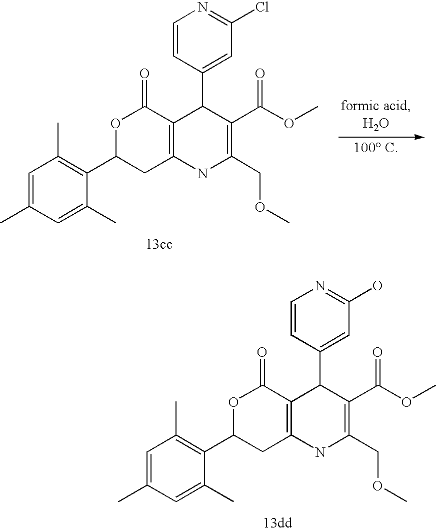

Synthesis of 4-(2-Hydroxy-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester (13dd): 50 mg (4-(2-Chloro-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester was dissolved in 1 mL of 85% formic acid and 1 mL of water. The mixture was stirred at 90° C. for 20 hours. The reaction mixture was concentrated to dryness. The crude product was purified by preparatory HPLC to yield 24 mg of colorless solid. LC-MSD, m/z for C₂₆H₂₇N₂O₅C [M+H]+: 465.5, [M+2H]+: 466.5. Reverse phase HPLC gradient acetonitrile 0.1% TFA 20-95% in 4 min: 2.667 min.

### Example 98

### Preparation of 2-Methyl-5-oxo-4-(3-oxo-3,4-dihydro-2H-benzo[1,4]oxazin-8-yl)-7-(2,4,6-trimethyl-phenyl)-1,5,7,8-tetrahydro-4H-pyrano[4,3-b]pyridine-3-carboxylic acid methyl ester

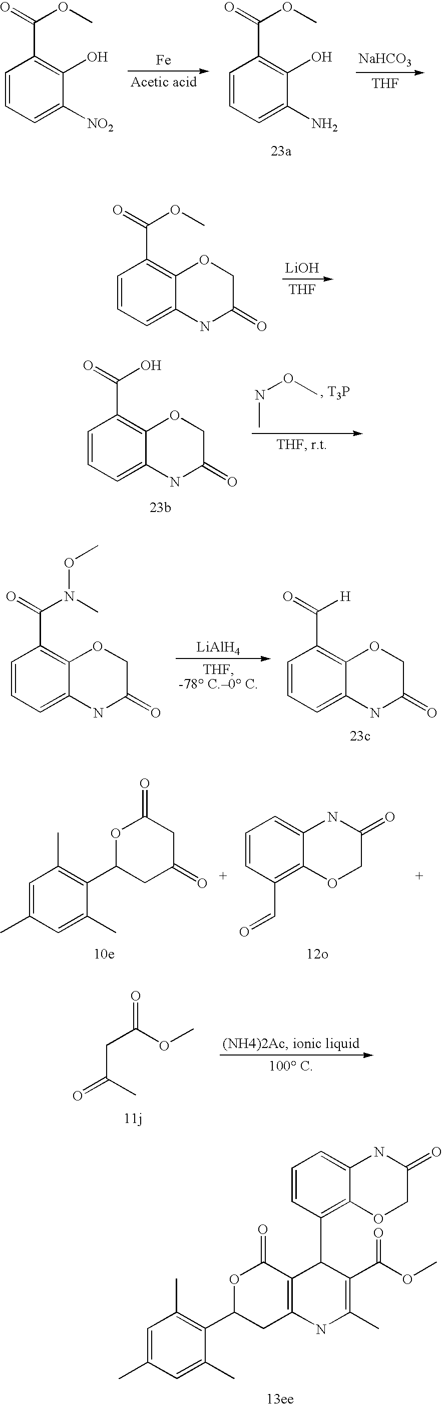

3-Amino-2-hydroxy-benzoic acid methyl ester (23a): 2.0 g of 2-hydroxy-3-nitro-methyl benzoic acid was dissolved in 30 mL of acetic acid. 2.0 g of iron powder was added into the solution and the mixture was stirred at 45° C. for 2 hours. The reaction mixture was filtered through a thin pad of silica gel and washed with 200 mL of ethyl acetate. The filtrate was concentrated to near dryness then the crude was re-dissolved in 100 mL of dichloromethane. The solution was washed with sat. aqueous NaHCO3 (100 mL, 3 times), sat. aqueous NaCl (100 mL, 2 times). The organic layer was collected and concentrated to dryness. The crude product was purified by flash chromatograph to yield 1.67 g of 3-anino-2-hydroxy-benzoic acid methyl ester as colorless solid. [M+H]+: 168.1, [M+2H]+: 169.1.

3-Oxo-3,4-dihydro-2H-benzo[1,4]oxazine-8-carboxylic acid (23b): 1.67 of 3-amino-2-hydroxy-benzoic acid methyl ester was dissolved in 15 mL of tetrahydrofuran and 25 mL of sat. aqueous NaHCO₃. The mixture was stirred at r. t. for 2 hours, then warmed to 85° C. and stirred for 4 hours. The mixture was diluted with 100 mL of water then extracted with ethyl acetate (100 ml, 2 times). The organic layers were collected and combined, concentrated to dryness. The crude was dissolved in 40 mL of tetrahydrofuran. 500 mg of lithium hydroxide was added and the mixture was stirred at r. t. for 18 hours. The mixture was diluted with 100 ml of water then extracted with ethyl acetate (100 mL, 2 times). The organic layers were combined and concentrated to dryness. The crude was purified by flash chromatograph to yield 1.72 g of colorless solid. [M+H]+: 194.2, [M+2H]+: 195.2.

3-Oxo-3,4-dihydro-2H-benzo[1,4]oxazine-8-carbaldehyde (23c): 260 mg (1.34 mmol) of 3-Oxo-3,4-dihydro-2H-benzo[1,4]oxazine-8-carboxylic acid, 156 mg (1.68 mmol) of methoxymethylamine, 432 mg (3.36 mmol) of diisopropylethylamine, and 1.26 g (50%, 2 mmol) of propylphosphonic anhydride in ethyl acetate solution were dissolved in 12 mL of dichloromethane and the mixture was stirred at r. t. for 2 hours. The reaction solution was diluted with 100 mL of ethyl acetate, washed with water (100 mL, 2 times), brine (100 mL, 3 times). The organic layer was concentrated to dryness. The crude was dissolved in 20 mL of tetrahydrofuran. 3 mL (1.0 M, 3 mmol) of diisobutylaluminumhydride was added under nitrogen at −78° C. The mixture was stirred and warmed slowly to r. t. The reaction was quenched by adding 10 mL of sat. aqueous NaHCO₃ and extracted with ethyl acetate (20 mL, 2 times). The organic layers were combined and concentrated to dryness. The crude was purified by flash chromatograph to yield 200 mg of colorless solid.

Synthesis of 2-Methoxymethyl-5-oxo-4-(3-oxo-3,4-dihydro-2H-benzo[1,4]oxazin-8-yl)-7-(2,4,6-trimethyl-phenyl)-1,5,7,8-tetrahydro-4H-pyrano[4,3-b]pyridine-3-carboxylic acid methyl ester (13ee): The compound was synthesized analogous to example 1 with 177 mg (1.0 mmol) of 3-Oxo-3,4-dihydro-2H-benzo[1,4]oxazine-8-carbaldehyde, 232 mg (1.0 mmol) of 6-(2,4,6-Trimethyl-phenyl)-dihydro-pyran-2,4-dione, 116 mg (1.0 mmol) of 3-oxo-butyric acid methylester, 85 mg (1.1 mmol) of ammonium acetate and 20 mg of ionic liquid. The crude product was purified by flash chromatograph to yield 111 mg of colorless solid. LC-MSD, m/z for C₂₆H₂₇N₂O₅Cl [M+H]+: 489.5, [M+2H]+: 490.5 Reverse phase HPLC gradient acetonitrile 0.1% TFA 20-95% in 4 min: 2.432 min.

### Example 99

### Preparation of 2-tert-butoxymethyl-4-(1H-indol-4-yl)-5-oxo-7,7-dipropyl-1,5,7,8-tetrahydro-4H-pyrano[4,3-b]pyridine-3-carboxylic acid ethyl ester

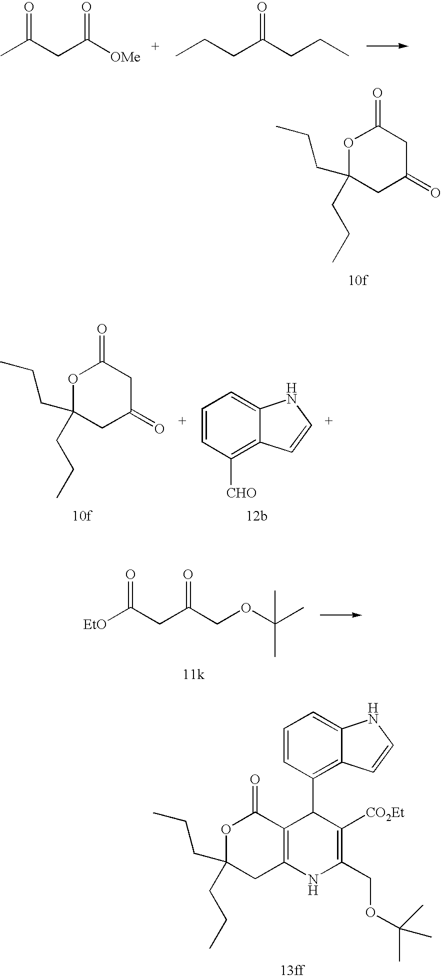

Synthesis of 6,6-dipropyl-dihydro-pyran-2,4-dione (10f): Experimental conditions were used analogous to those described in the literature (J. Med. Chem. 1998, 41, 3467-3476) with methyl acetoacetate (2.79 mL, 25.83 mmol), 4-heptanone (3 mL, 21.35 mmol), sodium hydride (1.08 g, 27.11 mmol, 60% dispersion in oil), n-butyl lithium (13.1 mL, 26.26 mmol) and THF (46 mL) to obtain 1.30 g of 6,6-dipropyl-dihydro-pyran-2,4-dione as a white solid.

Synthesis of 2-tert-butoxymethyl-4-(1H-indol-4-yl)-5-oxo-7,7-dipropyl-1,5,7,8-tetrahydro-4H-pyrano[4,3-b]pyridine-3-carboxylic acid ethyl ester (13ff): Experimental conditions analogous to those described previously were used with 4-tert-butoxy-3-oxo-butyric acid ethyl ester (165.6 mg, 0.819 mmol), 6,6-dipropyl-dihydro-pyran-2,4-dione (162.4 mg, 0.819 mmol), indole 4-carboxyaldehyde (118.9 mg, 0.819 mmol), ammonium acetate (82 mg, 1.06 mmol), and 2 drops of ionic liquid. The crude product was purified by flash column chromatography using a gradient of 20-100% ethyl acetate in hexane and further purified on the reverse phase HPLC with a C 18 column, gradient of 20-70% acetonitrile −0.1% TFA to afford 41 mg of 2-tert-butoxymethyl-4-(1H-indol-4-yl)-5-oxo-7,7-dipropyl-1,5,7,8-tetrahydro-4H-pyrano[4,3-b]pyridine-3-carboxylic acid ethyl ester. MS (ES) M+H expect 508.3, found 509.2.

### Example 100

### Preparation of 2-tert-butoxymethyl-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester

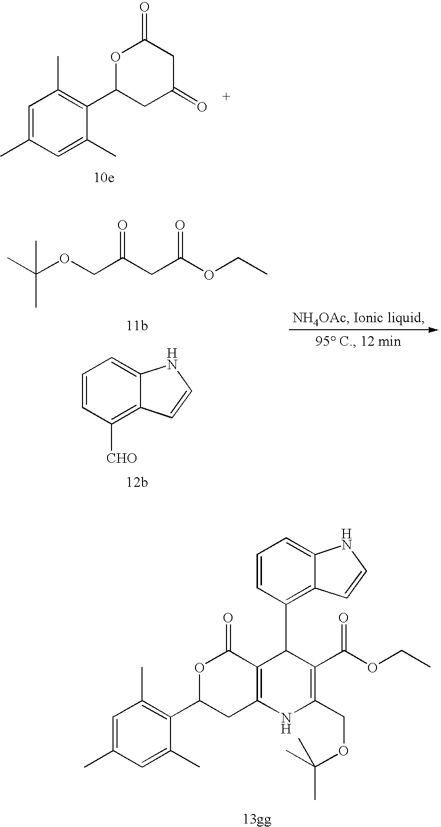

Synthesis of 2-tert-butoxymethyl-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester (13gg): This compound was synthesized by using general Hantschz procedure. LC-MS (method: 20-100-5 min) retention time (Rt)=2.65 min. MS calc'd for C₃₃H₃₈N₂O₅ (MH+): 543.2. Found 543.2

### Example 101

### Preparation of 2-tert-Butoxymethyl-4-(2,3-dihydro-1H-indol-4-yl)-5-oxo-7-(2,4,6-trimethyl-phenyl)-1,5,7,8-tetrahydro-4H-pyrano[4,3-b]pyridine-3-carboxylic acid methyl ester

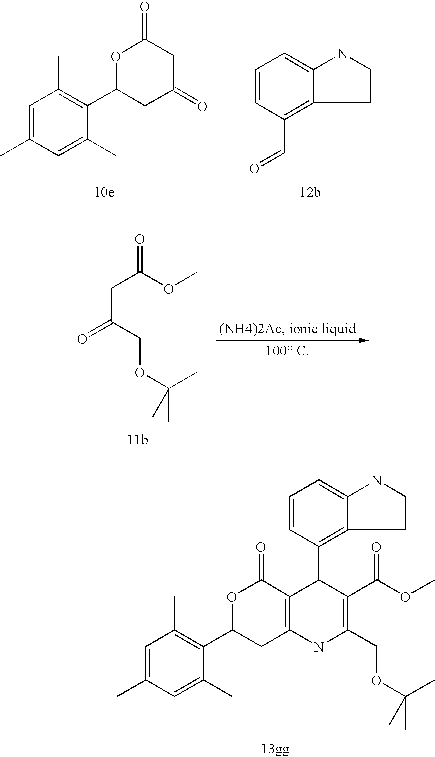

Synthesis of 2-tert-Butoxymethyl-4-(2,3-dihydro-1H-indol-4-yl)-5-oxo-7-(2,4,6-trimethyl-phenyl)-1,5,7,8-tetrahydro-4H-pyrano[4,3-b]pyridine-3-carboxylic acid methyl ester (13gg): The compound was synthesized analogous to example 1 with 147 mg (1.0 mmol) of 2,3-Dihydro-1H-indole-4-carbaldehyde, 232 mg (1.0 mmol) of 6-(2,4,6-Trimethyl-phenyl)-dihydro-pyran-2,4-dione, 202 mg (1.0 mmol) of 4-tert-Butoxy-3-oxo-butyric acid methyl ester, 85 mg (1.1 mmol) of ammonium acetate and 20 mg of ionic liquid. The crude product was purified by flash chromatograph to yield 93 mg of colorless solid. LC-MSD, m/z for C₂₆H₂₇N₂O₅Cl [M+H]+: 545.7, [M+2H]+: 546.7 Reverse phase HPLC gradient acetonitrile 0.1% TFA 20-95% in 4 min: 2.109 min.

### Example 102

### Preparation of 4-(2-Amino-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester

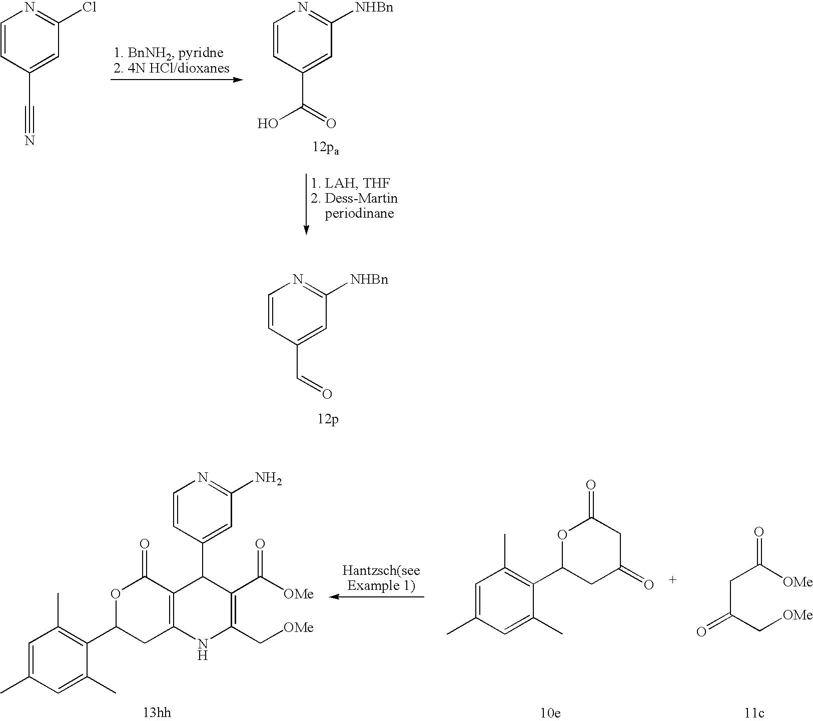

Synthesis of 2-Benzylamino-isonicotinic acid (12pₐ): 2-Chloroisonicotinonitrile (1 g, 7.2 mmol) and benzyl amine (1.6 mL, 14.4 mmol) were dissolved in pyridine (20 mL) and heated to 80° C. for 2 hours. The solvent was then removed and the product purified by column chromatography (1:4 EtOAc/hexanes). The product (367 mg, 1.8 mmol) was then heated to 80° C. in 4N HCl (4 mL) for 2 hours. The reaction was neutralized with sat'd NaHCO₃(aq) and extracted with CH₂Cl₂.

2-Benzylamino-pyridine-4-carbaldehyde (12p): The product from a. was then dissolved in THF (5 mL) and cooled to 0° C. LAH (200 mg, 5.3 mmol) was then added in portions over 10 minutes and the reaction was allowed to come to room temperature. After 1 hour, the reaction was quenched with sat'd Na₂SO₄(aq), filtered, and concentrated. Purification was performed using column chromatography (1:1 EtOAc/hexanes). The product (135 mg, 0.63 mmol) was then dissolved in CH₂Cl₂ (5 mL) and cooled to 0° C. Dess-Martin periodinane (401 mg, 0.94 mmol) was then added and the reaction was allowed to come to room temperature and stir for 2 hours. The reaction was then washed with Na₂S₂O₃ and then brine. The organic layer was dried over Na₂SO₄ and concentrated to give product.

Synthesis of 4-(2-Amino-pyridin-4-yl)-2-methoxymethyl-5-oxo-7-(2,4,6-trimethyl-phenyl)-1,5,7,8-tetrahydro-4H-pyrano[4,3-b]pyridine-3-carboxylic acid methyl ester (13hh): The Hantzsch reaction was then performed with lactone (98 mg, 0.42 mmol), β-ketoester (70 mg, 0.42 mmol), NH₄OAc (70 mg, 0.91 mmol) and ionic liquid as described in Example 1. The product 320-24 was then purified by prep TLC (MS calc'd for C₂₆H₃₀N₃O₅ (MH⁺): 464.2. Found 464.2.

### Example 103

### Preparation of 2-tert-Butoxymethyl-4-(3-carboxymethyl-phenyl)-5-oxo-7-(2,4,6-trimethyl-phenyl)-1,5,7,8-tetrahydro-4H-pyrano[4,3-b]pyridine-3-carboxylic acid ethyl ester

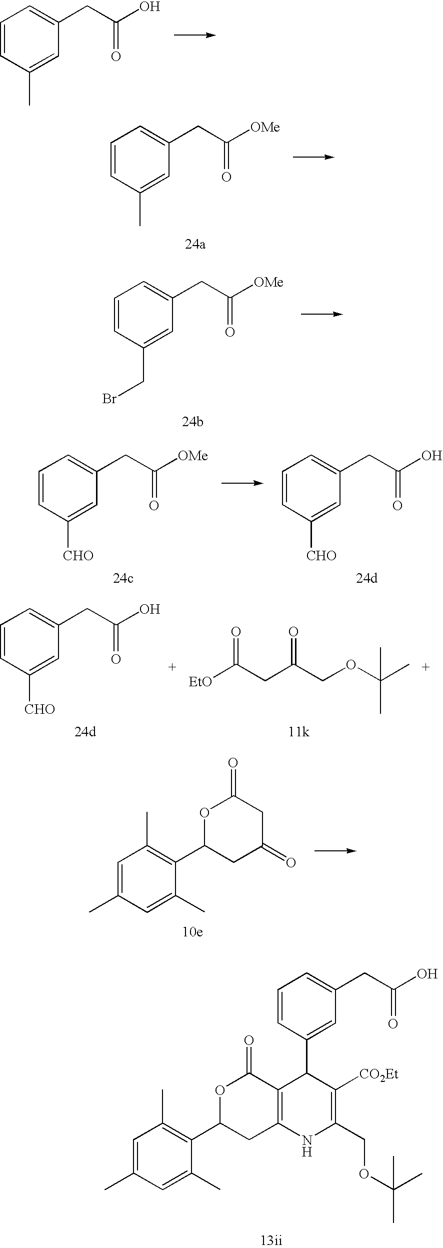

Synthesis of m-tolylacetic acid methyl ester (24a): Trimethylsilyldiazomethane (16.6 mL, 33.29 mmol) was added to a solution of m-tolylacetic acid 5 g (33.29 mmol) in methanol at 0° C. and stirred for 20 minutes. Acetic acid was added until the yellow color disappeared and the reaction was concentrated to yield 5.47 g of m-tolylacetic acid methyl ester as a yellow oil.

Synthesis of (3-Bromomethyl-phenyl)-acetic acid methyl ester (24b): A solution of m-tolylacetic acid methyl ester (3.20 g, 19.5 mmol), N-bromosuccinimide (3.47 g, 19.5 mmol), a catalytic amount of dibenzoyl peroxide, and carbon tetrachloride (160 mL) was refluxed overnight. The solid was filtered while the reaction was still hot, the filtrate collected and concentrated The crude product was purified by flash column chromatography eluting with 20% ethyl acetate in hexane to afford 3.11 g of (3-Bromomethyl-phenyl)-acetic acid methyl ester.

Synthesis of (3-Formyl-phenyl)-acetic acid methyl ester (24c): A solution of (3-Bromomethyl-phenyl)-acetic acid methyl ester (1 g, 4.11 mmol), sodium periodate (0.94 g, 4.11 mmol) and N,N dimethylformamide (58 mL) was refluxed for 2 hours. The reaction was quenched with water and extracted with diethyl ether (40 mL×3). The organic layers were dried over magnesium sulfate, filtered and concentrated in vacuo to give 0.279 g of (3-Formyl-phenyl)-acetic acid methyl ester.

Synthesis of (3-Formyl-phenyl)-acetic acid (24d): Lithium hydroxide (89.3 mg, 3.72 mmol) was added to a solution of (3-Formyl-phenyl)-acetic acid methyl ester (221 mg, 1.24 mmol) in 50 mL water/methanol (5/1) and stirred at room temperature overnight. After the methanol was removed in vacuo the reaction was acidified to pH1 with 1M HCl. The aqueous layer was extracted with dichloromethane, and chloroform. The combined organic layers were dried over magnesium sulfate, filtered and concentrated in vacuo to afford 181.9 mg of (3-Formyl-phenyl)-acetic acid.

Synthesis of 2-tert-Butoxymethyl-4-(3-carboxymethyl-phenyl)-5-oxo-7-(2,4,6-trimethyl-phenyl)-1,5,7,8-tetrahydro-4H-pyrano[4,3-b]pyridine-3-carboxylic acid ethyl ester (13ii): Experimental conditions analogous to those described previously were used with 4-tert-butoxy-3-oxo-butyric acid ethyl ester (222.5 mg, 1.10 mmol), 6-(2,4,6-Trimethyl-phenyl)-dihydro-pyran-2,4-dione (255.5 mg, 0.1.10 mmol), (3-Formyl-phenyl)-acetic acid (181 mg, 1.10 mmol), ammonium acetate (110 mg, 1.43 mmol), and 2 drops of ionic liquid. The crude product was purified by flash column chromatography using a gradient of 20-100% ethyl acetate in hexane and further purified on the reverse phase HPLC with a C 18 column, gradient of 20-70% acetonitrile −0.1% TFA to afford 22.8 mg of 2-tert-butoxymethyl-4-(3-carboxymethyl-phenyl)-5-oxo-7-(2,4,6-trimethyl-phenyl)-1,5,7,8-tetrahydro-4H-pyrano[4,3-b]pyridine-3-carboxylic acid ethyl ester as a white solid.

### Example 104

### Preparation of 2-tert-Butoxymethyl-4-(3-carbamoylmethyl-phenyl)-5-oxo-7-(2,4,6-trimethyl-phenyl)-1,5,7,8-tetrahydro-4H-pyrano[4,3-b]pyridine-3-carboxylic acid ethyl ester

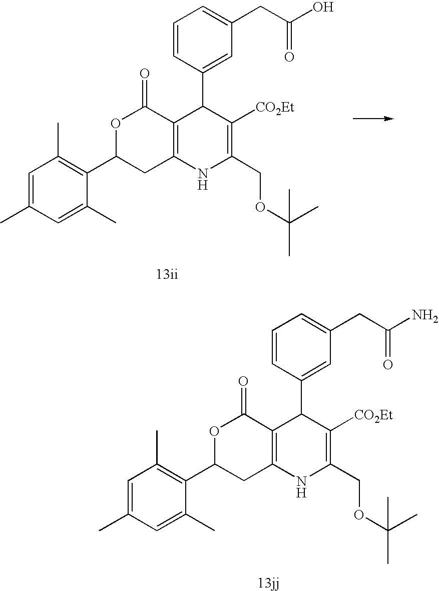

Preparation of 2-tert-Butoxymethyl-4-(3-carbamoylmethyl-phenyl)-5-oxo-7-(2,4,6-trimethyl-phenyl)-1,5,7,8-tetrahydro-4H-pyrano[4,3-b]pyridine-3-carboxylic acid ethyl ester (13jj): Oxayl chloride (12.1 mL, 0.139 mmol) was added to a solution of 2-tert-butoxymethyl-4-(3-carboxymethyl-phenyl)-5-oxo-7-(2,4,6-trimethyl-phenyl)-1,5,7,8-tetrahydro-4H-pyrano[4,3-b]pyridine-3-carboxylic acid ethyl ester (60 mg, 0.107 mmol) in 1 mL of dichloromethane and stirred at room temperature for 1.5 hours. An excess of 2 M ammonia in methanol was added and stirred overnight at room temperature. The reaction was concentrated, then dissolved in dichloromethane and washed with saturated sodium bicarbonate. The combined organic layers were dried over magnesium sulfate, filtered and concentrated in vacuo. The crude product was purified by flash column chromatography eluting with 100% ethyl acetate to yield 31.5 mg of 2-tert-Butoxymethyl-4-(3-carbamoylmethyl-phenyl)-5-oxo-7-(2,4,6-trimethyl-phenyl)-1,5,7,8-tetrahydro-4H-pyrano[4,3-b]pyridine-3-carboxylic acid ethyl ester. MS (ES) M+H expect 560.3, found 561.1.

### Example 105

### Preparation of 4-(3-Hydroxy-phenyl)-5-oxo-2-piperidin-1-ylmethyl-7-(2,4,6-trimethyl-phenyl)-1,5,7,8-tetrahydro-4H-pyrano[4,3-b]pyridine-3-carboxylic acid ethyl ester

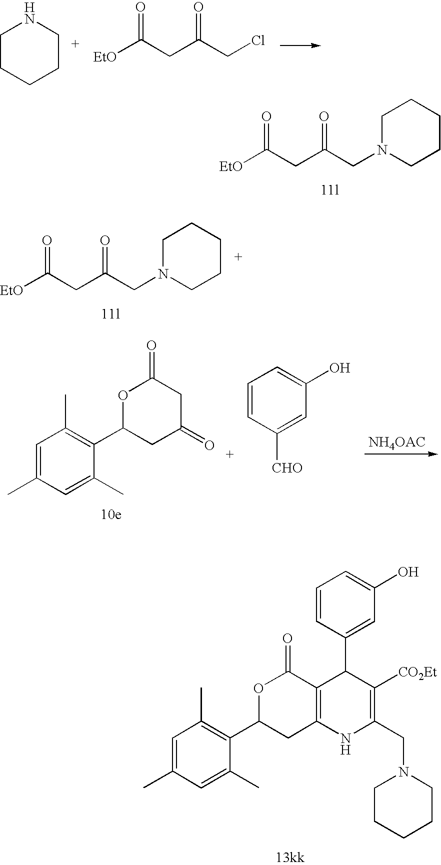

Synthesis of 3-Oxo-4-piperidin-1-yl-butyric acid ethyl ester (11l): Experimental conditions analogous to those described previously were used with Ethyl 4-chloroacetoacetate (1.65 mL, 12.15 mmol), piperidine (1.2 mL, 12.15 mmol), and sodium hydride (1.0 g, 24.30 mmol, 60% dispersion in oil) to yield 2.6 g of 3-Oxo-4-piperidin-1-yl-butyric acid ethyl ester.

Synthesis of 4-(3-Hydroxy-phenyl)-5-oxo-2-piperidin-1-ylmethyl-7-(2,4,6-trimethyl-phenyl)-1,5,7,8-tetrahydro-4H-pyrano[4,3-b]pyridine-3-carboxylic acid ethyl ester (13kk): Experimental conditions analogous to those described previously were used with 3-Oxo-4-piperidin-1-yl-butyric acid ethyl ester (174.7 mg, 0.819 mmol), 6-(2,4,6-Trimethyl-phenyl)-dihydro-pyran-2,4-dione (190.2 mg, 0.819 mmol), 3-hydroxybenzaldehyde (100 mg, 0.819 mmol), ammonium acetate (82.1 mg, 1.06 mmol), and 2 drops of ionic liquid. The crude product was purified by flash column chromatography using a gradient of 20-100% ethyl acetate in hexane and further purified on the reverse phase HPLC with a C 18 column, gradient of 20-70% acetonitrile −0.1% TFA to afford 50 mg of 4-(3-Hydroxy-phenyl)-5-oxo-2-piperidin-1-ylmethyl-7-(2,4,6-trimethyl-phenyl)-1,5,7,8-tetrahydro-4H-pyrano[4,3-b]pyridine-3-carboxylic acid ethyl ester as a white solid. MS (ES) M+H expect 530.3, found 531.3.

### Example 106

### Preparation of 4-(1H-Indol-4-yl)-2-((S)-1-methyl-pyrrolidin-2-yl)-5-oxo-7-(2,4,6-trimethyl-phenyl)-1,5,7,8-tetrahydro-4H-pyrano[4,3-b]pyridine-3-carboxylic acid ethyl ester

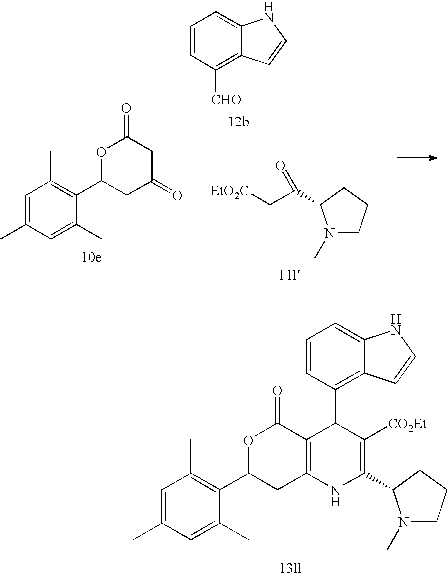

Synthesis of 4-(1H-Indol-4-yl)-2-((S)-1-methyl-pyrrolidin-2-yl)-5-oxo-7-(2,4,6-trimethyl-phenyl)-1,5,7,8-tetrahydro-4H-pyrano[4,3-b]pyridine-3-carboxylic acid ethyl ester (13ll): A mixture 489 mg (2.11 mmol) of 6-(2,4,6-trimethyl-phenyl)-dihydro-pyran-2,4-dione, 306 mg (2.11 mmol) of 1H-indole-4-carbaldehyde, 504 mg (2.53 mmol) 3-((S)-1-methyl-pyrrolidin-2-yl)-3-oxo-propionic acid ethyl ester, 195 mg (2.53 mmol) of ammonium acetate and 72 mg (0.32 mmol) of 1-butyl-3-methyl-3H-imidazol-1-ium tetrafluoroborate were heated at 100° C. for 30 min. Chromatographic purification resulted in isolation of 12 mg of a mixture of diastereoisomeric products as a colorless oil. LC-MSD, m/z for C₃₃H₃₈N₃O₄ [M+H]+=540.2, HPLC retention time: 2.0 min.

### Example 107

### Preparation of 2-(1,1-Dioxo-1lambda*6*-thiomorpholin-4-ylmethyl)-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester

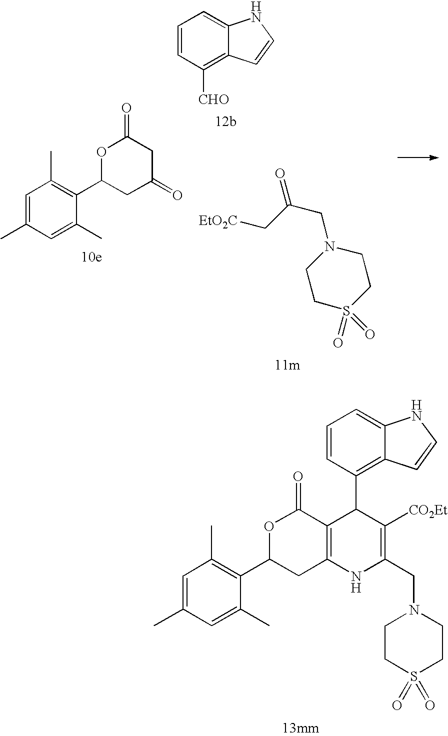

Synthesis of 2-(1,1-Dioxo-1lambda*6*-thiomorpholin-4-ylmethyl)-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester (13 mm): A mixture 81 mg (0.35 mmol) of 6-(2,4,6-trimethyl-phenyl)-dihydro-pyran-2,4-dione, 51 mg (0.35 mmol) of 1H-indole-4-carbaldehyde, 110 mg (0.42 mmol) 4-(1,1-dioxo-1lambda*6*-thiomorpholin-4-yl)-3-oxo-butyric acid ethyl ester, 32 mg (0.42 mmol) of ammonium acetate and 12 mg (0.05 mmol) of 1-butyl-3-methyl-3H-imidazol-1-ium tetrafluoroborate were heated at 100° C. for 30 min. Chromatographic purification resulted in isolation of 49 mg of a mixture of diastereoisomeric products as a colorless oil. LC-MSD, m/z for C₃₃H₃₈N₃O₆S [M+H]+=604.2, HPLC retention time: 2.4 min.

### Example 108

### Preparation of 4-(1H-Indol-4-yl)-5-oxo-2-pyrrolidin-1-yl-7-(2,4,6-trimethyl-phenyl)-1,5,7,8-tetrahydro-4H-pyrano[4,3-b]pyridine-3-carboxylic acid ethyl ester

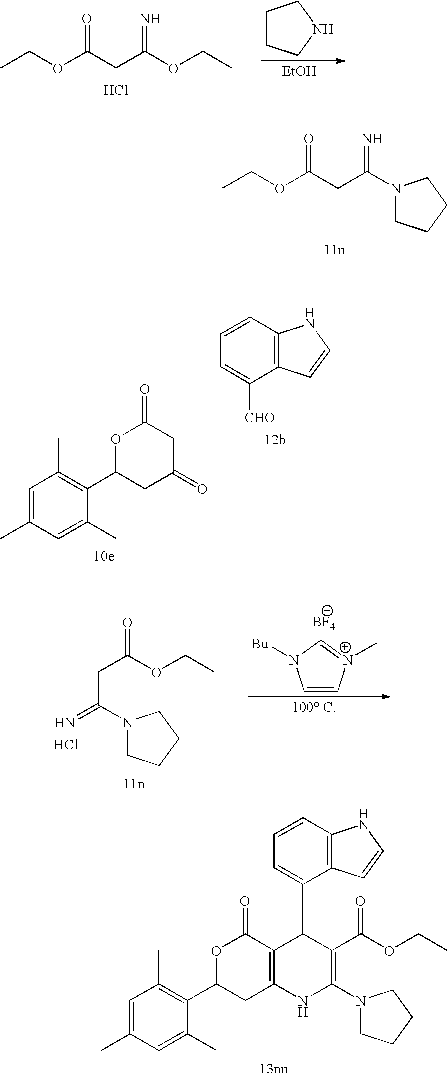

Synthesis of 3-Imino-3-pyrrolidin-1-yl-propionic acid ethyl ester (11n): Ethyl 3-amino-3-ethoxyacrylate hydrochloride (564 mg, 2.9 mmol) was diluted with 1.2 mL absolute EtOH and then treated with 253 μL (3 mmol) pyrrolidine. The slurry was stirred 2 hours at room temperature at which point LCMS showed complete conversion to the amidinoacetate. The slurry was then concentrated in vacuo to a colorless viscous oil and used immediately in the subsequent Hantzsch reaction. MS (ES) M+H expect 185.1, found 185.1.

Synthesis of 4-(1H-Indol-4-yl)-5-oxo-2-pyrrolidin-1-yl-7-(2,4,6-trimethyl-phenyl)-1,5,7,8-tetrahydro-4H-pyrano[4,3-b]pyridine-3-carboxylic acid ethyl ester (13nn): The 20-mL scintillation vial containing the amidinoacetate slurry was charged with 700 mg (3 mmol) of 6-(2,4,6-trimethylphenyl)-2,4-diketotetrahydropyran (lactone) and 436 mg (3 mmol) of indole-4-carboxaldehyde. Approximately 8 drops of 1-butyl-3-methylimidazolium tetrafluoroborate was added and the reaction mixture stirred at 100° C. for 1-2 hours. After completion (determined by LCMS), the reaction slurry was diluted with 20 mL EtOAc and 5 mL saturated NaHCO₃. The organic layer was collected and the aqueous was further extracted with 3×EtOAc. The combined organic layers were dried over Na₂SO₄, filtered, and concentrated to an orange-black foam. The product was purified in 25-80% EtOAc in hexanes followed by preparatory HPLC (20-95% CH₃CN/H₂O). MS (ES) M+H expect 526.2, found 526.2.

### Example 109

### Preparation of 2-Amino-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester

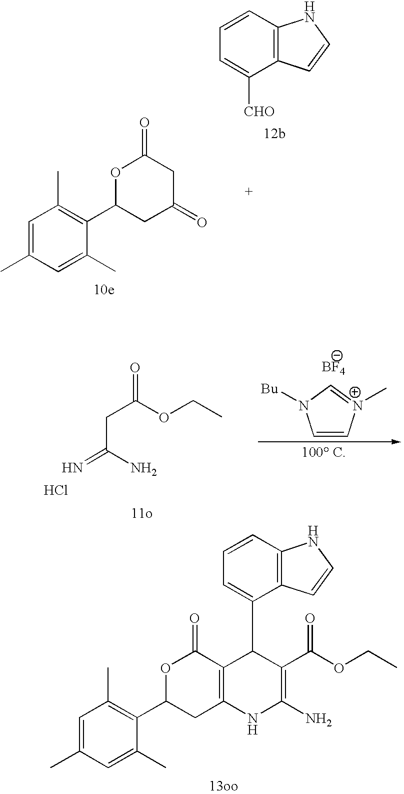

Synthesis of 2-Amino-4-(1H-indol-4-yl)-5-oxo-7-(2,4,6-trimethyl-phenyl)-1,5,7,8-tetrahydro-4H-pyrano[4,3-b]pyridine-3-carboxylic acid ethyl ester (13oo): Ethyl amidinoacetate acetic acid salt is added to a 25-mL flask followed by lactone (1 equiv) and indole-4-carboxaldehyde (1 equiv). 1-Butyl-3-methylimidazolium tetrafluoroborate (approx 2-3 drops/mmol of substrate) is added and the reaction mixture stirred at 100° C. for 1-2 hours. After completion (determined by LCMS), the reaction slurry is diluted with EtOAc and saturated NaHCO₃. The organic layer is collected and the aqueous layer further extracted with 3×EtOAc. The product is purified in 25-80% EtOAc in hexanes. MS (ES) M+H expect 472.2, found 472.2.

### Example 110

### Preparation of 4-(3-Hydroxy-phenyl)-2-methyl-5-oxo-7-(2,4,6-trimethyl-phenyl)-7,8-dihydro-5H-pyrano[4,3-b]pyridine-3-carboxylic acid ethyl ester

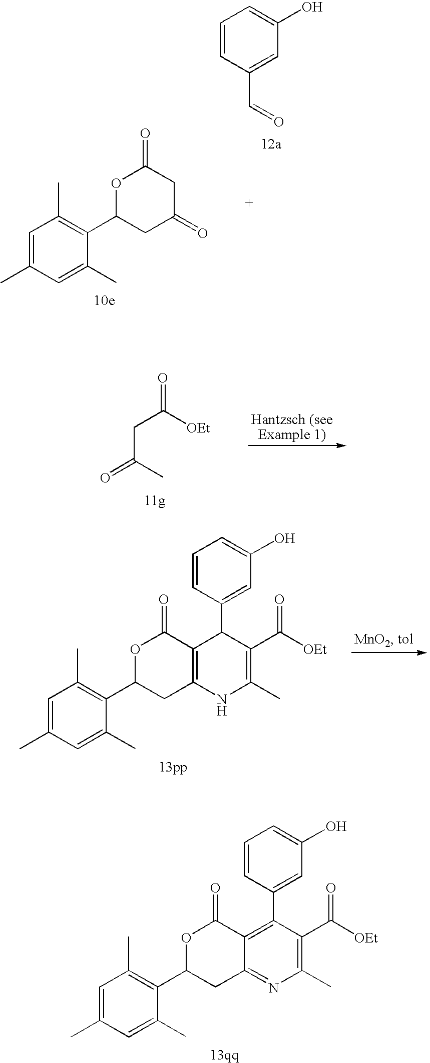

Synthesis of 4-(3-Hydroxy-phenyl)-2-methyl-5-oxo-7-(2,4,6-trimethyl-phenyl)-1,5,7,8-tetrahydro-4H-pyrano[4,3-b]pyridine-3-carboxylic acid ethyl ester (13pp): The Hantzsch reaction was performed as described in Example 1 to give product.

Synthesis of 4-(3-Hydroxy-phenyl)-2-methyl-5-oxo-7-(2,4,6-trimethyl-phenyl)-7,8-dihydro-5H-pyrano[4,3-b]pyridine-3-carboxylic acid ethyl ester (13qq): The product from above (23 mg, 0.05 mmol) was then dissolved in toluene (1 mL) and MnO₂ (23 mg, 0.26 mmol) was then added. The reaction was heated to 110 C for 24 hours after which the MnO₂ was filtered out and the product was purified by prep TLC. (MS calc'd for C₂₇H₂₈NO₅ (MH⁺): 446.2. Found 446.1.

### Example 111

### Preparation of 4-(3-Hydroxy-phenyl)-2-methyl-5-oxo-7-(2,4,6-trimethyl-phenyl)-1,5,7,8-tetrahydro-4H-pyrano[4,3-b]pyridine-3-carboxylic acid dimethylamide

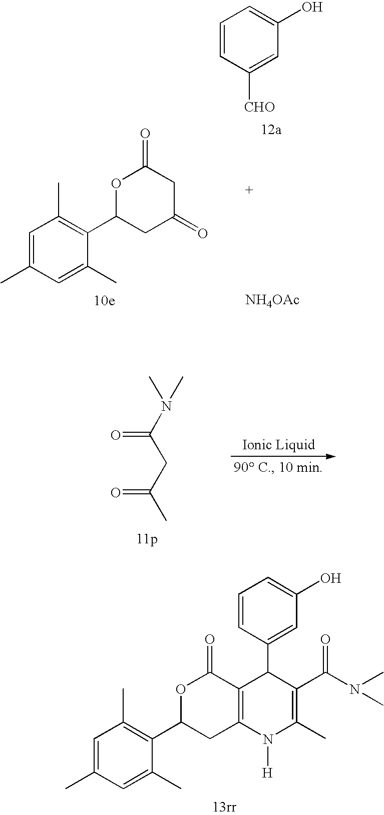

Synthesis of 4-(3-Hydroxy-phenyl)-2-methyl-5-oxo-7-(2,4,6-trimethyl-phenyl)-1,5,7,8-tetrahydro-4H-pyrano[4,3-b]pyridine-3-carboxylic acid dimethylamide (13rr): To a small vial was added 3-hydroxy-benzaldehyde (53 mg, 0.43 mmol) followed by 6-(2,4,6-trimethyl-phenyl)-dihydropyran-2,4-dione (100 mg, 0.43 mmol), ammonium acetate (50 mg, 0.65 mmol), N,N-dimethyl-3-oxo-butyramide (56 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (80% ethyl acetate/hexanes) to get the desired product (102 mg, 53%) as a solid. MS (ES) M+H expected=447.2, found=447.2.

### Example 112

### Preparation of 3-Benzenesulfonyl-4-(3-hydroxy-phenyl)-2-methoxymethyl-7-(2,4,6-trimethyl-phenyl)-1,4,7,8-tetrahydro-pyrano[4,3-b]pyridin-5-one

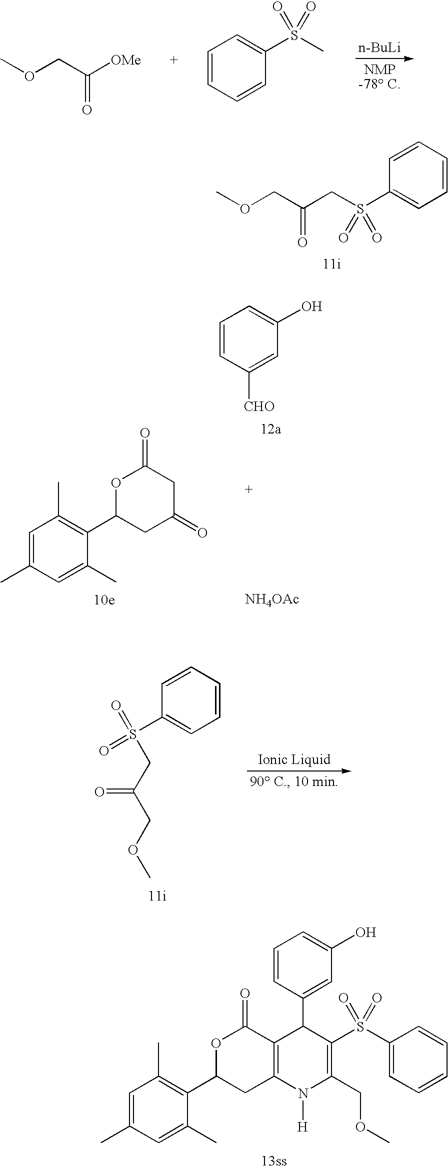

Synthesis of 1-Benzenesulfonyl-3-methoxy-propan-2-one (11i): Methanesulfonyl-benzene (180 mg, 1.15 mmol) was dissolved in 4 mL of THF and cooled to −78° C. To this mixture was added butyllithium (2.5 M solution in hexanes, 0.46 mL, 1.15 mmol). After stirring for 10 minutes at −78° C., 1-methyl-2-pyrrolidinone (NMP, 0.5 mL) was added. After another 10 minutes, a solution of methoxy-acetic acid methyl ester (120 mg, 1.15 mmol) in 1 mL of THF was added, and the reaction mixture was warmed up to room temperature. The reaction was quenched with sat. NH₄Cl solution and extracted with ethyl acetate. The organic layers were combined and washed with brine and dried over MgSO₄. Solvent was then removed and the residue was purified by column chromatography (80% ethyl acetate/hexanes). The product (77 mg, 55%) was obtained as a solid. MS (ES) M+H expected=229.0, found=229.1.

Synthesis of 3-Benzenesulfonyl-4-(3-hydroxy-phenyl)-2-methoxymethyl-7-(2,4,6-trimethyl-phenyl)-1,4,7,8-tetrahydro-pyrano[4,3-b]pyridin-5-one (13ss): To a small vial was added 3-hydroxy-benzaldehyde (53 mg, 0.43 mmol) followed by 6-(2,4,6-trimethyl-phenyl)-dihydropyran-2,4-dione (100 mg, 0.43 mmol), ammonium acetate (50 mg, 0.65 mmol), 1-benzenesulfonyl-3-methoxy-propan-2-one (98 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (70% ethyl acetate/hexanes) to get the desired product (117 mg, 50%) as a yellow solid. MS (ES) M+H expected=546.2, found=546.2.

### Example 113

### Preparation of 2-tert-Butoxymethyl-4-(1H-indazol-4-yl)-5-oxo-7-(2,4,6-trimethyl-phenyl)-1,5,7,8-tetrahydro-4H-pyrano[4,3-b]pyridine-3-carboxylic acid ethyl ester

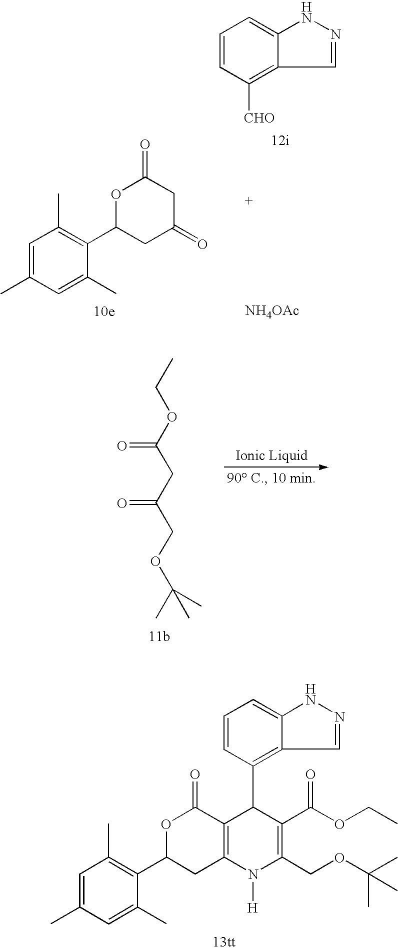

Synthesis of 2-tert-Butoxymethyl-4-(1H-indazol-4-yl)-5-oxo-7-(2,4,6-trimethyl-phenyl)-1,5,7,8-tetrahydro-4H-pyrano[4,3-b]pyridine-3-carboxylic acid ethyl ester (13tt): To a small vial was added 1H-Indazole-4-carbaldehyde (63 mg, 0.43 mmol) followed by 6-(2,4,6-trimethyl-phenyl)-dihydropyran-2,4-dione (100 mg, 0.43 mmol), ammonium acetate (50 mg, 0.65 mmol), 4-tert-butoxy-3-oxo-butyric acid ethyl ester (87 mg, 0.43 mmol) and 1-butyl-3-methylimidazolium tetrafluoroborate (9.7 μL, 0.052 mmol). The reaction mixture was then heated at 90° C. for 10 minutes, cooled down to room temperature, and then loaded on column (50% ethyl acetate/hexanes) to get the desired product (121 mg, 50%) as a yellow solid. MS (ES) M+H expected=544.3, found=544.4.

### Example 114

### Preparation of 4-(3-Hydroxy-phenyl)-2-methoxymethyl-5-oxo-7-(2,4,6-trimethyl-phenyl)-1,4,5,6,7,8-hexahydro-[1,7]naphthyridine-3-carboxylic acid methyl ester

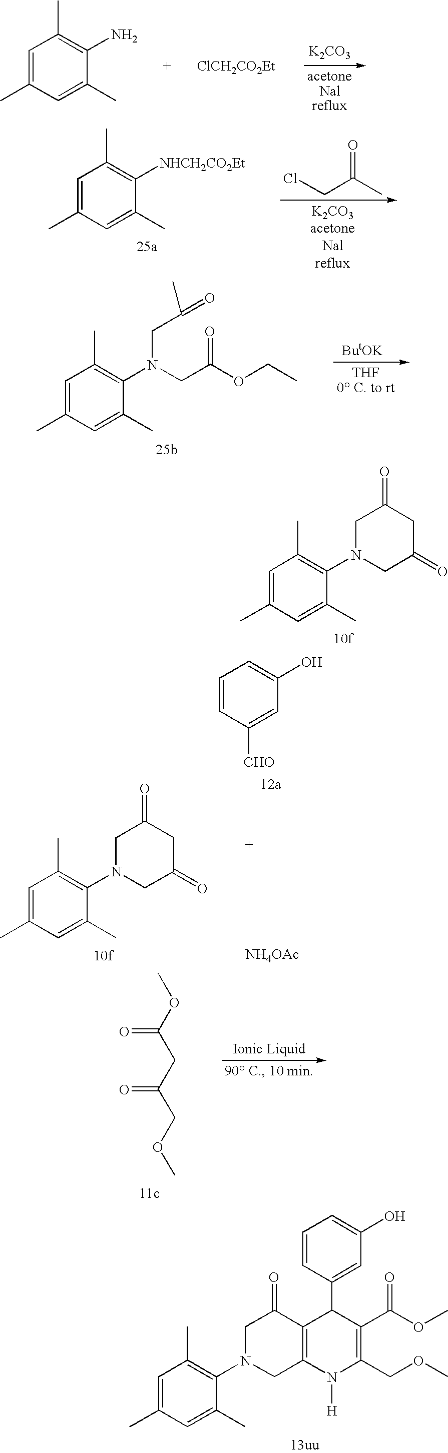

Synthesis of (2,4,6-Trimethyl-phenylamino)-acetic acid ethyl ester (25a): 2,4,6-Trimethyl-phenylamine (2.0 g, 14.81 mmol) was dissolved in 10 mL of acetone. Ethyl chloroacetate (2.2 g, 17.77 mmol) was added, followed by potassium carbonate (8.2 g, 59.24 mmol) and sodium iodide (2.4 g, 16.29 mmol). The reaction mixture was heated to reflux for 3 hours and cooled to room temperature. Water was added and the mixture was extracted with ethyl acetate, and the combined organic layers were combined and washed with brine, and dried over MgSO₄. Solvent was then removed and the residue was used directly for the next step.

Synthesis of [(2-Oxo-propyl)-(2,4,6-trimethyl-phenyl)-amino]-acetic acid ethyl ester (25b): To a solution of the ester obtained above (3.0 g, 13.57 mmol) in 10 mL of acetone was added chloroacetone (1.3 mL, 16.28 mmol) followed by potassium carbonate (7.5 g, 54.28 mmol) and sodium iodide (2.2 g, 14.93 mmol). The reaction mixture was heated to reflux for 3 hours and cooled to room temperature. Water was added and the mixture was extracted with ethyl acetate, and the combined organic layers were combined and washed with brine, and dried over MgSO₄. Solvent was then removed and the residue was purified by column chromatography (50% ethyl acetate/hexanes). The product (2.6 g, 70%) was obtained as a liquid. MS (ES) M+H expected=278.2, found=278.3.

Synthesis of 1-(2,4,6-Trimethyl-phenyl)-piperidine-3,5-dione (10f): [(2-Oxo-propyl)-(2,4,6-trimethyl-phenyl)-amino]-acetic acid ethyl ester (1.4 g, 5.05 mmol) was dissolved in 6 mL of THF, cooled to 0° C., and then a solution of potassium tert-butoxide in THF (1.0 M, 6.1 mL, 6.06 mmol) was added dropwise. The reaction mixture was then warmed up to room temperature and stirred for one hour. The reaction was then quenched with saturated ammonium chloride solution and extracted with ethyl acetate. The organic layers were combined, washed with brine and dried over MgSO₄. Solvent was then removed and the residue was purified by column chromatography (75% ethyl acetate/hexanes). The product (0.93 g, 80%) was obtained as a white solid. MS (ES) M+H expected=232.1, found=232.1.

Synthesis of 4-(3-Hydroxy-phenyl)-2-methoxymethyl-5-oxo-7-(2,4,6-trimethyl-phenyl)-1,4,5,6,7,8-hexahydro-[1,7]naphthyridine-3-carboxylic acid methyl ester (13uu): To a small vial was added 3-hydroxy-benzaldehyde (42 mg, 0.35 mmol) followed by 1-(2,4,6-trimethyl-phenyl)-piperidine-3,5-dione (80 mg, 0.35 mmol), ammonium acetate (40 mg, 0.52 mmol), 4-methoxy-3-oxo-butyric acid methyl ester (50 mg, 0.35 mmol) and 1-butyl-3-methylimidazolium tetrafluoroborate (7.8 μL, 0.042 mmol). The reaction mixture was then heated at 90° C. for 10 minutes, cooled down to room temperature, and then loaded on column (50% ethyl acetate/hexanes) to get the desired product (72 mg, 45%) as a yellow solid. MS (ES) M+H expected=463.2, found=463.2.

### Example 115

### Preparation of 7-Benzyl-2-tert-butoxymethyl-4-(3-hydroxy-phenyl)-5-oxo-1,4,5,6,7,8-hexahydro-[1,7]naphthyridine-3-carboxylic acid ethyl ester

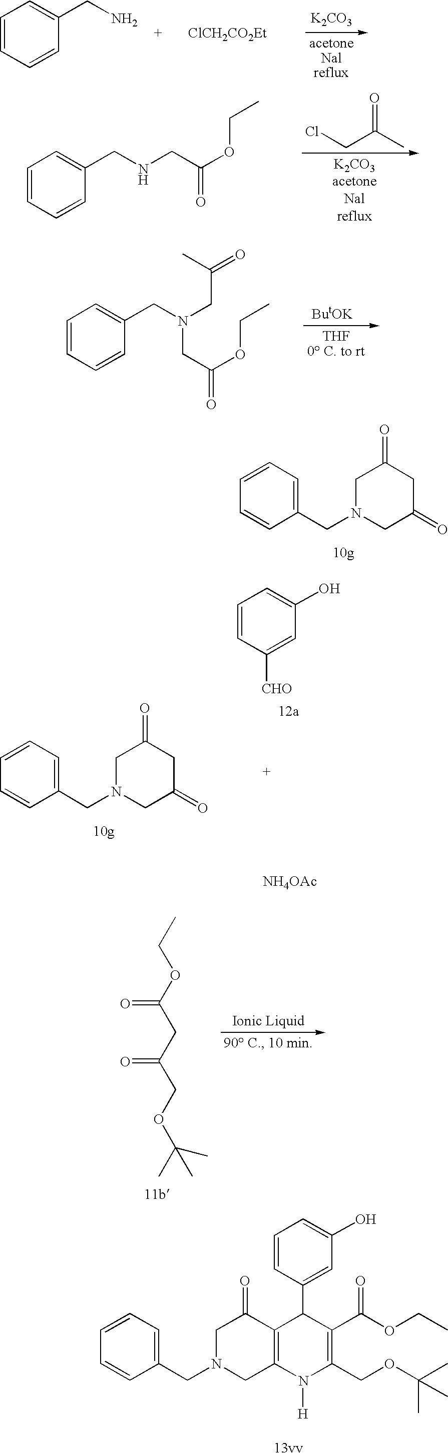

Same as Example 114 above.

### Example 116

### Preparation of 5-(1H-indol-4-yl)-7-methoxymethyl-4-oxo-2-phenyl-3,4,5,8-tetrahydropyrido[2,3-d]pyrimidine-6-carboxylic acid methyl ester

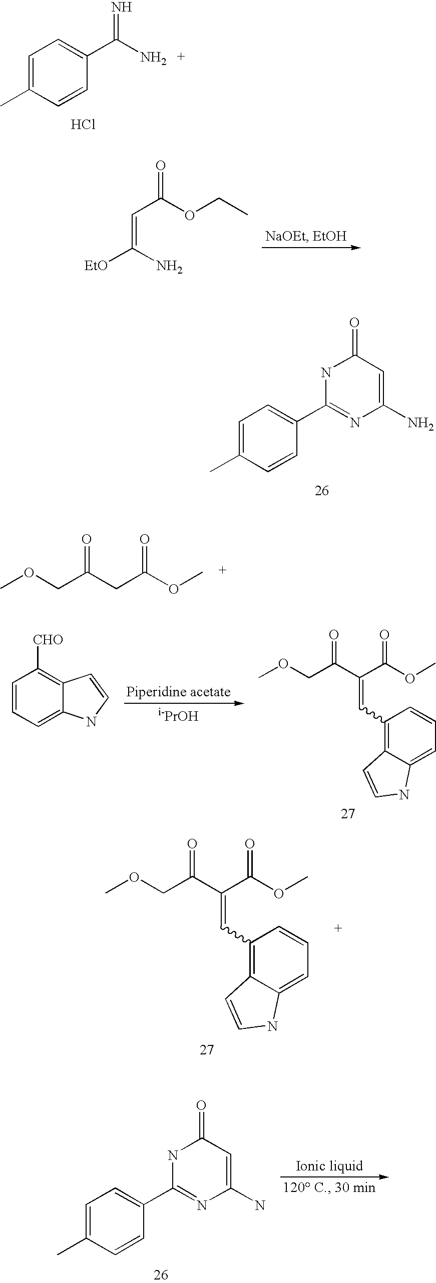

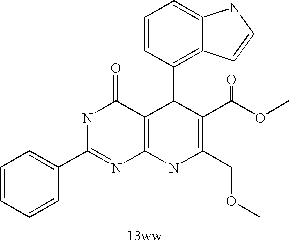

Synthesis of 6-amino-2-p-tolyl-3H-pyrimidin-4-one (26): To a suspension of 4-methylbenzamidine hydrochloride (0.5 g, 2.93 mmol) in absolute ethanol (5 mL) were added NaOEt (0.3 g, 4.39 mmol) followed by 3-amino-3-ethoxy-acrylic acid ethyl ester (0.46 g, 2.93 mmol) and the reaction mixture was heated to reflux for 48 hours. Excess solvent was removed in vacuum; diluted saturated aqueous NH₄Cl solution and extracted with (3×20 mL). The combined organic layers were washed with brine, dried (Na₂SO₄) and concentrated to give the crude 6-amino-2-p-tolyl-3H-pyrimidin-4-one which was used in the next step without further purification. LC-MS (method: 0-100-5 min) retention time (Rt)=1.92 min. MS calc'd for C₁₁H₁₁N₃O (MH⁺): 202.0. Found 202.0.

Synthesis of 2-(1H-indol-4-ylmethylene)-4-methoxy-3-oxobutyric acid methyl ester (27): To a solution of 4-methoxy-3-oxo-butyric acid methylester (1 g, 6.88 mmol) in anhydrous isopropanol (10 mL) were added 1H-indole-4-carbaldehyde (0.3 g, 4.39 mmol) followed by catalytic amount of piperidine acetate (0.1 g, 0.68 mmol) and the reaction mixture was stirred at ambient temperature for 6 hours. The separated yellow solid was filtered to get 2-(1H-indol-4-ylmethylene)-4-methoxy-3-oxo-butyric acid methyl ester in 75% yield (1.41 g). LC-MS (method: 20-100-5 min) retention time (Rt)=1.61 min. MS calc'd for C₁₅H₁₅NO₄ (MH⁺): 273.1. Found 273.1.

Synthesis of 5-(1H-indol-4-yl)-7-methoxymethyl-4-oxo-2-phenyl-3,4,5,8-tetrahydropyrido[2,3-d]pyrimidine-6-carboxylic acid methyl ester (13ww): The above compound was synthesized by using general Hantschz procedure. LC-MS (method: 20-100-5 min) retention time (Rt)=2.38 min. MS calc'd for C₂₅H₂₂N₄O₄ (MH⁺): 457.1. Found 457.1.

### Example 117

Materials and Methods

A. Cells

1. C5a Receptor Expressing Cells

a) C5aR L1.2

L1.2 cells (ATCC #HB-12624) were transfected with a mammalian expression vector constitutively expressing the human C5a receptor (Genbank accession # NM_{—}001736). A stable cell line was produced by selection with G418 for the co-expressed resistance marker. These cells were cultured as a suspension in RPMI-1640 medium supplemented with 2 mM L-glutamine, 1.5 g/L sodium bicarbonate, 4.5 g/L glucose, 10 mM HEPES, 1 mM sodium pyruvate, and 10% FBS. Cells were grown under 5% CO₂/95% air, 100% humidity at 37° C. and subcultured twice weekly at 1:6 (cells were cultured at a density range of 1×10⁵ to 2×10⁶ cells/mL) and harvested at 1×10⁶ cells/mL. L1.2 C5aR cells express C5a receptor and can be used in C5aR ligand binding and functional assays.

b) Isolated Human Neutrophils

Neutrophils were isolated from fresh human blood using density separation and centrifigation. Briefly, whole blood is incubated with equal parts 3% dextran and allowed to separate for 45 minutes. After separation, the top layer is then layered on top of 15 mls of Ficoll (15 mls of Ficoll for every 30 mls of blood suspension) and centrifuged for 30 minutes at 400×g with no brake. The pellet at the bottom of the tube is then isolated and resuspended into PharmLyse RBC Lysis Buffer (BD Biosciences, San Jose, Calif.) after which the sample is again centrifuged for 10 minutes at 400×g with brake. The remaining cell pellet is resuspended as appropriate and consists of isolated neutrophils.

B. Assays

1. Inhibition of C5aR Ligand Binding

C5aR expressing cells were centrifuged and resuspended in assay buffer (20 mM HEPES pH 7.1, 140 mM NaCl, 1 mM CaCl₂, 5 mM MgCl₂, and with 0.2% bovine serum albumin) to a concentration of 5×10⁶ cells/mL for L1.2 C5aR cells and 1×10⁵ for neutrophils. Binding assays were set up as follows. 0.1 mL of cells (5×10⁵ L1.2 C5aR cells/well or 1×10⁵ neutrophils) was added to the assay plates containing the compounds, giving a final concentration of ˜2-10 μM each compound for screening (or part of a dose response for compound IC₅₀ determinations). Then 0.1 mL of ¹²⁵I labeled C5a (obtained from Perkin Elmer Life Sciences, Boston, Mass.) diluted in assay buffer to a final concentration of ˜50 pM, yielding ˜30,000 cpm per well, was added (using ¹²⁵I labeled C5a with L1.2 C5aR cells and with neutrophils), the plates sealed and incubated for approximately 3 hours at 4° C. on a shaker platform. Reactions were aspirated onto GF/B glass filters pre-soaked in 0.3% polyethyleneimine (PEI) solution, on a vacuum cell harvester (Packard Instruments; Meriden, Conn.). Scintillation fluid (40 μl; Microscint 20, Packard Instruments) was added to each well, the plates were sealed and radioactivity measured in a Topcount scintillation counter (Packard Instruments). Control wells containing either diluent only (for total counts) or excess C5a (1 82 g/mL, for non-specific binding) were used to calculate the percent of total inhibition for compound. The computer program Prism from GraphPad, Inc. (San Diego, Calif.) was used to calculate IC₅₀ values. IC₅₀ values are those concentrations required to reduce the binding of radiolabeled C5a to the receptor by 50%. (For further descriptions of ligand binding and other functional assays, see Dairaghi, et al., J. Biol. Chem. 274:21569-21574 (1999), Penfold, et al., Proc. Natl. Acad. Sci. USA. 96:9839-9844 (1999), and Dairaghi, et al, J. Biol. Chem. 272:28206-28209 (1997)).

2. Calcium Mobilization

To detect the release of intracellular stores of calcium, cells (L1.2 C5aR or neutrophils) were incubated with 3 μM of INDO-1AM dye (Molecular Probes; Eugene, Oreg.) in cell media for 45 minutes at room temperature and washed with phosphate buffered saline (PBS). After INDO-1AM loading, the cells were resuspended in flux buffer (Hank's balanced salt solution (HBSS) and 1% FBS). Calcium mobilization was measured using a Photon Technology International spectrophotometer (Photon Technology International; New Jersey) with excitation at 350 nm and dual simultaneous recording of fluorescence emission at 400 nm and 490 nm. Relative intracellular calcium levels were expressed as the 400 nm/490 nm emission ratio. Experiments were performed at 37° C. with constant mixing in cuvettes each containing 10⁶ cells in 2 mL of flux buffer. The chemokine ligands may be used over a range from 1 to 100 nM. The emission ratio was plotted over time (typically 2-3 minutes). Candidate ligand blocking compounds (up to 10 μM) were added at 10 seconds, followed by chemokines at 60 seconds (i.e., C5a; R&D Systems; Minneapolis, Minn.) and control chemokine (i.e., SDF-1α; R&D Systems; Minneapolis, Minn.) at 150 seconds.

3. Chemotaxis Assays

Chemotaxis assays were performed using 5 μm pore polycarbonate, polyvinylpyrrolidone-coated filters in 96-well chemotaxis chambers (Neuroprobe; Gaithersburg, Md.) using chemotaxis buffer (Hank's balanced salt solution (HBSS) and 1% FBS). C5aR ligands (i.e., C5a, R&D Systems; Minneapolis, Minn.) are use to evaluate compound mediated inhibition of C5aR mediated migration. Other chemokines (i.e., SDF-1α; R&D Systems; Minneapolis, Minn.) are used as specificity controls. The lower chamber was loaded with 29 μl of chemokine (i.e., 0.03 nM C5a) and varying amounts of compound; the top chamber contained 100,000 L1.2 C5aR or neutrophil cells in 20 μl. The chambers were incubated 1.5 hours at 37° C., and the number of cells in the lower chamber quantified either by direct cell counts in five high powered fields per well or by the CyQuant assay (Molecular Probes), a fluorescent dye method that measures nucleic acid content and microscopic observation.

C. Identification of Inhibitors of C5aR

1. Assay

To evaluate small organic molecules that prevent the C5a receptor from binding ligand, an assay was employed that detected radioactive ligand (i.e, C5a) binding to cells expressing C5aR on the cell surface (for example, L1.2 C5AR cells or isolated human neutrophils). For compounds that inhibited binding, whether competitive or not, fewer radioactive counts are observed when compared to uninhibited controls.

Equal numbers of cells were added to each well in the plate. The cells were then incubated with radiolabeled C5a. Unbound ligand was removed by washing the cells, and bound ligand was determined by quantifying radioactive counts. Cells that were incubated without any organic compound gave total counts; non-specific binding was determined by incubating the cells with unlabeled ligand and labeled ligand. Percent inhibition was determined by the equation:
% inhibition=(1−[(sample cpm)−(nonspecific cpm)]/[(total cpm)−(nonspecific cpm)])×100.

2. Dose Response Curves

To ascertain a candidate compound's affinity for C5aR as well as confirm its ability to inhibit ligand binding, inhibitory activity was titered over a 1×10⁻¹⁰ to 1×10⁻⁴ M range of compound concentrations. In the assay, the amount of compound was varied; while cell number and ligand concentration were held constant.

3. C5aR Functional Assays

C5aR is a seven transmembrane, G-protein linked receptor. A hallmark of signaling cascades induced by the ligation of some such receptors is the pulse-like release of calcium ions from intracellular stores. Calcium mobilization assays were performed to determine if the candidate C5aR inhibitory compounds were able to also block aspects of C5aR signaling. Candidate compounds able to inhibit ligand binding and signaling with an enhanced specificity over other chemokine and non-chemokine receptors were desired.

Calcium ion release in response to C5aR chemokine ligands (i.e., C5a) was measured using the calcium indicator INDO-1. L1.2 C5AR cells or neutrophils were loaded with INDO-1/AM and assayed for calcium release in response to C5aR ligand (i.e., C5a) addition. To control for specificity, non-C5aR ligands, specifically SDF-1 alpha, are added, which also signals via a seven transmembrane receptor. Without compound, a pulse of fluorescent signal will be seen upon C5a addition. If a compound specifically inhibits C5aR-C5a signaling, then little or no signal pulse will be seen upon C5a addition, but a pulse will be observed upon SDF-1 alpha addition. However, if a compound non-specifically inhibits signaling, then no pulse will be seen upon both C5a and SDF-1 alpha addition.

One of the primary functions of chemoattractant proteins, such a C5a, is their ability to mediate the migration of chemoattractant receptor-expressing cells, such as white blood cells. To confirm that a candidate compound modulated not only C5aR specific binding and signaling (at least as determined by calcium mobilization assays), but also C5aR mediated migration, a chemotaxis assay was employed. L1.2 C5aR cells, as wells as freshly isolated neutrophils, are used as targets for chemoattractant by C5aR ligands (i.e., C5a). Cells are place in the top compartment of a microwell chemotaxis chamber with increasing concentrations of a candidate compound, while a fixed amount of C5a (0.03 nM ) is loaded in the lower chamber. In the absence of compound modulator, cells proceed to move chemotactically into the lower chamber in response to the chemoattractant; if a compound modulates C5aR function, then the majority of cells will remain in the upper chamber. To ascertain the affinity of a candidate compound for C5aR as well as to confirm its ability to inhibit C5aR mediated cell migration, inhibitory activity was titered over a 1×10⁻¹⁰ to 1×10⁻⁴ M range of compound concentrations in this chemotaxis assay. In this assay, the amount of compound was varied; while cell number and chemotractic agonist concentrations were held constant. After the chemotaxis chambers were incubated 1.5 hours at 37° C., the responding cells in the lower chamber were quantified by labeling with the CyQuant assay (Molecular Probes), a fluorescent dye method that measures nucleic acid content, and by measuring with a Spectrafluor Plus (Tecan). The computer program Prism from GraphPad, Inc. (San Diego, Calif.) was used to calculate IC₅₀ values. IC₅₀ values are those compound concentrations required to inhibit the number of cells responding to a CCR1 agonist by 50%.

D. In Vivo Efficacy Models

The compounds of interest can be evaluated for potential efficacy in treating a C5a mediated conditions by determining the efficacy of the compound in an animal model. In addition to the models described below, other suitable animal models for studying the compound of interest can be found in Mizuno, M. et al., Expert Opin. Investig. Drugs (2005), 14(7), 807-821, which is incorporated herein by reference in its entirety.

1. Rheumatoid Arthritis Models

a) Rabbit Model of Destructive Joint Inflammation

To study the effects of candidate compounds on inhibiting the inflammatory response of rabbits to an intra-articular injection of the bacterial membrane component lipopolysaccharide (LPS), a rabbit model of destructive joint inflammation is used. This study design mimics the destructive joint inflammation seen in arthritis. Intra-articular injection of LPS causes an acute inflammatory response characterized by the release of cytokines and chemokines, many of which have been identified in rheumatoid arthritic joints. Marked increases in leukocytes occur in synovial fluid and in synovium in response to elevation of these chemotactic mediators. Selective antagonists of chemokine receptors have shown efficacy in this model (see Podolin, et al., J. Immunol. 169(11):6435-6444 (2002)).

A rabbit LPS study is conducted essentially as described in Podolin, et al. ibid., female New Zealand rabbits (approximately 2 kilograms) are treated intra-articularly in one knee with LPS (10 ng) together with either vehicle only (phosphate buffered saline with 1% DMSO) or with addition of candidate compound (dose 1=50 μM or dose 2=100 μM) in a total volume of 1.0 mL. Sixteen hours after the LPS injection, knees are lavaged and cells counts are performed. Beneficial effects of treatment were determined by histopathologic evaluation of synovial inflammation. Inflammation scores are used for the histopathologic evaluation: 1-minimal, 2-mild, 3-moderate, 4-moderate-marked.

b) Evaluation of a Compound in a Rat Model of Collagen Induced Arthritis

A 17 day developing type II collagen arthritis study is conducted to evaluate the effects of a candidate compound on arthritis induced clinical ankle swelling. Rat collagen arthritis is an experimental model of polyarthritis that has been widely used for preclinical testing of numerous anti-arthritic agents (see Trentham, et al., J. Exp. Med. 146(3):857-868 (1977), Bendele, et al., Toxicologic Pathol. 27:134-142 (1999), Bendele, et al., Arthritis Rheum. 42:498-506 (1999)). The hallmarks of this model are reliable onset and progression of robust, easily measurable polyarticular inflammation, marked cartilage destruction in association with pannus formation and mild to moderate bone resorption and periosteal bone proliferation.

Female Lewis rats (approximately 0.2 kilograms) are anesthetized with isoflurane and injected with Freund's Incomplete Adjuvant containing 2 mg/mL bovine type II collagen at the base of the tail and two sites on the back on days 0 and 6 of this 17 day study. A candidate compound is dosed daily in a sub-cutaneous manner from day 0 till day 17 at a efficacious dose. Caliper measurements of the ankle joint-diameter were taken, and reducing joint swelling is taken as a measure of efficacy.

2. Rat Model of Sepsis

To study the effect of compounds of interest on inhibiting the generalized inflammatory response that is associated with a sepsis like disease, the Cecal Ligation and Puncture (CLP) rat model of sepsis is used. A Rat CLP study is conducted essentially as described in Fujimura N, et al. (American Journal Respiratory Critical Care Medicine 2000; 161: 440-446). Briefly described here, Wistar Albino Rats of both sexes weighing between 200-250 g are fasted for twelve hours prior to experiments. Animals are kept on normal 12 hour light and dark cycles and fed standard rat chow up until 12 hours prior to experiment. Then animals are split into four groups; (i) two sham operation groups and (ii) two CLP groups. Each of these two groups (i.e., (i) and (ii)) is split into vehicle control group and test compound group. Sepsis is induced by the CLP method. Under brief anesthesia a midline laparotomy is made using minimal dissection and the cecum is ligated just below the ileocaecal valve with 3-0 silk, so the intestinal continuity is maintained. The antimesinteric surface of the cecum is perforated with an 18 gauge needle at two locations 1 cm apart and the cecum is gently squeezed until fecal matter is extruded. The bowel is then returned to the abdomen and the incision is closed. At the end of the operation, all rats are resuscitated with saline, 3 ml/100 g body weight, given subcutaneously. Postoperatively, the rats are deprived of food, but have free access to water for the next 16 hours until they are sacrificed. The sham operated groups are given a laparotomy and the cecum is manipulated but not ligated or perforated. Beneficial effects of treatment are measured by histopathological scoring of tissues and organs as well as measurement of several key indicators of hepatic function, renal function, and lipid peroxidation. To test for hepatic function aspartate transaminase (AST) and alanine transaminase (ALT) are measured. Blood urea nitrogen and creatinine concentrations are studied to assess renal function. Pro-inflammatory cytokines such as TNF-alpha and IL-b 1beta are also assayed by ELISA for serum levels.

3. Mouse SLE Model of Experimental Lupus Nephritis.

To study the effect of compounds of interest on a Systemic Lupus Erythematosus (SLE), the MRL/lpr murine SLE model is used. The MRL/Mp-Tmfrsf6^{lpr/lpr} strain (MRL/lpr) is a commonly used mouse model of human SLE. To test compounds efficacy in this model male MRL/lpr mice are equally divided between control and C5aR antagonists groups at 13 weeks of age. Then over the next 6 weeks compound or vehicle is administered to the animals via osmotic pumps to maintain coverage and minimize stress effects on the animals. Serum and urine samples are collected bi-weekly during the six weeks of disease onset and progression. In a minority of these mice glomerulosclerosis develops leading to the death of the animal from renal failure. Following mortality as an indicator of renal failure is one of the measured criteria and successful treatment will usually result in a delay in the onset of sudden death among the test groups. In addition, the presence and magnitude of renal disease may also be monitored continuously with blood urea nitrogen (BUN) and albuminuria measurements. Tissues and organs were also harvested at 19 weeks and subjected to histopathology and immunohistochemistry and scored based on tissue damage and cellular infiltration.

4. Rat Model of COPD

Smoke induced airway inflammation in rodent models may be used to assess efficacy of compounds in Chronic Obstructive Pulmonary Disease (COPD). Selective antagonists of chemokines have shown efficacy in this model (see, Stevenson, et al., Am. J. Physiol Lung Cell Mol Physiol. 288 L514-L522, (2005)).

An acute rat model of COPD is conducted as described by Stevenson et al. A compound of interest is administered either systemically via oral or IV dosing; or locally with nebulized compound. Male Sprague-Dawley rats (350-400 g) are placed in Perspex chambers and exposed to cigarette smoke drawn in via a pump (50 mL every 30 seconds with fresh air in between). Rats are exposed for a total period of 32 minutes. Rats are sacrificed up to 7 days after initial exposure. Any beneficial effects of treatment are assessed by a decrease inflammatory cell infiltrate, decreases in chemokine and cytokine levels.

In a chronic model, mice or rats are exposed to daily tobacco smoke exposures for up to 12 months. Compound is administered systemically via once daily oral dosing, or potentially locally via nebulized compound. In addition to the inflammation observed with the acute model (Stevensen et al.), animals may also exhibit other pathologies similar to that seen in human COPD such as emphysema (as indicated by increased mean linear intercept) as well as altered lung chemistry (see Martorana et al, Am. J. Respir. Crit Care Med. 172(7): 848-53.

5. Mouse EAE Model of Multiple Scleroris

Experimental autoimmune encephalomyelitis (EAE) is a model of human multiple sclerosis. Variations of the model have been published, and are well known in the field. In a typical protocol, C57BL/6 (Charles River Laboratories) mice are used for the EAE model. Mice are immunized with 200 ug myelin oligodendrocyte glycoprotein (MOG) 35-55 (Peptide International) emulsified in Complete Freund's Adjuvant (CFA) containing 4 mg/ml Mycobacterium tuberculosis (Sigma-Aldrich) s.c. on day 0. In addition, on day 0 and day 2 animals are given 200 ng of pertussis toxin (Calbiochem) i.v. Clinical scoring is based on a scale of 0-5: 0, no signs of disease; 1, flaccid tail; 2, hind limb weakness; 3, hind limb paralysis; 4, forelimb weakness or paralysis; 5, moribund. Dosing of the compounds of interest to be assessed can be initiated on day 0 (prophylactic) or day 7 (therapeutic, when histological evidence of disease is present but few animals are presenting clinical signs) and dosed once or more per day at concentrations appropriate for their activity and pharmacokinetic properties, e.g. 100 mg/kg s.c. Efficacy of compounds can be assessed by comparisons of severity (maximum mean clinical score in presence of compound compared to vehicle), or by measuring a decrease in the number of macrophages (F4/80 positive) isolated from spinal cords. Spinal cord mononuclear cells can be isolated via discontinuous Percoll-gradient. Cells can be stained using rat anti-mouse F4/80-PE or rat IgG2b-PE (Caltag Laboratories) and quantitated by FACS analysis using 10 ul of Polybeads per sample (Polysciences).

1. A compound having the formula 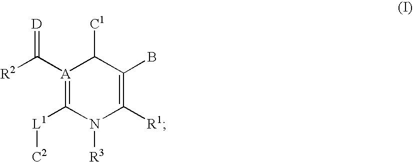
and pharmaceutically acceptable salts thereof; wherein
A is nitrogen or carbon;
B is selected from the group consisting of halogen, —CN, —NO₂, —C(═NOR^{a})R^{c}, —C(═NR^{c})R^{c}, —CO₂R^{a}, —CONR^{a}R^{b}, —C(O)R^{a}, —OC(O)NR^{a}R^{b}, —NR^{b}C(O)R^{a}, —NR^{b}C(O)₂R^{c}, —NR^{a}—C(O)NR^{a}R^{b}, —NH—C(NH₂)═NH, —NR^{c}C(NH₂)═NH, —NH—C(NH₂)═NR^{c}, —NHC(NHR^{c})═NH, —NR^{a}C(O)NR^{a}R^{b}, —C(NR^{a}W)═NW, —N(W)C(R^{a})═NW, —X¹C(NR^{a}W)═NW, —X¹N(W)C(R^{a})═NW, —X¹—(NR^{a}R^{b}), —X¹—(OR^{a}), —X¹—(SR^{a}), —S(O)R^{c}, —S(O)₂R^{c}, —NR^{a}S(O)₂R^{c}, —S(O)₂NR^{a}R^{b}, —NR^{a}S(O)₂R^{c} and —NR^{a}S(O)₂NR^{a}R^{b}; wherein X¹ is a C₁₋₄ alkylene, C₁₋₄ heteroalkylene, C₂₋₄ alkenylene or C₂₋₄ alkynylene; each R^{a} and R^{b} is independently selected from hydrogen, C₁₋₈ alkyl, C₁₋₈ haloalkyl, C₂₋₈ heteroalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl, or when attached to the same nitrogen atom can be combined with the nitrogen atom to form a five or six-membered ring having from 0 to 2 additional heteroatoms as ring members selected from N, O or S; R^{c} is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl and aryloxy-C₁₋₄ alkyl, wherein W is independently selected from the group consisting of —R^{c}, —CN, —CO₂R^{e} and —NO₂, and wherein the aliphatic portions of X¹, R^{a}, R^{b} and R^{c} are optionally further substituted with from one to three members selected from the group consisting of halogen, —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{o}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —R^{m}, —CN, —NO₂, —NH₂, —NHR^{m}, —N(R^{m})₂, —NR^{m}S(O)NH₂ and —NR^{m}S(O)₂NHR^{m}, wherein R^{m} is unsubstituted C₁₋₆ alkyl;
R¹ is selected from the group consisting of hydrogen, amino, C₁₋₈ alkylamino, C₁₋₈ dialkylamino, C₁₋₈ alkoxy, C₁₋₈ alkyl, C₁₋₈ haloalkyl, C₃₋₆ cycloalkyl, C₃₋₆ heterocycloalkyl, C₃₋₆ cycloalkyl-C₁₋₈ alkyl, C₃₋₆ heterocycloalkyl-C₁₋₆ alkyl, and wherein each R¹ substituent is optionally substituted with from one to three members selected from the group consisting of halogen, —OH, —ORⁿ, —OC(O)NHRⁿ, —OC(O)N(Rⁿ)₂, —SH, —SRⁿ, —S(O)Rⁿ, —S(O)₂Rⁿ, —SO₂NH₂, —S(O)₂NHRⁿ, —S(O)₂N(Rⁿ)₂, —NHS(O)₂Rⁿ, —NRⁿS(O)₂Rⁿ, —C(O)NH₂, —C(O)NHRⁿ, —C(O)N(Rⁿ)₂, —C(O)Rⁿ, —NHC(O)Rⁿ, —NRⁿC(O)Rⁿ, —NHC(O)NH₂, —NRⁿC(O)NH₂, —NRⁿC(O)NHRⁿ, —NHC(O)NHRⁿ, —NRⁿC(O)N(Rⁿ)₂, —NHC(O)N(Rⁿ)₂, —CO₂H, —CO₂Rⁿ, —NHCO₂Rⁿ, —NRⁿCO₂Rⁿ, —Rⁿ, —CN, —NO₂, —NH₂, —NHRⁿ, —N(Rⁿ)₂, —NRⁿS(O)NH₂ and —NRⁿS(O)₂NHRⁿ, wherein each Rⁿ is independently an unsubstituted C₁₋₆ alkyl;
C¹ and C² are each independently aryl, aryl-C₁₋₄ alkyl, heteroaryl, heteroaryl-C₁₋₄ alkyl, cycloalkyl, cycloalkyl-C₁₋₄ alkyl, heterocycloalkyl or heterocycloalkyl-C₁₋₄ alkyl, wherein the heterocycloalkyl group has from 1-3 heteroatoms selected from N, O and S; C¹ and C² has from 0 to 7 R⁴ substituents selected from the group consisting of halogen, cyano, heteroaryl, —NO₂, —CO₂R^{d}, —C(O)NR^{d}R^{e}, —C(O)R^{d}, —S(O)R^{f}, —S(O)₂R^{f}, —OC(O)R^{d}, —NR^{d}—C(O)NR^{d}R^{e}, —NH—C(NH₂)═NH, —NR^{f}C(NH₂)═NH, —NH—C(NH₂)═NR^{f}, —NH—C(NHR^{f})═NH, —NR^{d}S(O)₂R^{f}, —NR^{d}S(O)₂R^{f}, —NR^{d}S(O)₂NR^{d}R^{e}, —N₃, —R^{f}, —C(NOR^{d})R^{e}, —C(NR^{d}V)═NV, —N(V)C(R^{d})═NV, —X²C(NOR^{d})R^{e}, —X²C(NR^{d}V)═NV, —X²N(V)C(R^{d})═NV, —X²NR^{d}R^{e}, —X²SR^{d}, —X²CN, —X²NO₂, —X²CO₂R^{d}, —X²CONR^{d}R^{e}, —X²C(O)R^{d}, —X²OC(O)NR^{d}R^{e}, —X²NR^{e}C(O)R^{d}, —X²NR^{e}C(O)₂R^{f}, —X²NR^{d}C(O)NR^{c}R^{e}, —X²NH—C(NH₂)═NH, —X²NR^{f}C(NH₂)═NH, —X²NH—C(NH₂)═NR^{f}, —X²NH—C(NHR^{f})═NH, —X²S(O)R^{f}, —X²S(O)₂R^{f}, —X²NR^{c}S(O)₂R^{f}, —X²S(O)₂NR^{d}R^{e}, —X²N₃, —NR^{d}R^{e}, —OR^{d}, —SR^{d}, —NR^{e}C(O)R^{d}, —NR^{e}C(O)₂R^{f}, —S(O)₂NR^{d}R^{e}, —X²OR^{d}, —O—X²OR^{d}, —O—X²NR^{d}R^{e} and —NR^{e}—X²CO₂R^{d}; optionally any two substituents located on adjacent atoms of C¹ or C² are combined to form a 5- to 7-membered ring; and wherein X² is C₁₋₄ alkylene, C₁₋₄ heteroalkylene, C₂₋₄ alkenylene or C₂₋₄ alkynylene and each R^{d} and R^{e} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, aryl-C₁₋₄ alkyl, and optionally, R^{d} and R^{e} when attached to the same nitrogen atom are optionally combined to form a five or six-membered ring having from 0 to 2 additional heteroatoms as ring members; and each R^{f} is independently selected from the group consisting of C₁₋₈ alkyl, C₁₋₈ haloalkyl, C₃₋₆ cycloalkyl, C₂₋₈ alkenyl, C₂₋₈ alkynyl, aryl and heteroaryl, and each of X², R^{d}, R^{e} and R^{f} is optionally further substituted with from one to three members selected from the group consisting of halogen, —OH, —OR^{o}, —OC(O)NHR^{o}, —OC(O)N(R^{o})₂, —SH, —SR^{o}, —S(O)R^{o}, —S(O)₂R^{o}, —SO₂NH₂, —S(O)₂NHR^{o}, —S(O)₂N(R^{o})₂, —NHS(O)₂R^{o}, —NR^{o}S(O)₂R^{o}, —C(O)NH₂, —C(O)NHR^{o}, —C(O)N(R^{o})₂, —C(O)R^{o}, —NHC(O)R^{o}, —NR^{o}C(O)R^{o}, —NHC(O)NH₂, —NR^{o}C(O)NH₂, —NR^{o}C(O)NHR^{o}, —NHC(O)NHR^{o}, —NR^{o}C(O)N(R^{o})₂, —NHC(O)N(R^{o})₂, —CO₂H, —CO₂R^{o}, —NHCO₂R^{o}, —NR^{o}CO₂R^{o}, —CN, —NO₂, —NH₂, —NHR^{o}, —N(R^{o})₂, —NR^{o}S(O)NH₂ and —NR^{o}S(O)₂NHR^{o}, wherein each R^{o} is independently an unsubstituted C₁₋₆ alkyl; and wherein V is independently selected from the group consisting of —R^{f}, —CN, —CO₂R^{e} and —NO₂;
L¹ is a direct bond, C₁₋₈ alkylene, C₂₋₈ alkenylene, C₂₋₈ alkynylene, C₁₋₈ heteroalkylene, —S—, —S(O)—, —S(O)₂13 , —O—, —NH—, or —NR^{j}—; wherein R^{j} is C₁₋₆ alkyl, C₁₋₆ acyl or C₁₋₆ haloalkyl; wherein the aliphatic portions of L¹ is optionally further substituted with from one to three members selected from the group consisting of halogen, —OH, —OR^{p}, —OC(O)NHR^{p}, —OC(O)N(R^{p})₂, —SH, —SR^{p}, —S(O)R^{p}, —S(O)₂R^{p}, —SO₂NH₂, —S(O)₂NHR^{p}, —S(O)₂N(R^{p})₂, —NHS(O)₂R^{p}, —NR^{t}S(O)₂R^{p}, —C(O)NH₂, —C(O)NHR^{p}, —C(O)N(R^{p})₂, —C(O)R^{p}, —NHC(O)R^{p}, —NR^{p}C(O)R^{p}, —NHC(O)NH₂, —NR^{p}C(O)NH₂, —NR^{p}C(O)NHR^{p}, —NHC(O)NHR^{p}, —NR^{p}C(O)N(R^{p})₂, —NHC(O)N(R^{p})₂, —CO₂H, —CO₂R^{p}, —NHCO₂R^{p}, —NR^{p}CO₂R^{p}, —CN, —NO₂, —NH₂, —NHR^{p}, —N(R^{p})₂, —NR^{p}S(O)NH₂ and —NR^{p}S(O)₂NHR^{p}, wherein each R^{p} is independently an unsubstituted C₁₋₆ alkyl or C₁₋₆ haloalkyl;
R² is selected from the group consisting of —OR^{g}, —NR^{g}R^{h} and —Rⁱ, each R^{g} and R^{h} is independently selected from hydrogen, C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl and aryloxy-C₁₋₄ alkyl, or when attached to the same nitrogen atom are optionally combined to form a five or six-membered ring having from 0 to 2 additional heteroatoms as ring members; Rⁱ is independently selected from the group consisting of C₁₋₈ alkyl, C₁₋₈ haloalkyl, C₃₋₆ cycloalkyl, C₂₋₈ alkenyl, C₂₋₈ alkynyl, aryl, heteroaryl, heteroaryl-C₁₋₄ alkyl, aryl-C₁₋₄ alkyl, heteroaryl-C₁₋₄ heteroalkyl, aryl-C₁₋₄ heteroalkyl and aryloxy-C₁₋₄ alkyl, wherein the aliphatic portions of R^{g}, R^{h} and Rⁱ are optionally further substituted with from one to three members selected from the group consisting of halogen, —OH, —OR^{q}, —OC(O)NHR^{q}, —OC(O)N(R^{q})₂, —SH, —SR^{q}—S(O)R^{q}, —S(O)₂R^{q}, —SO₂NH₂, —S(O)₂NHR^{q}, —S(O)₂N(R^{q})₂, —NHS(O)₂R^{q}, —NR^{q}S(O)₂R^{q}, —C(O)NH₂, —C(O)NHR^{q}, —C(O)N(R^{q})₂, —C(O)R^{q}, —NHC(O)R^{q}, —NR^{q}C(O)R^{q}, —NHC(O)NH₂, —NR^{q}C(O)NH₂, —NR^{q}C(O)NHR^{q}, —NHC(O)NHR^{q}, —NR^{q}C(O)N(R^{q})₂, —NHC(O)N(R^{q})₂, —CO₂H, —CO₂R^{q}, —NHCO₂R^{q}, —NR^{q}CO₂R^{q}, —CN, —NO₂, —NH₂, —NHR^{q}, —N(R^{q})₂, —NR^{q}S(O)NH₂ and —NR^{q}S(O)₂NHR^{q}, wherein R^{q} is unsubstituted C₁₋₆ alkyl;
Optionally, the substituents R² and L¹, together with the atoms to which they are attached, are combined to form a 6-membered carbocyclic or heterocyclic ring;
R³ is hydrogen, C₁₋₆ alkyl, C₁₋₆ haloalkyl, C₁₋₆ acyl, C₃₋₈ cycloalkyl, aryl, heteroaryl, aryl-C₁₋₄ alkyl, heteroaryl-C₁₋₄ alkyl or C₂₋₆ alkenyl, wherein the aliphatic portions of R³ are optionally substituted with 1 to 3 substituents selected from the group consisting of —OH, —OR^{s}, —OC(O)NHR^{s}, —OC(O)N(R^{s})₂, —SH, —SR^{s}, —S(O)R^{s}, —S(O)₂R^{s}, —SO₂NH₂, —S(O)₂NHR^{s}, —S(O)₂N(R^{s})₂, —NHS(O)₂R^{s}, —NR^{s}S(O)₂R^{s}, —C(O)NH₂, —C(O)NHR^{s}, —C(O)N(R^{s})₂, —C(O)R^{s}, —NHC(O)R^{s}, —NR^{s}C(O)R^{s}, —NHC(O)NH₂, —NR^{s}C(O)NH₂, —NR^{s}C(O)NHR^{s}, —NHC(O)NHR^{s}, —NR^{s}C(O)N(R^{s})₂, —NHC(O)N(R^{s})₂, —CO₂H, —CO₂R^{s}, —NHCO₂R^{s}, —NR^{s}CO₂R^{s}, —CN, —NO₂, —NH₂, —NHR^{s}, —N(R^{s})₂, —NR^{s}S(O)NH₂ and —NR^{s}S(O)₂NHR^{s}, wherein each R^{s} is independently an unsubstituted C₁₋₆ alkyl; and
D is O, S or N—OR^{t}, wherein R^{t} is selected from the group consisting of hydrogen, C₁₋₈ alkyl, C₁₋₈ haloalkyl and C₁₋₈ heteroalkyl;
with the proviso that the compound is other than those set forth in paragraph 82.

2. The compound of claim 1, wherein R² and L¹ are combined to form a 6-membered carbocyclic or heterocyclic ring.

3. The compound of claim 2, wherein D is O.

4. The compound of claim 2, wherein A is carbon.

5. The compound of claim 4, having formula

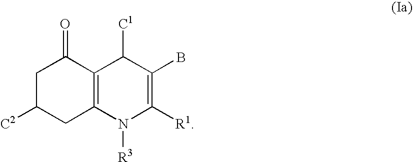

6. The compound of claim 4, having formula

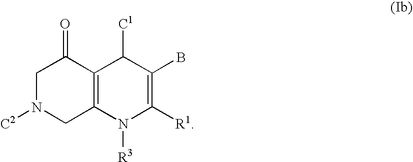

7. The compound of claim 4, having formula

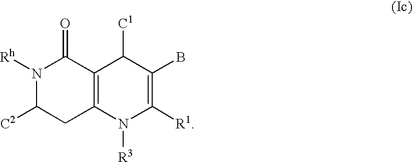

8. The compound of claim 1, having formula 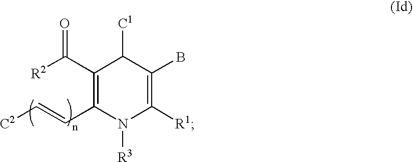
wherein the subscript n is an integer from 1-3.

9. The compound of claim 4, having formula

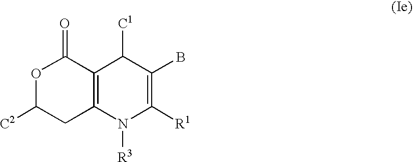

10. The compound of claim 4, having formula

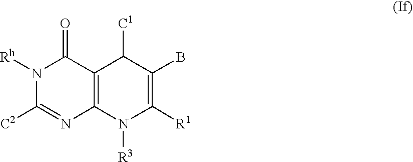

11. The compound of claim 7, wherein R³ is hydrogen.

12. The compound of claim 7, wherein B is selected from the group consisting of —CN, CO₂R^{a}, CONR^{a}R^{b} and —C(O)R^{a}.

13. The compound of claim 12, wherein R^{a} and R^{b} are each independently selected from the group consisting of H, Me, Et, i-Pr, t-Bu, CH₂CH₂OCH₃, CH₂CH₂NHCH₃, CH₂CH₂N(CH₃)₂, cycloalkyl, heterocycloalkyl, alkoxyalkoxy alkyl.

14. The compound of claim 12, wherein B is —CO₂R^{a}, wherein R^{a} is an optionally substituted member selected from the group consisting of C₁₋₈ alkyl, C₃₋₈ cycloalkyl, C₂₋₈ heteroalkyl and aryloxy-C₁₋₄ alkyl.

15. The compound of claim 14, wherein R^{a} is selected from the group consisting of H, Me, Et, i-Pr, t-Bu, CH₂CH₂OCH₃, CH₂CH₂NHCH₃, CH₂CH₂N(CH₃)₂, cycloalkyl, heterocycloalkyl, alkoxyalkoxy alkyl.

16. The compound of claim 7, wherein R¹ is selected from the group consisting of C₁₋₈ alkyl, C₁₋₈ haloalkyl, C₁₋₈ alkoxy, C₃₋₈ cycloalkyl-C₁₋₈ alkyl, C₃₋₆ heterocycloalkyl, C₃₋₆heterocycloalkyl-C₁₋₆ alkyl, amino; each of which is optionally substituted with from 1-3 members selected from the group consisting of —ORⁿ, —CO₂Rⁿ, —N(Rⁿ)₂, —C(O)N(Rⁿ)₂, and —C(O)NHRⁿ.

17. The compound of claim 16, wherein R¹ is selected from the group consisting of —CH₃, —CF₃, cyclopentyl, cyclopentylethyl and —NH₂.

18. The compound of claim 7, wherein C¹ is a member selected from the group consisting phenyl, 2-pyridyl, 3-pyridyl, 4-pyridyl, 2-thienyl, 3-thienyl, 2-furanyl, 3-furanyl, 3-indolyl, 4-indolyl, 5-indolyl, 6-indolyl, 7-indolyl, 3-dihydro-1H-indolyl, 3-indazolyl, 4-indazolyl, 5-indazolyl, 6-indazolyl, 7-indazolyl, 1H-benzotriazolyl, 2-oxo-2,3-dihydro-benzooxazolyl, 2-oxo-2,3-dihydro-benzothiazolyl, 2-oxo-1H-benzoimidazoly, 3-oxo-3,4-dihydro-2H-benzo[1,4]oxazinyl, 5-azabenzpyrazolyl, benzopyrrolidinyl, 2-pyrrolyl and 3-pyrrolyl; each of which is optionally substituted with from 1-3 R⁴ substituents.

19. The compound of claim 18, wherein C¹ is a phenyl group optionally substituted with 1 to 2 substituents selected from the group consisting of —OH, —NO₂, —NR^{d}R^{e}, halogen, C₁₋₈ alkyl, C₁₋₈ haloalkyl, C₁₋₈ alkoxy, C₂₋₈ alkenyl, X²OR^{d}, —NR^{d}—C(O)NR^{d}R^{e}, —NR^{d}S(O)₂NR^{d}R^{e}, —NR^{d}S(O)₂R^{f}, X²CO₂R^{d}, X²C(O)NR^{d}R^{e}, —C(O)NR^{d}R^{e}, NR^{e}C(O)R^{d} and heterocycloalkyl; and optionally any two substituents located on adjacent atoms of C¹ are combined to form a 5- to 6-membered ring.

20. The compound of claim 18, wherein C¹ is a monocyclic ring selected from the group consisting of phenyl, pyridyl, furanyl, thienyl and pyrrolyl; each of which is optionally substituted with from 1-2 R⁴ substituents.

21. The compound of claim 18, wherein C¹ is a bicyclic ring selected from the group consisting of indolyl, indazolyl, 1H-benzotriazolyl, 2-oxo-2,3-dihydro-benzooxazolyl, 2-oxo-2,3-dihydro-benzothiazolyl, 2-oxo-2,3-dihydro-1H-benzoimidazolyl, 3-oxo-3,4-dihydro-2H-benzo[1,4]oxazinyl and 5-azabenzpyrazolyl, benzopyrrolidinyl; each of which is optionally substituted with from 1-3 R⁴ substituents.

22. The compound of claim 18, wherein C¹ is selected from the group consisting of:

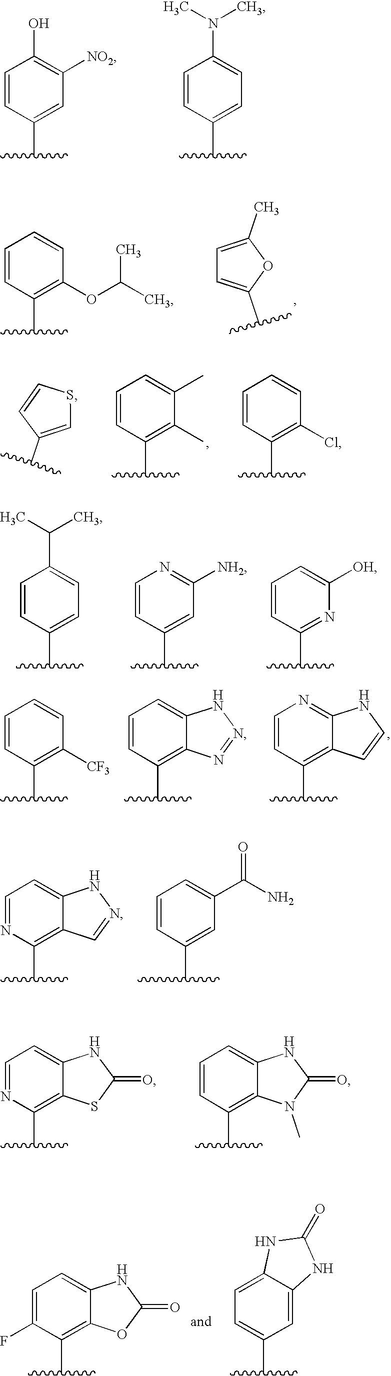

23. The compound of claim 19, wherein C¹ is a member selected from the group consisting of:

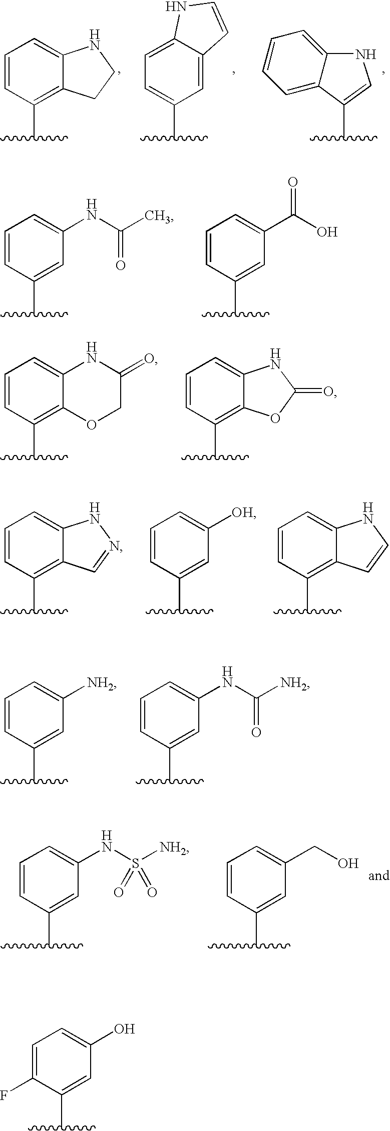

24. The compound of claim 7, wherein C² is a substituted member selected from the group consisting of phenyl, 2-pyridyl, 3-pyridyl, 4-pyridyl, 2-thienyl, 3-thienyl, 2-furanyl, 3-furanyl, 2-pyrrolyl, 3-pyrrolyl, 3-indolyl, 4-indolyl, 5-indolyl, 6-indolyl and 7-indolyl; each of which is optionally substituted with from 1-3 R⁴ substituents.

25. The compound of claim 24, wherein C² is a phenyl group optionally having 1 to 5 substituents selected from the group consisting of halogen, cyano, —NR^{d}R^{e}, C₁₋₈ alkyl, C₁₋₈ alkoxy, C₁₋₈ haloalkyl, C₃₋₆ cycloalkyl, C₂₋₈ alkenyl, C₂₋₈ alkynyl, aryl-C₁₋₄ alkyl, and optionally any two substituents located on adjacent atoms of C² are combined to form a 5- to 6-membered ring.

26. The compound of claim 24, wherein C² is selected from the group consisting of:

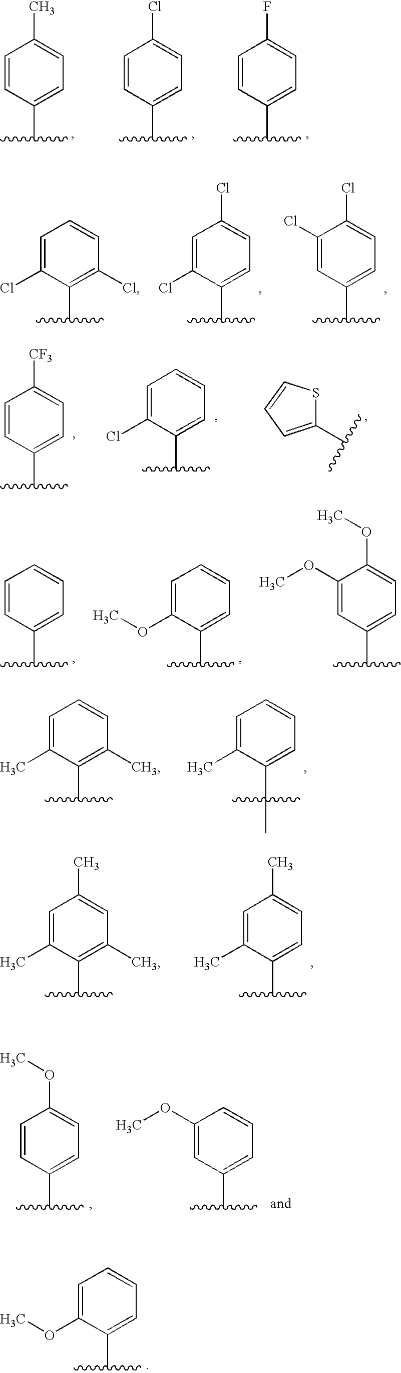

27. The compound of claim 7, wherein R³ is selected from the group consisting of hydrogen, optionally substituted C₃₋₈ cycloalkyl and optionally substituted C₁₋₆ alkyl.

28. The compound of claim 7, wherein B is selected from the group consisting of halogen, —CN, —CO₂R^{a}, —CONR^{a}R^{b}, and —C(O)R^{a}; R¹ is C₁₋₈ alkyl or C₁₋₈ haloalkyl; C¹ and C² are each independently an optionally substituted phenyl; and R³ is hydrogen or C₁₋₆ alkyl.

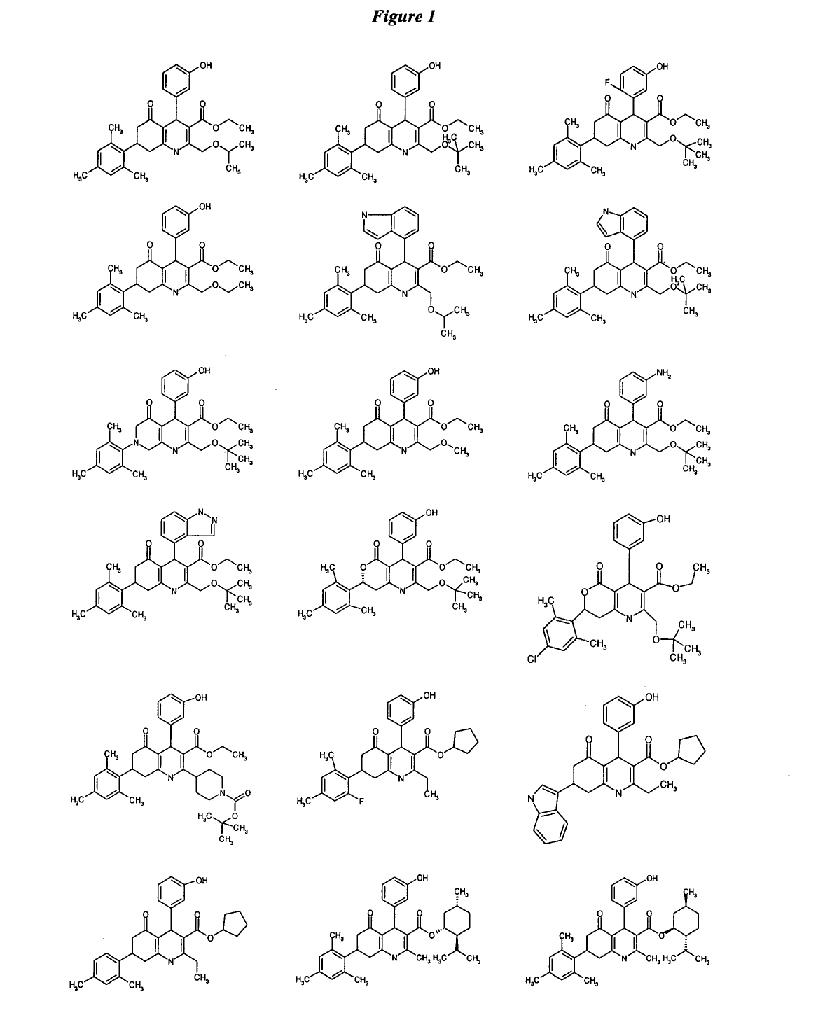

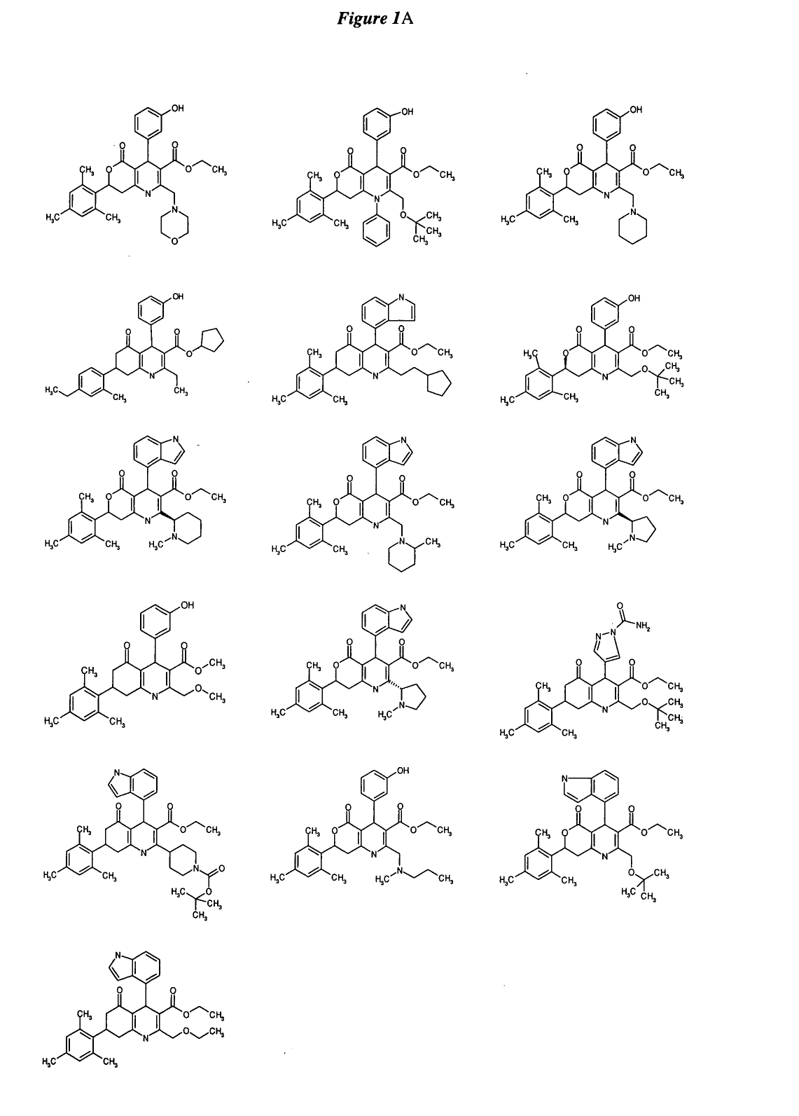

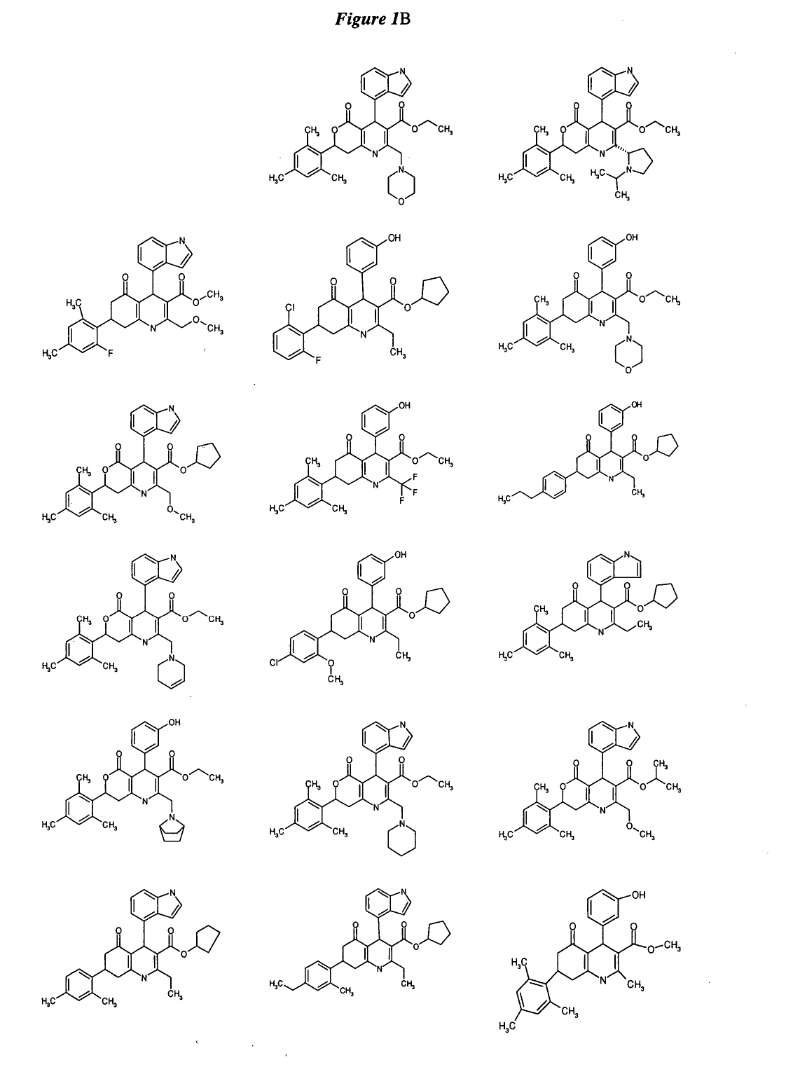

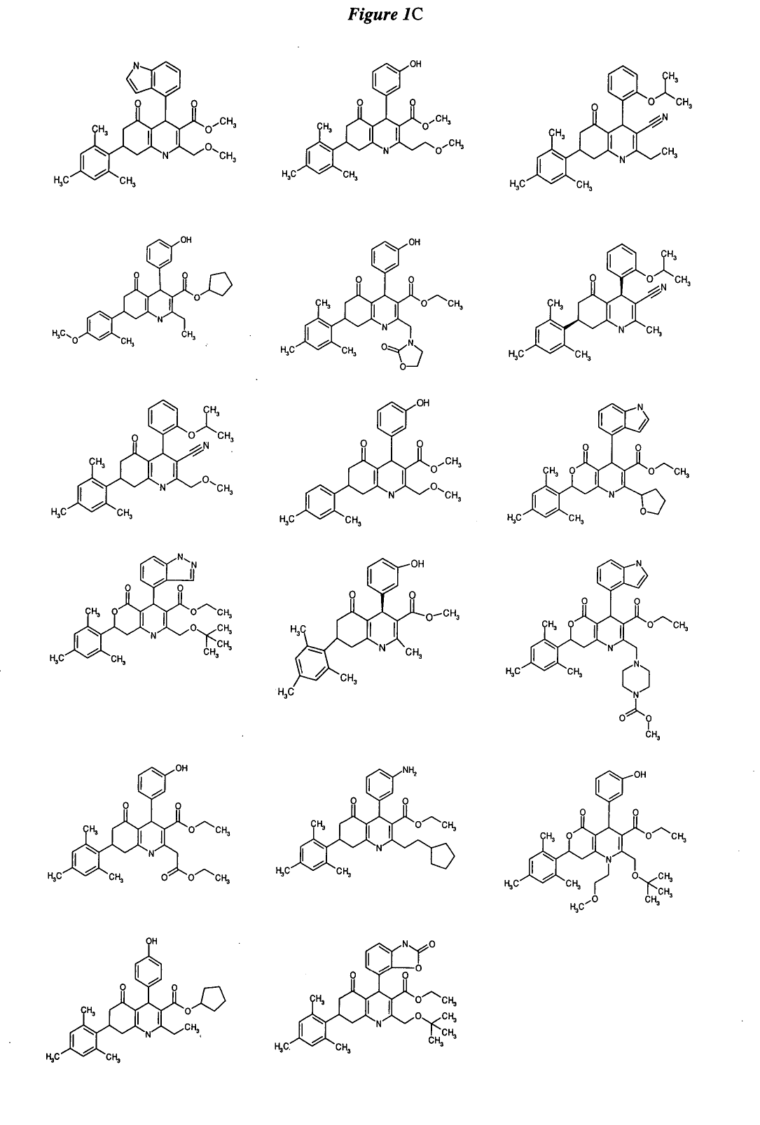

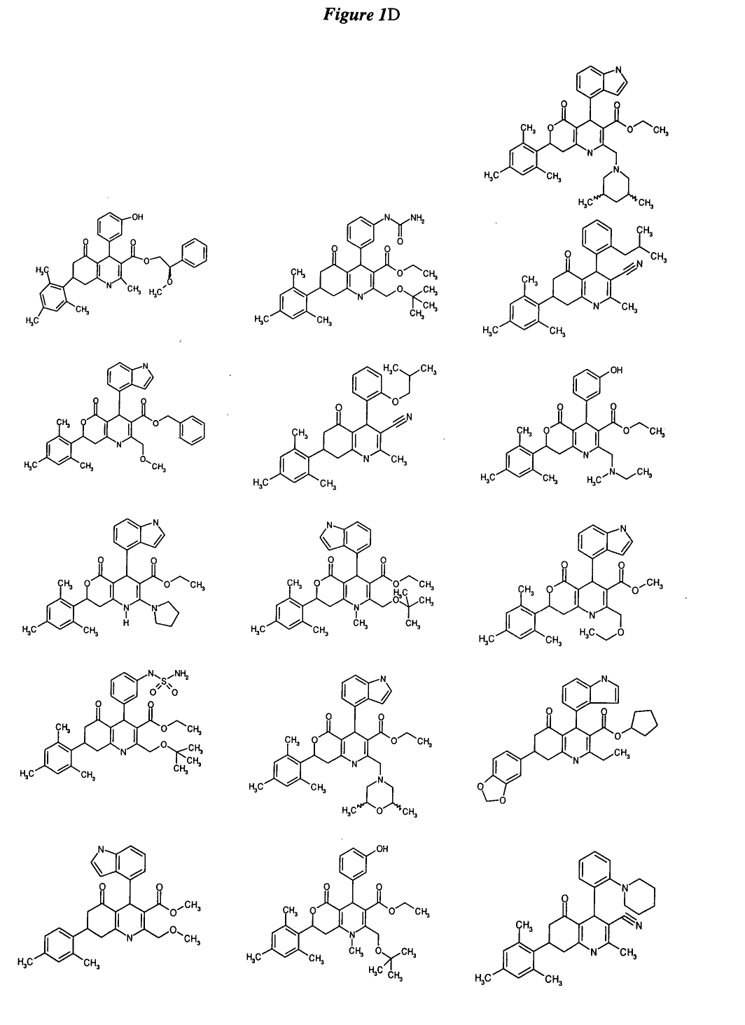

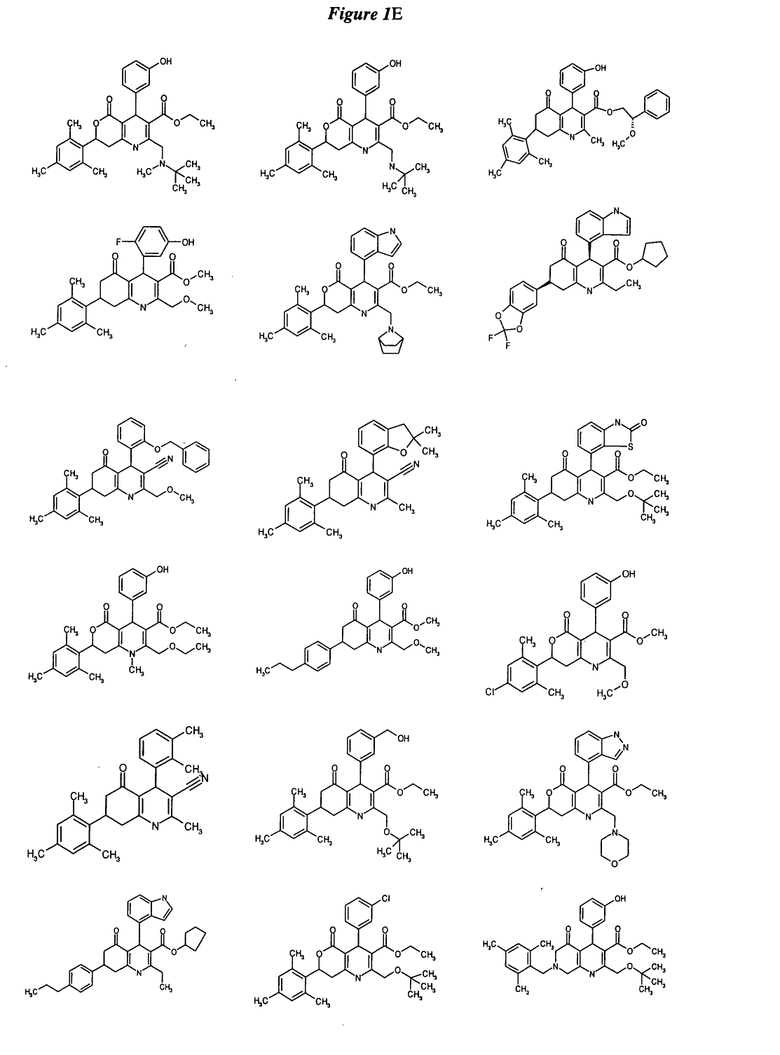

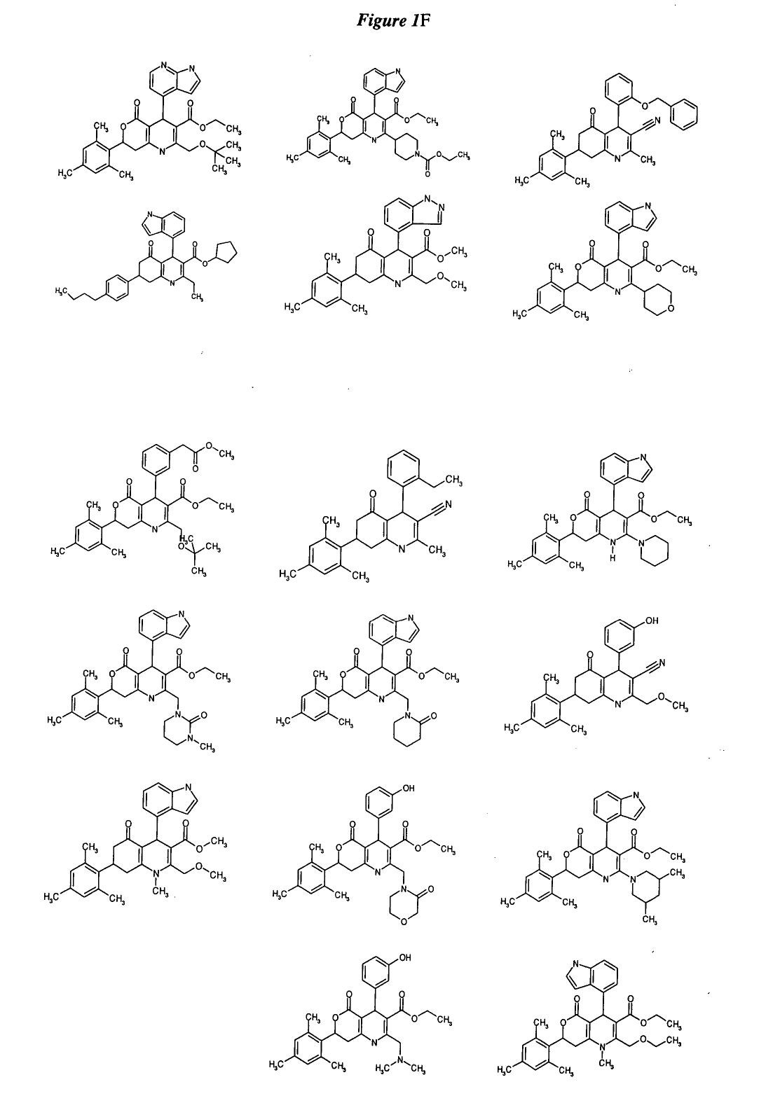

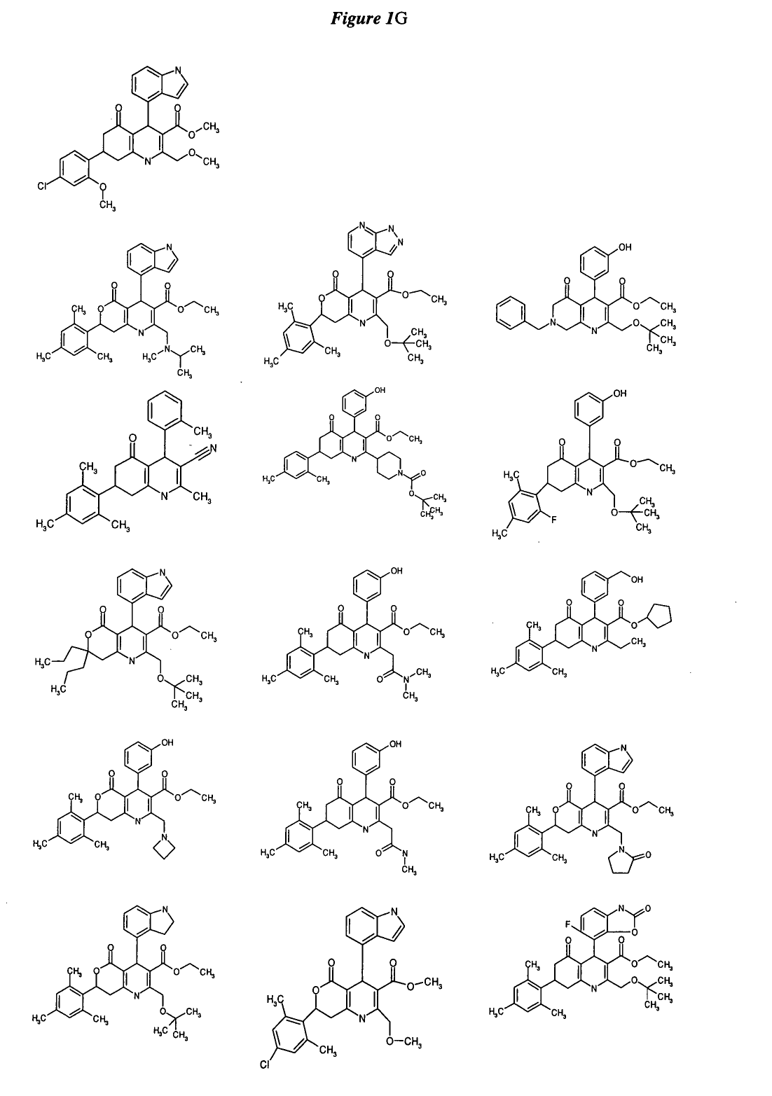

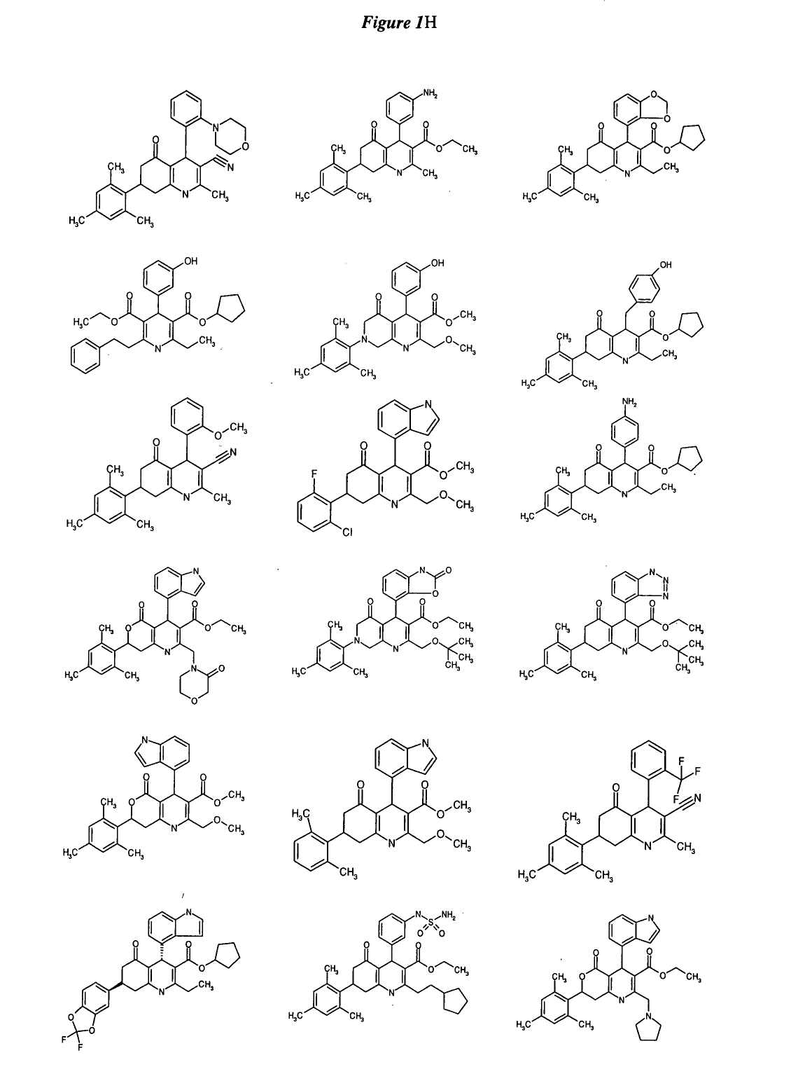

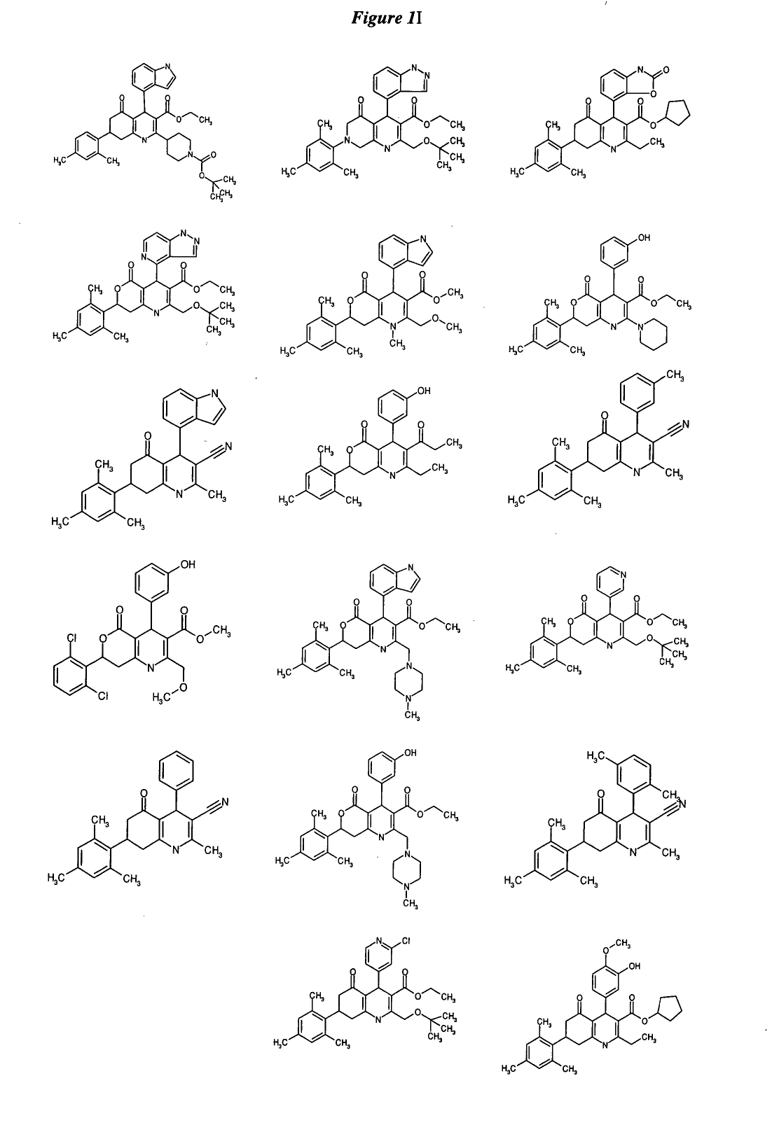

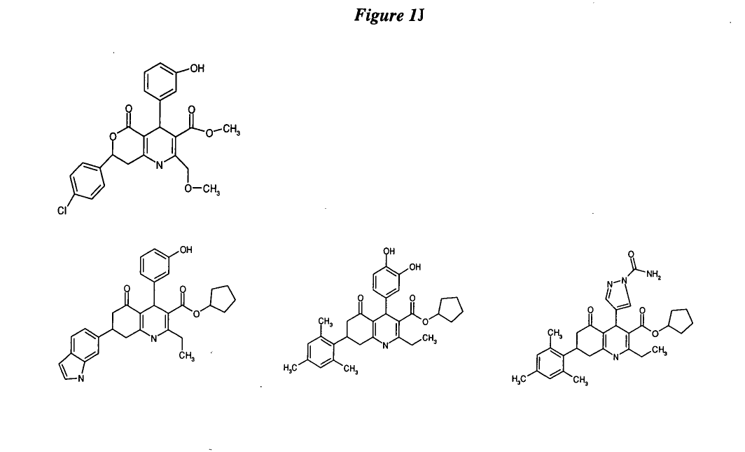

29. The compound of claim 1, wherein said compound is selected from the group set forth in FIG. 1.

30. The compound of claim 1, wherein said compound is selected from the group in Table 1.

31. A pharmaceutical composition comprising a pharmaceutically acceptable excipient or carrier and a compound of claim 1.

32. A method for treating a mammal suffering from or susceptible to a disease or disorder involving pathologic activation of C5a receptors, said method comprising: administering to the mammal an effective amount of a compound of claim 1.

33. A method of inhibiting C5a receptor-mediated cellular chemotaxis comprising contacting mammalian white blood cells with a C5a receptor modulatory amount of a compound of claim 1.

34. The method of claim 32, wherein the disease or disorder is an inflammatory disease or disorder.

35. The method of claim 34, wherein the disease or disorder is selected from the group consisting of neutropenia, sepsis, septic shock, Alzheimer's disease, multiple sclerosis, stroke, inflammatory bowel disease, chronic obstructive pulmonary disorder, inflammation associated with burns, lung injury, osteoarthritis, atopic dermatitis, chronic urticaria, ischemia-reperfusion injury, acute respiratory distress syndrome, systemic inflammatory response syndrome, multiple organ dysfunction syndrome, tissue graft rejection and hyperacute rejection of transplanted organs.

36. The method of claim 32, wherein the disease or disorder is a cardiovascular or cerebrovascular disorder.

37. The method of claim 36, wherein the disease or disorder is selected from the group consisting of myocardial infarction, coronary thrombosis, vascular occlusion, post-surgical vascular reocclusion, artherosclerosis, traumatic central nervous system injury and ischemic heart disease.

38. The method of claim 32, wherein the disease or disorder is an autoimmune disorder.

39. The method of claim 38, wherein the disease or disorder is selected from the group consisting of rheumatoid arthritis, systemic lupus erythematosus, Guillain-Barre syndrome, pancreatitis, lupus nephritis, lupus glomerulonephritis, psoriasis, Crohn's disease, vasculitis, irritable bowel syndrome, dermatomyositis, multiple sclerosis, bronchial asthma, pemphigus, pemphigoid, scleroderma, myasthenia gravis, autoimmune hemolytic and thrombocytopenic states, Goodpasture's syndrome, immunovasculitis, tissue graft rejection and hyperacute rejection of transplanted organs.

40. The method of claim 32, wherein the disease or disorder is a pathologic sequelae associated with a member selected from the group consisting of insulin-dependent diabetes, mellitus, lupus nephropathy, Heyman nephritis, membranous nephritis, glomerulonephritis, contact sensitivity responses, and inflammation resulting from contact of blood with artificial surfaces.
